SEQUENCE LISTING (320 sequences)

SEQ ID NO: 1 - DNA, 478 bp, Diabrotica virgifera
ggtttcatgacccctgagagaaagaagaaacttaggttactgttgagaaagaaagccgccgaagaattaaagaaagaacaagaacgcaaagcagccgaaaggaggcgtatcattgaagaaaggtgcggtaaacccaaacttgtcgatgacgcaaatgaaggcccattaaaacaagtatgtgagggatatcacagacgtattgtagacctagaaaataagaaatttgacctcgaaaaagaagtggaattcagagattttcagatctccgaattgaacagccaagtaaacgaccttagaggcaaattcgtcaaaccaaccttgaagaaggtatccaaatacgaaaacaaattcgccaaacttcaaaagaaggcagctgaatttaacttccgtaaccaactcaaagttgtcaagaagaaagaattcaccttagaagaagaagacaaagaaaagaaaccagactggtcaaagaagggagacg

SEQ ID NO: 2 - DNA, 529 bp, Diabrotica virgifera
agaagttgccgctttagtcgtagacaatggatccggtatgtgcaaagctggttttgctggggatgatgcacctcgtgctgtattcccttcaattgttggacgcccaagacatcagggtgtgatggtaggaatgggacaaaaagattcctatgtaggtgatgaagctcaaagtaaaagaggtatccttaccttaaaataccccatcgagcacggaatagtcacaaactgggatgatatggagaaaatttggcatcatacattctacaatgaactcagagtagccccagaagaacaccctgttctgttgacagaagctcctctcaaccccaaggccaacagggaaaagatgacacaaataatgtttgaaactttcaacaccccagccatgtatgttgccatccaggctgtactctccttgtatgcatctggtcgtacaactggtattgtgttggattctggtgatggtgtatcccacactgtcccaatctatgaaggttatgctcttcctcatgcaatccttcgtttggac

SEQ ID NO: 3 - DNA, 592 bp, Diabrotica virgifera
accggcctttgtatgtcttgggatatgtgcctaaagacgatagattatacctcgtagataaagagttgcgcgtagtaagctaccaattacttctttctgttcttgaatatcaaactgccgtcatgagaagagactttccaacagcagacagagtacttccgtccattcctaaggagcacagaacgagagtggcacatttcttagaaaagcaaggcttcaaacagcaagctttggccgtaagtacagatccagagcacagattcgagctggcagtagcattagaggatcttaatatagccaaaactctagctcaagaagcgaacagtccgcaaaagtggaatcaactagcagaattggcagctgctactaataatgtaagcgtagccaaggaatgtatgcaaaaagcgcaagattatggaggcttgttgcttcttgctacgagctccggtgatgaaaatttagtccgtactctaggagaaacgacacaagctgaaagcaaacataacttagcatttttgtcacacttgttagtaggtgatttaaacaaatgtctagacattcttattaataccggtagattgccagaagctgc

SEQ ID NO: 4 - DNA, 594 bp, Diabrotica virgifera
gaaagcagttggaagatggccgtactctctcagactacaacattcaaaaagagtctaccctccatttggtacttcgtcttagaggaggtatgcagatttttgttaaaactttaactggaaagaccatcacccttgaagtagaaccttctgataccatcgaaaatgtcaaagccaaaattcaagacaaagaaggtattccaccagatcaacaaagattaatctttgccggaaagcaattggaagatggtcgtacactctcagactacaacattcaaaaggaatctaccctccatttggtacttcgtcttagaggaggtatgcaaatctttgtaaaaacactcactggtaagaccatcaccctcgaggttgaaccatcagataccatcgagaatgtcaaagctaaaattcaagacaaagaaggtattccaccagatcaacagagattaatcttcgctggaaagcagttggaagatggccgtactctctcagactacaatattcagaaagagtctaccctccatttggtacttcgtcttagaggaggtatgcaaatctttgtaaaaactctcactggtaagaccatcaccctcgagg

SEQ ID NO: 5 - DNA, 547 bp, Diabrotica virgifera
taccccattgaacacggaattatcactaactgggacgatatggaaaagatctggcatcacaccttctacaatgaacttagagtagcccccgaagaacatcccattcttttgactgaagctccacttaacccaaaagccaacagagaaaagatgactcaaatcatgtttgaaactttcaatacccctgccatgtatgttgccattcaagctgtattgtctctgtacgcttccggtcgtaccactggtattgtacttgattctggagatggtgtatcccacacagtacccatctatgaaggttacgctctcccacacgccatcttgcgtttggacttggccggtagagacttgactgactaccttatgaagatcttaaccgaaagaggttactctttcaccaccacagctgaaagagaaatagttcgtgacatcaaggaaaaattgtgctatgtagctttggacttcgaacaggaaatggccacagcagccagctccacctccttagaaaagagttatgaacttcctgacggtcaagtcatcaccattg

SEQ ID NO: 6 - DNA, 592 bp, Diabrotica virgifera
aggaaaggaaggacccaagaccaaacaaccatcgagatacatccgttttatctacaatcgcgtcatattggaatgtccttctgtaagagctgctgcagtctccgccatggcacaattcggagcctcttgtcccgatttgttagaaaatatccaaatattactttcgaggtgtcagatggattcagacgatgaagttagggacagagctacatattatagtaatatacttaacaaaaatgataaaagtttatacaacaattacattttggattctttgcaggtttcaattccttcactagaaagatcgcttagagaatacattcaaaatccaactgacgaaccatttgacattaagtccgtacctgtagcagcagtgccaacagcagaagaacgagaagttaaaaacaaatctgaaggactgctagtctctcaaggtccagtccgacctcctccggtgtctagagaagaaaacttcgccgaaaaacttagtaacgttccgggtatacaacagttaggacctttgttcaaaacttccgacgtcgttgaactcactgaatctgaaacagagtattttgtccgctgtatcaagcac

SEQ ID NO: 7 - DNA, 500 bp, Diabrotica virgifera
tactaggcgaaagtgccgtcggtaagtcgagtttggtactgaggttcgtcaaaggacagttccacgaataccaggagagtaccataggagcagctttccttacacaaaccatatgcctcgacgatacaactgttaaatttgaaatttgggacacagcgggtcaagaaaggtaccacagtttagctcctatgtactataggggcgcacaggcagctatagtcgtctacgacataaccaatcaagacacattcggcagggcgaaaacgtgggtgaaggaacttcaaaggcaggccagtccgacgatcgtgatagctttggccggcaacaagcaagatttggccaacaaacgtatggtagaatacgaagaggcgcagacgtatgctgacgaaaacggcttactttttatggaaacttccgcaaagacggcaatgaacgtcaacgatatatttttagcaatagctaagaaactgcccaagaatgaacaaaccacaggtcaaggc

SEQ ID NO: 8 - DNA, 189 bp, Diabrotica virgifera
cgatgaggttgaaggaaggagaaaaattttgatggggcgaaaaagcattaccaggacatatcttcgtggaaatgctgttcctgcgtatgtgataataatccttgtaggaattggtcaaatcatcctgggagggatattgtacgttgcattgaggaagaagatcattgctgcacctgtaacggcatcata

SEQ ID NO: 9 - DNA, 393 bp, Diabrotica virgifera
tgaggtcgagccctcagatactatcgaaaatgtgaaagctaaaatccaggataaagaaggaattcccccagaccagcaacgtctcatcttcgctggaaaacaactcgaagatggtcgtaccttgtctgactataatattcaaaaagaatcaacccttcacttggtgttgagattgagaggaggtgctaagaaacgtaagaagaagaattactccacccccaagaaaatcaagcacaagaagaagaaggttaagttagctgtattgaaattttataaggttgacgaaaatggtaaaatccaccgattgagacgtgaatgccccgctgaacaatgtggagctggtgtcttcatggcagccatggaagacaggcattactgtggcaagtgcggtta

SEQ ID NO: 10 - DNA, 524 bp, Diabrotica virgifera
tctttgcctttggctacgattcgaggagggcattcaagaatggctccagaaattggattcagaacaattttacatcggagtatatgtacttatagtcgcttcactgatcgtcatgattgtgtcctttataggatgtattagtgccctgcaggagagtaccatggcccttttagtgtacatcggcacccaagtgctcagttttatattcggtttatccggttcggcggttcttctggataacagcgccagagattcccacttccaaccgaggatccgagagagtatgcgacgtcttatcatgaatgctcatcacgaccaatccagacaaacactagccatgattcaggaaaatgttggttgctgcggagctgatggcgcaacagactacctctctcttcagcagccccttccaagtcagtgcagagacaccgttactggaaacccattcttccacggatgtgtagatgaactcacctggttcttcgaagaaaaatgtggttggatagcaggtttagctatggcga

SEQ ID NO: 11 - DNA, 267 bp, Diabrotica virgifera
tcgtgctggtttacaatttcctgtaggtcgtattcatcgtttattgagaaaaggaaattatgccgaaagagttggtgctggagctcctgtatacttggcagctgttatggaatatttagctgctgaagttttggaattggcaggaaatgcagctagagataacaaaaagacccgtataattcctagacatttacaattggccataagaaatgacgaggaattgaacaaattactgtcaggagttaccatcgcccaaggtggagtatt

SEQ ID NO: 12 - DNA, 558 bp, Diabrotica virgifera
gtgcatgaagttggatggtgtagatctgcccccaccaattagcttcgacattgcggaagagcaaccgttaccaccttgccaacagacgttcttatgtaatggtgatggaggatccatagtgcgacagtttctcgagctgtatttcgtaatatatgattcagataataggcagtcccttcttcaggcatatcacgaaaaagccacattttcaatgacaatggcctacccgtacggctattccaaagacagtaaaggagtatcgtggttgaattggtatgccaccgataatagaaatttattacgagttcaagatccagacagaagaaacaagttgttaagacagggacaagttgctgtagtttcgttcttgcaagatatgccgcacacgaagcacgatattcacagttttacagtagatttgacagtttttacaccccagatgttatgtttgacagtggctggtatgtttaaagaattgaaaagtggccacaaagtacctcctttaagatatttcttcagaacccttgtaattgtacctgctggatcaggtttttgcat

SEQ ID NO: 13 - DNA, 582 bp, Diabrotica virgifera
gattcggagccctacaatgaaatgcaactaaaaatggatttagaaaagggtgaggttaaagtaaaaataagagcattagaaaaaataattcacatgattctggcaggagaaaggttgccgaatggatttctaatgaccatcataagaaacgttttacctttacaagatcatttggcaaaaaaactattattgattttctgggaaatagttccaaaaacaaatccagaaggtaaactactacaagagatgattttggtatgtgatgcctatagaaaagatctgcaacacccaaatgaatttttgagaggttctacacttcgcttcttgtgcaaactgaaggaaccagaattgttggaaccattaatgcccagtattagagcttgtttggatcataggcacagctatgtgaggaggaatgctgtactggcaatttttaccatttacaaaaattttgaagccctcattccagatgctcctgaactgatctccaattatttggatggtgagcaagacatgtcttgtaaaagaaatgcgtttttaatgcttcttcatgctgaccaagaaagggcgttgtcgtatttg

SEQ ID NO: 14 - DNA, 458 bp, Diabrotica virgifera
ttggcaaaaaggaaatgaggatattgatggtaggactcgatgcagctggtaaaaccacaattttatataaacttaaattaggagaaattgtaacaactattccaacaattggatttaatgtggagactgtagaatataagaacattagttttacagtatgggatgtaggtggtcaagataaaattaggccattgtggagacactatttccaaaacacacaaggcctaattttcgtagtagacagtaacgacagggaacgtatcactgaggctaaagatgaattaatgcgtatgttggccgaagatgaacttagagatgccgtacttctcattttcgccaacaaacaagatttgcccaatgcaatgaacgctgcagaaatcaccgacaaactcggtctccattcactacgcaaccgcaactggtacattcaagctacctgtgcaactagcggagatggt

SEQ ID NO: 15 - DNA, 592 bp, Diabrotica virgifera
ggtcgcgaaagaacagaaagattttttgaccgtatctgccgatggatgcgtacaagtaacaaaacctctcgaccgagatccgcctttcggtagcccaacacgacaagtcttcatctatgctcgtgataatgatggaggcacaaattcattgttggccactgcagaaattgaaattattttaatagatataaacgataatgctccctttttaaatgttacagaaattgtttattatgaaaaccaggatccaggttttataggtaacctaagtgccgatgattacgatggtcctgataatggacctccgtttgcttttcgattatcagacactgcttcagatagtattagatcgaaattttccattatcggaaaccagcttttcgctttagaaatgtttgatagagaagagcaaaaatattatgacattgccattgacattacagatagtggagtacctccactaacaggaactagtattcttagagttataatcggagatgtaaatgataatccagctacagacggaaacagcacgatctttgtgtataagtacgtcaatgggccagaaaatttcatggaaatcggacgtgta

SEQ ID NO: 16 - DNA, 214 bp, Diabrotica virgifera
gcatggggtatcatccagttgggtttcatgggtgtattctattacattggggctgtggctttagcagaagatattccagaggttgagtttaagggcgatttagacaaattttatagcgacgtcaacacgggtttcacacagaatgcttacaactgctggattgctgctctcctatacctgataacattagcagtatcagctcaccaattctggg

SEQ ID NO: 17 - DNA, 459 bp, Diabrotica virgifera
tagatgccgcaggtaaaaccacaatcttatacaaattgaagcttggtgaaatcgtaactacaataccaaccatcggcttcaatgtagaaaccgttgagtacaagaatatatctttcacggtatgggatgtaggtggccagacgagaatcagaaaactctggagacactatttcgccaacactgatggactcatttttgtggttgattccaacgaccgagaccgtatcgcggaagccgaagaagaattgcacaatatgttaggagaggacgatttaagagactgcattttgttaatattcgccaacaaacaagatttaccgaactcgatgtccactgctgaattgaccgataagcttaagttgcacactttgaagaataggaggtggtacatacaagccacatgtgctactcaagggaatggtttgtacgaaggactagattggttgtcgaatgaattgg

SEQ ID NO: 18 - DNA, 536 bp, Diabrotica virgifera
gctaccctagcgtccactgtaacactgatttttgccctctactactgcctcacgggaaaaggagagcaagttagtttagcatggttattgttgaatgtgtctccccacatgtgggcaggtctaggaattggccttgctgtatcattatcagttgtaggagctgctgcaggaattcacactacaggagtcagtatcgtaggagctggtgttaaagcccccagaatcaaaaccaaaaatttaatttctattattttctgtgaagctgtggctatctatgggttaattatggctatagtactctgtggaagttggaagaatttcgatgtagacctattcaacctcaaaactcataactttgctcaaaaccattatggatcacatgttatttttggatccggtttaactgttggatttgtaaatctattatgtggattttgtgttggagtagttggttctggtgcagccatttctgatgcagccaattcatcattattcgtcaaaattttgattattgagatttttggaagtgccattgg

SEQ ID NO: 19 - DNA, 547 bp, Diabrotica virgifera
accttatgggaaagcgtgtggacttttctgcacgtactgtcatcacaccagatcccaatttacgtatcgaccaagtaggagtgcctagaagtattgctcaaaacatgacgtttccagaaatcgtcacacctttcaattttgacaaaatgttggaattggtacagagaggtaattctcagtatccaggagctaagtatatcatcagagacaatggagagaggattgatttacgtttccacccaaaaccgtcagatttacatttgcagtgtggttataaggtagaaagacacatcagagacggcgatctagtaatcttcaaccgtcaaccaaccctccacaagatgagtatgatgggccacagagtcaaagtcttaccctggtcgacgttccgtatgaatctctcgtgcacctctccctacaacgccgattttgacggcgacgaaatgaacctccatgtgccccaaagtatggaaactcgagctgaagtcgaaaacctccacatcactcccaggcaaatcattactccgcaagctaaccaacccgtcat

SEQ ID NO: 20 - DNA, 564 bp, Diabrotica virgifera
cggatctctatttgggggatcaagtcgtattgaagatgcagtggaatgttacacaagagctgcaaacctttttaaaatggccaagagctgggatgctgccggtaaagccttttgtgaggctgctaatttgcattccagaactggtgctcgtcatgacgctgccactaattatatagatgctgcaaattgttacaaaaaagccgatgtatttgaggctgtaaactgctttataaaagctatagacatttataccgaaatgggtcgctttacaatggctgcaaaacaccatcagactattgcagaaatgtatgagactgatgctgtggacatcgaaagggctgttcaacactatgaacaggcggctgattacttcagaggagaagaaagcaatgcttccgccaataagtgtcttcttaaagtggctcaatatgcagcccaacttgaaaactatgaaaaagcagtgggaatttatcaagaagtggcttatgcagctctggaaagctctcttttaaaatacagtgcaaaggaatacttattcagagctgccctttgtcacctttgtgt

SEQ ID NO: 21 - DNA, 564 bp, Diabrotica virgifera
tgctgattggagattcaggagtaggaaaatcttgtcttctactgagatttgcagatgatacctacacagaaagctatattagtaccattggcgtagattttaaaatcaggacaatcgatttagatggaaagacaattaaattgcaaatttgggatacagcaggtcaggaaaggtttagaacgattacatcaagttattaccgaggagcacatggtattattgtagtgtacgattgcacagaccaagattcattcaataacgttaaacagtggctcgaagaaatcgaccgttatgcgtgtgacaatgtaaacaaattactggtagggaataaaagcgatttgacaactaagaaagttgtcgacttcactacagccaaggagtatgccgaccaattgggtataccatttttggaaacctcagctaagaatgcaaccaatgtagaacaggcctttatgactatggccgctgaaataaaaaatagagtaggacctccatcttctgcggtagaccaaggaaataaggttaggttcgatcaaagtcgcccagtcgaaacaaccaaatccg

SEQ ID NO: 22 - DNA, 502 bp, Diabrotica virgifera
gagctatagtggactgcgggttcgaacatccttcagaagttcaacatgaatgtattcctcaagctgtcattggcatggatattctgtgccaagctaaatccggtatgggaaaaacggctgtttttgtattagctacactccaagtaatagatcctacagaaaatgttgtatatgttctcgtcatgtgccataccagagagttagccttccagataagcaaagagtacgaacgtttcagtaaatatatgcccaatattaaagtaggggtcttctttggtggcttgcctatccagaaagatgaggaaacgttaaaaaataattgcccgcatatcgttgtgggtactccaggaagaattttagcattggtcagatcgaaaaaacttaatctcaaacatctaaagcattttattttggatgaatgtgataaaatgttggagttattagacatgagacgtgatgttcaagaaatatatcgtaacactccccacgaaaaacaagtcat

SEQ ID NO: 23 - DNA, 505 bp, Diabrotica virgifera
tggtagatttggctaacctcgtatattggtgtttaggtcttaatattccgtacgttagtttctatgattataaaggtaatttaaaaaagcatgaagagaagttgcaacaaattgtagaatccagaaaatcagagaatatcaacataatttggcacacccatgcagaacaaaggcataaaaatggatttttgggtccaaaaatccacgtaaaagtgttaacacacgcggacggaaagcaaagtatagtaaatgttactaaaaaattagctctaaataaagaaaaagacattagtaaagaaaaaattagtgaattactattaaggcagtatgaatttccagatccagaaatggctattatttgtggaaagaaactgaacatttataattatcctccttggcagttaagactcacagaattctttaaagtcaacaaagtcaacaacatcacattcccagtgtttgtggaaaaattggaaaagtacagcaaatgtgaacagagggtggg

SEQ ID NO: 24 - DNA, 410 bp, Diabrotica virgifera
aaccagggatctatttgcccgcaagtttaaaaaacgtggtgtaattccactttccacatatttgagagtctacaaagttggagatattgtagatatcaagggtaatggtgcagttcaaaagggtatgccccacaaagtgtaccatggtaagacaggacgtgttttcaatgttactgcacatgcattaggtgtaattgtaaacaaaagggttcgaggaagaatcatccccaaaagaatcaatctccgtattgaacatgtaaaccactccaagtgtcgtcaagacttcttgcaaagagtaaaatccaacgaaaagctacgtaaagaagctaaagaaaagaacattaaagtagaacttaggagacaacctgcccaacctaggccagcacatattgttagcggaaaggttccag

SEQ ID NO: 25 - DNA, 467 bp, Diabrotica virgifera
tgatggtaatagagtgctggctaaatactacgataaagatatatttcctacagcaaaagagcagaaagcttttgagaaaaatttgttcaataaaactcatagggcagacgcagaaattatcatgttggatggtttaacttgtgtgtatagaagtaatgtagatttattcttttatgttatgggcagttcacatgaaaatgagctaattttaatgagtgttttaaattgcttgtatgactcagtaagtcaaatattgaagaaaaatatgcaaaaacgagctgtcttggaatcactagatattgttatgctggctatggatgaaattgttgatggaggaataattatagattctgattcaagttcagtagtatctagaatagcattaaggactgatgatattccattaggagaacaaactgtagctcaggtattccaaacggccaaagaacagctgaaatggtcattgc

SEQ ID NO: 26 - DNA, 368 bp, Diabrotica virgifera
caccgaagaacaaattgctgaattcaaagaagctttctcactattcgataaagatggtgatggtacaattacgactaaagaattaggaacagtaatgagatctctaggacaaaatccaacagaggctgaattacaggatatgatcaatgaagtagatgccgatggtaacggcacgatcgatttcccagaatttttaacgatgatggcacgtaaaatgaaagataccgatagtgaggaagaaattcgtgaagcattccgagtgttcgacaaagacggcaatggtttcatctcagcagcagaattgcgccacgtcatgaccaacttgggtgaaaaattgacagacgaagaagtcgatgaaatgattcggg

SEQ ID NO: 27 - DNA, 430 bp, Diabrotica virgifera
ttcgcacaagatgactttggtggtgaaaatgttgatctatatgacgatgtaatatccgctcctcctggaaataatgacaacccaggtgattcaaatcatcatgctcctcctggtgctggtgaagatggtggaggtaattttgttgggtcaggaggagcacccaataatataaattcttctggaagaagacatcagctgtatgttggaaatctgacttggtggacaactgatcaagatatagaaaatgcagtgcatgatataggggtaaccgacttccatgaagttaagttttttgaacacagagcaaatggtcaatccaagggattctgtgtcatatctttgggatctgagggaagcatgagactctgcctggaactcctatctaaaaaagagatcaatggccaaaatccccttgttacccttcccacaa

SEQ ID NO: 28 - DNA, 505 bp, Diabrotica virgifera
taggaatggcattttcaggcttaatagctgatgcaaggcaaatcgttgagattgctagaaaagaagcatcaaattatagacatcaatatggttcaaatattcctcttaaatacctaaatgatagagtaagcatgtacatgcatgcatacactttatacagtgctgttagaccatttggttgcagtgtcatcttggccagttatgaagatagtgacccatctatgtatctgattgatccatctggagttagctatggatactttggatgtgctacaggtaaagcaaaacagtctgcaaagactgaaatagaaaaattgaagatggggaatctaacatgcaaagaacttgttaaagaagcagccaaaatcatttatttggtccatgatgagctgaaggataagaattttgaactggaactttcatgggtatgcaaagatacgaatggtttacataccaaagtgcctgaatcagtgtttgctgatgcagaaaaagctgccaaacaagc

SEQ ID NO: 29 - DNA, 548 bp, Diabrotica virgifera
tccatctagaggagcccaaatgatgatgaaatccaggctaaagggagcccaaaagggacatagtttattaaagaagaaagctgatgctttacaaatgagatttagaatgattttgaacaaaattattgagaccaaaactctcatgggtgaagtaatgaaagaagctgccttttctttagctgaagcaaagtttgcaactggtgacttcaatcaagttgttcttcaaaatgtcaccaaggctcaaataaaaataagaactaagaaagacaacgttgctggtgttactttaccagtgtttgaatgctaccaagatggtacagatacatatgagttggctggtttggctaggggaggtcaacaattgacaaaactcaagaagaattatcaaagtgctgttaaactgttggttgaattagcctctttgcaaacttcttttgtaactcttgatgatgtaatcaaaataacaaacagaagagtcaatgccattgaacatgttatcattccaagaatagagcgtactttggcttacatcatatccgaactggacg

SEQ ID NO: 30 - DNA, 521 bp, Diabrotica virgifera
gatctggaagcgctagttgcaaaagtagacgaaatgagaacccaaagagccatgctatgggctcaacttcgagaatctattcaccaagacgatattacaagttcccttgtaacgaaacaaccaaatcagtcgctggaacagctgttccagcaagaacttcaaaagcatcaaaatctgatttcgttgattgaacaaaacacctcggcacaagaaaacattaagagcgccttagtcgattcttacgcttacgctgtaaattcaagaaaatacatccaagatatactccaaaagagaaccacaaccataacgtcactgatagcatcgttcgactcttacgaagacttattggcaaaagctaacaaagggatagagttttactcaaaacttgaaacgaacgtatccaagttactgcaaagaataaggagtacctgcaaagttcaacaagaagagcgagatcagatgatgtcgactgcgcaagtgcctcaatgggagagtcatacgtcacttgccgctcctaaact

SEQ ID NO: 31 - DNA, 20 bp, Diabrotica virgifera
ggtttcatgacccctgagag

SEQ ID NO: 32 - DNA, 20 bp, Diabrotica virgifera
cgtctcccttctttgaccag

SEQ ID NO: 33 - DNA, 20 bp, Diabrotica virgifera
agaagttgccgctttagtcg

SEQ ID NO: 34 - DNA, 20 bp, Diabrotica virgifera
gtccaaacgaaggattgcat

SEQ ID NO: 35 - DNA, 20 bp, Diabrotica virgifera
accggcctttgtatgtcttg

SEQ ID NO: 36 - DNA, 20 bp, Diabrotica virgifera
gcagcttctggcaatctacc

SEQ ID NO: 37 - DNA, 20 bp, Diabrotica virgifera
gaaagcagttggaagatggc

SEQ ID NO: 38 - DNA, 20 bp, Diabrotica virgifera
cctcgagggtgatggtctta

SEQ ID NO: 39 - DNA, 20 bp, Diabrotica virgifera
taccccattgaacacggaat

SEQ ID NO: 40 - DNA, 20 bp, Diabrotica virgifera
caatggtgatgacttgaccg

SEQ ID NO: 41 - DNA, 20 bp, Diabrotica virgifera
aggaaaggaaggacccaaga

SEQ ID NO: 42 - DNA, 20 bp, Diabrotica virgifera
gtgcttgatacagcggacaa

SEQ ID NO: 43 - DNA, 20 bp, Diabrotica virgifera
tactaggcgaaagtgccgtc

SEQ ID NO: 44 - DNA, 20 bp, Diabrotica virgifera
gccttgacctgtggtttgtt

SEQ ID NO: 45 - DNA, 20 bp, Diabrotica virgifera
cgatgaggttgaaggaagga

SEQ ID NO: 46 - DNA, 20 bp, Diabrotica virgifera
tatgatgccgttacaggtgc

SEQ ID NO: 47 - DNA, 20 bp, Diabrotica virgifera
tgaggtcgagccctcagata

SEQ ID NO: 48 - DNA, 20 bp, Diabrotica virgifera
taaccgcacttgccacagta

SEQ ID NO: 49 - DNA, 20 bp, Diabrotica virgifera
tctttgcctttggctacgat

SEQ ID NO: 50 - DNA, 20 bp, Diabrotica virgifera
tcgccatagctaaacctgct

SEQ ID NO: 51 - DNA, 21 bp, Diabrotica virgifera
tcgtgctggtttacaatttcc

SEQ ID NO: 52 - DNA, 20 bp, Diabrotica virgifera
aatactccaccttgggcgat

SEQ ID NO: 53 - DNA, 20 bp, Diabrotica virgifera
gtgcatgaagttggatggtg

SEQ ID NO: 54 - DNA, 20 bp, Diabrotica virgifera
atgcaaaaacctgatccagc

SEQ ID NO: 55 - DNA, 20 bp, Diabrotica virgifera
gattcggagccctacaatga

SEQ ID NO: 56 - DNA, 20 bp, Diabrotica virgifera
caaatacgacaacgcccttt

SEQ ID NO: 57 - DNA, 20 bp, Diabrotica virgifera
ttggcaaaaaggaaatgagg

SEQ ID NO: 58 - DNA, 20 bp, Diabrotica virgifera
accatctccgctagttgcac

SEQ ID NO: 59 - DNA, 20 bp, Diabrotica virgifera
ggtcgcgaaagaacagaaag

SEQ ID NO: 60 - DNA, 20 bp, Diabrotica virgifera
tacacgtccgatttccatga

SEQ ID NO: 61 - DNA, 20 bp, Diabrotica virgifera
gcatggggtatcatccagtt

SEQ ID NO: 62 - DNA, 20 bp, Diabrotica virgifera
cccagaattggtgagctgat

SEQ ID NO: 63 - DNA, 20 bp, Diabrotica virgifera
tagatgccgcaggtaaaacc

SEQ ID NO: 64 - DNA, 20 bp, Diabrotica virgifera
ccaattcattcgacaaccaa

SEQ ID NO: 65 - DNA, 20 bp, Diabrotica virgifera
gctaccctagcgtccactgt

SEQ ID NO: 66 - DNA, 20 bp, Diabrotica virgifera
ccaatggcacttccaaaaat

SEQ ID NO: 67 - DNA, 20 bp, Diabrotica virgifera
accttatgggaaagcgtgtg

SEQ ID NO: 68 - DNA, 20 bp, Diabrotica virgifera
atgacgggttggttagcttg

SEQ ID NO: 69 - DNA, 20 bp, Diabrotica virgifera
cggatctctatttgggggat

SEQ ID NO: 70 - DNA, 20 bp, Diabrotica virgifera
acacaaaggtgacaaagggc

SEQ ID NO: 71 - DNA, 21 bp, Diabrotica virgifera
tgctgattggagattcaggag

SEQ ID NO: 72 - DNA, 20 bp, Diabrotica virgifera
cggatttggttgtttcgact

SEQ ID NO: 73 - DNA, 20 bp, Diabrotica virgifera
gagctatagtggactgcggg

SEQ ID NO: 74 - DNA, 20 bp, Diabrotica virgifera
atgacttgtttttcgtgggg

SEQ ID NO: 75 - DNA, 21 bp, Diabrotica virgifera
tggtagatttggctaacctcg

SEQ ID NO: 76 - DNA, 20 bp, Diabrotica virgifera
cccaccctctgttcacattt

SEQ ID NO: 77 - DNA, 20 bp, Diabrotica virgifera
aaccagggatctatttgccc

SEQ ID NO: 78 - DNA, 20 bp, Diabrotica virgifera
ctggaacctttccgctaaca

SEQ ID NO: 79 - DNA, 22 bp, Diabrotica virgifera
tgatggtaatagagtgctggct

SEQ ID NO: 80 - DNA, 21 bp, Diabrotica virgifera
gcaatgaccatttcagctgtt

SEQ ID NO: 81 - DNA, 20 bp, Diabrotica virgifera
caccgaagaacaaattgctg

SEQ ID NO: 82 - DNA, 20 bp, Diabrotica virgifera
cccgaatcatttcatcgact

SEQ ID NO: 83 - DNA, 20 bp, Diabrotica virgifera
ttcgcacaagatgactttgg

SEQ ID NO: 84 - DNA, 20 bp, Diabrotica virgifera
ttgtgggaagggtaacaagg

SEQ ID NO: 85 - DNA, 20 bp, Diabrotica virgifera
taggaatggcattttcaggc

SEQ ID NO: 86 - DNA, 20 bp, Diabrotica virgifera
gcttgtttggcagctttttc

SEQ ID NO: 87 - DNA, 20 bp, Diabrotica virgifera
tccatctagaggagcccaaa

SEQ ID NO: 88 - DNA, 20 bp, Diabrotica virgifera
cgtccagttcggatatgatg

SEQ ID NO: 89 - DNA, 20 bp, Diabrotica virgifera
gatctggaagcgctagttgc

SEQ ID NO: 90 - DNA, 20 bp, Diabrotica virgifera
agtttaggagcggcaagtga

SEQ ID NO: 91 - DNA, 633 bp, Diabrotica virgifera
atggcggacgatgagagaaagaaactggaggaggaaaagaagaggaaacaggccgaaattgaacgcaaaagggccgaggtcagggctcgtatggaagaggcctcaaaagccaagaaggccaagaaaggtttcatgacccctgagagaaagaagaaacttaggttactgttgagaaagaaagccgccgaagaattaaagaaagaacaagaacgcaaagcagccgaaaggaggcgtatcattgaagaaaggtgcggtaaacccaaacttgtcgatgacgcaaatgaaggcccattaaaacaagtatgtgagggatatcacagacgtattgtagacctagaaaataagaaatttgacctcgaaaaagaagtggaattcagagattttcagatctccgaattgaacagccaagtaaacgaccttagaggcaaattcgtcaaaccaaccttgaagaaggtatccaaatacgaaaacaaattcgccaaacttcaaaagaaggcagctgaatttaacttccgtaaccaactcaaagttgtcaagaagaaagaattcaccttagaagaagaagacaaagaaaagaaaccagactggtcaaagaagggagacgaaaagaaggtacaagaggctgaagcatga

SEQ ID NO: 92 - DNA, 603 bp, Diabrotica virgifera
atgtgtgaagaagaagttgccgctttagtcgtagacaatggatccggtatgtgcaaagctggttttgctggggatgatgcacctcgtgctgtattcccttcaattgttggacgcccaagacatcagggtgtgatggtaggaatgggacaaaaagattcctatgtaggtgatgaagctcaaagtaaaagaggtatccttaccttaaaataccccatcgagcacggaatagtcacaaactgggatgatatggagaaaatttggcatcatacattctacaatgaactcagagtagccccagaagaacaccctgttctgttgacagaagctcctctcaaccccaaggccaacagggaaaagatgacacaaataatgtttgaaactttcaacaccccagccatgtatgttgccatccaggctgtactctccttgtatgcatctggtcgtacaactggtattgtgttggattctggtgatggtgtatcccacactgtcccaatctatgaaggttatgctcttcctcatgcaatccttcgtttggacttagctggtagagacttgactgattacctcatgaaaattttgactgaacgtggctactctttc

SEQ ID NO: 93 - DNA, 2742 bp, Diabrotica virgifera
atgccacttcgattagatataaaaagaaagctaacagctcgctcagaccgggtaaaatgtgtggatcttcaccctacagaaccttggatgctgtgttctctttacagcggaaatataaacgtttggaacaccgaaaatcagcaactggttaagacttttgaagtatgtgatgtacctgttcggacagctaagtttttgcccaggaagaactggatagtcagtgggtctgatgatatgcagattcgagttttcaattacaataccttagatcgggtacattcttttgaggctcattcggattatgtgagatgtattgtcgtacaccctacacaaccttatatattaacaagtagtgatgatatgcttatcaagctttggaattgggaaaaagcatgggcttgtcagcaagttttcgaaggacacactcattatattatgcaaatcgccataaatccaaaagacaacaacacatttgccagtgcatccctagatagaacattgaaagtatggcaattgggagcgtccacagcgaatttcacactagaaggtcatgagaaaggcgttaactgtgtggactattatcacggtggagataaaccttatttaatctcaggcgctgatgatagattagtaaaaatctgggattatcaaaacaaaacttgtgttcaaactttggaaggacatgctcaaaatgtaaccgctgcatgtttccatccagaacttcctgtagctcttactggaagtgaagatggtactgtcagagtgtggcatgccaacacccataggttagaaagtagcttaaattatggctttgaaagagtatggactattttctgcctaaagggatccaataacgtggcattgggttatgatgaaggtagcattttggttaaagttggtagagaagaaccagctgttagtatggatgccagtggaggcaaaattatttgggccagacactctgaacttcaacaggcaaatctcaaggcgttagctgaaggtgcggaaataagagatggagaacgccttccagtttctgtaaaagatatgggtgcttgcgagatataccctcagacaattcaacacaatcccaatggccgttttgttgttgtctgtggggatggagaatacataatctacacagcaatggctttaagaaacaaagcgtttggtagcgcacaagaatttgtgtgggctcaagattccagcgaatatgccatcagagaatccggatctactatcagaatttttaagaatttcaaagagaagaagaattttaagtccgattttggagctgaaggtatatacggtggataccttttgggagtcaaatcggtttctggtttgactttctatgattgggaaactctcgatttagtcagaagaatcgagatacaaccaaaagcagtttactggtcagatagtggtaaattagtatgtttggccacagaagatagctactttattctttcttatgattctgatgaagttcaaaaagccagagataacaatcaggttgcggatgatggagtagaatcggctttcaatcttctaggtgaaataaacgaatcagtgcgaactggtctctgggtaggcgactgttttatctacacgaattctgttaatcgtatcaactacttcgttggaggtgaactggttacaattgctcatttggaccggcctttgtatgtcttgggatatgtgcctaaagacgatagattatacctcgtagataaagagttgcgcgtagtaagctaccaattacttctttctgttcttgaatatcaaactgccgtcatgagaagagactttccaacagcagacagagtacttccgtccattcctaaggagcacagaacgagagtggcacatttcttagaaaagcaaggcttcaaacagcaagctttggccgtaagtacagatccagagcacagattcgagctggcagtagcattagaggatcttaatatagccaaaactctagctcaagaagcgaacagtccgcaaaagtggaatcaactagcagaattggcagctgctactaataatgtaagcgtagccaaggaatgtatgcaaaaagcgcaagattatggaggcttgttgcttcttgctacgagctccggtgatgaaaatttagtccgtactctaggagaaacgacacaagctgaaagcaaacataacttagcatttttgtcacacttgttagtaggtgatttaaacaaatgtctagacattcttattaataccggtagattgccagaagctgcatttttcgccagatcttaccttcctgataagattacagaagtcgtggaactgtggaagactcagttatcttcagtcaatcaaaaagctggacagagccttgccgatcctaaaaactacgaaaatctgttccctggtttacaagaggcggtggtagctcagaaatttttggaacagcagaataaaggtttagcgcccgcaagagttgccaccaccattcctcctaatcacgacaggaatgttgtagccgaagttcaagcacaatcgaaacacgatgtaccatcatttagttcttcgtttatttcatcagaaatagaagcacaaacaaggagttctgctaaacctgaagaatcttcaaacattatacagctggaccaagatgacgacgatatcgatttagatttggacggtgtaaatatcgatgagaacattgacacgacggatatcaacatcgatgatgatttgctgagtgattga

SEQ ID NO: 94 - DNA, 1146 bp, Diabrotica virgifera
atgcagatctttgtaaaaacactcactggtaaaaccatcaccctcgaggttgaaccatcagataccatcgagaatgtcaaagctaaaattcaagacaaagaaggtattccaccagatcaacagagattaatctttgctggaaagcagttagaagatggccgtactctctcagactacaacattcagaaagaatctacactacacttagtgcttcgtcttagaggaggtatgcacatctttgtaaaaactctcactggtaagaccatcacccttgaggttgaaccatcagataccatcgagaatgtcaaagctaaaattcaagacaaagaaggtattccaccagatcaacagagattaatctttgctggaaagcagttggaagatggccgtactctctcagactacaacattcaaaaagagtctaccctccatttggtacttcgtcttagaggaggtatgcagatttttgttaaaactttaactggaaagaccatcacccttgaagtagaaccttctgataccatcgaaaatgtcaaagccaaaattcaagacaaagaaggtattccaccagatcaacaaagattaatctttgccggaaagcaattggaagatggtcgtacactctcagactacaacattcaaaaggaatctaccctccatttggtacttcgtcttagaggaggtatgcaaatctttgtaaaaacactcactggtaagaccatcaccctcgaggttgaaccatcagataccatcgagaatgtcaaagctaaaattcaagacaaagaaggtattccaccagatcaacagagattaatcttcgctggaaagcagttggaagatggccgtactctctcagactacaatattcagaaagagtctaccctccatttggtacttcgtcttagaggaggtatgcaaatctttgtaaaaactctcactggtaagaccatcaccctcgaggttgaaccatcagataccatcgagaatgtcaaagctaaaattcaagacaaagaaggtattccaccagatcaacaaagattaatctttgccggaaagcagttggaagatggccgtactctctcagactacaacattcaaaaagagtctacccttcacttggtacttcgtttaagaggaggaaattaa

SEQ ID NO: 95 - DNA, 1131 bp, Diabrotica virgifera
atgtgtgacgacgatgtagcggctcttgtcgtcgacaatggctccggaatgtgcaaagccggtttcgccggtgatgacgcccctcgtgctgtctttccatccatcgtaggtcgtcccagacaccaaggtgtcatggtgggtatgggtcaaaaagactcctacgtaggagacgaagcccaaagcaaaagaggtatcctcaccttaaaataccccattgaacacggaattatcactaactgggacgatatggaaaagatctggcatcacaccttctacaatgaacttagagtagcccccgaagaacatcccattcttttgactgaagctccacttaacccaaaagccaacagagaaaagatgactcaaatcatgtttgaaactttcaatacccctgccatgtatgttgccattcaagctgtattgtctctgtacgcttccggtcgtaccactggtattgtacttgattctggagatggtgtatcccacacagtacccatctatgaaggttacgctctcccacacgccatcttgcgtttggacttggccggtagagacttgactgactaccttatgaagatcttaaccgaaagaggttactctttcaccaccacagctgaaagagaaatagttcgtgacatcaaggaaaaattgtgctatgtagctttggacttcgaacaggaaatggccacagcagccagctccacctccttagaaaagagttatgaacttcctgacggtcaagtcatcaccattggtaatgaaaggttccgttgccctgaagctctcttccaaccttccttcttgggtatggaatcttgcggtatccacgaaactgtctacaactccatcatgaagtgcgatgtcgacatccgtaaagacttgtacgccaacactgtcctttctggaggtaccacaatgtaccctggtattgccgatcgtatgcaaaaggaaatcactgccttggctccatcaaccatcaaaatcaagatcatcgctcccccagaaagaaagtactccgtttggatcggtggctccatcttggcctccctctccaccttccaacagatgtggatctccaaacaagaatacgacgaatccggccctggaattgttcaccgcaaatgcttctaa

SEQ ID NO: 96 - DNA, 2640 bp, Diabrotica virgifera
atgggtacttttaaaagagatactcatgatgaggacgggggatcaagtgcttttcaaaatctggagaaaactactgttttgcaggaagctagagtttttaatgaaactagtgtaaatccaagaaaatgtacaccgatactaacaaaactgttgtacttattgaaccagggtgaaactttaagtgccaaagaggccacagatgttttctttgccatgaccaaactgttccaatcaaaagatgtaatattgagaaggatggtttatttgggaattaaagaactcagttctgttgctgatgatgtcattattgtaacatccagtcttacaaaagatatgactggtaaagaagacatgtacagagcagctgctataagagcattatgcagtattactgatgctactatgcttcaagctatagaacgttatatgaagcaagctattgtagatagaaacgcagctgtcagttcagcagcactaattagttcattacatatgagcaaattagctccagatgtagtaaaaagatgggtaaatgaagctcaggaagcagtaaatagtgataatgcaatggtacagtatcacgcattaggtcttctataccatattaggaagactgataagctagcagtgacaaaattgatttccaagctgaattcaatgggtttaaagagcccttatgctttgtgtatgttgataagaatcactgcaaaacttttagaagaagaggaccaagagtcactcctcaactccccatatacaataatatttacaatgggcttaaggaacaaatctgaaatggtggtgtatgaagctgcacatgccatggttaacctgaagttcacgagtagtaatgtgctagcacccgctataagtgttctacaactattttgtggatctcctaaagccacactcagatttgctgctgttagaactttaaatcaagtggccaccacccaccctgcgtcagtgacagcttgtaatttggatctagaaaatttgattactgatcctaataggtcaattgctacactggccattactactcttttgaaaacaggtgccgaatcttctgttgacagactaatgaaacaaatcgctacttttgtatctgaaatcagtgatgaatttaaagtggttgtcattcaggcaattaaggtattagctttgaaatttccaaggaaacatagcacgcttatgaatttcctatccgccatgttaagagatgagggaggtttagaatataaagcatccatagcagataccattataaccctaatcgaagataatcccgaagctaaagaatctggtttggcgcatctttgcgagttcattgaagactgtgaacatgtttctttggctgtgagaatcttgcatttgttaggaaaggaaggacccaagaccaaacaaccatcgagatacatccgttttatctacaatcgcgtcatattggaatgtccttctgtaagagctgctgcagtctccgccatggcacaattcggagcctcttgtcccgatttgttagaaaatatccaaatattactttcgaggtgtcagatggattcagacgatgaagttagggacagagctacatattatagtaatatacttaacaaaaatgataaaagtttatacaacaattacattttggattctttgcaggtttcaattccttcactagaaagatcgcttagagaatacattcaaaatccaactgacgaaccatttgacattaagtccgtacctgtagcagcagtgccaacagcagaagaacgagaagttaaaaacaaatctgaaggactgctagtctctcaaggtccagtccgacctcctccggtgtctagagaagaaaacttcgccgaaaaacttagtaacgttccgggtatacaacagttaggacctttgttcaaaacttccgacgtcgttgaactcactgaatctgaaacagagtattttgtccgctgtatcaagcactgtttcaaacatcacatcgtcctccaattcgattgtctgaataccttgccagaccagcttttagaaaacgttagagtggagatagacgccggtgaaaccttcgaaattttggcagaaataccttgtgaaaagttgcactataacgaaaccggtaccacatatgtagtagttaagttgcctgatgatgatctccccaactctgttggtacgtgtggagccgtgttgaagttcttagtgaaagattgtgatccatcaacgggaataccagattctgatgagggttacgatgatgaatatacactggaagacatcgaaataacattaggggaccaaattcaaaaagtaagcaaagtaaattgggctgcagcctgggaagaagctgcagctacttatgtagaaaaagaggatacatactccttgaccatcaatacgctaagtggcgctgttaagaatattattcagttcttgggattacagcctgcggaaaggactgacagagtaccggagggtaaatctacgcacacattacttcttgctggtgtattcaggggaggtattgacatactagtaagagcgaaactagctttgggcgaatgtgttacgatgcaactaacagtcaggtcgccagatcctgacgttgctgagcttataacttcaactgtaggttaa

SEQ ID NO: 97 - DNA, 651 bp, Diabrotica virgifera
atggcggcaaacagaactggacctgctcagagaccaaatggcgctacccaaggaaagatatgtcagttcaaactggtcctactaggcgaaagtgccgtcggtaagtcgagtttggtactgaggttcgtcaaaggacagttccacgaataccaggagagtaccataggagcagctttccttacacaaaccatatgcctcgacgatacaactgttaaatttgaaatttgggacacagcgggtcaagaaaggtaccacagtttagctcctatgtactataggggcgcacaggcagctatagtcgtctacgacataaccaatcaagacacattcggcagggcgaaaacgtgggtgaaggaacttcaaaggcaggccagtccgacgatcgtgatagctttggccggcaacaagcaagatttggccaacaaacgtatggtagaatacgaagaggcgcagacgtatgctgacgaaaacggcttactttttatggaaacttccgcaaagacggcaatgaacgtcaacgatatatttttagcaatagctaagaaactgcccaagaatgaacaaaccacaggtcaaggcggcagtgcccaaggcaggcggctagcggagggcgattcgggcgccaaggcacccggaaattgttgcaagtga

SEQ ID NO: 98 - DNA, 279 bp, Diabrotica virgifera
atgaagtttttaagatcgacagtgtgctacattgccatcttggcaattctctttaccctctgtgccgatgaggttgaaggaaggagaaaaattttgatggggcgaaaaagcattaccaggacatatcttcgtggaaatgctgttcctgcgtatgtgataataatccttgtaggaattggtcaaatcatcctgggagggatattgtacgttgcattgaggaagaagatcattgctgcacctgtaacggcatcatatgcagtggctagacaagaaccataa

SEQ ID NO: 99 - DNA, 474 bp, Diabrotica virgifera
atgcagatcttcgttaaaaccttaacgggtaagaccatcactcttgaggtcgagccctcagatactatcgaaaatgtgaaagctaaaatccaggataaagaaggaattcccccagaccagcaacgtctcatcttcgctggaaaacaactcgaagatggtcgtaccttgtctgactataatattcaaaaagaatcaacccttcacttggtgttgagattgagaggaggtgctaagaaacgtaagaagaagaattactccacccccaagaaaatcaagcacaagaagaagaaggttaagttagctgtattgaaattttataaggttgacgaaaatggtaaaatccaccgattgagacgtgaatgccccgctgaacaatgtggagctggtgtcttcatggcagccatggaagacaggcattactgtggcaagtgcggttacactcttgtcttctccaaaccaggagatgagaaatag

SEQ ID NO: 100 - DNA, 747 bp, Diabrotica virgifera
atgatgtccaaagcagacacacaggaagatgcctccttcgccaaattggaaaatcagattgctatcatcaaatacgtaatactctttaccaacgttttgcaatgggctctcggtgcagcaatcttcgctctttgcctttggctacgattcgaggagggcattcaagaatggctccagaaattggattcagaacaattttacatcggagtatatgtacttatagtcgcttcactgatcgtcatgattgtgtcctttataggatgtattagtgccctgcaggagagtaccatggcccttttagtgtacatcggcacccaagtgctcagttttatattcggtttatccggttcggcggttcttctggataacagcgccagagattcccacttccaaccgaggatccgagagagtatgcgacgtcttatcatgaatgctcatcacgaccaatccagacaaacactagccatgattcaggaaaatgttggttgctgcggagctgatggcgcaacagactacctctctcttcagcagccccttccaagtcagtgcagagacaccgttactggaaacccattcttccacggatgtgtagatgaactcacctggttcttcgaagaaaaatgtggttggatagcaggtttagctatggcgatatgcatgattaacgtccttagtattgttttatctacggtactcatccaggcattgaaaaaagaagaagaagcatccgattcatacaggagatag

SEQ ID NO: 101 - DNA, 375 bp, Diabrotica virgifera
atgtctggacgtggcaagggaggcaaagtaaagggaaaagcaaagtcccgatcaaatcgtgctggtttacaatttcctgtaggtcgtattcatcgtttattgagaaaaggaaattatgccgaaagagttggtgctggagctcctgtatacttggcagctgttatggaatatttagctgctgaagttttggaattggcaggaaatgcagctagagataacaaaaagacccgtataattcctagacatttacaattggccataagaaatgacgaggaattgaacaaattactgtcaggagttaccatcgcccaaggtggagtattgcctaatatacaagcagtactgttacctaaaaaaactgaaaagaaagcttaa

SEQ ID NO: 102 - DNA, 864 bp, Diabrotica virgifera
atgaagttggatggtgtagatctgcccccaccaattagcttcgacattgcggaagagcaaccgttaccaccttgccaacagacgttcttatgtaatggtgatggaggatccatagtgcgacagtttctcgagctgtatttcgtaatatatgattcagataataggcagtcccttcttcaggcatatcacgaaaaagccacattttcaatgacaatggcctacccgtacggctattccaaagacagtaaaggagtatcgtggttgaattggtatgccaccgataatagaaatttattacgagttcaagatccagacagaagaaacaagttgttaagacagggacaagttgctgtagtttcgttcttgcaagatatgccgcacacgaagcacgatattcacagttttacagtagatttgacagtttttacaccccagatgttatgtttgacagtggctggtatgtttaaagaattgaaaagtggccacaaagtacctcctttaagatatttcttcagaacccttgtaattgtacctgctggatcaggtttttgcatagcaaatgaagaacttcacatatccaatgcaactccggaccaagcaaaagatgctttcaagaccaccgttaatgtagctccggcaccagcccctgtgattacctctcctggacccagtataccacaacccgctgtgccagatgatgctacaaaacaagaaatggtaaaacagatgtccgcagtatccggaatgaatctcgagtggtcgctacagtgtctcgaagaaacacaatgggactaccagaaagccataatggtattccaaaatttaaacgcacaaggtgttgtaccacaagcagcatttattaaa

SEQ ID NO: 103 - DNA, 2868 bp, Diabrotica virgifera
atgactgcggtagaacaaccttgttacacactaataaacttgccaacagattcggagccctacaatgaaatgcaactaaaaatggatttagaaaagggtgaggttaaagtaaaaataagagcattagaaaaaataattcacatgattctggcaggagaaaggttgccgaatggatttctaatgaccatcataagaaacgttttacctttacaagatcatttggcaaaaaaactattattgattttctgggaaatagttccaaaaacaaatccagaaggtaaactactacaagagatgattttggtatgtgatgcctatagaaaagatctgcaacacccaaatgaatttttgagaggttctacacttcgcttcttgtgcaaactgaaggaaccagaattgttggaaccattaatgcccagtattagagcttgtttggatcataggcacagctatgtgaggaggaatgctgtactggcaatttttaccatttacaaaaattttgaagccctcattccagatgctcctgaactgatctccaattatttggatggtgagcaagacatgtcttgtaaaagaaatgcgtttttaatgcttcttcatgctgaccaagaaagggcgttgtcgtatttggcatcatgtttagatcaagtaaattcatttggagatattctacaactggtcatcgttgagttgatatataaggtgtgtcattccaatcctgcggaaagatctagatttattagatgtatatataacttgttgaactcaagcagtcctgctgtcaggtacgaagctgcaggaactttagtcaccctctccagtgccccgactgccgttaaagctgctgctagctgttacattgagttaattatcaaagaaagtgacaacaatgtaaaactcatcgttttggacaggctgatagcacttaaggagcttcctaatcacgaaagaattctgcaggatttagttatggacatactgagagtactctctgctcctgacttagaagtccgcaagaagactttaagtctagcccttgaattagtctcttcacggaacatagaagaaatggtattagtattaacaaaggaagtgagtaaaacggtagacagtgaacatgaggatacaggaaagtacaggcaattgttagtaaggactctacattcgtgttccattaagttcccagatatcgcacgtagtgttataccagtcttgattgaatttttatccgataataatgaactggctgccacagatgtattgctgttcttaagggaagccatacagaagtttaaagaattgcaaccgttaattattgagaaactcatcgaaactttcaaagacattaaattggtcaaagtccatagagcagcaatttggattttgggagaatacgcgagtactgcttccgatatagaagttatcgttggagaaattaacagattgttgggtgaaggatccctcgttgaagctgagcagaagttaatagcaggagatacggaagagaatgctcctgcacctgctgcaggcgccaccactttagttacttccgatggaacatatgctacccaatcagctttcaacactgtcagccaaaccactaaagaagcacgacctcctctaagacaatacctcatggatggtgattttttcatcggagcctctttggcatctacattaaccaaactgtctttgcggtatgaggacctcacctctcctgctgctagcaatggattcaatgccaaaattatgcttattatggctggaattcttcacttgggaaaatcaggacttcccacaaaatcaataaccaacgacgataaagaccacattctgttctgtttacgagtcctatctgatcgttctccaatcattgttgaaattttcaaaaaattgtgccgctcggcactaaatgagatgcttctagctaaggaatcggtagaagcgatctcgcaaaagagcaaagaaaaaaacaagcgtacgattcaaactgacgacgctataagcttcctgcaattagagacagataaaagtggagagctaggagaaaacgtattcgagatgtcgctgtcacaagctttagtaggaggtcgaacgggaggtggcgaatcagtattaagttccaataaattagataaaatcacacaactgactggtttttccgatccagtttattccgaagcatacgttcacgtgaatcagtacgatatcgtgcttgatgtcttaatcgtaaaccaaactaacgatactttacaaaactgcacgctagagctggctactttaggcgatttgaagttggtagagaagccacaacctgtcgtattggcgcccaaagacttttgcaacattaaagctaacgtgaaagtggcctcaactgaaaacggaattatatttggcaacattgtgtatgatgtcataggagcggggtcagataggaatgttgtagttttgaatgatatacacatagatataatggactatatagtgcctgctagttgtacagatagcgagtttatgagaatgtgggcggaatttgaatgggaaaataaggtaaccgttaacacacccctcacggaactttcagaatacctcgaacatctactcaaaagcacaaatttgaaatgtttaacatcagaaaaagctctgagcgggcagtgtggttttatggcagccaatttatatgcaaaatccatttttggagaagacgctttggccaacttaagtatagagaaaccttttaataaacccgatgcgccagtaagcggtcatattagaataagggccaaaagtcagggcatggccttaagtttaggagacaaagtcaatatgacacagaagagcacacaacataaagtagtagctgcataa

SEQ ID NO: 104 - DNA, 549 bp, Diabrotica virgifera
atgggtaatgtgtttgcaaatttattcaaaggcctctttggcaaaaaggaaatgaggatattgatggtaggactcgatgcagctggtaaaaccacaattttatataaacttaaattaggagaaattgtaacaactattccaacaattggatttaatgtggagactgtagaatataagaacattagttttacagtatgggatgtaggtggtcaagataaaattaggccattgtggagacactatttccaaaacacacaaggcctaattttcgtagtagacagtaacgacagggaacgtatcactgaggctaaagatgaattaatgcgtatgttggccgaagatgaacttagagatgccgtacttctcattttcgccaacaaacaagatttgcccaatgcaatgaacgctgcagaaatcaccgacaaactcggtctccattcactacgcaaccgcaactggtacattcaagctacctgtgcaactagcggagatggtctctatgaaggtctggactggttgtccaatcaattaaagaacgccaatcgctag

SEQ ID NO: 105 - DNA, 4425 bp, Diabrotica virgifera
atgggacggttgcactgtttattttgtattttcctatgttttaccgtcatcaacacgcagacaacgaatatacatggattctcggaaaattccgtggatacatttctatcacctcatgggaaaagtgcaaaattcgtgcaccaaaatcacaaacccaaaattgaaaattgtcagaactacaaaccctcggtgaaagaagaacagccaggcggaacgtacgtaacaacggttaccgctatcgatgacgatcctagggagggaggaggaacaattagttacaaactaattcatagagaaggagaacatgttttatttgacatagacaacgttactggtgttttgacaactatccagccatttgatcgggatgaaccagtaaggcagaaggaactttatgtaaccgtacaagcttcagacaacggcaggccaccattagcagatgtctgtacatttacagttaccattaccgacattaatgataatgcgccacagcttgataaactgaaatacgatgcacaagtttctgaagatttaaaagtaggaagtgaagtgatgagagtttttgcttacgacattgatgatggggaaaattcaagattatcgtataacttttcaaacgaaaatgctcaattcacccagtatttcaggatagatcgagatactggcgttgtgtatttaaaggaagctttaacagacaaaaagaatactagatttaacagtgctgtttatgtagccgataatggcgttaacgatcaagaaggccaaaaagattcaaccgctaagatatctataacagtagtagggtctgataaacagcctcccagatttactcaaaaaatgcctgatggaatcttggagatccccgaagattttaaagacttttctaaacatattgtcacagtcgaagcaacgtccaacattgcggatccacaacttgcttttgaattggtgaagggaaagacatatcaaaccaataaagaccaaacgtttcttttggaggcagaaggaaataaagcgcacataaagctagtgcgtccactggattatgaaacagtaacggaatatactctaactattcgagtaaaaaacaaagatttaatggattcttccataaatataccaattaaagtattagatgttaatgatgaaattcctaatttccttgaatttcttaaaggtagtgtcgtggaaaatgacaagccaggtgcacaagcgattcaagtaagagcaatcgataaagacggaactgctgctaacaacattgtgagctatgaactcgttgacaatacagatttgtttgcaataaaccgatctacgggagtaattacgtcgagagtggagtttgatcgtgaaactgtacctctatatcacgtaaacgttaaagcttatgataactctccgtctgctttgtataacacgacattgcctaacattgtaattcagacattccaaatcagtatagaagatcaaaatgacaacaaacctgtatttactcatccaatttatcagttcagtaatattactgagcttgctgataaatcgagtattgttggtgaagtcaaagctttagataatgacacggcttcagttataagttatagtattacaaatggaaatattgacgatgcgtttatgattgaaaattctaccggcagaataagagttaatggaaaactggattacgagaaaatcgaacaatacaacttaaccgttcgcgcatttgatggggcatttgaagattttgcaattgttttaatttccatacttaatgaaaatgacgaacctccagtttttgacgactatatcagagaaattcaaattaaagaggaagaacctatgatatccggatgcgttgttagagtgactgctcatgatccagatattaaagacaggcatgctgatcaacacatagtatatgaggtcgcgaaagaacagaaagattttttgaccgtatctgccgatggatgcgtacaagtaacaaaacctctcgaccgagatccgcctttcggtagcccaacacgacaagtcttcatctatgctcgtgataatgatggaggcacaaattcattgttggccactgcagaaattgaaattattttaatagatataaacgataatgctccctttttaaatgttacagaaattgtttattatgaaaaccaggatccaggttttataggtaacctaagtgccgatgattacgatggtcctgataatggacctccgtttgcttttcgattatcagacactgcttcagatagtattagatcgaaattttccattatcggaaaccagcttttcgctttagaaatgtttgatagagaagagcaaaaatattatgacattgccattgacattacagatagtggagtacctccactaacaggaactagtattcttagagttataatcggagatgtaaatgataatccagctacagacggaaacagcacgatctttgtgtataagtacgtcaatgggccagaaaatttcatggaaatcggacgtgtatatgttacagacctagacgattgggatttaaatgacaaagtctttgttcaagaagataactttgatgaatttgtgttaaaccagcataacaacggtatgattctgatgaaaccaacaacggctgagggaacttatgaggttcattacagggtcactgaaacccatgaacccacaatacacgaacatacagttaatgcaatagtcacgattacagttaaagtacttccagaggaagcggttgtaaaatcaggatcaattcgattgagaggaacaactaaggaagaattcatagaaaattcattgaatggaaagagcaaaagagacatattacaccaagaactctccaaaatattaaatacatctttagcgaatgttgatgtatttactgttttaaattcaccccaccagaatagttcgtttgtggatgttcgattttctgctcatggatctccatattatgctccagagaaactcgaaaacaaagttacagatcatcaaatggagcttgaacaaaaattagatgtggaattctacatgatcaacgtaaacgagtgccttaacgaaacaacgtgtggagctgaaaactcatgtacgaacaaattaaacataacacgagaaccagctgtagtgtttactaacagaacatcctttgtcggtgtaaatgcatttattgatcctgtgtgtgccgctttaccaagagatgttatggaatgtttcaacggaggcgtccttatcgaaaacacagcgtgtaattgtcctgcaggatttgaaggaccacattgtgaaatcctagctataggatttacaggaactggttgggctatgtatccatcctttgacgctacaaacaggactgagattatactgcatattttatcacaaactgataatggtttgatattttacaatggacctttaaatataagacaaacttctttgtctaaagattatatatcattagaacttaaagacggatatccattacttcaaatttgcaccggctcaagcactcaagaaatttatctgaaagagcgcattcacaaattgagcgatggatcgttacacaaaataaaaataggatctggatttgacgatatatccctggaagtagacgactgtggaacaacgtgttcaatttggactaataaactacataaaggtgttatccgagcaaatggcccccttcaactgggaggtatgaaaaacagattcaccgatcaagaattcaaacgaatttgggaccatttgccaccgactgccacccgtttctctggttgtattagaaatttgacgtataatgaattttactacaacctcggtgcaccttctgatgcattccaagcgtatcccgactgtaactatgcagtgatgcaagctgtgactttcggtatcgactccaatttcttggttgctattctggtttgtgtagcaattttgataattcttcttctggcagtagttgtacatagacgtaaacacgacaactttaacgaaaaagaaatcgatgatactcgcgaaaacattatcaactacgaagatgaaggtggcggcgaatgtgacaccaactacgacctgtctgttttccatcagaacaacattgtggacgaaaaaccattgatgagagacaaccccgatgtacctgcagatataagtggctttttagataacaagaaagacaactgtgataaagaccccgataatttgccttatgacgacgttcgccattatgcctacgagggagacggaaatagcaccggatccttatcttctctcgcttcatgtacggacgaaggagatttaaagttcaactacttatcaagttttggacccagattcagaaagttagccgacatgtatggagaagatccaagcgatgaagactcacacgatggaaacgaagaatcctggtgctag

SEQ ID NO: 106 - DNA, 291 bp, Diabrotica virgifera
atgcctttctgtggtcccaaattgtccctctgcggcctgattatcagtgcatggggtatcatccagttgggtttcatgggtgtattctattacattggggctgtggctttagcagaagatattccagaggttgagtttaagggcgatttagacaaattttatagcgacgtcaacacgggtttcacacagaatgcttacaactgctggattgctgctctcctatacctgataacattagcagtatcagctcaccaattctgggccaacaacagatcatcattgaacgtctaa

SEQ ID NO: 107 - DNA, 540 bp, Diabrotica virgifera
atgggtcttaccatatcagcagtgtttaataggttgtttagtaaaaagcctatgagaattttaatggtaggattagatgccgcaggtaaaaccacaatcttatacaaattgaagcttggtgaaatcgtaactacaataccaaccatcggcttcaatgtagaaaccgttgagtacaagaatatatctttcacggtatgggatgtaggtggccagacgagaatcagaaaactctggagacactatttcgccaacactgatggactcatttttgtggttgattccaacgaccgagaccgtatcgcggaagccgaagaagaattgcacaatatgttaggagaggacgatttaagagactgcattttgttaatattcgccaacaaacaagatttaccgaactcgatgtccactgctgaattgaccgataagcttaagttgcacactttgaagaataggaggtggtacatacaagccacatgtgctactcaagggaatggtttgtacgaaggactagattggttgtcgaatgaattggccaagtga

SEQ ID NO: 108 - DNA, 618 bp, Diabrotica virgifera
atgcggtacactttgagttacatcggtgctaccctagcgtccactgtaacactgatttttgccctctactactgcctcacgggaaaaggagagcaagttagtttagcatggttattgttgaatgtgtctccccacatgtgggcaggtctaggaattggccttgctgtatcattatcagttgtaggagctgctgcaggaattcacactacaggagtcagtatcgtaggagctggtgttaaagcccccagaatcaaaaccaaaaatttaatttctattattttctgtgaagctgtggctatctatgggttaattatggctatagtactctgtggaagttggaagaatttcgatgtagacctattcaacctcaaaactcataactttgctcaaaaccattatggatcacatgttatttttggatccggtttaactgttggatttgtaaatctattatgtggattttgtgttggagtagttggttctggtgcagccatttctgatgcagccaattcatcattattcgtcaaaattttgattattgagatttttggaagtgccattggtctcttcggtctgattgttggagtatacttgacgtcaagaggctctatggtttaa

SEQ ID NO: 109 - DNA, 5694 bp, Diabrotica virgifera
atggctaccaacgatagtaaagctccgttgaggacagttaaaagagtgcaatttggaatacttagtccagatgaaattagacgaatgtcagtcacagaagggggcatccgcttcccagaaaccatggaagcaggccgccccaaactatgcggtcttatggaccccagacaaggtgtcatagacagaagctcaagatgccagacatgtgccggaaatatgacagaatgtcctggacatttcggacatatcgagctggcaaaaccagttttccacgtaggattcgtaacaaaaacaataaagatcttgagatgcgtttgcttcttttgcagtaaattattagtcagtccaaataatccgaaaattaaagaagttgtaatgaaatcaaagggacagccacgtaaaagattagctttcgtttatgatctgtgtaaaggtaaaaatatttgtgaaggtggagatgaaatggatgtgggtaaagaaagcgaagatcccaataaaaaagcaggccatggtggttgtggtcgatatcaaccaaatatcagacgtgccggtttagatttaacagcagaatggaaacacgtcaatgaagacacacaagaaaagaaaatcgcactatctgccgaacgtgtctgggaaatcctaaaacatatcacagatgaagaatgtttcattcttggtatggatcccaaatttgctagaccagattggatgatagtaacggtacttcctgttcctcccctagcagtacgacctgctgtagttatgcacggatctgcaaggaatcaggatgatatcactcacaaattggccgacattatcaaggcgaataacgaattacagaagaacgagtctgcaggtgcagccgctcatataatcacagaaaatattaagatgttgcaatttcacgtcgccactttagttgacaacgatatgccgggaatgccgagagcaatgcaaaaatctggaaaacccctaaaagctatcaaagctcggctgaaaggtaaagaaggaaggattcgaggtaaccttatgggaaagcgtgtggacttttctgcacgtactgtcatcacaccagatcccaatttacgtatcgaccaagtaggagtgcctagaagtattgctcaaaacatgacgtttccagaaatcgtcacacctttcaattttgacaaaatgttggaattggtacagagaggtaattctcagtatccaggagctaagtatatcatcagagacaatggagagaggattgatttacgtttccacccaaaaccgtcagatttacatttgcagtgtggttataaggtagaaagacacatcagagacggcgatctagtaatcttcaaccgtcaaccaaccctccacaagatgagtatgatgggccacagagtcaaagtcttaccctggtcgacgttccgtatgaatctctcgtgcacctctccctacaacgccgattttgacggcgacgaaatgaacctccatgtgccccaaagtatggaaactcgagctgaagtcgaaaacctccacatcactcccaggcaaatcattactccgcaagctaaccaacccgtcatgggtattgtacaagatacgttgacagctgttaggaagatgacaaaaagggatgtattcatcgagaaggaacaaatgatgaatatattgatgttcttgccaatttgggatggtaaaatgccccgtccagccatcctcaaacccaaaccgttgtggacaggaaaacagatattttccctgatcattcctggcaatgtaaatatgatacgtacccattctacgcatccagacgacgaggacgacggtccctataaatggatatcgccaggagatacgaaagttatggtagaacatggagaattggtcatgggtatattgtgtaagaaaagtcttggaacatcagcaggttccctgctgcatatttgtatgttggaattaggacacgaagtgtgtggtagattttatggtaacattcaaactgtaatcaacaactggttgttgttagaaggtcacagcatcggtattggagacaccattgccgatcctcagacttacacagaaattcagagagccatcaggaaagccaaagaagatgtaatagaagtcatccagaaagctcacaacatggaactggaaccgactcccggtaatacgttgcgtcagactttcgaaaatcaagtaaacagaattctaaacgacgctcgtgacaaaactggtggttccgctaagaaatctttgactgaatacaataacctaaaggctatggtcgtatcgggatccaagggatccaacattaatatttcccaggttattgcttgcgtgggtcaacagaacgtagaaggtaaacgtattccatttggcttcagaaaacgcacgttgccgcacttcatcaaggacgattacggtcctgaatccagaggtttcgtagaaaattcgtatcttgccggtctcactccttcggagttctatttccacgctatgggaggtcgtgaaggtcttatcgatactgctgtaaaaactgccgaaactggttacatccagcgtcgtctgatcaaggctatggagagtgtaatggtacactacgacggtaccgtaagaaattctgtaggacaacttatccagttgagatacggtgaggacggactctgtggagagatggtagagtttcaatatttagcaacggtcaaattaagtaacaaggcgtttgagagaaaattcagatttgatccgagtaatgaaaggtatttgagaagagttttcaatgaagaagttatcaagcaactgatgggttcaggggaagtcatttccgaacttgagagagaatgggaacaactccagaaagacagagaagccttaagacaaatcttccctagcggagaatccaaagtagtactcccctgtaatttacaacgtatgatctggaatgtacaaaaaattttccacataaacaaacgagccccgacagacctgtccccgttaagagttatccaaggcgttcgagaattactcaggaaatgcgtcatcgtagctggcgaggatcgtctgtccaaacaagccaacgaaaacgcaacgttactcttccagtgtctagtcagatcgaccctctgcaccaaatgcgtttctgaagaattcaggctcagcaccgaagccttcgagtggttgataggagaaatcgagacgaggttccaacaagcccaagccaatcctggagaaatggtgggcgctctggccgcgcagtcactgggagaacccgctactcagatgacactgaacactttccattttgctggtgtatcctccaagaacgtaaccctgggtgtacctagattaaaggaaattattaatatttccaagaaacccaaggctccatctctaaccgtgtttttaactggtgcggctgctagagatgcggaaaaagcgaagaatgtgttatgcagacttgaacacaccactcttcgtaaagtaaccgccaacaccgccatctattacgatcctgacccacaaaataccgtcattcctgaggatcaggagttcgttaacgtctactatgaaatgcccgatttcgatcctacccgtatatcgccgtggttgcttcgtatcgaactggacagaaagagaatgacagataagaaactaactatggaacaaattgctgaaaagatcaacgctgggttcggggacgatttgaattgtattttcaacgacgacaatgctgaaaagttggtgctgcgtatcagaatcatgaacagcgacgatggaaaattcggagaaggtgctgatgaggacgtagacaaaatggatgacgacatgtttttgagatgcatcgaagcgaacatgctgagcgatatgaccttgcaaggtatagaagcgatttccaaggtatacatgcacttgccacagactgactcgaaaaaaaggatcgtcatcactgaaacaggcgaatttaaggccatcgcagaatggctattggaaactgacggtaccagcatgatgaaagtactgtcagaaagagacgtcgatccggtcaggacgttttctaacgacatttgtgaaatattttcggtacttggtatcgaggctgtgcgtaagtctgtagagaaggaaatgaacgctgtcctttcgttctacggtctgtatgtaaactatcgccatcttgccttgctttgtgacgtaatgacagccaaaggtcacttaatggccatcacccgtcacggtatcaacagacaagacactggagctctgatgaggtgttccttcgaggaaactgtagatgtattgatggacgctgccagtcatgcggaggtcgacccaatgagaggagtatctgaaaacattatcctcggtcaactaccaagaatgggcacaggctgcttcgatcttttgctggacgccgaaaaatgtaaaatgggaattgccatacctcaagcgcacagcagcgatctaatggcttcaggaatgttctttggattagccgctacacccagcagtatgagtccaggtggtgctatgaccccatggaatcaagcagctacaccatacgttggcagtatctggtctccacagaatttaatgggcagtggaatgacaccaggtggtgccgctttctccccatcagctgcgtcagatgcatcaggaatgtcaccagcttatggcggttggtcaccaacaccacaatctcctgcaatgtcgccatatatggcttctccacatggacaatcgccttcctacagtccatcaagtccagcgttccaacctacttcaccatccatgacgccgacctctcctggatattctcccagttctcctggttattcacctaccagtctcaattacagtccaacgagtcccagttattcacccacttctcagagttactccccaacctcacctagttactcaccgacttctccaaattattcacctacttccccaagctacagtccaacatcccctaactattcaccaacatctcccaactattcacccacttcacctagttatccttcaacttcgccaggttacagccccacttcacgcagctactcacccacatctcctagttactcaggaacttcgccctcttattcaccaacttcgccaagttactcccctacttctcctagttattcgccgtcgtctcctaattactctcccacttctccaaattacagtcccacttctcctaattactcaccgtcctctcctaggtacacgcccggttctcctagtttttccccaagttcgaacagttactctcccacatctcctcaatattctccaacatctccaagttattcgccttcttcgcccaaatattcaccaacttcccccaattattcgccaacatctccatcattttctggaggaagtccacaatattcacccacatcaccgaaatactctccaacctcgcccaattacactctgtcgagtccgcagcacactccaacaggtagcagtcgatattcaccgtcaacttcgagttattctcctaattcgcccaattattcaccgacgtctccacaatactccatccacagtacaaaatattcccctgcaagtcctacattcacacccaccagtcctagtttctctcccgcttcacccgcatattcgcctcaacctatgtattcaccttcttctcctaattattctcccactagtcccagtcaagacactgactaa

SEQ ID NO: 110 - DNA, 876 bp, Diabrotica virgifera
atgtcgtcaaatattcaaaaggcccagcagttgatggcggatgcagaaaagaaagtaacatctcgaggtttcttcggatctctatttgggggatcaagtcgtattgaagatgcagtggaatgttacacaagagctgcaaacctttttaaaatggccaagagctgggatgctgccggtaaagccttttgtgaggctgctaatttgcattccagaactggtgctcgtcatgacgctgccactaattatatagatgctgcaaattgttacaaaaaagccgatgtatttgaggctgtaaactgctttataaaagctatagacatttataccgaaatgggtcgctttacaatggctgcaaaacaccatcagactattgcagaaatgtatgagactgatgctgtggacatcgaaagggctgttcaacactatgaacaggcggctgattacttcagaggagaagaaagcaatgcttccgccaataagtgtcttcttaaagtggctcaatatgcagcccaacttgaaaactatgaaaaagcagtgggaatttatcaagaagtggcttatgcagctctggaaagctctcttttaaaatacagtgcaaaggaatacttattcagagctgccctttgtcacctttgtgttgatgtactcaatgcacaacatgctatagaaagctatatttcaaggtatcccgcatttcaagattcccgtgaatacaaacttttgaaaaccctcatagaaaacatcgaagagcaaaacgtagatggatatacagaagccgtcaaagattacgattcaatttctcgtcttgatcagtggtatactacaattcttttacgtattaagaaacaagtaagcgaaagccctgacttacgttaa

SEQ ID NO: 111 - DNA, 609 bp, Diabrotica virgifera
atgaatcccgagtatgattatttattcaaacttctgctgattggagattcaggagtaggaaaatcttgtcttctactgagatttgcagatgatacctacacagaaagctatattagtaccattggcgtagattttaaaatcaggacaatcgatttagatggaaagacaattaaattgcaaatttgggatacagcaggtcaggaaaggtttagaacgattacatcaagttattaccgaggagcacatggtattattgtagtgtacgattgcacagaccaagattcattcaataacgttaaacagtggctcgaagaaatcgaccgttatgcgtgtgacaatgtaaacaaattactggtagggaataaaagcgatttgacaactaagaaagttgtcgacttcactacagccaaggagtatgccgaccaattgggtataccatttttggaaacctcagctaagaatgcaaccaatgtagaacaggcctttatgactatggccgctgaaataaaaaatagagtaggacctccatcttctgcggtagaccaaggaaataaggttaggttcgatcaaagtcgcccagtcgaaacaaccaaatccggttgctgctga

SEQ ID NO: 112 - DNA, 789 bp, Diabrotica virgifera
atggcagacgctgatgatctattagattatgaagatgaggaacagacagaacaaaccgcaactgaaacggcaactacagaggtacagaaaaagggtgtcaagggcacatatgtatcaatacacagttctgggtttagagattttctgttaaaaccagcaattctcagagctatagtggactgcgggttcgaacatccttcagaagttcaacatgaatgtattcctcaagctgtcattggcatggatattctgtgccaagctaaatccggtatgggaaaaacggctgtttttgtattagctacactccaagtaatagatcctacagaaaatgttgtatatgttctcgtcatgtgccataccagagagttagccttccagataagcaaagagtacgaacgtttcagtaaatatatgcccaatattaaagtaggggtcttctttggtggcttgcctatccagaaagatgaggaaacgttaaaaaataattgcccgcatatcgttgtgggtactccaggaagaattttagcattggtcagatcgaaaaaacttaatctcaaacatctaaagcattttattttggatgaatgtgataaaatgttggagttattagacatgagacgtgatgttcaagaaatatatcgtaacactccccacgaaaaacaagtcatgatgttcagtgccaccttaagtaaagaaattagaccagtttgcaagaaatttatgcaagatgtaattcaaaattcttataatacacaattttgtaatgacgcacccactcgcaatgtttga

SEQ ID NO: 113 - DNA, 738 bp, Diabrotica virgifera
atgccggtcattgatggttataaagtactttacattttattacacagtttatatacaatttttgaaaatatttggaggactcttttatttatttatcaaaattgtataagggttataaaccctgaatctacattcgatgatgctgaccagttaaagaaaagactgtctagactaacaaaaaagcctcaacatttaactatcattattggtgtggaagaatattcattggtagatttggctaacctcgtatattggtgtttaggtcttaatattccgtacgttagtttctatgattataaaggtaatttaaaaaagcatgaagagaagttgcaacaaattgtagaatccagaaaatcagagaatatcaacataatttggcacacccatgcagaacaaaggcataaaaatggatttttgggtccaaaaatccacgtaaaagtgttaacacacgcggacggaaagcaaagtatagtaaatgttactaaaaaattagctctaaataaagaaaaagacattagtaaagaaaaaattagtgaattactattaaggcagtatgaatttccagatccagaaatggctattatttgtggaaagaaactgaacatttataattatcctccttggcagttaagactcacagaattctttaaagtcaacaaagtcaacaacatcacattcccagtgtttgtggaaaaattggaaaagtacagcaaatgtgaacagagggtgggaaaataa

SEQ ID NO: 114 - DNA, 480 bp, Diabrotica virgifera
atgaccaactctaaaggttaccgccgaggaaccagggatctatttgcccgcaagtttaaaaaacgtggtgtaattccactttccacatatttgagagtctacaaagttggagatattgtagatatcaagggtaatggtgcagttcaaaagggtatgccccacaaagtgtaccatggtaagacaggacgtgttttcaatgttactgcacatgcattaggtgtaattgtaaacaaaagggttcgaggaagaatcatccccaaaagaatcaatctccgtattgaacatgtaaaccactccaagtgtcgtcaagacttcttgcaaagagtaaaatccaacgaaaagctacgtaaagaagctaaagaaaagaacattaaagtagaacttaggagacaacctgcccaacctaggccagcacatattgttagcggaaaggttccagcacaggtgcttgctcctatcccatatgaattcattgcttag

SEQ ID NO: 115 - DNA, 537 bp, Diabrotica virgifera
atggaaggaatactactggaaccaacattgtataccataaaaggtattgctatattggactatgatggtaatagagtgctggctaaatactacgataaagatatatttcctacagcaaaagagcagaaagcttttgagaaaaatttgttcaataaaactcatagggcagacgcagaaattatcatgttggatggtttaacttgtgtgtatagaagtaatgtagatttattcttttatgttatgggcagttcacatgaaaatgagctaattttaatgagtgttttaaattgcttgtatgactcagtaagtcaaatattgaagaaaaatatgcaaaaacgagctgtcttggaatcactagatattgttatgctggctatggatgaaattgttgatggaggaataattatagattctgattcaagttcagtagtatctagaatagcattaaggactgatgatattccattaggagaacaaactgtagctcaggtattccaaacggccaaagaacagctgaaatggtcattgctgaaataa

SEQ ID NO: 116 - DNA, 450 bp, Diabrotica virgifera
atggctgaccaactcaccgaagaacaaattgctgaattcaaagaagctttctcactattcgataaagatggtgatggtacaattacgactaaagaattaggaacagtaatgagatctctaggacaaaatccaacagaggctgaattacaggatatgatcaatgaagtagatgccgatggtaacggcacgatcgatttcccagaatttttaacgatgatggcacgtaaaatgaaagataccgatagtgaggaagaaattcgtgaagcattccgagtgttcgacaaagacggcaatggtttcatctcagcagcagaattgcgccacgtcatgaccaacttgggtgaaaaattgacagacgaagaagtcgatgaaatgattcgggaggccgatatcgatggtgatggtcaagtcaattacgaagagttcgtcaccatgatgacttcaaagtga

SEQ ID NO: 117 - DNA, 2013 bp, Diabrotica virgifera
atgatgcaagcaaacaatcgagtcccacctataaagttggaaaacgatatagatctttacgccgatgatatcgaggatttcgcacaagatgactttggtggtgaaaatgttgatctatatgacgatgtaatatccgctcctcctggaaataatgacaacccaggtgattcaaatcatcatgctcctcctggtgctggtgaagatggtggaggtaattttgttgggtcaggaggagcacccaataatataaattcttctggaagaagacatcagctgtatgttggaaatctgacttggtggacaactgatcaagatatagaaaatgcagtgcatgatataggggtaaccgacttccatgaagttaagttttttgaacacagagcaaatggtcaatccaagggattctgtgtcatatctttgggatctgagggaagcatgagactctgcctggaactcctatctaaaaaagagatcaatggccaaaatccccttgttacccttcccacaaaacaagctcttagtaactttgaaagtcagtctaaaacacgcccttctcctactaataattctaactcacgtcctccccatcctaataataatgttcattcaggtcctatgcagaattatggaggtagaatgcctatgaacccttccatgcgtcccatgcccccaggtatgcaaggtgctccaagaatgcagggtccacctggatttaatggaccaccaaacatgaatcagcaaccccccaggttccaaggtaatccacaatggaatggacctagacctaatggtcctgggcccaatatgggaatgagacccatggggccacctcatggacaacaagggcccccaagaccaccaatgcagggaccaccgcagcaaggtcctccaagaggaatgccgccacaaggtccaccgcagatgcgtccagaatggaatcgaccaccaatgcaacaagggtaccctcaaggcccgccgcatatgcaaggacctaacatgggtccaagaggtccaccccaaatgggaccacccggggcgcctcaacagcaaggaccagctccgcacgtaaatccagcattctttcaacaaggaggaggaccaccgcccccaatgcaacacatgcctggaccagggcccgtcatgcctcctcaaggacccccgcaaggtccaccacacggacccgttggacctccacacggcccaccattgggtccagcgaatgttccgcctcatggaccacctcacggatatggtccacctgcagcgatgccacagccgccatacggtggcccacctccagaccaccgcgctgagattcctcagttaacagagcaagagtttgaggatataatgtcccggaatagaacagtttccagttcggcgattgggcgggccgtatccgacgccgcagctggagaatttgcaagcgccattgagactttggttactgctatttcactcatcaaacaatccaaagtggctaacgacgatcgttgcaagatccttataagttcgctgcaagatactttgcgtggtgtcgaagacaaaagctacagctccagccgcagagaccggtcaagatccagggacagatcacatagaagaactagaagagaacgatcctcgtcacggtacagagacagaagcagagagagggagcgtgaacgcgatagagatcgtgatcgtgaacgtgacagatattatgatagatacagcgaaagagaaagagaccgagatcgttcaagaagcagagaaagaacagaaagggatagagaacgagattatagagaccgggaacccgaagagacagataaagaaaaatctaaagtatccagagtctcaagatcaagaaacaaatctccggaacctgtcgaacctagcagcgaggtaccgaaatcatcccgctattatgaggataggtatcgggaacgagagagagaaggtcgacgagagagcgatcgcgaaagagaaagagatagaagaggggaagacagccataggtctcgacactag

SEQ ID NO: 118 - DNA, 765 bp, Diabrotica virgifera
atgagttctattggaactgggtacgatttatcagcttcccaattctctcctgatggaagagtatttcaagttgaatatgcaatgaaagcagttgaaaatagtggcaccgtaataggcctccgaggtacagatggcattgtattggctgctgaaaagctcattatgtcaaaattgcatgaaccaagtacaaataaacgaattttcaacattgataaacacataggaatggcattttcaggcttaatagctgatgcaaggcaaatcgttgagattgctagaaaagaagcatcaaattatagacatcaatatggttcaaatattcctcttaaatacctaaatgatagagtaagcatgtacatgcatgcatacactttatacagtgctgttagaccatttggttgcagtgtcatcttggccagttatgaagatagtgacccatctatgtatctgattgatccatctggagttagctatggatactttggatgtgctacaggtaaagcaaaacagtctgcaaagactgaaatagaaaaattgaagatggggaatctaacatgcaaagaacttgttaaagaagcagccaaaatcatttatttggtccatgatgagctgaaggataagaattttgaactggaactttcatgggtatgcaaagatacgaatggtttacataccaaagtgcctgaatcagtgtttgctgatgcagaaaaagctgccaaacaagcaatggaagcagattcagaatcagatacagaagatatgtaa

SEQ ID NO: 119 - DNA, 744 bp, Diabrotica virgifera
atggcttcaaaagacagattgatgatttttccatctagaggagcccaaatgatgatgaaatccaggctaaagggagcccaaaagggacatagtttattaaagaagaaagctgatgctttacaaatgagatttagaatgattttgaacaaaattattgagaccaaaactctcatgggtgaagtaatgaaagaagctgccttttctttagctgaagcaaagtttgcaactggtgacttcaatcaagttgttcttcaaaatgtcaccaaggctcaaataaaaataagaactaagaaagacaacgttgctggtgttactttaccagtgtttgaatgctaccaagatggtacagatacatatgagttggctggtttggctaggggaggtcaacaattgacaaaactcaagaagaattatcaaagtgctgttaaactgttggttgaattagcctctttgcaaacttcttttgtaactcttgatgatgtaatcaaaataacaaacagaagagtcaatgccattgaacatgttatcattccaagaatagagcgtactttggcttacatcatatccgaactggacgagttagaaagagaggagttctatagattaaagaagatccaggacaaaaagaagatcagcagagcaaaggccgagaaacaaaaacaagctcttctccaagctgggctacttaaagagtcccaggcaaacatgcttttggatgagggcgatgaagatctacttttctag

SEQ ID NO: 120 - DNA, 4818 bp, Diabrotica virgifera
atggaggcggctcccaagttaccgatgctctcgttcgagttaaatacttgtacagagaacgtccactttggcccccagttaaaacagtatattgctgctttttatggtgaagatccagaatcctacattacagaaatcagcaatcttgaatccttaagatcagctgcagttcgaccatcaacggatgtaaatggtgtacaactgttgaaaaagtatttctgtcagcttcgttttctcaaatctaggtttcccatggaagagaatcaagatgctgcagttctattttcatggaaaaataatgaattagacataacttcaacatccagtgatatcagatatgaattaatggtaataatgtataacattggagccttacacacttttcttggagccaacgactcaagaaacaatccggatggtatgaaaatggcatgtactcattttcaatgtgctgcatgggcttttcaaaacgtaaaagaaaagtaccaccaattcatatcaaacatctcattggtagaactggttcatttttttcaacaagtctgtttagctcaggctcaggagtgtatattagagaagagcatgtttgacaataggaaacctaccatcattgcaaaagttgctatccaagtctacagttattacagacagtctttacgtgtcttggaatcagtaaatgaagcctactttagggataaaacctacaaggagtggatgaaatatcttcaattcaagctgacctactacaaatgcatctcgttcctattccaagggcaacaagctgaggaacaacagaaaatgggagaaagggttgcattctatcaagctgcatgtgaacagctggacgaggcaaagaaaattgctgctacattaaaaaaccaacaccaccagcaagaaataaatgagggactagcattcactactgatgtggttgaaggtaaaagaaaagcagctaaaaatgaaaatgagttcatctaccatgaatcagtgcctgataaagaccaattgccagaggttaagggtgcttcattagtcaaaggaataccattcagtataaatgatatagaagtttcaggaccagatattttctcccgattggtcccaatggaggcacacgaagcagcttccttgtacagcgagaagaaagctcagagattaagacagatcggggaacttattgaaaataaagatcaaacattggctgaatttatgtcgtcaatgcagctagatctattgaccaagatgcaccaggctactggaataccgcaggagttgattgatagagcagcggctctatctgctaaacctaacgccattcaagatcttataagtgctatgggaaagctatctaatatataccaagacgttgaagcaagtttgaatgagattgattctttattaaaggccgaagaacaaagtgaacaaaagtaccaagaaacgattggtaaaagaccaccgagcattttagctacagatttaactagggaagcggcaaaatacagggaggctcatactaaagcgaacgactcaaaccaaactttacacagggcgatgatggctcacgtggctaatctgaaaatactccaacaaccgctaaagcagctgcaacatcagctgccctttgtcgagtttccaaatccaaatatcgacgaaaaatctttgaaagatctggaagcgctagttgcaaaagtagacgaaatgagaacccaaagagccatgctatgggctcaacttcgagaatctattcaccaagacgatattacaagttcccttgtaacgaaacaaccaaatcagtcgctggaacagctgttccagcaagaacttcaaaagcatcaaaatctgatttcgttgattgaacaaaacacctcggcacaagaaaacattaagagcgccttagtcgattcttacgcttacgctgtaaattcaagaaaatacatccaagatatactccaaaagagaaccacaaccataacgtcactgatagcatcgttcgactcttacgaagacttattggcaaaagctaacaaagggatagagttttactcaaaacttgaaacgaacgtatccaagttactgcaaagaataaggagtacctgcaaagttcaacaagaagagcgagatcagatgatgtcgactgcgcaagtgcctcaatgggagagtcatacgtcacttgccgctcctaaactgaaagattacttggactccaggaagaagagtgctgcgtattcggagccgagtgttcaaccacaacagccaactttaagttactcagctgctatggatctgcctcctggtattaggccgactccagttggatcagaaataacggatgtaccgaaaaatattcaaggtgaaccacaaggttatattccatataattaccaacaaccttctgttcctgcctcacagaatattgatgaagagactattaaaaaaatgaacgcattgatgccaggtgctaagacgtcagtgcctagtcagtacggatacagcaactacattccaccaacataccctcaaagtgcgtaccaaccaggtaatcagtcttacggaaaagaaactccagatattaactcaccgtacgaccctaccaaggcgttcacggctactactaacgcttatcgttcggtgcagagctcctcaactcaaggatacgtaccgtacgcagaatctaacgtttcgaatgttgacagagttggatatcctagcaggtatcagtaccaacaagtacctgagatagctactactccagctgatcccaatattaatgcgtactacccacatgggtactcaccgagccagaatttaccgaatgctaatactcaacatattaccggccaactgaagtaccattcggtggagtacgcttcttctgtgccgaacaacatcaattataacagctctacctactcgtcgccgctttctaatatgtctagtaccaattcctcaaatcctagtaacttgaataattcttacgagtactactatgacccgaataccagtagtggtgcagtaccgaatgcttcaaagcctcaacagtcgagcgccagctctgcaaacccgagtaccgctatgaacaactacaattattactacaatacaagtaccagcggtagtgtagcagcggatacttcaaaaatacaacaacaacaacagtacccaggtactcagatgagtcaagcgcagtactatcccgccaatgccagttattactcaaccagtacttacaataccaacgtccaaggtggtaccaatccctcgtacgcaactggacaaacatataatcaagtgacaccagtgacctctcaaaatgtttctcaaaattacaactttaaccaagttggttctggagcaggacaccagcatcagtactactcgtccgctaacgccgcagtaccatcccaacaagctgtaaataacagttcattaccaaactacggatacgatcagtattacggcaacaactataattccagtcaaccgagtacctacagcgcaaaccaagcacctcctgcagcacaagctgctccaagtaatattcctgctgccaccaaatcctcctctaatgtggatctgctcagtggcttggacttcagcataagccaagctcctctagtgcctcaacaaaacattacgataaaaccccaagaaaaggaaacaaaaccaccggctgtttcttctgaaaccaaaaaccaagatccaacaccagtaaccacgcccaaacaacccactggaccagaagtaaagcgcttgtacgtcaaaatcctgccgagcaaacccttaaacaacgatgatgtgaagaaattgttcggccaagagctggacaggtatgagaagttcgtggagaccttgacccacaaaactttgagcggtccgaccactctggatattaaatggaaggagatccaagaccagcaggattgcgagccgcagaagaagatcatttccgtcgctagatgttatcctatgaagaataggttcccggatatcttgccttacgacttttccagggtggagttgtgcgatagtaaagatgattatatcaacgcttcatacattaaggatatctcgccatatgctccgtcatttattgttacacaagtgccgttgtcttcaactgttggtgatatgtggacgatgattagagaacaacaggtcgaactgatcctctgtttggtaaacgacaatgagatcggtgaagatatttactggcccaaagaaaaaggcagtagtcttaacatacttaacatggtcataacgttgcaaaacgttatagttaagtctcattggactgaaagactgatagcgataaacttacctgaaaaacgggagtcccgtgtgataatgcatctacaatttacatcgtggcctggcagcttgtttccaacaaatcctgaaccgttcgtcagctacaccttggaatccatcaacctataccaacaacagaagaccaacacccatccggtggtggtccattgttcatctggcataggaagaagcggcctgctctgtttactgacagctgctatgttcgatgctgccaacaatgctaactcgataccagatcttacagctttgagtatcaagttgtccaattgcaggaagaatattctcagagatcgagagcatttgaagtttggttacgaaagttttttggcgtatattaggcatatagtttgtgaagataaagccagaaagaaactgaacgagatccagcccaaggttaaggaggaaccactggaaccacctgtcatagttccagaaccaaatatagatcctttaagtactttagacccattttgggctagtaaaagataa

SEQ ID NO: 121 - RNA, 478 nt, Diabrotica virgifera
gguuucaugaccccugagagaaagaagaaacuuagguuacuguugagaaagaaagccgccgaagaauuaaagaaagaacaagaacgcaaagcagccgaaaggaggcguaucauugaagaaaggugcgguaaacccaaacuugucgaugacgcaaaugaaggcccauuaaaacaaguaugugagggauaucacagacguauuguagaccuagaaaauaagaaauuugaccucgaaaaagaaguggaauucagagauuuucagaucuccgaauugaacagccaaguaaacgaccuuagaggcaaauucgucaaaccaaccuugaagaagguauccaaauacgaaaacaaauucgccaaacuucaaaagaaggcagcugaauuuaacuuccguaaccaacucaaaguugucaagaagaaagaauucaccuuagaagaagaagacaaagaaaagaaaccagacuggucaaagaagggagacg

SEQ ID NO: 122 - RNA, 529 nt, Diabrotica virgifera
agaaguugccgcuuuagucguagacaauggauccgguaugugcaaagcugguuuugcuggggaugaugcaccucgugcuguauucccuucaauuguuggacgcccaagacaucagggugugaugguaggaaugggacaaaaagauuccuauguaggugaugaagcucaaaguaaaagagguauccuuaccuuaaaauaccccaucgagcacggaauagucacaaacugggaugauauggagaaaauuuggcaucauacauucuacaaugaacucagaguagccccagaagaacacccuguucuguugacagaagcuccucucaaccccaaggccaacagggaaaagaugacacaaauaauguuugaaacuuucaacaccccagccauguauguugccauccaggcuguacucuccuuguaugcaucuggucguacaacugguauuguguuggauucuggugaugguguaucccacacugucccaaucuaugaagguuaugcucuuccucaugcaauccuucguuuggac

SEQ ID NO: 123 - RNA, 592 nt, Diabrotica virgifera
accggccuuuguaugucuugggauaugugccuaaagacgauagauuauaccucguagauaaagaguugcgcguaguaagcuaccaauuacuucuuucuguucuugaauaucaaacugccgucaugagaagagacuuuccaacagcagacagaguacuuccguccauuccuaaggagcacagaacgagaguggcacauuucuuagaaaagcaaggcuucaaacagcaagcuuuggccguaaguacagauccagagcacagauucgagcuggcaguagcauuagaggaucuuaauauagccaaaacucuagcucaagaagcgaacaguccgcaaaaguggaaucaacuagcagaauuggcagcugcuacuaauaauguaagcguagccaaggaauguaugcaaaaagcgcaagauuauggaggcuuguugcuucuugcuacgagcuccggugaugaaaauuuaguccguacucuaggagaaacgacacaagcugaaagcaaacauaacuuagcauuuuugucacacuuguuaguaggugauuuaaacaaaugucuagacauucuuauuaauaccgguagauugccagaagcugc

SEQ ID NO: 124 - RNA, 594 nt, Diabrotica virgifera
gaaagcaguuggaagauggccguacucucucagacuacaacauucaaaaagagucuacccuccauuugguacuucgucuuagaggagguaugcagauuuuuguuaaaacuuuaacuggaaagaccaucacccuugaaguagaaccuucugauaccaucgaaaaugucaaagccaaaauucaagacaaagaagguauuccaccagaucaacaaagauuaaucuuugccggaaagcaauuggaagauggucguacacucucagacuacaacauucaaaaggaaucuacccuccauuugguacuucgucuuagaggagguaugcaaaucuuuguaaaaacacucacugguaagaccaucacccucgagguugaaccaucagauaccaucgagaaugucaaagcuaaaauucaagacaaagaagguauuccaccagaucaacagagauuaaucuucgcuggaaagcaguuggaagauggccguacucucucagacuacaauauucagaaagagucuacccuccauuugguacuucgucuuagaggagguaugcaaaucuuuguaaaaacucucacugguaagaccaucacccucgagg

SEQ ID NO: 125 - RNA, 547 nt, Diabrotica virgifera
uaccccauugaacacggaauuaucacuaacugggacgauauggaaaagaucuggcaucacaccuucuacaaugaacuuagaguagcccccgaagaacaucccauucuuuugacugaagcuccacuuaacccaaaagccaacagagaaaagaugacucaaaucauguuugaaacuuucaauaccccugccauguauguugccauucaagcuguauugucucuguacgcuuccggucguaccacugguauuguacuugauucuggagaugguguaucccacacaguacccaucuaugaagguuacgcucucccacacgccaucuugcguuuggacuuggccgguagagacuugacugacuaccuuaugaagaucuuaaccgaaagagguuacucuuucaccaccacagcugaaagagaaauaguucgugacaucaaggaaaaauugugcuauguagcuuuggacuucgaacaggaaauggccacagcagccagcuccaccuccuuagaaaagaguuaugaacuuccugacggucaagucaucaccauug

SEQ ID NO: 126 - RNA, 592 nt, Diabrotica virgifera
aggaaaggaaggacccaagaccaaacaaccaucgagauacauccguuuuaucuacaaucgcgucauauuggaauguccuucuguaagagcugcugcagucuccgccauggcacaauucggagccucuugucccgauuuguuagaaaauauccaaauauuacuuucgaggugucagauggauucagacgaugaaguuagggacagagcuacauauuauaguaauauacuuaacaaaaaugauaaaaguuuauacaacaauuacauuuuggauucuuugcagguuucaauuccuucacuagaaagaucgcuuagagaauacauucaaaauccaacugacgaaccauuugacauuaaguccguaccuguagcagcagugccaacagcagaagaacgagaaguuaaaaacaaaucugaaggacugcuagucucucaagguccaguccgaccuccuccggugucuagagaagaaaacuucgccgaaaaacuuaguaacguuccggguauacaacaguuaggaccuuuguucaaaacuuccgacgucguugaacucacugaaucugaaacagaguauuuuguccgcuguaucaagcac

SEQ ID NO: 127 - RNA, 500 nt, Diabrotica virgifera
uacuaggcgaaagugccgucgguaagucgaguuugguacugagguucgucaaaggacaguuccacgaauaccaggagaguaccauaggagcagcuuuccuuacacaaaccauaugccucgacgauacaacuguuaaauuugaaauuugggacacagcgggucaagaaagguaccacaguuuagcuccuauguacuauaggggcgcacaggcagcuauagucgucuacgacauaaccaaucaagacacauucggcagggcgaaaacgugggugaaggaacuucaaaggcaggccaguccgacgaucgugauagcuuuggccggcaacaagcaagauuuggccaacaaacguaugguagaauacgaagaggcgcagacguaugcugacgaaaacggcuuacuuuuuauggaaacuuccgcaaagacggcaaugaacgucaacgauauauuuuuagcaauagcuaagaaacugcccaagaaugaacaaaccacaggucaaggc

SEQ ID NO: 128 - RNA, 189 nt, Diabrotica virgifera
cgaugagguugaaggaaggagaaaaauuuugauggggcgaaaaagcauuaccaggacauaucuucguggaaaugcuguuccugcguaugugauaauaauccuuguaggaauuggucaaaucauccugggagggauauuguacguugcauugaggaagaagaucauugcugcaccuguaacggcaucaua

SEQ ID NO: 129 - RNA, 393 nt, Diabrotica virgifera
ugaggucgagcccucagauacuaucgaaaaugugaaagcuaaaauccaggauaaagaaggaauucccccagaccagcaacgucucaucuucgcuggaaaacaacucgaagauggucguaccuugucugacuauaauauucaaaaagaaucaacccuucacuugguguugagauugagaggaggugcuaagaaacguaagaagaagaauuacuccacccccaagaaaaucaagcacaagaagaagaagguuaaguuagcuguauugaaauuuuauaagguugacgaaaaugguaaaauccaccgauugagacgugaaugccccgcugaacaauguggagcuggugucuucauggcagccauggaagacaggcauuacuguggcaagugcgguua

SEQ ID NO: 130 - RNA, 524 nt, Diabrotica virgifera
ucuuugccuuuggcuacgauucgaggagggcauucaagaauggcuccagaaauuggauucagaacaauuuuacaucggaguauauguacuuauagucgcuucacugaucgucaugauuguguccuuuauaggauguauuagugcccugcaggagaguaccauggcccuuuuaguguacaucggcacccaagugcucaguuuuauauucgguuuauccgguucggcgguucuucuggauaacagcgccagagauucccacuuccaaccgaggauccgagagaguaugcgacgucuuaucaugaaugcucaucacgaccaauccagacaaacacuagccaugauucaggaaaauguugguugcugcggagcugauggcgcaacagacuaccucucucuucagcagccccuuccaagucagugcagagacaccguuacuggaaacccauucuuccacggauguguagaugaacucaccugguucuucgaagaaaaaugugguuggauagcagguuuagcuauggcga

SEQ ID NO: 131 - RNA, 267 nt, Diabrotica virgifera
ucgugcugguuuacaauuuccuguaggucguauucaucguuuauugagaaaaggaaauuaugccgaaagaguuggugcuggagcuccuguauacuuggcagcuguuauggaauauuuagcugcugaaguuuuggaauuggcaggaaaugcagcuagagauaacaaaaagacccguauaauuccuagacauuuacaauuggccauaagaaaugacgaggaauugaacaaauuacugucaggaguuaccaucgcccaagguggaguauu

SEQ ID NO: 132 - RNA, 558 nt, Diabrotica virgifera
gugcaugaaguuggaugguguagaucugcccccaccaauuagcuucgacauugcggaagagcaaccguuaccaccuugccaacagacguucuuauguaauggugauggaggauccauagugcgacaguuucucgagcuguauuucguaauauaugauucagauaauaggcagucccuucuucaggcauaucacgaaaaagccacauuuucaaugacaauggccuacccguacggcuauuccaaagacaguaaaggaguaucgugguugaauugguaugccaccgauaauagaaauuuauuacgaguucaagauccagacagaagaaacaaguuguuaagacagggacaaguugcuguaguuucguucuugcaagauaugccgcacacgaagcacgauauucacaguuuuacaguagauuugacaguuuuuacaccccagauguuauguuugacaguggcugguauguuuaaagaauugaaaaguggccacaaaguaccuccuuuaagauauuucuucagaacccuuguaauuguaccugcuggaucagguuuuugcau

SEQ ID NO: 133 - RNA, 582 nt, Diabrotica virgifera
gauucggagcccuacaaugaaaugcaacuaaaaauggauuuagaaaagggugagguuaaaguaaaaauaagagcauuagaaaaaauaauucacaugauucuggcaggagaaagguugccgaauggauuucuaaugaccaucauaagaaacguuuuaccuuuacaagaucauuuggcaaaaaaacuauuauugauuuucugggaaauaguuccaaaaacaaauccagaagguaaacuacuacaagagaugauuuugguaugugaugccuauagaaaagaucugcaacacccaaaugaauuuuugagagguucuacacuucgcuucuugugcaaacugaaggaaccagaauuguuggaaccauuaaugcccaguauuagagcuuguuuggaucauaggcacagcuaugugaggaggaaugcuguacuggcaauuuuuaccauuuacaaaaauuuugaagcccucauuccagaugcuccugaacugaucuccaauuauuuggauggugagcaagacaugucuuguaaaagaaaugcguuuuuaaugcuucuucaugcugaccaagaaagggcguugucguauuug

SEQ ID NO: 134 - RNA, 458 nt, Diabrotica virgifera
uuggcaaaaaggaaaugaggauauugaugguaggacucgaugcagcugguaaaaccacaauuuuauauaaacuuaaauuaggagaaauuguaacaacuauuccaacaauuggauuuaauguggagacuguagaauauaagaacauuaguuuuacaguaugggauguagguggucaagauaaaauuaggccauuguggagacacuauuuccaaaacacacaaggccuaauuuucguaguagacaguaacgacagggaacguaucacugaggcuaaagaugaauuaaugcguauguuggccgaagaugaacuuagagaugccguacuucucauuuucgccaacaaacaagauuugcccaaugcaaugaacgcugcagaaaucaccgacaaacucggucuccauucacuacgcaaccgcaacugguacauucaagcuaccugugcaacuagcggagauggu

SEQ ID NO: 135 - RNA, 592 nt, Diabrotica virgifera
ggucgcgaaagaacagaaagauuuuuugaccguaucugccgauggaugcguacaaguaacaaaaccucucgaccgagauccgccuuucgguagcccaacacgacaagucuucaucuaugcucgugauaaugauggaggcacaaauucauuguuggccacugcagaaauugaaauuauuuuaauagauauaaacgauaaugcucccuuuuuaaauguuacagaaauuguuuauuaugaaaaccaggauccagguuuuauagguaaccuaagugccgaugauuacgaugguccugauaauggaccuccguuugcuuuucgauuaucagacacugcuucagauaguauuagaucgaaauuuuccauuaucggaaaccagcuuuucgcuuuagaaauguuugauagagaagagcaaaaauauuaugacauugccauugacauuacagauaguggaguaccuccacuaacaggaacuaguauucuuagaguuauaaucggagauguaaaugauaauccagcuacagacggaaacagcacgaucuuuguguauaaguacgucaaugggccagaaaauuucauggaaaucggacgugua

SEQ ID NO: 136 - RNA, 214 nt, Diabrotica virgifera
gcaugggguaucauccaguuggguuucauggguguauucuauuacauuggggcuguggcuuuagcagaagauauuccagagguugaguuuaagggcgauuuagacaaauuuuauagcgacgucaacacggguuucacacagaaugcuuacaacugcuggauugcugcucuccuauaccugauaacauuagcaguaucagcucaccaauucuggg

SEQ ID NO: 137 - RNA, 459 nt, Diabrotica virgifera
uagaugccgcagguaaaaccacaaucuuauacaaauugaagcuuggugaaaucguaacuacaauaccaaccaucggcuucaauguagaaaccguugaguacaagaauauaucuuucacgguaugggauguagguggccagacgagaaucagaaaacucuggagacacuauuucgccaacacugauggacucauuuuugugguugauuccaacgaccgagaccguaucgcggaagccgaagaagaauugcacaauauguuaggagaggacgauuuaagagacugcauuuuguuaauauucgccaacaaacaagauuuaccgaacucgauguccacugcugaauugaccgauaagcuuaaguugcacacuuugaagaauaggaggugguacauacaagccacaugugcuacucaagggaaugguuuguacgaaggacuagauugguugucgaaugaauugg

SEQ ID NO: 138 - RNA, 536 nt, Diabrotica virgifera
gcuacccuagcguccacuguaacacugauuuuugcccucuacuacugccucacgggaaaaggagagcaaguuaguuuagcaugguuauuguugaaugugucuccccacaugugggcaggucuaggaauuggccuugcuguaucauuaucaguuguaggagcugcugcaggaauucacacuacaggagucaguaucguaggagcugguguuaaagcccccagaaucaaaaccaaaaauuuaauuucuauuauuuucugugaagcuguggcuaucuauggguuaauuauggcuauaguacucuguggaaguuggaagaauuucgauguagaccuauucaaccucaaaacucauaacuuugcucaaaaccauuauggaucacauguuauuuuuggauccgguuuaacuguuggauuuguaaaucuauuauguggauuuuguguuggaguaguugguucuggugcagccauuucugaugcagccaauucaucauuauucgucaaaauuuugauuauugagauuuuuggaagugccauugg

SEQ ID NO: 139 - RNA, 547 nt, Diabrotica virgifera
accuuaugggaaagcguguggacuuuucugcacguacugucaucacaccagaucccaauuuacguaucgaccaaguaggagugccuagaaguauugcucaaaacaugacguuuccagaaaucgucacaccuuucaauuuugacaaaauguuggaauugguacagagagguaauucucaguauccaggagcuaaguauaucaucagagacaauggagagaggauugauuuacguuuccacccaaaaccgucagauuuacauuugcagugugguuauaagguagaaagacacaucagagacggcgaucuaguaaucuucaaccgucaaccaacccuccacaagaugaguaugaugggccacagagucaaagucuuacccuggucgacguuccguaugaaucucucgugcaccucucccuacaacgccgauuuugacggcgacgaaaugaaccuccaugugccccaaaguauggaaacucgagcugaagucgaaaaccuccacaucacucccaggcaaaucauuacuccgcaagcuaaccaacccgucau

SEQ ID NO: 140 - RNA, 564 nt, Diabrotica virgifera
cggaucucuauuugggggaucaagucguauugaagaugcaguggaauguuacacaagagcugcaaaccuuuuuaaaauggccaagagcugggaugcugccgguaaagccuuuugugaggcugcuaauuugcauuccagaacuggugcucgucaugacgcugccacuaauuauauagaugcugcaaauuguuacaaaaaagccgauguauuugaggcuguaaacugcuuuauaaaagcuauagacauuuauaccgaaaugggucgcuuuacaauggcugcaaaacaccaucagacuauugcagaaauguaugagacugaugcuguggacaucgaaagggcuguucaacacuaugaacaggcggcugauuacuucagaggagaagaaagcaaugcuuccgccaauaagugucuucuuaaaguggcucaauaugcagcccaacuugaaaacuaugaaaaagcagugggaauuuaucaagaaguggcuuaugcagcucuggaaagcucucuuuuaaaauacagugcaaaggaauacuuauucagagcugcccuuugucaccuuugugu

SEQ ID NO: 141 - RNA, 564 nt, Diabrotica virgifera
ugcugauuggagauucaggaguaggaaaaucuugucuucuacugagauuugcagaugauaccuacacagaaagcuauauuaguaccauuggcguagauuuuaaaaucaggacaaucgauuuagauggaaagacaauuaaauugcaaauuugggauacagcaggucaggaaagguuuagaacgauuacaucaaguuauuaccgaggagcacaugguauuauuguaguguacgauugcacagaccaagauucauucaauaacguuaaacaguggcucgaagaaaucgaccguuaugcgugugacaauguaaacaaauuacugguagggaauaaaagcgauuugacaacuaagaaaguugucgacuucacuacagccaaggaguaugccgaccaauuggguauaccauuuuuggaaaccucagcuaagaaugcaaccaauguagaacaggccuuuaugacuauggccgcugaaauaaaaaauagaguaggaccuccaucuucugcgguagaccaaggaaauaagguuagguucgaucaaagucgcccagucgaaacaaccaaauccg

SEQ ID NO: 142 - RNA, 502 nt, Diabrotica virgifera
gagcuauaguggacugcggguucgaacauccuucagaaguucaacaugaauguauuccucaagcugucauuggcauggauauucugugccaagcuaaauccgguaugggaaaaacggcuguuuuuguauuagcuacacuccaaguaauagauccuacagaaaauguuguauauguucucgucaugugccauaccagagaguuagccuuccagauaagcaaagaguacgaacguuucaguaaauauaugcccaauauuaaaguaggggucuucuuugguggcuugccuauccagaaagaugaggaaacguuaaaaaauaauugcccgcauaucguuguggguacuccaggaagaauuuuagcauuggucagaucgaaaaaacuuaaucucaaacaucuaaagcauuuuauuuuggaugaaugugauaaaauguuggaguuauuagacaugagacgugauguucaagaaauauaucguaacacuccccacgaaaaacaagucau

SEQ ID NO: 143 - RNA, 505 nt, Diabrotica virgifera
ugguagauuuggcuaaccucguauauugguguuuaggucuuaauauuccguacguuaguuucuaugauuauaaagguaauuuaaaaaagcaugaagagaaguugcaacaaauuguagaauccagaaaaucagagaauaucaacauaauuuggcacacccaugcagaacaaaggcauaaaaauggauuuuuggguccaaaaauccacguaaaaguguuaacacacgcggacggaaagcaaaguauaguaaauguuacuaaaaaauuagcucuaaauaaagaaaaagacauuaguaaagaaaaaauuagugaauuacuauuaaggcaguaugaauuuccagauccagaaauggcuauuauuuguggaaagaaacugaacauuuauaauuauccuccuuggcaguuaagacucacagaauucuuuaaagucaacaaagucaacaacaucacauucccaguguuuguggaaaaauuggaaaaguacagcaaaugugaacagaggguggg

SEQ ID NO: 144 - RNA, 410 nt, Diabrotica virgifera
aaccagggaucuauuugcccgcaaguuuaaaaaacgugguguaauuccacuuuccacauauuugagagucuacaaaguuggagauauuguagauaucaaggguaauggugcaguucaaaaggguaugccccacaaaguguaccaugguaagacaggacguguuuucaauguuacugcacaugcauuagguguaauuguaaacaaaaggguucgaggaagaaucauccccaaaagaaucaaucuccguauugaacauguaaaccacuccaagugucgucaagacuucuugcaaagaguaaaauccaacgaaaagcuacguaaagaagcuaaagaaaagaacauuaaaguagaacuuaggagacaaccugcccaaccuaggccagcacauauuguuagcggaaagguuccag

SEQ ID NO: 145 - RNA, 467 nt, Diabrotica virgifera
ugaugguaauagagugcuggcuaaauacuacgauaaagauauauuuccuacagcaaaagagcagaaagcuuuugagaaaaauuuguucaauaaaacucauagggcagacgcagaaauuaucauguuggaugguuuaacuuguguguauagaaguaauguagauuuauucuuuuauguuaugggcaguucacaugaaaaugagcuaauuuuaaugaguguuuuaaauugcuuguaugacucaguaagucaaauauugaagaaaaauaugcaaaaacgagcugucuuggaaucacuagauauuguuaugcuggcuauggaugaaauuguugauggaggaauaauuauagauucugauucaaguucaguaguaucuagaauagcauuaaggacugaugauauuccauuaggagaacaaacuguagcucagguauuccaaacggccaaagaacagcugaaauggucauugc

SEQ ID NO: 146 - RNA, 368 nt, Diabrotica virgifera
caccgaagaacaaauugcugaauucaaagaagcuuucucacuauucgauaaagauggugaugguacaauuacgacuaaagaauuaggaacaguaaugagaucucuaggacaaaauccaacagaggcugaauuacaggauaugaucaaugaaguagaugccgaugguaacggcacgaucgauuucccagaauuuuuaacgaugauggcacguaaaaugaaagauaccgauagugaggaagaaauucgugaagcauuccgaguguucgacaaagacggcaaugguuucaucucagcagcagaauugcgccacgucaugaccaacuugggugaaaaauugacagacgaagaagucgaugaaaugauucggg

SEQ ID NO: 147 - RNA, 430 nt, Diabrotica virgifera
uucgcacaagaugacuuugguggugaaaauguugaucuauaugacgauguaauauccgcuccuccuggaaauaaugacaacccaggugauucaaaucaucaugcuccuccuggugcuggugaagaugguggagguaauuuuguugggucaggaggagcacccaauaauauaaauucuucuggaagaagacaucagcuguauguuggaaaucugacuugguggacaacugaucaagauauagaaaaugcagugcaugauauagggguaaccgacuuccaugaaguuaaguuuuuugaacacagagcaaauggucaauccaagggauucugugucauaucuuugggaucugagggaagcaugagacucugccuggaacuccuaucuaaaaaagagaucaauggccaaaauccccuuguuacccuucccacaa

SEQ ID NO: 148 - RNA, 505 nt, Diabrotica virgifera
uaggaauggcauuuucaggcuuaauagcugaugcaaggcaaaucguugagauugcuagaaaagaagcaucaaauuauagacaucaauaugguucaaauauuccucuuaaauaccuaaaugauagaguaagcauguacaugcaugcauacacuuuauacagugcuguuagaccauuugguugcagugucaucuuggccaguuaugaagauagugacccaucuauguaucugauugauccaucuggaguuagcuauggauacuuuggaugugcuacagguaaagcaaaacagucugcaaagacugaaauagaaaaauugaagauggggaaucuaacaugcaaagaacuuguuaaagaagcagccaaaaucauuuauuugguccaugaugagcugaaggauaagaauuuugaacuggaacuuucauggguaugcaaagauacgaaugguuuacauaccaaagugccugaaucaguguuugcugaugcagaaaaagcugccaaacaagc

SEQ ID NO: 149 - RNA, 548 nt, Diabrotica virgifera
uccaucuagaggagcccaaaugaugaugaaauccaggcuaaagggagcccaaaagggacauaguuuauuaaagaagaaagcugaugcuuuacaaaugagauuuagaaugauuuugaacaaaauuauugagaccaaaacucucaugggugaaguaaugaaagaagcugccuuuucuuuagcugaagcaaaguuugcaacuggugacuucaaucaaguuguucuucaaaaugucaccaaggcucaaauaaaaauaagaacuaagaaagacaacguugcugguguuacuuuaccaguguuugaaugcuaccaagaugguacagauacauaugaguuggcugguuuggcuaggggaggucaacaauugacaaaacucaagaagaauuaucaaagugcuguuaaacuguugguugaauuagccucuuugcaaacuucuuuuguaacucuugaugauguaaucaaaauaacaaacagaagagucaaugccauugaacauguuaucauuccaagaauagagcguacuuuggcuuacaucauauccgaacuggacg

SEQ ID NO: 150 - RNA, 521 nt, Diabrotica virgifera
gaucuggaagcgcuaguugcaaaaguagacgaaaugagaacccaaagagccaugcuaugggcucaacuucgagaaucuauucaccaagacgauauuacaaguucccuuguaacgaaacaaccaaaucagucgcuggaacagcuguuccagcaagaacuucaaaagcaucaaaaucugauuucguugauugaacaaaacaccucggcacaagaaaacauuaagagcgccuuagucgauucuuacgcuuacgcuguaaauucaagaaaauacauccaagauauacuccaaaagagaaccacaaccauaacgucacugauagcaucguucgacucuuacgaagacuuauuggcaaaagcuaacaaagggauagaguuuuacucaaaacuugaaacgaacguauccaaguuacugcaaagaauaaggaguaccugcaaaguucaacaagaagagcgagaucagaugaugucgacugcgcaagugccucaaugggagagucauacgucacuugccgcuccuaaacu

SEQ ID NO: 151 - RNA, 633 nt, Diabrotica virgifera
auggcggacgaugagagaaagaaacuggaggaggaaaagaagaggaaacaggccgaaauugaacgcaaaagggccgaggucagggcucguauggaagaggccucaaaagccaagaaggccaagaaagguuucaugaccccugagagaaagaagaaacuuagguuacuguugagaaagaaagccgccgaagaauuaaagaaagaacaagaacgcaaagcagccgaaaggaggcguaucauugaagaaaggugcgguaaacccaaacuugucgaugacgcaaaugaaggcccauuaaaacaaguaugugagggauaucacagacguauuguagaccuagaaaauaagaaauuugaccucgaaaaagaaguggaauucagagauuuucagaucuccgaauugaacagccaaguaaacgaccuuagaggcaaauucgucaaaccaaccuugaagaagguauccaaauacgaaaacaaauucgccaaacuucaaaagaaggcagcugaauuuaacuuccguaaccaacucaaaguugucaagaagaaagaauucaccuuagaagaagaagacaaagaaaagaaaccagacuggucaaagaagggagacgaaaagaagguacaagaggcugaagcauga

SEQ ID NO: 152 - RNA, 603 nt, Diabrotica virgifera
augugugaagaagaaguugccgcuuuagucguagacaauggauccgguaugugcaaagcugguuuugcuggggaugaugcaccucgugcuguauucccuucaauuguuggacgcccaagacaucagggugugaugguaggaaugggacaaaaagauuccuauguaggugaugaagcucaaaguaaaagagguauccuuaccuuaaaauaccccaucgagcacggaauagucacaaacugggaugauauggagaaaauuuggcaucauacauucuacaaugaacucagaguagccccagaagaacacccuguucuguugacagaagcuccucucaaccccaaggccaacagggaaaagaugacacaaauaauguuugaaacuuucaacaccccagccauguauguugccauccaggcuguacucuccuuguaugcaucuggucguacaacugguauuguguuggauucuggugaugguguaucccacacugucccaaucuaugaagguuaugcucuuccucaugcaauccuucguuuggacuuagcugguagagacuugacugauuaccucaugaaaauuuugacugaacguggcuacucuuuc

SEQ ID NO: 153 - RNA, 2742 nt, Diabrotica virgifera
augccacuucgauuagauauaaaaagaaagcuaacagcucgcucagaccggguaaaauguguggaucuucacccuacagaaccuuggaugcuguguucucuuuacagcggaaauauaaacguuuggaacaccgaaaaucagcaacugguuaagacuuuugaaguaugugauguaccuguucggacagcuaaguuuuugcccaggaagaacuggauagucagugggucugaugauaugcagauucgaguuuucaauuacaauaccuuagaucggguacauucuuuugaggcucauucggauuaugugagauguauugucguacacccuacacaaccuuauauauuaacaaguagugaugauaugcuuaucaagcuuuggaauugggaaaaagcaugggcuugucagcaaguuuucgaaggacacacucauuauauuaugcaaaucgccauaaauccaaaagacaacaacacauuugccagugcaucccuagauagaacauugaaaguauggcaauugggagcguccacagcgaauuucacacuagaaggucaugagaaaggcguuaacuguguggacuauuaucacgguggagauaaaccuuauuuaaucucaggcgcugaugauagauuaguaaaaaucugggauuaucaaaacaaaacuuguguucaaacuuuggaaggacaugcucaaaauguaaccgcugcauguuuccauccagaacuuccuguagcucuuacuggaagugaagaugguacugucagaguguggcaugccaacacccauagguuagaaaguagcuuaaauuauggcuuugaaagaguauggacuauuuucugccuaaagggauccaauaacguggcauuggguuaugaugaagguagcauuuugguuaaaguugguagagaagaaccagcuguuaguauggaugccaguggaggcaaaauuauuugggccagacacucugaacuucaacaggcaaaucucaaggcguuagcugaaggugcggaaauaagagauggagaacgccuuccaguuucuguaaaagauaugggugcuugcgagauauacccucagacaauucaacacaaucccaauggccguuuuguuguugucuguggggauggagaauacauaaucuacacagcaauggcuuuaagaaacaaagcguuugguagcgcacaagaauuugugugggcucaagauuccagcgaauaugccaucagagaauccggaucuacuaucagaauuuuuaagaauuucaaagagaagaagaauuuuaaguccgauuuuggagcugaagguauauacgguggauaccuuuugggagucaaaucgguuucugguuugacuuucuaugauugggaaacucucgauuuagucagaagaaucgagauacaaccaaaagcaguuuacuggucagauagugguaaauuaguauguuuggccacagaagauagcuacuuuauucuuucuuaugauucugaugaaguucaaaaagccagagauaacaaucagguugcggaugauggaguagaaucggcuuucaaucuucuaggugaaauaaacgaaucagugcgaacuggucucuggguaggcgacuguuuuaucuacacgaauucuguuaaucguaucaacuacuucguuggaggugaacugguuacaauugcucauuuggaccggccuuuguaugucuugggauaugugccuaaagacgauagauuauaccucguagauaaagaguugcgcguaguaagcuaccaauuacuucuuucuguucuugaauaucaaacugccgucaugagaagagacuuuccaacagcagacagaguacuuccguccauuccuaaggagcacagaacgagaguggcacauuucuuagaaaagcaaggcuucaaacagcaagcuuuggccguaaguacagauccagagcacagauucgagcuggcaguagcauuagaggaucuuaauauagccaaaacucuagcucaagaagcgaacaguccgcaaaaguggaaucaacuagcagaauuggcagcugcuacuaauaauguaagcguagccaaggaauguaugcaaaaagcgcaagauuauggaggcuuguugcuucuugcuacgagcuccggugaugaaaauuuaguccguacucuaggagaaacgacacaagcugaaagcaaacauaacuuagcauuuuugucacacuuguuaguaggugauuuaaacaaaugucuagacauucuuauuaauaccgguagauugccagaagcugcauuuuucgccagaucuuaccuuccugauaagauuacagaagucguggaacuguggaagacucaguuaucuucagucaaucaaaaagcuggacagagccuugccgauccuaaaaacuacgaaaaucuguucccugguuuacaagaggcggugguagcucagaaauuuuuggaacagcagaauaaagguuuagcgcccgcaagaguugccaccaccauuccuccuaaucacgacaggaauguuguagccgaaguucaagcacaaucgaaacacgauguaccaucauuuaguucuucguuuauuucaucagaaauagaagcacaaacaaggaguucugcuaaaccugaagaaucuucaaacauuauacagcuggaccaagaugacgacgauaucgauuuagauuuggacgguguaaauaucgaugagaacauugacacgacggauaucaacaucgaugaugauuugcugagugauuga

SEQ ID NO: 154 - RNA, 1146 nt, Diabrotica virgifera
augcagaucuuuguaaaaacacucacugguaaaaccaucacccucgagguugaaccaucagauaccaucgagaaugucaaagcuaaaauucaagacaaagaagguauuccaccagaucaacagagauuaaucuuugcuggaaagcaguuagaagauggccguacucucucagacuacaacauucagaaagaaucuacacuacacuuagugcuucgucuuagaggagguaugcacaucuuuguaaaaacucucacugguaagaccaucacccuugagguugaaccaucagauaccaucgagaaugucaaagcuaaaauucaagacaaagaagguauuccaccagaucaacagagauuaaucuuugcuggaaagcaguuggaagauggccguacucucucagacuacaacauucaaaaagagucuacccuccauuugguacuucgucuuagaggagguaugcagauuuuuguuaaaacuuuaacuggaaagaccaucacccuugaaguagaaccuucugauaccaucgaaaaugucaaagccaaaauucaagacaaagaagguauuccaccagaucaacaaagauuaaucuuugccggaaagcaauuggaagauggucguacacucucagacuacaacauucaaaaggaaucuacccuccauuugguacuucgucuuagaggagguaugcaaaucuuuguaaaaacacucacugguaagaccaucacccucgagguugaaccaucagauaccaucgagaaugucaaagcuaaaauucaagacaaagaagguauuccaccagaucaacagagauuaaucuucgcuggaaagcaguuggaagauggccguacucucucagacuacaauauucagaaagagucuacccuccauuugguacuucgucuuagaggagguaugcaaaucuuuguaaaaacucucacugguaagaccaucacccucgagguugaaccaucagauaccaucgagaaugucaaagcuaaaauucaagacaaagaagguauuccaccagaucaacaaagauuaaucuuugccggaaagcaguuggaagauggccguacucucucagacuacaacauucaaaaagagucuacccuucacuugguacuucguuuaagaggaggaaauuaa

SEQ ID NO: 155 - RNA, 1131 nt, Diabrotica virgifera
augugugacgacgauguagcggcucuugucgucgacaauggcuccggaaugugcaaagccgguuucgccggugaugacgccccucgugcugucuuuccauccaucguaggucgucccagacaccaaggugucaugguggguaugggucaaaaagacuccuacguaggagacgaagcccaaagcaaaagagguauccucaccuuaaaauaccccauugaacacggaauuaucacuaacugggacgauauggaaaagaucuggcaucacaccuucuacaaugaacuuagaguagcccccgaagaacaucccauucuuuugacugaagcuccacuuaacccaaaagccaacagagaaaagaugacucaaaucauguuugaaacuuucaauaccccugccauguauguugccauucaagcuguauugucucuguacgcuuccggucguaccacugguauuguacuugauucuggagaugguguaucccacacaguacccaucuaugaagguuacgcucucccacacgccaucuugcguuuggacuuggccgguagagacuugacugacuaccuuaugaagaucuuaaccgaaagagguuacucuuucaccaccacagcugaaagagaaauaguucgugacaucaaggaaaaauugugcuauguagcuuuggacuucgaacaggaaauggccacagcagccagcuccaccuccuuagaaaagaguuaugaacuuccugacggucaagucaucaccauugguaaugaaagguuccguugcccugaagcucucuuccaaccuuccuucuuggguauggaaucuugcgguauccacgaaacugucuacaacuccaucaugaagugcgaugucgacauccguaaagacuuguacgccaacacuguccuuucuggagguaccacaauguacccugguauugccgaucguaugcaaaaggaaaucacugccuuggcuccaucaaccaucaaaaucaagaucaucgcucccccagaaagaaaguacuccguuuggaucgguggcuccaucuuggccucccucuccaccuuccaacagauguggaucuccaaacaagaauacgacgaauccggcccuggaauuguucaccgcaaaugcuucuaa

SEQ ID NO: 156 - RNA, 2640 nt, Diabrotica virgifera
auggguacuuuuaaaagagauacucaugaugaggacgggggaucaagugcuuuucaaaaucuggagaaaacuacuguuuugcaggaagcuagaguuuuuaaugaaacuaguguaaauccaagaaaauguacaccgauacuaacaaaacuguuguacuuauugaaccagggugaaacuuuaagugccaaagaggccacagauguuuucuuugccaugaccaaacuguuccaaucaaaagauguaauauugagaaggaugguuuauuugggaauuaaagaacucaguucuguugcugaugaugucauuauuguaacauccagucuuacaaaagauaugacugguaaagaagacauguacagagcagcugcuauaagagcauuaugcaguauuacugaugcuacuaugcuucaagcuauagaacguuauaugaagcaagcuauuguagauagaaacgcagcugucaguucagcagcacuaauuaguucauuacauaugagcaaauuagcuccagauguaguaaaaagauggguaaaugaagcucaggaagcaguaaauagugauaaugcaaugguacaguaucacgcauuaggucuucuauaccauauuaggaagacugauaagcuagcagugacaaaauugauuuccaagcugaauucaauggguuuaaagagcccuuaugcuuuguguauguugauaagaaucacugcaaaacuuuuagaagaagaggaccaagagucacuccucaacuccccauauacaauaauauuuacaaugggcuuaaggaacaaaucugaaauggugguguaugaagcugcacaugccaugguuaaccugaaguucacgaguaguaaugugcuagcacccgcuauaaguguucuacaacuauuuuguggaucuccuaaagccacacucagauuugcugcuguuagaacuuuaaaucaaguggccaccacccacccugcgucagugacagcuuguaauuuggaucuagaaaauuugauuacugauccuaauaggucaauugcuacacuggccauuacuacucuuuugaaaacaggugccgaaucuucuguugacagacuaaugaaacaaaucgcuacuuuuguaucugaaaucagugaugaauuuaaagugguugucauucaggcaauuaagguauuagcuuugaaauuuccaaggaaacauagcacgcuuaugaauuuccuauccgccauguuaagagaugagggagguuuagaauauaaagcauccauagcagauaccauuauaacccuaaucgaagauaaucccgaagcuaaagaaucugguuuggcgcaucuuugcgaguucauugaagacugugaacauguuucuuuggcugugagaaucuugcauuuguuaggaaaggaaggacccaagaccaaacaaccaucgagauacauccguuuuaucuacaaucgcgucauauuggaauguccuucuguaagagcugcugcagucuccgccauggcacaauucggagccucuugucccgauuuguuagaaaauauccaaauauuacuuucgaggugucagauggauucagacgaugaaguuagggacagagcuacauauuauaguaauauacuuaacaaaaaugauaaaaguuuauacaacaauuacauuuuggauucuuugcagguuucaauuccuucacuagaaagaucgcuuagagaauacauucaaaauccaacugacgaaccauuugacauuaaguccguaccuguagcagcagugccaacagcagaagaacgagaaguuaaaaacaaaucugaaggacugcuagucucucaagguccaguccgaccuccuccggugucuagagaagaaaacuucgccgaaaaacuuaguaacguuccggguauacaacaguuaggaccuuuguucaaaacuuccgacgucguugaacucacugaaucugaaacagaguauuuuguccgcuguaucaagcacuguuucaaacaucacaucguccuccaauucgauugucugaauaccuugccagaccagcuuuuagaaaacguuagaguggagauagacgccggugaaaccuucgaaauuuuggcagaaauaccuugugaaaaguugcacuauaacgaaaccgguaccacauauguaguaguuaaguugccugaugaugaucuccccaacucuguugguacguguggagccguguugaaguucuuagugaaagauugugauccaucaacgggaauaccagauucugaugaggguuacgaugaugaauauacacuggaagacaucgaaauaacauuaggggaccaaauucaaaaaguaagcaaaguaaauugggcugcagccugggaagaagcugcagcuacuuauguagaaaaagaggauacauacuccuugaccaucaauacgcuaaguggcgcuguuaagaauauuauucaguucuugggauuacagccugcggaaaggacugacagaguaccggaggguaaaucuacgcacacauuacuucuugcugguguauucaggggagguauugacauacuaguaagagcgaaacuagcuuugggcgaauguguuacgaugcaacuaacagucaggucgccagauccugacguugcugagcuuauaacuucaacuguagguuaa

SEQ ID NO: 157 - RNA, 651 nt, Diabrotica virgifera
auggcggcaaacagaacuggaccugcucagagaccaaauggcgcuacccaaggaaagauaugucaguucaaacugguccuacuaggcgaaagugccgucgguaagucgaguuugguacugagguucgucaaaggacaguuccacgaauaccaggagaguaccauaggagcagcuuuccuuacacaaaccauaugccucgacgauacaacuguuaaauuugaaauuugggacacagcgggucaagaaagguaccacaguuuagcuccuauguacuauaggggcgcacaggcagcuauagucgucuacgacauaaccaaucaagacacauucggcagggcgaaaacgugggugaaggaacuucaaaggcaggccaguccgacgaucgugauagcuuuggccggcaacaagcaagauuuggccaacaaacguaugguagaauacgaagaggcgcagacguaugcugacgaaaacggcuuacuuuuuauggaaacuuccgcaaagacggcaaugaacgucaacgauauauuuuuagcaauagcuaagaaacugcccaagaaugaacaaaccacaggucaaggcggcagugcccaaggcaggcggcuagcggagggcgauucgggcgccaaggcacccggaaauuguugcaaguga

SEQ ID NO: 158 - RNA, 279 nt, Diabrotica virgifera
augaaguuuuuaagaucgacagugugcuacauugccaucuuggcaauucucuuuacccucugugccgaugagguugaaggaaggagaaaaauuuugauggggcgaaaaagcauuaccaggacauaucuucguggaaaugcuguuccugcguaugugauaauaauccuuguaggaauuggucaaaucauccugggagggauauuguacguugcauugaggaagaagaucauugcugcaccuguaacggcaucauaugcaguggcuagacaagaaccauaa

SEQ ID NO: 159 - RNA, 474 nt, Diabrotica virgifera
augcagaucuucguuaaaaccuuaacggguaagaccaucacucuugaggucgagcccucagauacuaucgaaaaugugaaagcuaaaauccaggauaaagaaggaauucccccagaccagcaacgucucaucuucgcuggaaaacaacucgaagauggucguaccuugucugacuauaauauucaaaaagaaucaacccuucacuugguguugagauugagaggaggugcuaagaaacguaagaagaagaauuacuccacccccaagaaaaucaagcacaagaagaagaagguuaaguuagcuguauugaaauuuuauaagguugacgaaaaugguaaaauccaccgauugagacgugaaugccccgcugaacaauguggagcuggugucuucauggcagccauggaagacaggcauuacuguggcaagugcgguuacacucuugucuucuccaaaccaggagaugagaaauag

SEQ ID NO: 160 - RNA, 747 nt, Diabrotica virgifera
augauguccaaagcagacacacaggaagaugccuccuucgccaaauuggaaaaucagauugcuaucaucaaauacguaauacucuuuaccaacguuuugcaaugggcucucggugcagcaaucuucgcucuuugccuuuggcuacgauucgaggagggcauucaagaauggcuccagaaauuggauucagaacaauuuuacaucggaguauauguacuuauagucgcuucacugaucgucaugauuguguccuuuauaggauguauuagugcccugcaggagaguaccauggcccuuuuaguguacaucggcacccaagugcucaguuuuauauucgguuuauccgguucggcgguucuucuggauaacagcgccagagauucccacuuccaaccgaggauccgagagaguaugcgacgucuuaucaugaaugcucaucacgaccaauccagacaaacacuagccaugauucaggaaaauguugguugcugcggagcugauggcgcaacagacuaccucucucuucagcagccccuuccaagucagugcagagacaccguuacuggaaacccauucuuccacggauguguagaugaacucaccugguucuucgaagaaaaaugugguuggauagcagguuuagcuauggcgauaugcaugauuaacguccuuaguauuguuuuaucuacgguacucauccaggcauugaaaaaagaagaagaagcauccgauucauacaggagauag

SEQ ID NO: 161 - RNA, 375 nt, Diabrotica virgifera
augucuggacguggcaagggaggcaaaguaaagggaaaagcaaagucccgaucaaaucgugcugguuuacaauuuccuguaggucguauucaucguuuauugagaaaaggaaauuaugccgaaagaguuggugcuggagcuccuguauacuuggcagcuguuauggaauauuuagcugcugaaguuuuggaauuggcaggaaaugcagcuagagauaacaaaaagacccguauaauuccuagacauuuacaauuggccauaagaaaugacgaggaauugaacaaauuacugucaggaguuaccaucgcccaagguggaguauugccuaauauacaagcaguacuguuaccuaaaaaaacugaaaagaaagcuuaa

SEQ ID NO: 162 - RNA, 864 nt, Diabrotica virgifera
augaaguuggaugguguagaucugcccccaccaauuagcuucgacauugcggaagagcaaccguuaccaccuugccaacagacguucuuauguaauggugauggaggauccauagugcgacaguuucucgagcuguauuucguaauauaugauucagauaauaggcagucccuucuucaggcauaucacgaaaaagccacauuuucaaugacaauggccuacccguacggcuauuccaaagacaguaaaggaguaucgugguugaauugguaugccaccgauaauagaaauuuauuacgaguucaagauccagacagaagaaacaaguuguuaagacagggacaaguugcuguaguuucguucuugcaagauaugccgcacacgaagcacgauauucacaguuuuacaguagauuugacaguuuuuacaccccagauguuauguuugacaguggcugguauguuuaaagaauugaaaaguggccacaaaguaccuccuuuaagauauuucuucagaacccuuguaauuguaccugcuggaucagguuuuugcauagcaaaugaagaacuucacauauccaaugcaacuccggaccaagcaaaagaugcuuucaagaccaccguuaauguagcuccggcaccagccccugugauuaccucuccuggacccaguauaccacaacccgcugugccagaugaugcuacaaaacaagaaaugguaaaacagauguccgcaguauccggaaugaaucucgaguggucgcuacagugucucgaagaaacacaaugggacuaccagaaagccauaaugguauuccaaaauuuaaacgcacaagguguuguaccacaagcagcauuuauuaaa

SEQ ID NO: 163 - RNA, 2868 nt, Diabrotica virgifera
augacugcgguagaacaaccuuguuacacacuaauaaacuugccaacagauucggagcccuacaaugaaaugcaacuaaaaauggauuuagaaaagggugagguuaaaguaaaaauaagagcauuagaaaaaauaauucacaugauucuggcaggagaaagguugccgaauggauuucuaaugaccaucauaagaaacguuuuaccuuuacaagaucauuuggcaaaaaaacuauuauugauuuucugggaaauaguuccaaaaacaaauccagaagguaaacuacuacaagagaugauuuugguaugugaugccuauagaaaagaucugcaacacccaaaugaauuuuugagagguucuacacuucgcuucuugugcaaacugaaggaaccagaauuguuggaaccauuaaugcccaguauuagagcuuguuuggaucauaggcacagcuaugugaggaggaaugcuguacuggcaauuuuuaccauuuacaaaaauuuugaagcccucauuccagaugcuccugaacugaucuccaauuauuuggauggugagcaagacaugucuuguaaaagaaaugcguuuuuaaugcuucuucaugcugaccaagaaagggcguugucguauuuggcaucauguuuagaucaaguaaauucauuuggagauauucuacaacuggucaucguugaguugauauauaaggugugucauuccaauccugcggaaagaucuagauuuauuagauguauauauaacuuguugaacucaagcaguccugcugucagguacgaagcugcaggaacuuuagucacccucuccagugccccgacugccguuaaagcugcugcuagcuguuacauugaguuaauuaucaaagaaagugacaacaauguaaaacucaucguuuuggacaggcugauagcacuuaaggagcuuccuaaucacgaaagaauucugcaggauuuaguuauggacauacugagaguacucucugcuccugacuuagaaguccgcaagaagacuuuaagucuagcccuugaauuagucucuucacggaacauagaagaaaugguauuaguauuaacaaaggaagugaguaaaacgguagacagugaacaugaggauacaggaaaguacaggcaauuguuaguaaggacucuacauucguguuccauuaaguucccagauaucgcacguaguguuauaccagucuugauugaauuuuuauccgauaauaaugaacuggcugccacagauguauugcuguucuuaagggaagccauacagaaguuuaaagaauugcaaccguuaauuauugagaaacucaucgaaacuuucaaagacauuaaauuggucaaaguccauagagcagcaauuuggauuuugggagaauacgcgaguacugcuuccgauauagaaguuaucguuggagaaauuaacagauuguugggugaaggaucccucguugaagcugagcagaaguuaauagcaggagauacggaagagaaugcuccugcaccugcugcaggcgccaccacuuuaguuacuuccgauggaacauaugcuacccaaucagcuuucaacacugucagccaaaccacuaaagaagcacgaccuccucuaagacaauaccucauggauggugauuuuuucaucggagccucuuuggcaucuacauuaaccaaacugucuuugcgguaugaggaccucaccucuccugcugcuagcaauggauucaaugccaaaauuaugcuuauuauggcuggaauucuucacuugggaaaaucaggacuucccacaaaaucaauaaccaacgacgauaaagaccacauucuguucuguuuacgaguccuaucugaucguucuccaaucauuguugaaauuuucaaaaaauugugccgcucggcacuaaaugagaugcuucuagcuaaggaaucgguagaagcgaucucgcaaaagagcaaagaaaaaaacaagcguacgauucaaacugacgacgcuauaagcuuccugcaauuagagacagauaaaaguggagagcuaggagaaaacguauucgagaugucgcugucacaagcuuuaguaggaggucgaacgggagguggcgaaucaguauuaaguuccaauaaauuagauaaaaucacacaacugacugguuuuuccgauccaguuuauuccgaagcauacguucacgugaaucaguacgauaucgugcuugaugucuuaaucguaaaccaaacuaacgauacuuuacaaaacugcacgcuagagcuggcuacuuuaggcgauuugaaguugguagagaagccacaaccugucguauuggcgcccaaagacuuuugcaacauuaaagcuaacgugaaaguggccucaacugaaaacggaauuauauuuggcaacauuguguaugaugucauaggagcggggucagauaggaauguuguaguuuugaaugauauacacauagauauaauggacuauauagugccugcuaguuguacagauagcgaguuuaugagaaugugggcggaauuugaaugggaaaauaagguaaccguuaacacaccccucacggaacuuucagaauaccucgaacaucuacucaaaagcacaaauuugaaauguuuaacaucagaaaaagcucugagcgggcagugugguuuuauggcagccaauuuauaugcaaaauccauuuuuggagaagacgcuuuggccaacuuaaguauagagaaaccuuuuaauaaacccgaugcgccaguaagcggucauauuagaauaagggccaaaagucagggcauggccuuaaguuuaggagacaaagucaauaugacacagaagagcacacaacauaaaguaguagcugcauaa

SEQ ID NO: 164 - RNA, 549 nt, Diabrotica virgifera
auggguaauguguuugcaaauuuauucaaaggccucuuuggcaaaaaggaaaugaggauauugaugguaggacucgaugcagcugguaaaaccacaauuuuauauaaacuuaaauuaggagaaauuguaacaacuauuccaacaauuggauuuaauguggagacuguagaauauaagaacauuaguuuuacaguaugggauguagguggucaagauaaaauuaggccauuguggagacacuauuuccaaaacacacaaggccuaauuuucguaguagacaguaacgacagggaacguaucacugaggcuaaagaugaauuaaugcguauguuggccgaagaugaacuuagagaugccguacuucucauuuucgccaacaaacaagauuugcccaaugcaaugaacgcugcagaaaucaccgacaaacucggucuccauucacuacgcaaccgcaacugguacauucaagcuaccugugcaacuagcggagauggucucuaugaaggucuggacugguuguccaaucaauuaaagaacgccaaucgcuag

SEQ ID NO: 165 - RNA, 4425 nt, Diabrotica virgifera
augggacgguugcacuguuuauuuuguauuuuccuauguuuuaccgucaucaacacgcagacaacgaauauacauggauucucggaaaauuccguggauacauuucuaucaccucaugggaaaagugcaaaauucgugcaccaaaaucacaaacccaaaauugaaaauugucagaacuacaaacccucggugaaagaagaacagccaggcggaacguacguaacaacgguuaccgcuaucgaugacgauccuagggagggaggaggaacaauuaguuacaaacuaauucauagagaaggagaacauguuuuauuugacauagacaacguuacugguguuuugacaacuauccagccauuugaucgggaugaaccaguaaggcagaaggaacuuuauguaaccguacaagcuucagacaacggcaggccaccauuagcagaugucuguacauuuacaguuaccauuaccgacauuaaugauaaugcgccacagcuugauaaacugaaauacgaugcacaaguuucugaagauuuaaaaguaggaagugaagugaugagaguuuuugcuuacgacauugaugauggggaaaauucaagauuaucguauaacuuuucaaacgaaaaugcucaauucacccaguauuucaggauagaucgagauacuggcguuguguauuuaaaggaagcuuuaacagacaaaaagaauacuagauuuaacagugcuguuuauguagccgauaauggcguuaacgaucaagaaggccaaaaagauucaaccgcuaagauaucuauaacaguaguagggucugauaaacagccucccagauuuacucaaaaaaugccugauggaaucuuggagauccccgaagauuuuaaagacuuuucuaaacauauugucacagucgaagcaacguccaacauugcggauccacaacuugcuuuugaauuggugaagggaaagacauaucaaaccaauaaagaccaaacguuucuuuuggaggcagaaggaaauaaagcgcacauaaagcuagugcguccacuggauuaugaaacaguaacggaauauacucuaacuauucgaguaaaaaacaaagauuuaauggauucuuccauaaauauaccaauuaaaguauuagauguuaaugaugaaauuccuaauuuccuugaauuucuuaaagguagugucguggaaaaugacaagccaggugcacaagcgauucaaguaagagcaaucgauaaagacggaacugcugcuaacaacauugugagcuaugaacucguugacaauacagauuuguuugcaauaaaccgaucuacgggaguaauuacgucgagaguggaguuugaucgugaaacuguaccucuauaucacguaaacguuaaagcuuaugauaacucuccgucugcuuuguauaacacgacauugccuaacauuguaauucagacauuccaaaucaguauagaagaucaaaaugacaacaaaccuguauuuacucauccaauuuaucaguucaguaauauuacugagcuugcugauaaaucgaguauuguuggugaagucaaagcuuuagauaaugacacggcuucaguuauaaguuauaguauuacaaauggaaauauugacgaugcguuuaugauugaaaauucuaccggcagaauaagaguuaauggaaaacuggauuacgagaaaaucgaacaauacaacuuaaccguucgcgcauuugauggggcauuugaagauuuugcaauuguuuuaauuuccauacuuaaugaaaaugacgaaccuccaguuuuugacgacuauaucagagaaauucaaauuaaagaggaagaaccuaugauauccggaugcguuguuagagugacugcucaugauccagauauuaaagacaggcaugcugaucaacacauaguauaugaggucgcgaaagaacagaaagauuuuuugaccguaucugccgauggaugcguacaaguaacaaaaccucucgaccgagauccgccuuucgguagcccaacacgacaagucuucaucuaugcucgugauaaugauggaggcacaaauucauuguuggccacugcagaaauugaaauuauuuuaauagauauaaacgauaaugcucccuuuuuaaauguuacagaaauuguuuauuaugaaaaccaggauccagguuuuauagguaaccuaagugccgaugauuacgaugguccugauaauggaccuccguuugcuuuucgauuaucagacacugcuucagauaguauuagaucgaaauuuuccauuaucggaaaccagcuuuucgcuuuagaaauguuugauagagaagagcaaaaauauuaugacauugccauugacauuacagauaguggaguaccuccacuaacaggaacuaguauucuuagaguuauaaucggagauguaaaugauaauccagcuacagacggaaacagcacgaucuuuguguauaaguacgucaaugggccagaaaauuucauggaaaucggacguguauauguuacagaccuagacgauugggauuuaaaugacaaagucuuuguucaagaagauaacuuugaugaauuuguguuaaaccagcauaacaacgguaugauucugaugaaaccaacaacggcugagggaacuuaugagguucauuacagggucacugaaacccaugaacccacaauacacgaacauacaguuaaugcaauagucacgauuacaguuaaaguacuuccagaggaagcgguuguaaaaucaggaucaauucgauugagaggaacaacuaaggaagaauucauagaaaauucauugaauggaaagagcaaaagagacauauuacaccaagaacucuccaaaauauuaaauacaucuuuagcgaauguugauguauuuacuguuuuaaauucaccccaccagaauaguucguuuguggauguucgauuuucugcucauggaucuccauauuaugcuccagagaaacucgaaaacaaaguuacagaucaucaaauggagcuugaacaaaaauuagauguggaauucuacaugaucaacguaaacgagugccuuaacgaaacaacguguggagcugaaaacucauguacgaacaaauuaaacauaacacgagaaccagcuguaguguuuacuaacagaacauccuuugucgguguaaaugcauuuauugauccugugugugccgcuuuaccaagagauguuauggaauguuucaacggaggcguccuuaucgaaaacacagcguguaauuguccugcaggauuugaaggaccacauugugaaauccuagcuauaggauuuacaggaacugguugggcuauguauccauccuuugacgcuacaaacaggacugagauuauacugcauauuuuaucacaaacugauaaugguuugauauuuuacaauggaccuuuaaauauaagacaaacuucuuugucuaaagauuauauaucauuagaacuuaaagacggauauccauuacuucaaauuugcaccggcucaagcacucaagaaauuuaucugaaagagcgcauucacaaauugagcgauggaucguuacacaaaauaaaaauaggaucuggauuugacgauauaucccuggaaguagacgacuguggaacaacguguucaauuuggacuaauaaacuacauaaagguguuauccgagcaaauggcccccuucaacugggagguaugaaaaacagauucaccgaucaagaauucaaacgaauuugggaccauuugccaccgacugccacccguuucucugguuguauuagaaauuugacguauaaugaauuuuacuacaaccucggugcaccuucugaugcauuccaagcguaucccgacuguaacuaugcagugaugcaagcugugacuuucgguaucgacuccaauuucuugguugcuauucugguuuguguagcaauuuugauaauucuucuucuggcaguaguuguacauagacguaaacacgacaacuuuaacgaaaaagaaaucgaugauacucgcgaaaacauuaucaacuacgaagaugaagguggcggcgaaugugacaccaacuacgaccugucuguuuuccaucagaacaacauuguggacgaaaaaccauugaugagagacaaccccgauguaccugcagauauaaguggcuuuuuagauaacaagaaagacaacugugauaaagaccccgauaauuugccuuaugacgacguucgccauuaugccuacgagggagacggaaauagcaccggauccuuaucuucucucgcuucauguacggacgaaggagauuuaaaguucaacuacuuaucaaguuuuggacccagauucagaaaguuagccgacauguauggagaagauccaagcgaugaagacucacacgauggaaacgaagaauccuggugcuag

SEQ ID NO: 166 - RNA, 291 nt, Diabrotica virgifera
augccuuucuguggucccaaauugucccucugcggccugauuaucagugcaugggguaucauccaguuggguuucauggguguauucuauuacauuggggcuguggcuuuagcagaagauauuccagagguugaguuuaagggcgauuuagacaaauuuuauagcgacgucaacacggguuucacacagaaugcuuacaacugcuggauugcugcucuccuauaccugauaacauuagcaguaucagcucaccaauucugggccaacaacagaucaucauugaacgucuaa

SEQ ID NO: 167 - RNA, 540 nt, Diabrotica virgifera
augggucuuaccauaucagcaguguuuaauagguuguuuaguaaaaagccuaugagaauuuuaaugguaggauuagaugccgcagguaaaaccacaaucuuauacaaauugaagcuuggugaaaucguaacuacaauaccaaccaucggcuucaauguagaaaccguugaguacaagaauauaucuuucacgguaugggauguagguggccagacgagaaucagaaaacucuggagacacuauuucgccaacacugauggacucauuuuugugguugauuccaacgaccgagaccguaucgcggaagccgaagaagaauugcacaauauguuaggagaggacgauuuaagagacugcauuuuguuaauauucgccaacaaacaagauuuaccgaacucgauguccacugcugaauugaccgauaagcuuaaguugcacacuuugaagaauaggaggugguacauacaagccacaugugcuacucaagggaaugguuuguacgaaggacuagauugguugucgaaugaauuggccaaguga

SEQ ID NO: 168 - RNA, 618 nt, Diabrotica virgifera
augcgguacacuuugaguuacaucggugcuacccuagcguccacuguaacacugauuuuugcccucuacuacugccucacgggaaaaggagagcaaguuaguuuagcaugguuauuguugaaugugucuccccacaugugggcaggucuaggaauuggccuugcuguaucauuaucaguuguaggagcugcugcaggaauucacacuacaggagucaguaucguaggagcugguguuaaagcccccagaaucaaaaccaaaaauuuaauuucuauuauuuucugugaagcuguggcuaucuauggguuaauuauggcuauaguacucuguggaaguuggaagaauuucgauguagaccuauucaaccucaaaacucauaacuuugcucaaaaccauuauggaucacauguuauuuuuggauccgguuuaacuguuggauuuguaaaucuauuauguggauuuuguguuggaguaguugguucuggugcagccauuucugaugcagccaauucaucauuauucgucaaaauuuugauuauugagauuuuuggaagugccauuggucucuucggucugauuguuggaguauacuugacgucaagaggcucuaugguuuaa

SEQ ID NO: 169 - RNA, 5694 nt, Diabrotica virgifera
auggcuaccaacgauaguaaagcuccguugaggacaguuaaaagagugcaauuuggaauacuuaguccagaugaaauuagacgaaugucagucacagaagggggcauccgcuucccagaaaccauggaagcaggccgccccaaacuaugcggucuuauggaccccagacaaggugucauagacagaagcucaagaugccagacaugugccggaaauaugacagaauguccuggacauuucggacauaucgagcuggcaaaaccaguuuuccacguaggauucguaacaaaaacaauaaagaucuugagaugcguuugcuucuuuugcaguaaauuauuagucaguccaaauaauccgaaaauuaaagaaguuguaaugaaaucaaagggacagccacguaaaagauuagcuuucguuuaugaucuguguaaagguaaaaauauuugugaagguggagaugaaauggauguggguaaagaaagcgaagaucccaauaaaaaagcaggccauggugguuguggucgauaucaaccaaauaucagacgugccgguuuagauuuaacagcagaauggaaacacgucaaugaagacacacaagaaaagaaaaucgcacuaucugccgaacgugucugggaaauccuaaaacauaucacagaugaagaauguuucauucuugguauggaucccaaauuugcuagaccagauuggaugauaguaacgguacuuccuguuccuccccuagcaguacgaccugcuguaguuaugcacggaucugcaaggaaucaggaugauaucacucacaaauuggccgacauuaucaaggcgaauaacgaauuacagaagaacgagucugcaggugcagccgcucauauaaucacagaaaauauuaagauguugcaauuucacgucgccacuuuaguugacaacgauaugccgggaaugccgagagcaaugcaaaaaucuggaaaaccccuaaaagcuaucaaagcucggcugaaagguaaagaaggaaggauucgagguaaccuuaugggaaagcguguggacuuuucugcacguacugucaucacaccagaucccaauuuacguaucgaccaaguaggagugccuagaaguauugcucaaaacaugacguuuccagaaaucgucacaccuuucaauuuugacaaaauguuggaauugguacagagagguaauucucaguauccaggagcuaaguauaucaucagagacaauggagagaggauugauuuacguuuccacccaaaaccgucagauuuacauuugcagugugguuauaagguagaaagacacaucagagacggcgaucuaguaaucuucaaccgucaaccaacccuccacaagaugaguaugaugggccacagagucaaagucuuacccuggucgacguuccguaugaaucucucgugcaccucucccuacaacgccgauuuugacggcgacgaaaugaaccuccaugugccccaaaguauggaaacucgagcugaagucgaaaaccuccacaucacucccaggcaaaucauuacuccgcaagcuaaccaacccgucauggguauuguacaagauacguugacagcuguuaggaagaugacaaaaagggauguauucaucgagaaggaacaaaugaugaauauauugauguucuugccaauuugggaugguaaaaugccccguccagccauccucaaacccaaaccguuguggacaggaaaacagauauuuucccugaucauuccuggcaauguaaauaugauacguacccauucuacgcauccagacgacgaggacgacggucccuauaaauggauaucgccaggagauacgaaaguuaugguagaacauggagaauuggucauggguauauuguguaagaaaagucuuggaacaucagcagguucccugcugcauauuuguauguuggaauuaggacacgaagugugugguagauuuuaugguaacauucaaacuguaaucaacaacugguuguuguuagaaggucacagcaucgguauuggagacaccauugccgauccucagacuuacacagaaauucagagagccaucaggaaagccaaagaagauguaauagaagucauccagaaagcucacaacauggaacuggaaccgacucccgguaauacguugcgucagacuuucgaaaaucaaguaaacagaauucuaaacgacgcucgugacaaaacuggugguuccgcuaagaaaucuuugacugaauacaauaaccuaaaggcuauggucguaucgggauccaagggauccaacauuaauauuucccagguuauugcuugcgugggucaacagaacguagaagguaaacguauuccauuuggcuucagaaaacgcacguugccgcacuucaucaaggacgauuacgguccugaauccagagguuucguagaaaauucguaucuugccggucucacuccuucggaguucuauuuccacgcuaugggaggucgugaaggucuuaucgauacugcuguaaaaacugccgaaacugguuacauccagcgucgucugaucaaggcuauggagaguguaaugguacacuacgacgguaccguaagaaauucuguaggacaacuuauccaguugagauacggugaggacggacucuguggagagaugguagaguuucaauauuuagcaacggucaaauuaaguaacaaggcguuugagagaaaauucagauuugauccgaguaaugaaagguauuugagaagaguuuucaaugaagaaguuaucaagcaacugauggguucaggggaagucauuuccgaacuugagagagaaugggaacaacuccagaaagacagagaagccuuaagacaaaucuucccuagcggagaauccaaaguaguacuccccuguaauuuacaacguaugaucuggaauguacaaaaaauuuuccacauaaacaaacgagccccgacagaccuguccccguuaagaguuauccaaggcguucgagaauuacucaggaaaugcgucaucguagcuggcgaggaucgucuguccaaacaagccaacgaaaacgcaacguuacucuuccagugucuagucagaucgacccucugcaccaaaugcguuucugaagaauucaggcucagcaccgaagccuucgagugguugauaggagaaaucgagacgagguuccaacaagcccaagccaauccuggagaaauggugggcgcucuggccgcgcagucacugggagaacccgcuacucagaugacacugaacacuuuccauuuugcugguguauccuccaagaacguaacccuggguguaccuagauuaaaggaaauuauuaauauuuccaagaaacccaaggcuccaucucuaaccguguuuuuaacuggugcggcugcuagagaugcggaaaaagcgaagaauguguuaugcagacuugaacacaccacucuucguaaaguaaccgccaacaccgccaucuauuacgauccugacccacaaaauaccgucauuccugaggaucaggaguucguuaacgucuacuaugaaaugcccgauuucgauccuacccguauaucgccgugguugcuucguaucgaacuggacagaaagagaaugacagauaagaaacuaacuauggaacaaauugcugaaaagaucaacgcuggguucggggacgauuugaauuguauuuucaacgacgacaaugcugaaaaguuggugcugcguaucagaaucaugaacagcgacgauggaaaauucggagaaggugcugaugaggacguagacaaaauggaugacgacauguuuuugagaugcaucgaagcgaacaugcugagcgauaugaccuugcaagguauagaagcgauuuccaagguauacaugcacuugccacagacugacucgaaaaaaaggaucgucaucacugaaacaggcgaauuuaaggccaucgcagaauggcuauuggaaacugacgguaccagcaugaugaaaguacugucagaaagagacgucgauccggucaggacguuuucuaacgacauuugugaaauauuuucgguacuugguaucgaggcugugcguaagucuguagagaaggaaaugaacgcuguccuuucguucuacggucuguauguaaacuaucgccaucuugccuugcuuugugacguaaugacagccaaaggucacuuaauggccaucacccgucacgguaucaacagacaagacacuggagcucugaugagguguuccuucgaggaaacuguagauguauugauggacgcugccagucaugcggaggucgacccaaugagaggaguaucugaaaacauuauccucggucaacuaccaagaaugggcacaggcugcuucgaucuuuugcuggacgccgaaaaauguaaaaugggaauugccauaccucaagcgcacagcagcgaucuaauggcuucaggaauguucuuuggauuagccgcuacacccagcaguaugaguccagguggugcuaugaccccauggaaucaagcagcuacaccauacguuggcaguaucuggucuccacagaauuuaaugggcaguggaaugacaccagguggugccgcuuucuccccaucagcugcgucagaugcaucaggaaugucaccagcuuauggcgguuggucaccaacaccacaaucuccugcaaugucgccauauauggcuucuccacauggacaaucgccuuccuacaguccaucaaguccagcguuccaaccuacuucaccauccaugacgccgaccucuccuggauauucucccaguucuccugguuauucaccuaccagucucaauuacaguccaacgagucccaguuauucacccacuucucagaguuacuccccaaccucaccuaguuacucaccgacuucuccaaauuauucaccuacuuccccaagcuacaguccaacauccccuaacuauucaccaacaucucccaacuauucacccacuucaccuaguuauccuucaacuucgccagguuacagccccacuucacgcagcuacucacccacaucuccuaguuacucaggaacuucgcccucuuauucaccaacuucgccaaguuacuccccuacuucuccuaguuauucgccgucgucuccuaauuacucucccacuucuccaaauuacagucccacuucuccuaauuacucaccguccucuccuagguacacgcccgguucuccuaguuuuuccccaaguucgaacaguuacucucccacaucuccucaauauucuccaacaucuccaaguuauucgccuucuucgcccaaauauucaccaacuucccccaauuauucgccaacaucuccaucauuuucuggaggaaguccacaauauucacccacaucaccgaaauacucuccaaccucgcccaauuacacucugucgaguccgcagcacacuccaacagguagcagucgauauucaccgucaacuucgaguuauucuccuaauucgcccaauuauucaccgacgucuccacaauacuccauccacaguacaaaauauuccccugcaaguccuacauucacacccaccaguccuaguuucucucccgcuucacccgcauauucgccucaaccuauguauucaccuucuucuccuaauuauucucccacuagucccagucaagacacugacuaa

SEQ ID NO: 170 - RNA, 876 nt, Diabrotica virgifera
augucgucaaauauucaaaaggcccagcaguugauggcggaugcagaaaagaaaguaacaucucgagguuucuucggaucucuauuugggggaucaagucguauugaagaugcaguggaauguuacacaagagcugcaaaccuuuuuaaaauggccaagagcugggaugcugccgguaaagccuuuugugaggcugcuaauuugcauuccagaacuggugcucgucaugacgcugccacuaauuauauagaugcugcaaauuguuacaaaaaagccgauguauuugaggcuguaaacugcuuuauaaaagcuauagacauuuauaccgaaaugggucgcuuuacaauggcugcaaaacaccaucagacuauugcagaaauguaugagacugaugcuguggacaucgaaagggcuguucaacacuaugaacaggcggcugauuacuucagaggagaagaaagcaaugcuuccgccaauaagugucuucuuaaaguggcucaauaugcagcccaacuugaaaacuaugaaaaagcagugggaauuuaucaagaaguggcuuaugcagcucuggaaagcucucuuuuaaaauacagugcaaaggaauacuuauucagagcugcccuuugucaccuuuguguugauguacucaaugcacaacaugcuauagaaagcuauauuucaagguaucccgcauuucaagauucccgugaauacaaacuuuugaaaacccucauagaaaacaucgaagagcaaaacguagauggauauacagaagccgucaaagauuacgauucaauuucucgucuugaucagugguauacuacaauucuuuuacguauuaagaaacaaguaagcgaaagcccugacuuacguuaa

SEQ ID NO: 171 - RNA, 609 nt, Diabrotica virgifera
augaaucccgaguaugauuauuuauucaaacuucugcugauuggagauucaggaguaggaaaaucuugucuucuacugagauuugcagaugauaccuacacagaaagcuauauuaguaccauuggcguagauuuuaaaaucaggacaaucgauuuagauggaaagacaauuaaauugcaaauuugggauacagcaggucaggaaagguuuagaacgauuacaucaaguuauuaccgaggagcacaugguauuauuguaguguacgauugcacagaccaagauucauucaauaacguuaaacaguggcucgaagaaaucgaccguuaugcgugugacaauguaaacaaauuacugguagggaauaaaagcgauuugacaacuaagaaaguugucgacuucacuacagccaaggaguaugccgaccaauuggguauaccauuuuuggaaaccucagcuaagaaugcaaccaauguagaacaggccuuuaugacuauggccgcugaaauaaaaaauagaguaggaccuccaucuucugcgguagaccaaggaaauaagguuagguucgaucaaagucgcccagucgaaacaaccaaauccgguugcugcuga

SEQ ID NO: 172 - RNA, 789 nt, Diabrotica virgifera
auggcagacgcugaugaucuauuagauuaugaagaugaggaacagacagaacaaaccgcaacugaaacggcaacuacagagguacagaaaaagggugucaagggcacauauguaucaauacacaguucuggguuuagagauuuucuguuaaaaccagcaauucucagagcuauaguggacugcggguucgaacauccuucagaaguucaacaugaauguauuccucaagcugucauuggcauggauauucugugccaagcuaaauccgguaugggaaaaacggcuguuuuuguauuagcuacacuccaaguaauagauccuacagaaaauguuguauauguucucgucaugugccauaccagagaguuagccuuccagauaagcaaagaguacgaacguuucaguaaauauaugcccaauauuaaaguaggggucuucuuugguggcuugccuauccagaaagaugaggaaacguuaaaaaauaauugcccgcauaucguuguggguacuccaggaagaauuuuagcauuggucagaucgaaaaaacuuaaucucaaacaucuaaagcauuuuauuuuggaugaaugugauaaaauguuggaguuauuagacaugagacgugauguucaagaaauauaucguaacacuccccacgaaaaacaagucaugauguucagugccaccuuaaguaaagaaauuagaccaguuugcaagaaauuuaugcaagauguaauucaaaauucuuauaauacacaauuuuguaaugacgcacccacucgcaauguuuga

SEQ ID NO: 173 - RNA, 738 nt, Diabrotica virgifera
augccggucauugaugguuauaaaguacuuuacauuuuauuacacaguuuauauacaauuuuugaaaauauuuggaggacucuuuuauuuauuuaucaaaauuguauaaggguuauaaacccugaaucuacauucgaugaugcugaccaguuaaagaaaagacugucuagacuaacaaaaaagccucaacauuuaacuaucauuauugguguggaagaauauucauugguagauuuggcuaaccucguauauugguguuuaggucuuaauauuccguacguuaguuucuaugauuauaaagguaauuuaaaaaagcaugaagagaaguugcaacaaauuguagaauccagaaaaucagagaauaucaacauaauuuggcacacccaugcagaacaaaggcauaaaaauggauuuuuggguccaaaaauccacguaaaaguguuaacacacgcggacggaaagcaaaguauaguaaauguuacuaaaaaauuagcucuaaauaaagaaaaagacauuaguaaagaaaaaauuagugaauuacuauuaaggcaguaugaauuuccagauccagaaauggcuauuauuuguggaaagaaacugaacauuuauaauuauccuccuuggcaguuaagacucacagaauucuuuaaagucaacaaagucaacaacaucacauucccaguguuuguggaaaaauuggaaaaguacagcaaaugugaacagagggugggaaaauaa

SEQ ID NO: 174 - RNA, 480 nt, Diabrotica virgifera
augaccaacucuaaagguuaccgccgaggaaccagggaucuauuugcccgcaaguuuaaaaaacgugguguaauuccacuuuccacauauuugagagucuacaaaguuggagauauuguagauaucaaggguaauggugcaguucaaaaggguaugccccacaaaguguaccaugguaagacaggacguguuuucaauguuacugcacaugcauuagguguaauuguaaacaaaaggguucgaggaagaaucauccccaaaagaaucaaucuccguauugaacauguaaaccacuccaagugucgucaagacuucuugcaaagaguaaaauccaacgaaaagcuacguaaagaagcuaaagaaaagaacauuaaaguagaacuuaggagacaaccugcccaaccuaggccagcacauauuguuagcggaaagguuccagcacaggugcuugcuccuaucccauaugaauucauugcuuag

SEQ ID NO: 175 - RNA, 537 nt, Diabrotica virgifera
auggaaggaauacuacuggaaccaacauuguauaccauaaaagguauugcuauauuggacuaugaugguaauagagugcuggcuaaauacuacgauaaagauauauuuccuacagcaaaagagcagaaagcuuuugagaaaaauuuguucaauaaaacucauagggcagacgcagaaauuaucauguuggaugguuuaacuuguguguauagaaguaauguagauuuauucuuuuauguuaugggcaguucacaugaaaaugagcuaauuuuaaugaguguuuuaaauugcuuguaugacucaguaagucaaauauugaagaaaaauaugcaaaaacgagcugucuuggaaucacuagauauuguuaugcuggcuauggaugaaauuguugauggaggaauaauuauagauucugauucaaguucaguaguaucuagaauagcauuaaggacugaugauauuccauuaggagaacaaacuguagcucagguauuccaaacggccaaagaacagcugaaauggucauugcugaaauaa

SEQ ID NO: 176 - RNA, 450 nt, Diabrotica virgifera
auggcugaccaacucaccgaagaacaaauugcugaauucaaagaagcuuucucacuauucgauaaagauggugaugguacaauuacgacuaaagaauuaggaacaguaaugagaucucuaggacaaaauccaacagaggcugaauuacaggauaugaucaaugaaguagaugccgaugguaacggcacgaucgauuucccagaauuuuuaacgaugauggcacguaaaaugaaagauaccgauagugaggaagaaauucgugaagcauuccgaguguucgacaaagacggcaaugguuucaucucagcagcagaauugcgccacgucaugaccaacuugggugaaaaauugacagacgaagaagucgaugaaaugauucgggaggccgauaucgauggugauggucaagucaauuacgaagaguucgucaccaugaugacuucaaaguga

SEQ ID NO: 177 - RNA, 2013 nt, Diabrotica virgifera
augaugcaagcaaacaaucgagucccaccuauaaaguuggaaaacgauauagaucuuuacgccgaugauaucgaggauuucgcacaagaugacuuugguggugaaaauguugaucuauaugacgauguaauauccgcuccuccuggaaauaaugacaacccaggugauucaaaucaucaugcuccuccuggugcuggugaagaugguggagguaauuuuguugggucaggaggagcacccaauaauauaaauucuucuggaagaagacaucagcuguauguuggaaaucugacuugguggacaacugaucaagauauagaaaaugcagugcaugauauagggguaaccgacuuccaugaaguuaaguuuuuugaacacagagcaaauggucaauccaagggauucugugucauaucuuugggaucugagggaagcaugagacucugccuggaacuccuaucuaaaaaagagaucaauggccaaaauccccuuguuacccuucccacaaaacaagcucuuaguaacuuugaaagucagucuaaaacacgcccuucuccuacuaauaauucuaacucacguccuccccauccuaauaauaauguucauucagguccuaugcagaauuauggagguagaaugccuaugaacccuuccaugcgucccaugcccccagguaugcaaggugcuccaagaaugcaggguccaccuggauuuaauggaccaccaaacaugaaucagcaaccccccagguuccaagguaauccacaauggaauggaccuagaccuaaugguccugggcccaauaugggaaugagacccauggggccaccucauggacaacaagggcccccaagaccaccaaugcagggaccaccgcagcaagguccuccaagaggaaugccgccacaagguccaccgcagaugcguccagaauggaaucgaccaccaaugcaacaaggguacccucaaggcccgccgcauaugcaaggaccuaacauggguccaagagguccaccccaaaugggaccacccggggcgccucaacagcaaggaccagcuccgcacguaaauccagcauucuuucaacaaggaggaggaccaccgcccccaaugcaacacaugccuggaccagggcccgucaugccuccucaaggacccccgcaagguccaccacacggacccguuggaccuccacacggcccaccauuggguccagcgaauguuccgccucauggaccaccucacggauaugguccaccugcagcgaugccacagccgccauacgguggcccaccuccagaccaccgcgcugagauuccucaguuaacagagcaagaguuugaggauauaaugucccggaauagaacaguuuccaguucggcgauugggcgggccguauccgacgccgcagcuggagaauuugcaagcgccauugagacuuugguuacugcuauuucacucaucaaacaauccaaaguggcuaacgacgaucguugcaagauccuuauaaguucgcugcaagauacuuugcguggugucgaagacaaaagcuacagcuccagccgcagagaccggucaagauccagggacagaucacauagaagaacuagaagagaacgauccucgucacgguacagagacagaagcagagagagggagcgugaacgcgauagagaucgugaucgugaacgugacagauauuaugauagauacagcgaaagagaaagagaccgagaucguucaagaagcagagaaagaacagaaagggauagagaacgagauuauagagaccgggaacccgaagagacagauaaagaaaaaucuaaaguauccagagucucaagaucaagaaacaaaucuccggaaccugucgaaccuagcagcgagguaccgaaaucaucccgcuauuaugaggauagguaucgggaacgagagagagaaggucgacgagagagcgaucgcgaaagagaaagagauagaagaggggaagacagccauaggucucgacacuag

SEQ ID NO: 178 - RNA, 765 nt, Diabrotica virgifera
augaguucuauuggaacuggguacgauuuaucagcuucccaauucucuccugauggaagaguauuucaaguugaauaugcaaugaaagcaguugaaaauaguggcaccguaauaggccuccgagguacagauggcauuguauuggcugcugaaaagcucauuaugucaaaauugcaugaaccaaguacaaauaaacgaauuuucaacauugauaaacacauaggaauggcauuuucaggcuuaauagcugaugcaaggcaaaucguugagauugcuagaaaagaagcaucaaauuauagacaucaauaugguucaaauauuccucuuaaauaccuaaaugauagaguaagcauguacaugcaugcauacacuuuauacagugcuguuagaccauuugguugcagugucaucuuggccaguuaugaagauagugacccaucuauguaucugauugauccaucuggaguuagcuauggauacuuuggaugugcuacagguaaagcaaaacagucugcaaagacugaaauagaaaaauugaagauggggaaucuaacaugcaaagaacuuguuaaagaagcagccaaaaucauuuauuugguccaugaugagcugaaggauaagaauuuugaacuggaacuuucauggguaugcaaagauacgaaugguuuacauaccaaagugccugaaucaguguuugcugaugcagaaaaagcugccaaacaagcaauggaagcagauucagaaucagauacagaagauauguaa

SEQ ID NO: 179 - RNA, 744 nt, Diabrotica virgifera
auggcuucaaaagacagauugaugauuuuuccaucuagaggagcccaaaugaugaugaaauccaggcuaaagggagcccaaaagggacauaguuuauuaaagaagaaagcugaugcuuuacaaaugagauuuagaaugauuuugaacaaaauuauugagaccaaaacucucaugggugaaguaaugaaagaagcugccuuuucuuuagcugaagcaaaguuugcaacuggugacuucaaucaaguuguucuucaaaaugucaccaaggcucaaauaaaaauaagaacuaagaaagacaacguugcugguguuacuuuaccaguguuugaaugcuaccaagaugguacagauacauaugaguuggcugguuuggcuaggggaggucaacaauugacaaaacucaagaagaauuaucaaagugcuguuaaacuguugguugaauuagccucuuugcaaacuucuuuuguaacucuugaugauguaaucaaaauaacaaacagaagagucaaugccauugaacauguuaucauuccaagaauagagcguacuuuggcuuacaucauauccgaacuggacgaguuagaaagagaggaguucuauagauuaaagaagauccaggacaaaaagaagaucagcagagcaaaggccgagaaacaaaaacaagcucuucuccaagcugggcuacuuaaagagucccaggcaaacaugcuuuuggaugagggcgaugaagaucuacuuuucuag

SEQ ID NO: 180 - RNA, 4818 nt, Diabrotica virgifera
auggaggcggcucccaaguuaccgaugcucucguucgaguuaaauacuuguacagagaacguccacuuuggcccccaguuaaaacaguauauugcugcuuuuuauggugaagauccagaauccuacauuacagaaaucagcaaucuugaauccuuaagaucagcugcaguucgaccaucaacggauguaaaugguguacaacuguugaaaaaguauuucugucagcuucguuuucucaaaucuagguuucccauggaagagaaucaagaugcugcaguucuauuuucauggaaaaauaaugaauuagacauaacuucaacauccagugauaucagauaugaauuaaugguaauaauguauaacauuggagccuuacacacuuuucuuggagccaacgacucaagaaacaauccggaugguaugaaaauggcauguacucauuuucaaugugcugcaugggcuuuucaaaacguaaaagaaaaguaccaccaauucauaucaaacaucucauugguagaacugguucauuuuuuucaacaagucuguuuagcucaggcucaggaguguauauuagagaagagcauguuugacaauaggaaaccuaccaucauugcaaaaguugcuauccaagucuacaguuauuacagacagucuuuacgugucuuggaaucaguaaaugaagccuacuuuagggauaaaaccuacaaggaguggaugaaauaucuucaauucaagcugaccuacuacaaaugcaucucguuccuauuccaagggcaacaagcugaggaacaacagaaaaugggagaaaggguugcauucuaucaagcugcaugugaacagcuggacgaggcaaagaaaauugcugcuacauuaaaaaaccaacaccaccagcaagaaauaaaugagggacuagcauucacuacugaugugguugaagguaaaagaaaagcagcuaaaaaugaaaaugaguucaucuaccaugaaucagugccugauaaagaccaauugccagagguuaagggugcuucauuagucaaaggaauaccauucaguauaaaugauauagaaguuucaggaccagauauuuucucccgauuggucccaauggaggcacacgaagcagcuuccuuguacagcgagaagaaagcucagagauuaagacagaucggggaacuuauugaaaauaaagaucaaacauuggcugaauuuaugucgucaaugcagcuagaucuauugaccaagaugcaccaggcuacuggaauaccgcaggaguugauugauagagcagcggcucuaucugcuaaaccuaacgccauucaagaucuuauaagugcuaugggaaagcuaucuaauauauaccaagacguugaagcaaguuugaaugagauugauucuuuauuaaaggccgaagaacaaagugaacaaaaguaccaagaaacgauugguaaaagaccaccgagcauuuuagcuacagauuuaacuagggaagcggcaaaauacagggaggcucauacuaaagcgaacgacucaaaccaaacuuuacacagggcgaugauggcucacguggcuaaucugaaaauacuccaacaaccgcuaaagcagcugcaacaucagcugcccuuugucgaguuuccaaauccaaauaucgacgaaaaaucuuugaaagaucuggaagcgcuaguugcaaaaguagacgaaaugagaacccaaagagccaugcuaugggcucaacuucgagaaucuauucaccaagacgauauuacaaguucccuuguaacgaaacaaccaaaucagucgcuggaacagcuguuccagcaagaacuucaaaagcaucaaaaucugauuucguugauugaacaaaacaccucggcacaagaaaacauuaagagcgccuuagucgauucuuacgcuuacgcuguaaauucaagaaaauacauccaagauauacuccaaaagagaaccacaaccauaacgucacugauagcaucguucgacucuuacgaagacuuauuggcaaaagcuaacaaagggauagaguuuuacucaaaacuugaaacgaacguauccaaguuacugcaaagaauaaggaguaccugcaaaguucaacaagaagagcgagaucagaugaugucgacugcgcaagugccucaaugggagagucauacgucacuugccgcuccuaaacugaaagauuacuuggacuccaggaagaagagugcugcguauucggagccgaguguucaaccacaacagccaacuuuaaguuacucagcugcuauggaucugccuccugguauuaggccgacuccaguuggaucagaaauaacggauguaccgaaaaauauucaaggugaaccacaagguuauauuccauauaauuaccaacaaccuucuguuccugccucacagaauauugaugaagagacuauuaaaaaaaugaacgcauugaugccaggugcuaagacgucagugccuagucaguacggauacagcaacuacauuccaccaacauacccucaaagugcguaccaaccagguaaucagucuuacggaaaagaaacuccagauauuaacucaccguacgacccuaccaaggcguucacggcuacuacuaacgcuuaucguucggugcagagcuccucaacucaaggauacguaccguacgcagaaucuaacguuucgaauguugacagaguuggauauccuagcagguaucaguaccaacaaguaccugagauagcuacuacuccagcugaucccaauauuaaugcguacuacccacauggguacucaccgagccagaauuuaccgaaugcuaauacucaacauauuaccggccaacugaaguaccauucgguggaguacgcuucuucugugccgaacaacaucaauuauaacagcucuaccuacucgucgccgcuuucuaauaugucuaguaccaauuccucaaauccuaguaacuugaauaauucuuacgaguacuacuaugacccgaauaccaguaguggugcaguaccgaaugcuucaaagccucaacagucgagcgccagcucugcaaacccgaguaccgcuaugaacaacuacaauuauuacuacaauacaaguaccagcgguaguguagcagcggauacuucaaaaauacaacaacaacaacaguacccagguacucagaugagucaagcgcaguacuaucccgccaaugccaguuauuacucaaccaguacuuacaauaccaacguccaaggugguaccaaucccucguacgcaacuggacaaacauauaaucaagugacaccagugaccucucaaaauguuucucaaaauuacaacuuuaaccaaguugguucuggagcaggacaccagcaucaguacuacucguccgcuaacgccgcaguaccaucccaacaagcuguaaauaacaguucauuaccaaacuacggauacgaucaguauuacggcaacaacuauaauuccagucaaccgaguaccuacagcgcaaaccaagcaccuccugcagcacaagcugcuccaaguaauauuccugcugccaccaaauccuccucuaauguggaucugcucaguggcuuggacuucagcauaagccaagcuccucuagugccucaacaaaacauuacgauaaaaccccaagaaaaggaaacaaaaccaccggcuguuucuucugaaaccaaaaaccaagauccaacaccaguaaccacgcccaaacaacccacuggaccagaaguaaagcgcuuguacgucaaaauccugccgagcaaacccuuaaacaacgaugaugugaagaaauuguucggccaagagcuggacagguaugagaaguucguggagaccuugacccacaaaacuuugagcgguccgaccacucuggauauuaaauggaaggagauccaagaccagcaggauugcgagccgcagaagaagaucauuuccgucgcuagauguuauccuaugaagaauagguucccggauaucuugccuuacgacuuuuccaggguggaguugugcgauaguaaagaugauuauaucaacgcuucauacauuaaggauaucucgccauaugcuccgucauuuauuguuacacaagugccguugucuucaacuguuggugauauguggacgaugauuagagaacaacaggucgaacugauccucuguuugguaaacgacaaugagaucggugaagauauuuacuggcccaaagaaaaaggcaguagucuuaacauacuuaacauggucauaacguugcaaaacguuauaguuaagucucauuggacugaaagacugauagcgauaaacuuaccugaaaaacgggagucccgugugauaaugcaucuacaauuuacaucguggccuggcagcuuguuuccaacaaauccugaaccguucgucagcuacaccuuggaauccaucaaccuauaccaacaacagaagaccaacacccauccgguggugguccauuguucaucuggcauaggaagaagcggccugcucuguuuacugacagcugcuauguucgaugcugccaacaaugcuaacucgauaccagaucuuacagcuuugaguaucaaguuguccaauugcaggaagaauauucucagagaucgagagcauuugaaguuugguuacgaaaguuuuuuggcguauauuaggcauauaguuugugaagauaaagccagaaagaaacugaacgagauccagcccaagguuaaggaggaaccacuggaaccaccugucauaguuccagaaccaaauauagauccuuuaaguacuuuagacccauuuugggcuaguaaaagauaa

SEQ ID NO: 181 - RNA, 888 nt, Diabrotica virgifera
auggcggacgaugagagaaagaaacuggaggaggaaaagaagaggaaacaggccgaaauugaacgcaaaagggccgaggucagggcucguauggaagaggccucaaaagccaagaaggccaagaaagguuucaugaccccugagagaaagaagaaacuuagguuacuguugagaaagaaagccgccgaagaauuaaagaaagaacaagaacgcaaagcagccgaaaggaggcguaucauugaagaaaggugcgguaaacccaaacuugucgaugacgcaaaugaaggcccauuaaaacaaguaugugagggauaucacagacguauuguagaccuagaaaauaagaaauuugaccucgaaaaagaaguggaauucagagauuuucagaucuccgaauugaacagccaaguaaacgaccuuagaggcaaauucgucaaaccaaccuugaagaagguauccaaauacgaaaacaaauucgccaaacuucaaaagaaggcagcugaauuuaacuuccguaaccaacucaaaguugucaagaagaaagaauucaccuuagaagaagaagacaaagaaaagaaaccagacuggucaaagaagggagacgaaaagaagguacaagaggcugaagcaugauuuuucuccuuuguuaaagcccuuuugucaacaucaagggauaugucguuauuucgaugaucccaucgugauuucgauaucuuaaauauauuuauuuuauucauuacuuuccagacuaaaagagugucuguccgcauguauauuauuuguuuauguauaacuuauuaaaaaaugugaaguauuguaaaaaaaaaaaaaaaaaaaaaaaaaaaaaaaaaaaaaaaaaaaaaaaaaagaaaaaaaaaaaaaaaaaaa

SEQ ID NO: 182 - RNA, 791 nt, Diabrotica virgifera
ccgugaucucgagcgguuuuuaacagagagggaaaauguaaucauauauaugguugaauucuugaagguuagacuucauuccagucuugugauauuuagugcuuacuugguacagcaguuucagugcugugcuuuagaauaauuuauuuuuuaacauuuauauagaaaucaaauacuaaccaaucaacaugugugaagaagaaguugccgcuuuagucguagacaauggauccgguaugugcaaagcugguuuugcuggggaugaugcaccucgugcuguauucccuucaauuguuggacgcccaagacaucagggugugaugguaggaaugggacaaaaagauuccuauguaggugaugaagcucaaaguaaaagagguauccuuaccuuaaaauaccccaucgagcacggaauagucacaaacugggaugauauggagaaaauuuggcaucauacauucuacaaugaacucagaguagccccagaagaacacccuguucuguugacagaagcuccucucaaccccaaggccaacagggaaaagaugacacaaauaauguuugaaacuuucaacaccccagccauguauguugccauccaggcuguacucuccuuguaugcaucuggucguacaacugguauuguguuggauucuggugaugguguaucccacacugucccaaucuaugaagguuaugcucuuccucaugcaauccuucguuuggacuuagcugguagagacuugacugauuaccucaugaaaauuuugacugaacguggcuacucuuuc

SEQ ID NO: 183 - RNA, 2927 nt, Diabrotica virgifera
uaauuuuugacaguucgaaaauggugauguguuuauaauucgugucaauuuagugaaauagcauucaauuuuuuguauaagaucucuucaaaaugccacuucgauuagauauaaaaagaaagcuaacagcucgcucagaccggguaaaauguguggaucuucacccuacagaaccuuggaugcuguguucucuuuacagcggaaauauaaacguuuggaacaccgaaaaucagcaacugguuaagacuuuugaaguaugugauguaccuguucggacagcuaaguuuuugcccaggaagaacuggauagucagugggucugaugauaugcagauucgaguuuucaauuacaauaccuuagaucggguacauucuuuugaggcucauucggauuaugugagauguauugucguacacccuacacaaccuuauauauuaacaaguagugaugauaugcuuaucaagcuuuggaauugggaaaaagcaugggcuugucagcaaguuuucgaaggacacacucauuauauuaugcaaaucgccauaaauccaaaagacaacaacacauuugccagugcaucccuagauagaacauugaaaguauggcaauugggagcguccacagcgaauuucacacuagaaggucaugagaaaggcguuaacuguguggacuauuaucacgguggagauaaaccuuauuuaaucucaggcgcugaugauagauuaguaaaaaucugggauuaucaaaacaaaacuuguguucaaacuuuggaaggacaugcucaaaauguaaccgcugcauguuuccauccagaacuuccuguagcucuuacuggaagugaagaugguacugucagaguguggcaugccaacacccauagguuagaaaguagcuuaaauuauggcuuugaaagaguauggacuauuuucugccuaaagggauccaauaacguggcauuggguuaugaugaagguagcauuuugguuaaaguugguagagaagaaccagcuguuaguauggaugccaguggaggcaaaauuauuugggccagacacucugaacuucaacaggcaaaucucaaggcguuagcugaaggugcggaaauaagagauggagaacgccuuccaguuucuguaaaagauaugggugcuugcgagauauacccucagacaauucaacacaaucccaauggccguuuuguuguugucuguggggauggagaauacauaaucuacacagcaauggcuuuaagaaacaaagcguuugguagcgcacaagaauuugugugggcucaagauuccagcgaauaugccaucagagaauccggaucuacuaucagaauuuuuaagaauuucaaagagaagaagaauuuuaaguccgauuuuggagcugaagguauauacgguggauaccuuuugggagucaaaucgguuucugguuugacuuucuaugauugggaaacucucgauuuagucagaagaaucgagauacaaccaaaagcaguuuacuggucagauagugguaaauuaguauguuuggccacagaagauagcuacuuuauucuuucuuaugauucugaugaaguucaaaaagccagagauaacaaucagguugcggaugauggaguagaaucggcuuucaaucuucuaggugaaauaaacgaaucagugcgaacuggucucuggguaggcgacuguuuuaucuacacgaauucuguuaaucguaucaacuacuucguuggaggugaacugguuacaauugcucauuuggaccggccuuuguaugucuugggauaugugccuaaagacgauagauuauaccucguagauaaagaguugcgcguaguaagcuaccaauuacuucuuucuguucuugaauaucaaacugccgucaugagaagagacuuuccaacagcagacagaguacuuccguccauuccuaaggagcacagaacgagaguggcacauuucuuagaaaagcaaggcuucaaacagcaagcuuuggccguaaguacagauccagagcacagauucgagcuggcaguagcauuagaggaucuuaauauagccaaaacucuagcucaagaagcgaacaguccgcaaaaguggaaucaacuagcagaauuggcagcugcuacuaauaauguaagcguagccaaggaauguaugcaaaaagcgcaagauuauggaggcuuguugcuucuugcuacgagcuccggugaugaaaauuuaguccguacucuaggagaaacgacacaagcugaaagcaaacauaacuuagcauuuuugucacacuuguuaguaggugauuuaaacaaaugucuagacauucuuauuaauaccgguagauugccagaagcugcauuuuucgccagaucuuaccuuccugauaagauuacagaagucguggaacuguggaagacucaguuaucuucagucaaucaaaaagcuggacagagccuugccgauccuaaaaacuacgaaaaucuguucccugguuuacaagaggcggugguagcucagaaauuuuuggaacagcagaauaaagguuuagcgcccgcaagaguugccaccaccauuccuccuaaucacgacaggaauguuguagccgaaguucaagcacaaucgaaacacgauguaccaucauuuaguucuucguuuauuucaucagaaauagaagcacaaacaaggaguucugcuaaaccugaagaaucuucaaacauuauacagcuggaccaagaugacgacgauaucgauuuagauuuggacgguguaaauaucgaugagaacauugacacgacggauaucaacaucgaugaugauuugcugagugauugaaaauaacuuuuuuacuuuaguauuaaaucuguauauucauuccuauucuuaagaaaaucuauaugaauuuuaauguuuuaauaguuaagaaau

SEQ ID NO: 184 - RNA, 1620 nt, Diabrotica virgifera
uucauuucgccauuuugauuauuucaauuuuagaaaacguucaauaagcagggugcagggucaagaaguaaaguguucuagaaauacagauuaaauuguuuccuuuguguuauugaaaggcaaaaauaaaauaaugcagaucuuuguaaaaacacucacugguaaaaccaucacccucgagguugaaccaucagauaccaucgagaaugucaaagcuaaaauucaagacaaagaagguauuccaccagaucaacagagauuaaucuuuagaaagcguucgaguucgauaagcaagcaggucaagaaguaaaguguucuagaaauacagauuaaauuguuuccuuuguguuauugaaaggcaaaaaucaaauaaugcagaucuuuguaaaaacacucacugguaaaaccaucacccucgagguugaaccaucagauaccaucgagaaugucaaagcuaaaauucaagacaaagaagguauuccaccagaucaacagagauuaaucuuugcuggaaagcaguuagaagauggccguacucucucagacuacaacauucagaaagaaucuacacuacacuuagugcuucgucuuagaggagguaugcacaucuuuguaaaaacucucacugguaagaccaucacccuugagguugaaccaucagauaccaucgagaaugucaaagcuaaaauucaagacaaagaagguauuccaccagaucaacagagauuaaucuuugcuggaaagcaguuggaagauggccguacucucucagacuacaacauucaaaaagagucuacccuccauuugguacuucgucuuagaggagguaugcagauuuuuguuaaaacuuuaacuggaaagaccaucacccuugaaguagaaccuucugauaccaucgaaaaugucaaagccaaaauucaagacaaagaagguauuccaccagaucaacaaagauuaaucuuugccggaaagcaauuggaagauggucguacacucucagacuacaacauucaaaaggaaucuacccuccauuugguacuucgucuuagaggagguaugcaaaucuuuguaaaaacacucacugguaagaccaucacccucgagguugaaccaucagauaccaucgagaaugucaaagcuaaaauucaagacaaagaagguauuccaccagaucaacagagauuaaucuucgcuggaaagcaguuggaagauggccguacucucucagacuacaauauucagaaagagucuacccuccauuugguacuucgucuuagaggagguaugcaaaucuuuguaaaaacucucacugguaagaccaucacccucgagguugaaccaucagauaccaucgagaaugucaaagcuaaaauucaagacaaagaagguauuccaccagaucaacaaagauuaaucuuugccggaaagcaguuggaagauggccguacucucucagacuacaacauucaaaaagagucuacccuucacuugguacuucguuuaagaggaggaaauuaauaugguugaaauuaagcacauuuuuauauuuucaauaaauaaauuauaaauuauuaaaaaaaaaaaaaaaaaaaaaaaaaaaaaaaaaaaaaaaaaaaaaaa

SEQ ID NO: 185 - RNA, 2430 nt, Diabrotica virgifera
cucgaaacaaaugacugugaaugaaaagcccuauagcaacagcaacaacaaauauuugaauuuucgguaaauaacguuuaugaaaaauaauaauaauuuuaauuguuacauaagacacauaagaguacauaagauuguauuauggaagaacgagauuuaguuucuaauagacgcagaucauugcauaacuauggcgaagagaaaccuguuugucgaaaauuuccggauaaugaugaacaaaauuuccaauauccuccagauuauauccacaaaguaaauacagaaacggaaaaauggaaugcucaccucaguaacaucuuagucgaccaacagagguacaaagaaaaaauacgugaacuuaaaacuguaguguacaaugaaaacguuuugucacaugaucagcaggaauuuuuaaaaucuauagacuuuaauacauacuugagacaaacugaaauauuuuguaaaaaggugcauuuagccgcagaacuauacaguuucaauaaaagugaaaaauaucaagaguuacagaaaaccauugaacauguucaagaaaucauugauaguaaacugaagacauuuaaaggucgcuguaccaccaaaccaaaaaaccauaaaucaugugugacgacgauguagcggcucuugucgucgacaauggcuccggaaugugcaaagccgguuucgccggugaugacgccccucgugcugucuuuccauccaucguaggucgucccagacaccaaggugucaugguggguaugggucaaaaagacuccuacguaggagacgaagcccaaagcaaaagagguauccucaccuuaaaauaccccauugaacacggaauuaucacuaacugggacgauauggaaaagaucuggcaucacaccuucuacaaugaacuuagaguagcccccgaagaacaucccauucuuuugacugaagcuccacuuaacccaaaagccaacagagaaaagaugacucaaaucauguuugaaacuuucaauaccccugccauguauguugccauucaagcuguauugucucuguacgcuuccggucguaccacugguauuguacuugauucuggagaugguguaucccacacaguacccaucuaugaagguuacgcucucccacacgccaucuugcguuuggacuuggccgguagagacuugacugacuaccuuaugaagaucuuaaccgaaagagguuacucuuucaccaccacagcugaaagagaaauaguucgugacaucaaggaaaaauugugcuauguagcuuuggacuucgaacaggaaauggccacagcagccagcuccaccuccuuagaaaagaguuaugaacuuccugacggucaagucaucaccauugguaaugaaagguuccguugcccugaagcucucuuccaaccuuccuucuuggguauggaaucuugcgguauccacgaaacugucuacaacuccaucaugaagugcgaugucgacauccguaaagacuuguacgccaacacuguccuuucuggagguaccacaauguacccugguauugccgaucguaugcaaaaggaaaucacugccuuggcuccaucaaccaucaaaaucaagaucaucgcucccccagaaagaaaguacuccguuuggaucgguggcuccaucuuggccucccucuccaccuuccaacagauguggaucuccaaacaagaauacgacgaauccggcccuggaauuguucaccgcaaaugcuucuaaacuacuuuauauauuuaucguauacauauuaaguacaauacugagaguuggagcaugaauguauguuuuuauuuaugguuauauauaugaugacuuguugauauuguaacaauaaauucauuuuguauuacucugguaauauuuuauuuaugagaacaaccagauugaagucguaaagagccaauaacaaccugaagauaucaauugucaauugcuacugaguaauaguuugagguacuuagcuccagcuccuuuucacacgaaaguaagaaguaucugagcgagaauacauucuguauuguacuuuuuaaauaugcgacuuuuguaacaucaauuucauuguaaaauaucaucaucauuguuuuuauggaccuacgugacaguaggagacaccugacaagacuucuuuuccugcuguguauacgcaccuuuacaaacauccugcuggccuaauuguaguccuguaagggacauccuuuguugggcauggaguuuguuugugggaauuguauugacuacuauuauauaccuacuuuuauuauuaaggcauuugaaaucguaaacuaaaauugguuguuuauauuuuauaugaguauuuuuaguaguagauaagauuucaauugcaaaacuaccuauguauguauucauuagaaauaaauucuuauuccaaaauaaaguuuug

SEQ ID NO: 186 - RNA, 2822 nt, Diabrotica virgifera
guuuauuuuggucgucggcugaugugacguguaaagaaauuaaaucaaauuauuuuaaaguuuuuaaauuaaaauggguacuuuuaaaagagauacucaugaugaggacgggggaucaagugcuuuucaaaaucuggagaaaacuacuguuuugcaggaagcuagaguuuuuaaugaaacuaguguaaauccaagaaaauguacaccgauacuaacaaaacuguuguacuuauugaaccagggugaaacuuuaagugccaaagaggccacagauguuuucuuugccaugaccaaacuguuccaaucaaaagauguaauauugagaaggaugguuuauuugggaauuaaagaacucaguucuguugcugaugaugucauuauuguaacauccagucuuacaaaagauaugacugguaaagaagacauguacagagcagcugcuauaagagcauuaugcaguauuacugaugcuacuaugcuucaagcuauagaacguuauaugaagcaagcuauuguagauagaaacgcagcugucaguucagcagcacuaauuaguucauuacauaugagcaaauuagcuccagauguaguaaaaagauggguaaaugaagcucaggaagcaguaaauagugauaaugcaaugguacaguaucacgcauuaggucuucuauaccauauuaggaagacugauaagcuagcagugacaaaauugauuuccaagcugaauucaauggguuuaaagagcccuuaugcuuuguguauguugauaagaaucacugcaaaacuuuuagaagaagaggaccaagagucacuccucaacuccccauauacaauaauauuuacaaugggcuuaaggaacaaaucugaaauggugguguaugaagcugcacaugccaugguuaaccugaaguucacgaguaguaaugugcuagcacccgcuauaaguguucuacaacuauuuuguggaucuccuaaagccacacucagauuugcugcuguuagaacuuuaaaucaaguggccaccacccacccugcgucagugacagcuuguaauuuggaucuagaaaauuugauuacugauccuaauaggucaauugcuacacuggccauuacuacucuuuugaaaacaggugccgaaucuucuguugacagacuaaugaaacaaaucgcuacuuuuguaucugaaaucagugaugaauuuaaagugguugucauucaggcaauuaagguauuagcuuugaaauuuccaaggaaacauagcacgcuuaugaauuuccuauccgccauguuaagagaugagggagguuuagaauauaaagcauccauagcagauaccauuauaacccuaaucgaagauaaucccgaagcuaaagaaucugguuuggcgcaucuuugcgaguucauugaagacugugaacauguuucuuuggcugugagaaucuugcauuuguuaggaaaggaaggacccaagaccaaacaaccaucgagauacauccguuuuaucuacaaucgcgucauauuggaauguccuucuguaagagcugcugcagucuccgccauggcacaauucggagccucuugucccgauuuguuagaaaauauccaaauauuacuuucgaggugucagauggauucagacgaugaaguuagggacagagcuacauauuauaguaauauacuuaacaaaaaugauaaaaguuuauacaacaauuacauuuuggauucuuugcagguuucaauuccuucacuagaaagaucgcuuagagaauacauucaaaauccaacugacgaaccauuugacauuaaguccguaccuguagcagcagugccaacagcagaagaacgagaaguuaaaaacaaaucugaaggacugcuagucucucaagguccaguccgaccuccuccggugucuagagaagaaaacuucgccgaaaaacuuaguaacguuccggguauacaacaguuaggaccuuuguucaaaacuuccgacgucguugaacucacugaaucugaaacagaguauuuuguccgcuguaucaagcacuguuucaaacaucacaucguccuccaauucgauugucugaauaccuugccagaccagcuuuuagaaaacguuagaguggagauagacgccggugaaaccuucgaaauuuuggcagaaauaccuugugaaaaguugcacuauaacgaaaccgguaccacauauguaguaguuaaguugccugaugaugaucuccccaacucuguugguacguguggagccguguugaaguucuuagugaaagauugugauccaucaacgggaauaccagauucugaugaggguuacgaugaugaauauacacuggaagacaucgaaauaacauuaggggaccaaauucaaaaaguaagcaaaguaaauugggcugcagccugggaagaagcugcagcuacuuauguagaaaaagaggauacauacuccuugaccaucaauacgcuaaguggcgcuguuaagaauauuauucaguucuugggauuacagccugcggaaaggacugacagaguaccggaggguaaaucuacgcacacauuacuucuugcugguguauucaggggagguauugacauacuaguaagagcgaaacuagcuuugggcgaauguguuacgaugcaacuaacagucaggucgccagauccugacguugcugagcuuauaacuucaacuguagguuaaguuuaaaggcuacguuaaugauuauauuguauuacaauuuuuccauauguauaaauauuuugauuuauuuaaauuuuauuagaaauuaaacaauuuuaaguaaaaaaaa

SEQ ID NO: 187 - RNA, 3701 nt, Diabrotica virgifera
gucguuuaguagaagugucauuuuugucugcucucguuuucaucuuuuguguauaaaauuggugcuaagguagauaaacuauauucgaauaggcguuuuuaugucgcaagaaaguguuuuguauauuugugauacgugaucucguagagggaguccagaaauucauaauuuauaguuaaguaaaaaauggcggcaaacagaacuggaccugcucagagaccaaauggcgcuacccaaggaaagauaugucaguucaaacugguccuacuaggcgaaagugccgucgguaagucgaguuugguacugagguucgucaaaggacaguuccacgaauaccaggagaguaccauaggagcagcuuuccuuacacaaaccauaugccucgacgauacaacuguuaaauuugaaauuugggacacagcgggucaagaaagguaccacaguuuagcuccuauguacuauaggggcgcacaggcagcuauagucgucuacgacauaaccaaucaagacacauucggcagggcgaaaacgugggugaaggaacuucaaaggcaggccaguccgacgaucgugauagcuuuggccggcaacaagcaagauuuggccaacaaacguaugguagaauacgaagaggcgcagacguaugcugacgaaaacggcuuacuuuuuauggaaacuuccgcaaagacggcaaugaacgucaacgauauauuuuuagcaauagcuaagaaacugcccaagaaugaacaaaccacaggucaaggcggcagugcccaaggcaggcggcuagcggagggcgauucgggcgccaaggcacccggaaauuguugcaagugaugguauacgccugcaggucgaguguuguuauuaaaccgucacgagaaaggacuggcaagugcagcggcacucuagugauaucuauguguaauaaagguccuucuauuaacaaaaaaaaauuaauaaaaaaauauauuaaacuucauauacacugucacauauuccauuuagaugaugaaaaacaaaagagcagaagcauuuuggucucuaacggucauguugaguuggaaugucucguggggcauuuaaauuugugauaauuguaccuauauuuuguuuuuuuuauauauauaauuaauauaucugacaguguaaguuaagcguaaacuguuuauaucgaucguauuagcagcaccaauuaaaaaaauaaauaaaauugaagaucuuuuuuauuguuuuuguaauuuuaacucuuuggggaagguuacaaaagaauugauaacguuguguugaaaacauugcauuauaauaaaguaauuccacccacuuacgaauuauuuugaaaggagaaucaaaaaugugucaauugucaccugucaucuuaugaugacacucuaugagagugggggcaauugcagcgcgcgcauacuauucagucuagcuucauuaugucaauuaaaugucgauguuuaaaauuucaucgauuaguuugacaguucccaaaugaaaaaacaguuacuuccgcauaaaaauguauugcuucaaaacuuacuuucaguggcauuucaguggguuuuagccaucuuauggagaguggucuaacacacuuucuagguacagcugugguuuuuaaaaggaaauauaaugaaucgcuuuuguuuucgaaauuuacauugggaaucccuuaguguccuuauauuugcccuacagaguuucauuuaaacugaaauuggguaauuuacauagaccuaaagcuuucacgucuauauagccuaauuagaccacaaaauugacuugcuuuacgugaaauguaugaaccauucaaacauauuuauaugacaacaauauaauauaauacaauauaauauaaauaacugacaacauuuaaaauuagaauuuuuacccacaauuuuucucugccaugugucaucuaguacaagaaguauggcaauuuagcccuacuccucuuauuguacauuucuuguaauuccucauuuucuuuuucaauuacuucgaucuuuuaaacuuaauucaccaaguuuauuugcaauauuguucuugaagucuauaguuugaauuccuucaauauauuuguaauguuuuucuucagugucaauccuugcauuccaugaaagaacacgaaaaagaugcagcacuuuauuuguccauauuccugcuacauaguuauuuuucgauuaacacauucacugccagcguauaguaaacuggcacuuuacuauacgcuaaacaaaaauuuggccuugcagauuuuccuaaaacacguagcagcuacgccacugaauggacuuaggacccccuaucguuucgcugguugcaauaguccgguaacuagagaagcaauuauuauauaaaaguaaauuuaaauaauuguauuuugcuugcaguacugcauuuuaauaauuaguuuucuuuacuacauacaauuguuaccuuuuaauaacauaaucuaaaucuuacuuuuuuuuuuuuauuuuuuugggcuauggccuugacaauuauccaguaaccaggacuaauauaauuggccaauauaauuaaaagugcgaauaauaguacagagcguagaaauaggggucgcuuugccgaacuugcacggucccaauaguaaacuggcacuuuacuauacgcuaaacaaaaauuuggccuugcauauuuuccuaaaacacguagcagcuacgccacugaauggacuuaggacccccuaucguuucgcugguugcaauaguccgguaacuagagacaaaagugggucauuuugcacacuucauauaauucucguuuucuguacuucacgaguauuuuuuuugucggauauguauugaauaguaucggugacaacacacugcccugcgugacaccccuuucaaaaccuaugugggaacaauauguaucuuuagagaacuuuuuguacccuuccccuaaaaacuaaauauuuuuucacuuagugcaauuuuaauauuagucucaccuuauuucuagaauacggucggucuaaauguauaaucucaacuuucagcuuauacuucgaaaaccgagaaguauaaacuguaagaauauuuaacguuauauguauuuuauuaggauaaugauuguugaaaguauguuucccaacacguguaaguuacauuucguacucuuuccauuuuuauuacaaaaaaaaauguuuauuuagauaaacuguugcggaacauacuuucucgaugcaugacugaaaagaacguuaacauuuauacguauuuguuuuuauuuugugucugaugugacaacccagaucuuagcgaaagcguagaggacaaaagaugaguuuuuaaguuucccauugaacuuuuuccguccuuaaaucaaauacaacaacuauucacuuaaguuuucuaaacauuuuugcuaauaugggccccugcaccauucccgacuuaacagucuucauuuguuuuuauuagguuuguucguaguacgauccaaacgaucggcaaccguugccaaccauuagacgcaugcgcaguuaacagugagugaucguagacuacgaacacgcaugcgucugaugguuggcaacgguugcugaucguucuacgaacaaaccuauuauaaaccgccucuuuuguagguaaaguaaaccuuauaaucauuuuccgagucuacaauuucuguaggacaucaagcauuguaaaguuuaaugaaau

SEQ ID NO: 188 - RNA, 671 nt, Diabrotica virgifera
cugcgauauagguguguauuucagcuggaauuuguaaugaaaaaaacguagaaauauauacuacaaugaaguuuuuaagaucgacagugugcuacauugccaucuuggcaauucucuuuacccucugugccgaugagguugaaggaaggagaaaaauuuugauggggcgaaaaagcauuaccaggacauaucuucguggaaaugcuguuccugcguaugugauaauaauccuuguaggaauuggucaaaucauccugggagggauauuguacguugcauugaggaagaagaucauugcugcaccuguaacggcaucauaugcaguggcuagacaagaaccauaaauuuuauuugucuagaauauuauuuucuaaauaugcaucuuuuuuaaauuauugucuacguaaauaauaagucuagaaauauauaaaaauuguauaaaaucauguaccuauauuuuucaauuuuuauaaaaaacaacccgaaauuuaauauuuuacugaauuaacauuaucauuucuaucuacacucaccggcacaaaauuccgucacccaaaauuuuugauuaagcuugacaauuuauaacuauauuauuugugcuccgauuuucaacuucauguugugcaucaguuuguagguacauguauugauauuguuugguauuauuccgg

SEQ ID NO: 189 - RNA, 693 nt, Diabrotica virgifera
guagucgguauguauuugaauuuaucuuaauuuguuuaauauuaguaguuaaauauuaaacuauaguuagaaguuguuauauaguggaccaguaguugacuccccaaaaugcagaucuucguuaaaaccuuaacggguaagaccaucacucuugaggucgagcccucagauacuaucgaaaaugugaaagcuaaaauccaggauaaagaaggaauucccccagaccagcaacgucucaucuucgcuggaaaacaacucgaagauggucguaccuugucugacuauaauauucaaaaagaaucaacccuucacuugguguugagauugagaggaggugcuaagaaacguaagaagaagaauuacuccacccccaagaaaaucaagcacaagaagaagaagguuaaguuagcuguauugaaauuuuauaagguugacgaaaaugguaaaauccaccgauugagacgugaaugccccgcugaacaauguggagcuggugucuucauggcagccauggaagacaggcauuacuguggcaagugcgguuacacucuugucuucuccaaaccaggagaugagaaauagauauauguccuuguauauuguuuaagaaaaaaauagaaaaaccuuuguuuauuugaauaaaauauucgaggaagaaaaaaaaaaaaaaaaaaaaaaaaaaaaaaaaaaaaa

SEQ ID NO: 190 - RNA, 2684 nt, Diabrotica virgifera
gaauuuaauuucaaagauuuauguucaguaagcuuuguuucaaagugcgguacuaucgguuucauuuuauaucucuuuauaacggugucgcgaguuuucuugugaaaaaugauguccaaagcagacacacaggaagaugccuccuucgccaaauuggaaaaucagauugcuaucaucaaauacguaauacucuuuaccaacguuuugcaaugggcucucggugcagcaaucuucgcucuuugccuuuggcuacgauucgaggagggcauucaagaauggcuccagaaauuggauucagaacaauuuuacaucggaguauauguacuuauagucgcuucacugaucgucaugauuguguccuuuauaggauguauuagugcccugcaggagaguaccauggcccuuuuaguguacaucggcacccaagugcucaguuuuauauucgguuuauccgguucggcgguucuucuggauaacagcgccagagauucccacuuccaaccgaggauccgagagaguaugcgacgucuuaucaugaaugcucaucacgaccaauccagacaaacacuagccaugauucaggaaaauguugguugcugcggagcugauggcgcaacagacuaccucucucuucagcagccccuuccaagucagugcagagacaccguuacuggaaacccauucuuccacggauguguagaugaacucaccugguucuucgaagaaaaaugugguuggauagcagguuuagcuauggcgauaugcaugauuaacguccuuaguauuguuuuaucuacgguacucauccaggcauugaaaaaagaagaagaagcauccgauucauacaggagauagauuuagugagauagagauauaauguaguaauuagaauuuaauguaucuucaacuaaauuacuuuuucuuuagagauauaccugaaauuguaaagaacaggaaaauuaaauaagaaccaaaaacuaaagugaaccaacaauaauugaacauuccaaaauacacuuuuuuuguuaaguuaacuaaacgacauaaauuuuucauuuuuuaaguuuuuuauuguuuuuuuuaguauuauaauuuggauaagguguuuuuauauuaaguguguaauuauaaaguuuuuuuauaggacggaaccuaaauuauauagaaucauacaauaaacuauugucugcuuauugaauuuggaaaauaaacauuugguauauauuaaaaauaauaauauaugucuuaaugaggaacuaaugaaaacgucuauacauuuuugaauuuaauaccaacagauauuguaauuauuaauuuuaauuaaucaacuccaagucaacaucuggaaagcaauagaaauuaaaguaauuaacuaacuaguaacauucuagcaaccuguacaugugguuguauuacucuguuuugacauugacaaaacuagcuuugugaucaguuaucucuagcaguaauaaacucuagcuguauuuuguuuuauauauuuguccaaagaauugguuuauuuuaaagcaaauauaaugguuuaacccaggggugggcaaacuuuuuuugguaagggccauaaaauauuuuugaucuauuaccgagagccgcaauauuuguuaccuuaacauauucgaauuuuuaacuuuuuacuaauuuuguuuacguguguugggggggggggaugguuaaauuaaauaaacacaaauaaacauauucaguacgauucaaagauuauucaaaaaaauuuaaaaccaaauauugaaaaauaagccaacgguggcaaauuuuuacaggcagcucuaagaaaaaauggauuuugcaguagaucaaugcauaugaaacaaaaaauucaaaaauauguuauuagcuuaugaguuucucgagguaacgcuguugaguuuuuuaguuuuaacgauuuuugaguuuuugauaucacucaaaauaccagucaaaauaaggaaauuuuuguaaaaagugugaaaaucaacauauuuauuauuguuaaccaaaacauaucucgauagaguuacuucacgaagugaccaaagaaaaucuaugaaaaauuuuagguggagcugucaaguagguaguuuuugaguuacaauguccaccgccuuugaaaaaagcaguuuugagaaaaacgcauuugguuugacaacuuuuauuucccuuuguuuuuugucugucaaaucguaaagugauguacgccggaauauguuuuugaaucguagaguaauuuauaaaagagacgagaacagcuguuguacguuucucccuacgcucaggcuacugcaaagcgagucgaagguagggauauuaaacaugcuuuacuuccgguaauuuuacuccagucaagcugaaaauuuuagagaguagucuugaaguuuauuuauuuauuuauuuauguacuuucauuuaaaauauaaaaaaaaaucaaacauuuaaaacaaaaaaaauuuucaaaccguaacccuccccucuaaacagcauucgcauuuaauuaagcaugguauuucccucaaauauauguguaucuauauauuuauuuauuuuuauuuuaucucugaaccuauuucuuuuuccuuaauauuguucugaagcuauuugcuuguaacaucuuaugcaauuuacuguuuuguuugagaaugggccacaguuugaguuugaaauuaaauuuauuu

SEQ ID NO: 191 - RNA, 458 nt, Diabrotica virgifera
gcuugaucuucaagguagaacaacgcaaguagaaaucuaaaacaugucuggacguggcaagggaggcaaaguaaagggaaaagcaaagucccgaucaaaucgugcugguuuacaauuuccuguaggucguauucaucguuuauugagaaaaggaaauuaugccgaaagaguuggugcuggagcuccuguauacuuggcagcuguuauggaauauuuagcugcugaaguuuuggaauuggcaggaaaugcagcuagagauaacaaaaagacccguauaauuccuagacauuuacaauuggccauaagaaaugacgaggaauugaacaaauuacugucaggaguuaccaucgcccaagguggaguauugccuaauauacaagcaguacuguuaccuaaaaaaacugaaaagaaagcuuaagaguuaguauuccuuuuuauccaacccggcccuuuucagg

SEQ ID NO: 192 - RNA, 2478 nt, Diabrotica virgifera
acuagguaaauauaccuuguauauuugucuauuaacgguauuauuuauauuauuuuaaagugcgcaugcguguugugacaguuuauccaauuuucuuacaauuucagguacccucgaacgcaacuaauguucuuuuagauuguuagccagauuucacacaauacguggucacgucaggccgccguauucaguuuuugauuucaaacaaacugacugcugucugcauaaaaauccaaaaaaucuuuugccaauauuguugugacauuuuauuacguauacucguaaaggcguaauaaucaacuugaaauugcaauaaaacuaaaagcuaaauaacgauuuuucuaguaauaucugucauacgaauuugaauacuggaaauagaugaaauuaguaucguauuuuuucuuaaauuuugucacccaugaauuaaaagcuucagagucaaauuagauacauuaauuuguaaugcagaaauuuaacuguaacuuccugucauggaacuugauuuugauauuugaugguacuuucuuucuauuguuacugauaaaagauuaccaauuaaaaaauauaauguucauuguauucacaugauguauuacauuuguugguacucuuuugcgacaauuucgaccaauguacauuuagauuuugagcuuuacagcaauggaacugaaauuaacguucaagauaucaauacaaauaaaaagcucaaaaucucuggacgauaagaacacuuucaucacaugccaaugcuugcuuaacuuuuuuaaauuaaauuuuuuuguuaaaaucaaugcaguaaaaguuuuuuauguaagauaucucucccuaguguggagauaaacuaaggcauauagaauaugacaguaguuauauucuauacaaaaaaaauuaaguauccaugugguggggguaaauguaugagggucaacaggaucgauacugucuguguauuaaauaaucuaugcucuuagcgaauguucccagguuaguguuauguaaucgugggccucaucggccucaacacuuuuuuuugucuacagugaaguaagaaagcgguuuccaaagugcaugaaguuggaugguguagaucugcccccaccaauuagcuucgacauugcggaagagcaaccguuaccaccuugccaacagacguucuuauguaauggugauggaggauccauagugcgacaguuucucgagcuguauuucguaauauaugauucagauaauaggcagucccuucuucaggcauaucacgaaaaagccacauuuucaaugacaauggccuacccguacggcuauuccaaagacaguaaaggaguaucgugguugaauugguaugccaccgauaauagaaauuuauuacgaguucaagauccagacagaagaaacaaguuguuaagacagggacaaguugcuguaguuucguucuugcaagauaugccgcacacgaagcacgauauucacaguuuuacaguagauuugacaguuuuuacaccccagauguuauguuugacaguggcugguauguuuaaagaauugaaaaguggccacaaaguaccuccuuuaagauauuucuucagaacccuuguaauuguaccugcuggaucagguuuuugcauagcaaaugaagaacuucacauauccaaugcaacuccggaccaagcaaaagaugcuuucaagaccaccguuaauguagcuccggcaccagccccugugauuaccucuccuggacccaguauaccacaacccgcugugccagaugaugcuacaaaacaagaaaugguaaaacagauguccgcaguauccggaaugaaucucgaguggucgcuacagugucucgaagaaacacaaugggacuaccagaaagccauaaugguauuccaaaauuuaaacgcacaagguguuguaccacaagcagcauuuauuaaaugauacgaagauuauguuaacuuugguuaauuaauugacaguuaguuauaucuuggcaaauguaaauaguaucuuaaauuauagcuaauuuuuaguuuuauuauuguuuaagguaauguuaguuuaagaugucgaauuuuaaguuuguuacauacgaaaaucaaaucgaaaaaauguuaauagucccuuagaaauaccgguagcccuuaucuuagaagaaaggugguagauuuuaaauaaaaacuggguuacuucacaaaaaaaugucuauuuuaauuaguugaucucgauaacuuaauaugauuauauaaauacuggccuaaccuaacaaaucggacugaaacuuuuaauauuaccuaaacaagcaagauccauuuagacuaaaguuugaaguuuuggaauuguauugaaauuugaacauuacauaaacgguuaauuuuauaugagaacuucuuagacaauaauacuaauuaacuauuuucaaugggaaauaagccacaauuuuaccaaaaaaaugauuuuauuaacguuucgacgcccaag

SEQ ID NO: 193 - RNA, 3274 nt, Diabrotica virgifera
caaaacgucaacaaucugacacgucuguaauguuuagccugacuuuugaguaaaauuaccgaaaaacauaauuaaaauugauuuauuaagaguacauaauaacguacgaaaaugacugcgguagaacaaccuuguuacacacuaauaaacuugccaacagauucggagcccuacaaugaaaugcaacuaaaaauggauuuagaaaagggugagguuaaaguaaaaauaagagcauuagaaaaaauaauucacaugauucuggcaggagaaagguugccgaauggauuucuaaugaccaucauaagaaacguuuuaccuuuacaagaucauuuggcaaaaaaacuauuauugauuuucugggaaauaguuccaaaaacaaauccagaagguaaacuacuacaagagaugauuuugguaugugaugccuauagaaaagaucugcaacacccaaaugaauuuuugagagguucuacacuucgcuucuugugcaaacugaaggaaccagaauuguuggaaccauuaaugcccaguauuagagcuuguuuggaucauaggcacagcuaugugaggaggaaugcuguacuggcaauuuuuaccauuuacaaaaauuuugaagcccucauuccagaugcuccugaacugaucuccaauuauuuggauggugagcaagacaugucuuguaaaagaaaugcguuuuuaaugcuucuucaugcugaccaagaaagggcguugucguauuuggcaucauguuuagaucaaguaaauucauuuggagauauucuacaacuggucaucguugaguugauauauaaggugugucauuccaauccugcggaaagaucuagauuuauuagauguauauauaacuuguugaacucaagcaguccugcugucagguacgaagcugcaggaacuuuagucacccucuccagugccccgacugccguuaaagcugcugcuagcuguuacauugaguuaauuaucaaagaaagugacaacaauguaaaacucaucguuuuggacaggcugauagcacuuaaggagcuuccuaaucacgaaagaauucugcaggauuuaguuauggacauacugagaguacucucugcuccugacuuagaaguccgcaagaagacuuuaagucuagcccuugaauuagucucuucacggaacauagaagaaaugguauuaguauuaacaaaggaagugaguaaaacgguagacagugaacaugaggauacaggaaaguacaggcaauuguuaguaaggacucuacauucguguuccauuaaguucccagauaucgcacguaguguuauaccagucuugauugaauuuuuauccgauaauaaugaacuggcugccacagauguauugcuguucuuaagggaagccauacagaaguuuaaagaauugcaaccguuaauuauugagaaacucaucgaaacuuucaaagacauuaaauuggucaaaguccauagagcagcaauuuggauuuugggagaauacgcgaguacugcuuccgauauagaaguuaucguuggagaaauuaacagauuguugggugaaggaucccucguugaagcugagcagaaguuaauagcaggagauacggaagagaaugcuccugcaccugcugcaggcgccaccacuuuaguuacuuccgauggaacauaugcuacccaaucagcuuucaacacugucagccaaaccacuaaagaagcacgaccuccucuaagacaauaccucauggauggugauuuuuucaucggagccucuuuggcaucuacauuaaccaaacugucuuugcgguaugaggaccucaccucuccugcugcuagcaauggauucaaugccaaaauuaugcuuauuauggcuggaauucuucacuugggaaaaucaggacuucccacaaaaucaauaaccaacgacgauaaagaccacauucuguucuguuuacgaguccuaucugaucguucuccaaucauuguugaaauuuucaaaaaauugugccgcucggcacuaaaugagaugcuucuagcuaaggaaucgguagaagcgaucucgcaaaagagcaaagaaaaaaacaagcguacgauucaaacugacgacgcuauaagcuuccugcaauuagagacagauaaaaguggagagcuaggagaaaacguauucgagaugucgcugucacaagcuuuaguaggaggucgaacgggagguggcgaaucaguauuaaguuccaauaaauuagauaaaaucacacaacugacugguuuuuccgauccaguuuauuccgaagcauacguucacgugaaucaguacgauaucgugcuugaugucuuaaucguaaaccaaacuaacgauacuuuacaaaacugcacgcuagagcuggcuacuuuaggcgauuugaaguugguagagaagccacaaccugucguauuggcgcccaaagacuuuugcaacauuaaagcuaacgugaaaguggccucaacugaaaacggaauuauauuuggcaacauuguguaugaugucauaggagcggggucagauaggaauguuguaguuuugaaugauauacacauagauauaauggacuauauagugccugcuaguuguacagauagcgaguuuaugagaaugugggcggaauuugaaugggaaaauaagguaaccguuaacacaccccucacggaacuuucagaauaccucgaacaucuacucaaaagcacaaauuugaaauguuuaacaucagaaaaagcucugagcgggcagugugguuuuauggcagccaauuuauaugcaaaauccauuuuuggagaagacgcuuuggccaacuuaaguauagagaaaccuuuuaauaaacccgaugcgccaguaagcggucauauuagaauaagggccaaaagucagggcauggccuuaaguuuaggagacaaagucaauaugacacagaagagcacacaacauaaaguaguagcugcauaaauaaaaacguuuuuuucuguuucaacccuuuucuucauuuuaaaucauauucuguaauuuaauuaauuuaauaucauauuacugguuacacauuguuagauuauaaauaucuuuuaaagaauaaaauuuauuuagcuuuuacaaagagcggaaagcaaucuuuaucuucuucuaauaagaucaacaucaaguuuucuggaaaauauaauauauaauuuaaagauguuuagcauuuauuagugaaauacauggcuuacuauuuugaugacaaagucaguguaauauagaaaagcag

SEQ ID NO: 194 - RNA, 3652 nt, Diabrotica virgifera
gguagaugaugaugacccuaauaaugaaaacaacucaagucuucuuuuuaaaaacugcaaaaaagaacccuacggaguccaccucugaauuagguccaaaucuuugaacaaaacuccuugcuucgucugaaagagaaguuccgagccauccagguucuccuaaaguuaaaucugguuggacuaauuccaagaaauuaaaacuuuuuaaagcccaagcuacaaugccuucguucucagauaggguuauaguacauguacuguagaccaugauuccaccuggcuucaauaauuuugcugcauuuucaaacaguuuucuuuguaauggaacgaaugacuugaucucguuuucugacgaccuauuggccaguuguggucuuuucccaagaacacuacacggugcaucuaaaaguauuuugucgaacgauuggggugcgaaugguggaccgucuauaacauuuuuggaacuuaaaguaucugaaauuauugcaguagaaucagcuugaaaacuauacacuuuggcgccgaaauccucacagcguuuuugaaguugggcaaccuucuuuggaguuuugucuauggcuauuaauauucccuuauucugcauuagcucugcuauaugaguaguuuuauucccaggagaagcacacauaucaaguacuacaucaccagguuugggauuaagauuaugaacacauacaauagaugguauauuuuguaguaaaauaucuccgacugguaauaaacuuucgcuuaucgguacacauccugaaauuguuucuguuacuucuacagcaauaccacugggcgcaauauuaucaguugcaaacaacuggugccucugcaucuuaacuaucccauuuccaaugaauauuuugaagucgucuucauauauuuucugaagucccuuuuuacacuuuuuugcaacaucggcguaaauacuaacguuuucguuuaucugacaaccugaaaccauuccuaauacuccuggggcaaauauaugagcaccccgaagaauugcugcagcacaaucagugucuacuauaauuucuuuaucaaacuuuuuaaagccugcuggacaauuuaaacugucaauuauuaucacauugguaaaagagggauguauguaaacauuugguguaucauuacaauuuucugcaaaauagguuuuuaauaccuuuaaaacuuuggcaguauuuguuuuuauuguauuuacucuuaucgaaguuauuuuuggugcagaacauaaccauugcuguauuuuuaauaauucuucauuugaaaccgauguuuuuguuuuggugacacuaucgucaagaaauaaauucguuauuaauuuauuguugaacguugucuuaaacggggaauuaggguaaaccaucuuaaccuuuaagauaauuuaaaaucaacguaaaauaauucuguaaacaaaauuaaagucacaaaacgugcauacuugauagguuagaaguugaugacuaaaaacuaaaaggucacgugucaugcgccaaccaaugcgacacgcugguucccaagacguggcaugagcuacgauucucuuucaaguccgccauauugacauucgacaacuuuuuugggaggacaggugaauguuauagcguuuuucaaaguguaagguguuuauuuucaaaaaguuuauaaaauaagcaaucacuauggguaauguguuugcaaauuuauucaaaggccucuuuggcaaaaaggaaaugaggauauugaugguaggacucgaugcagcugguaaaaccacaauuuuauauaaacuuaaauuaggagaaauuguaacaacuauuccaacaauuggauuuaauguggagacuguagaauauaagaacauuaguuuuacaguaugggauguagguggucaagauaaaauuaggccauuguggagacacuauuuccaaaacacacaaggccuaauuuucguaguagacaguaacgacagggaacguaucacugaggcuaaagaugaauuaaugcguauguuggccgaagaugaacuuagagaugccguacuucucauuuucgccaacaaacaagauuugcccaaugcaaugaacgcugcagaaaucaccgacaaacucggucuccauucacuacgcaaccgcaacugguacauucaagcuaccugugcaacuagcggagauggucucuaugaaggucuggacugguuguccaaucaauuaaagaacgccaaucgcuagaacauaggaaaagagaggagugcgcaaaaaagugugcgagugcagaugguuuuuuucuuguacucuugucgugucuacuaccucgcgcgcgcgcgaaacguuucguauaccauauguugcaauuuuuguguccgauggggaaauucgucauguuucacuuuggggguuauaaaauuggcuguuggcgacaagugguguaugaggugaauccgaaaauaguuuuaggcaauauaucauaugaaucucagcuuucuuuagguugagaguuguuuuuguaguaaaagaaccuuaaacauccuucauuggcgauuuauuauuuuuuaacugcaugaaaaucuuaguuuuaauguuuuuauauaacacuuuuagauuguacuaacuuuuaacauuccacaaauuucucauaaaauugugauaucuuuucuaauaccucuuauaucaaugaacuuauuuuugcaggccaaaauauuuucuuagaaaguaauuuuguaucaucuauaaaaauuuaauuuugggaauucaauuaaagacuuuaacuggcaaauuuuaaaauguuacaagguucuagucacguauuuagucuggacuugcacuuuauagcacuugucgcagaaagcaucuaacccccaaacacguauggugauguccuauuugcagaaaaugcacuauaauauacgauaguaauuauuuauuguccucuuucuggugaaaacaggcuuuuuguaacauauauuuuuuauuguuuguaauagguuaaaauguacaguagacacuuauauuuaaaauaaauauuauuuuuuaucuaucaauccuuuuuguaacauaugguaaggcuguucguauuuaaaaaauaaaacaagaacauguuuuugaaaaguuuugggcacggcucaaguauuuuauuguuuaaauuuaagaugaaguacgaaugggcgcgcccgaauccgacaagauauuguauagccccguauucguugucaaaccaacgggucuugcucugguaguuaauuucggcguuagaguucaagaaggaaguagauuuuuuuauacuuauaguaaauauauuaugggauauuuaaagacuggccgauuggcuuaccagacuuacgucccgcagaucuuaaccggcgaugaguauucgagguuuguuucuggucagauucgggcacgucguacuguacauaaaauacuuuuguuuacuucucaaauuuguaacucaguuguggauacuuugaggcguaauuuuauuuugacaaacuguggauuuaagguuacauuauaagugauuuauuuaaagaaauauguuuuugauuuaucguggacauucccuguuugucagcuguauuaucaaauaaauuuauacuauuaaaaaaaaaaaaaaaaaaaaaaaaaaaaaaaaaaaaaaaaaaaaaaaaaaaaaaaaaaaaaaa

SEQ ID NO: 195 - RNA, 7631 nt, Diabrotica virgifera
cuucacuuuuacggguaaacgugcggaagguacguuccaaguucgaaccuagcgcuugauguucuguguacacucuguacaauuaaaauggcuuccaaguucuuugugaguuaacuuuguuaggugcauuuuaguaaauuucugaauuggguuguuaaauuuagaguguuagugcauaguaacuauuaaauaguggccuuaaaaagugcucuuaaugaggauuauaauaagaaaaacgaacuuuuaacaagaacaacgaguguccauucuaauguuauuacaaguagcuacuaaaaauauugcaaaaaaaacuuucgguaacuaaaaugggacgguugcacuguuuauuuuguauuuuccuauguuuuaccgucaucaacacgcagacaacgaauauacauggauucucggaaaauuccguggauacauuucuaucaccucaugggaaaagugcaaaauucgugcaccaaaaucacaaacccaaaauugaaaauugucagaacuacaaacccucggugaaagaagaacagccaggcggaacguacguaacaacgguuaccgcuaucgaugacgauccuagggagggaggaggaacaauuaguuacaaacuaauucauagagaaggagaacauguuuuauuugacauagacaacguuacugguguuuugacaacuauccagccauuugaucgggaugaaccaguaaggcagaaggaacuuuauguaaccguacaagcuucagacaacggcaggccaccauuagcagaugucuguacauuuacaguuaccauuaccgacauuaaugauaaugcgccacagcuugauaaacugaaauacgaugcacaaguuucugaagauuuaaaaguaggaagugaagugaugagaguuuuugcuuacgacauugaugauggggaaaauucaagauuaucguauaacuuuucaaacgaaaaugcucaauucacccaguauuucaggauagaucgagauacuggcguuguguauuuaaaggaagcuuuaacagacaaaaagaauacuagauuuaacagugcuguuuauguagccgauaauggcguuaacgaucaagaaggccaaaaagauucaaccgcuaagauaucuauaacaguaguagggucugauaaacagccucccagauuuacucaaaaaaugccugauggaaucuuggagauccccgaagauuuuaaagacuuuucuaaacauauugucacagucgaagcaacguccaacauugcggauccacaacuugcuuuugaauuggugaagggaaagacauaucaaaccaauaaagaccaaacguuucuuuuggaggcagaaggaaauaaagcgcacauaaagcuagugcguccacuggauuaugaaacaguaacggaauauacucuaacuauucgaguaaaaaacaaagauuuaauggauucuuccauaaauauaccaauuaaaguauuagauguuaaugaugaaauuccuaauuuccuugaauuucuuaaagguagugucguggaaaaugacaagccaggugcacaagcgauucaaguaagagcaaucgauaaagacggaacugcugcuaacaacauugugagcuaugaacucguugacaauacagauuuguuugcaauaaaccgaucuacgggaguaauuacgucgagaguggaguuugaucgugaaacuguaccucuauaucacguaaacguuaaagcuuaugauaacucuccgucugcuuuguauaacacgacauugccuaacauuguaauucagacauuccaaaucaguauagaagaucaaaaugacaacaaaccuguauuuacucauccaauuuaucaguucaguaauauuacugagcuugcugauaaaucgaguauuguuggugaagucaaagcuuuagauaaugacacggcuucaguuauaaguuauaguauuacaaauggaaauauugacgaugcguuuaugauugaaaauucuaccggcagaauaagaguuaauggaaaacuggauuacgagaaaaucgaacaauacaacuuaaccguucgcgcauuugauggggcauuugaagauuuugcaauuguuuuaauuuccauacuuaaugaaaaugacgaaccuccaguuuuugacgacuauaucagagaaauucaaauuaaagaggaagaaccuaugauauccggaugcguuguuagagugacugcucaugauccagauauuaaagacaggcaugcugaucaacacauaguauaugaggucgcgaaagaacagaaagauuuuuugaccguaucugccgauggaugcguacaaguaacaaaaccucucgaccgagauccgccuuucgguagcccaacacgacaagucuucaucuaugcucgugauaaugauggaggcacaaauucauuguuggccacugcagaaauugaaauuauuuuaauagauauaaacgauaaugcucccuuuuuaaauguuacagaaauuguuuauuaugaaaaccaggauccagguuuuauagguaaccuaagugccgaugauuacgaugguccugauaauggaccuccguuugcuuuucgauuaucagacacugcuucagauaguauuagaucgaaauuuuccauuaucggaaaccagcuuuucgcuuuagaaauguuugauagagaagagcaaaaauauuaugacauugccauugacauuacagauaguggaguaccuccacuaacaggaacuaguauucuuagaguuauaaucggagauguaaaugauaauccagcuacagacggaaacagcacgaucuuuguguauaaguacgucaaugggccagaaaauuucauggaaaucggacguguauauguuacagaccuagacgauugggauuuaaaugacaaagucuuuguucaagaagauaacuuugaugaauuuguguuaaaccagcauaacaacgguaugauucugaugaaaccaacaacggcugagggaacuuaugagguucauuacagggucacugaaacccaugaacccacaauacacgaacauacaguuaaugcaauagucacgauuacaguuaaaguacuuccagaggaagcgguuguaaaaucaggaucaauucgauugagaggaacaacuaaggaagaauucauagaaaauucauugaauggaaagagcaaaagagacauauuacaccaagaacucuccaaaauauuaaauacaucuuuagcgaauguugauguauuuacuguuuuaaauucaccccaccagaauaguucguuuguggauguucgauuuucugcucauggaucuccauauuaugcuccagagaaacucgaaaacaaaguuacagaucaucaaauggagcuugaacaaaaauuagauguggaauucuacaugaucaacguaaacgagugccuuaacgaaacaacguguggagcugaaaacucauguacgaacaaauuaaacauaacacgagaaccagcuguaguguuuacuaacagaacauccuuugucgguguaaaugcauuuauugauccugugugugccgcuuuaccaagagauguuauggaauguuucaacggaggcguccuuaucgaaaacacagcguguaauuguccugcaggauuugaaggaccacauugugaaauccuagcuauaggauuuacaggaacugguugggcuauguauccauccuuugacgcuacaaacaggacugagauuauacugcauauuuuaucacaaacugauaaugguuugauauuuuacaauggaccuuuaaauauaagacaaacuucuuugucuaaagauuauauaucauuagaacuuaaagacggauauccauuacuucaaauuugcaccggcucaagcacucaagaaauuuaucugaaagagcgcauucacaaauugagcgauggaucguuacacaaaauaaaaauaggaucuggauuugacgauauaucccuggaaguagacgacuguggaacaacguguucaauuuggacuaauaaacuacauaaagguguuauccgagcaaauggcccccuucaacugggagguaugaaaaacagauucaccgaucaagaauucaaacgaauuugggaccauuugccaccgacugccacccguuucucugguuguauuagaaauuugacguauaaugaauuuuacuacaaccucggugcaccuucugaugcauuccaagcguaucccgacuguaacuaugcagugaugcaagcugugacuuucgguaucgacuccaauuucuugguugcuauucugguuuguguagcaauuuugauaauucuucuucuggcaguaguuguacauagacguaaacacgacaacuuuaacgaaaaagaaaucgaugauacucgcgaaaacauuaucaacuacgaagaugaagguggcggcgaaugugacaccaacuacgaccugucuguuuuccaucagaacaacauuguggacgaaaaaccauugaugagagacaaccccgauguaccugcagauauaaguggcuuuuuagauaacaagaaagacaacugugauaaagaccccgauaauuugccuuaugacgacguucgccauuaugccuacgagggagacggaaauagcaccggauccuuaucuucucucgcuucauguacggacgaaggagauuuaaaguucaacuacuuaucaaguuuuggacccagauucagaaaguuagccgacauguauggagaagauccaagcgaugaagacucacacgauggaaacgaagaauccuggugcuagacuaaauuccaugacuccuuagaaagugacauuuuuguacuuaaauucuuuuauguuaaccaaagauugcgaaauuuuuuggaauggaacugaacaaacuuguacauauuuuugaaaaaggauguuucaacuguuugcaauacaagaauuguuacguaugcguuucaaaacauauauuuacauguaaccguucuuguauuguaaauauuuuuauacuaucuuuauacugacacuuagauaauguuaucuauuaaguggcaagcaguuugguuaagaauaacuuugagucuucuaucccaagguacugugauuugaacaaaguuagaauaagcaacuuccauaccaucaauccacuucauuuuauucaacauacugguuuuccgaaacuaaagaagcagccuaauacauagaguucaauaacuuuuauuaaacaauccguauuaggugcacauauucaucgcgcacaaguacauauuguggaacuguacacugacgaggaacaaauauucaauaagaaucuuuaaagcgaaaaauauugaaauacacgaucagauuuaacauauuauugucuagcucaaaagaaaauaauuuuuaucuugacaauagacauuuuucauauauaagcuaguagauuuauaucaaaaucuauaaaaaaaguggcuuaaaaguuucuuugauuuuugcgauucucuugaaugacuucaguauuuuuuacaaaauuauaagacuguuaaucguuuaagcuggcagcaugguaagauacguaugugaaaacuuguauauuuuacaaaauaacugaaaauuaguauguugcgaaaaacaaauggaaacaaauauacagugaaugucgaauaguauugucaaaauuaaagucauaauuuuuaaagauuguccaaacuagguuguugauguauguuaaucucugaaugccauccuuuagccaucauuguguauaucagcuguuuccuuuugcaguuugugugguacuccugaauguacggaauaaucacauaguuuucacgaucuuuuacuuuucuuuucugcagcuaauuacagggugguuguucccuuaucaauauugaaauucuuauauaaucaguuuuuccagacuuagaaaaguuaaaaaaacagcaucauaucccugcuauguuuauucuucguuguaaggacacucuaauuauuuccaauuagagaucucaagauguuauguuuacuagcagguauugaacgaaaacaauaauaaaagcaaaccgaguaaucaacaauaguuacaauaccuacaauuuacuaugccuucguagcuuauauuggagagaugaguggaauuucucaauaggcgcauauacacauaauaauaagacacauauucaaauugguuggugguuaaauagcucaaaaacgauacaaaggaaacauauagguaacaacaaaauacaagguaaaaauagagguacuuacaaucaggacgcuccaacgaagucacuauuuuacgucuuuacacucgaaucgucugaaugcaucucuuuccuuauguccucgggguagaaucuugcaccucaagugguugaaaguuccuuucuucgucacauguggauuucgcuaauaugcgaacaaggaacaacacaggcgucaaaaaggaauaugauuucgaacuauuuaaaacaaucaaaucaauuuaucggaauuccaacacaagauuuauaucagcauuucaguaguaaucucaccauauuccaaauuugucuuuuuguauccaucuuauuuugucuuuaucuuacuuuauauuguuuuguuuauuuuuuauuuaucuguauccuuaucuuuuucucuuugacuaucacagcaaauuuuuguaauuuggauuuacaauuaguuaugacuuuaaugaaaacaauacuuaccaucucuuuccuuaaaucgugcgauauaauuuaucaacaauguaguaucauaccacaacaaugucuaauaugaaacauacgagaggccuuaugcauaugccuuaacacguuuaggcacaaauaccaauaaaaaccuuacacgguucggcaauaaaaugagagaaccgcuauuuuauauacacgcauuuuuucuauuuuuauuaggauuuuguuaguuuuguuauuacuuaguauaaauuuaggauaguuauuuuguauuuuuauucucacguagggcucagucgacguagucguuugaauaacauuuuauuuaaaacugcagguuugagaauagccauucauuggugccguauuuaaaaaaagauuuuaaauguuucuuuacuggauauaucgaaauacaguuuuuucuuuacguaaggcaguuagauuauuaaaauagucuugugaaagcguauagcucagauguuuuuacaauauaguuucuaaauuagucuuaaaaggcaacuauucuuauugcaauauuucccaagcuaaauucacucgagacuuuggacucauaaauauacaaugacugaguucuacuugauuuccguuucauugacuauuuaauauuuauuuuuacaagucuugaugaaaauuuggaaauacaugauaucaaagcaauuuauaaucguaauuaggcuaauaguauccuaaggugauaguauuuccugauuuucaucaauuauauauauauauuaguaucacaaaauacagucuguagagacccgugucauagauaaccuaaccuuccauuguuacacaucuuguuaacaagucgauuugaaguggugccgguugugaagaugcguaucuugauguuccacggcuaaaaucgguucguucacaaaagcuuuuauauauuuuuuauuuacuuuucuucuuaaauguccggcuugaauuuucaagauccagugcugguacacaggagggcgcgacg

SEQ ID NO: 196 - RNA, 1028 nt, Diabrotica virgifera
gucagugcugugugcugucacugucacuacucuagugucacgauuuuuuagcccugaaauugucagauuaucacaugacauuuugagaaaacuaaauuaguaagugaacacgaauuuaguucaaaaugccuuucuguggucccaaauugucccucugcggccugauuaucagugcaugggguaucauccaguuggguuucauggguguauucuauuacauuggggcuguggcuuuagcagaagauauuccagagguugaguuuaagggcgauuuagacaaauuuuauagcgacgucaacacggguuucacacagaaugcuuacaacugcuggauugcugcucuccuauaccugauaacauuagcaguaucagcucaccaauucugggccaacaacagaucaucauugaacgucuaagaaucuuuuaauuugcuuuuguauauauuuauuuacauuccuucuccagcuguagauuuuaaauguauagacccucaaauacuucagaguaccaauuuuucuguuaaacuuagcggacucguacaccaaagcaauauccaaacgcaacaugaaacacucaacaucccauaaauaucagagguuauguaucccuugaacgcgcuuaaaacuaccuaggaguccucgaacuuuguucaccgacgcaaugcggauaaggaucauccaccgaaaaccagaugcgacacucagugcauauuaucuuuccacaguguauguuauaugcgauuucgauaggguauugcuuugguguacgaguccgcuuaggucagucaauguagcauguuguuguuuuuaaaguuuucauugguacuaaaacuuuuguucaccaacguaguauuauaaaaaucauaguaauauaaauaacgcugcaauguuaugaaucuuaccaaauuauuuuaugguucuguuguauuagugauuugaauuuaaaaauuguacacauuuacuaugaaauaaauugauuuauuguuaaaaaaaaaaaaaaaaaaaaaaaaaaaaaaaaaaaaaaaaaaa

SEQ ID NO: 197 - RNA, 3012 nt, Diabrotica virgifera
auuuaugguacacaucacuaaaguauuaaucaccacacuuuauaaauucaaauuuuuagaguuggaaucuacaaaguuagugauaauuagaaccauuguggcauucagaaacggucuaaacuuucaauguuagcaaccuuuuguauauagcuacaauaccgauauaacagugaacugaaaaacucuaguguagugauacaaagcauuuaccaugggucuuaccauaucagcaguguuuaauagguuguuuaguaaaaagccuaugagaauuuuaaugguaggauuagaugccgcagguaaaaccacaaucuuauacaaauugaagcuuggugaaaucguaacuacaauaccaaccaucggcuucaauguagaaaccguugaguacaagaauauaucuuucacgguaugggauguagguggccagacgagaaucagaaaacucuggagacacuauuucgccaacacugauggacucauuuuugugguugauuccaacgaccgagaccguaucgcggaagccgaagaagaauugcacaauauguuaggagaggacgauuuaagagacugcauuuuguuaauauucgccaacaaacaagauuuaccgaacucgauguccacugcugaauugaccgauaagcuuaaguugcacacuuugaagaauaggaggugguacauacaagccacaugugcuacucaagggaaugguuuguacgaaggacuagauugguugucgaaugaauuggccaagugaauagguagauguugugaagaguagcuuuuauauuuuuuauugauaauguaaaauccuguuuuauuugccucaguuugguagaauucauacaauauugcuucuauguugauaacuuuauauguuucuaucauuaaaauauggauaauuuuauccaaaaucuagcuaaaccacuauauaacgcguuggcuguguuacgagacaaauagaccucguauuauacuaacaguuggcucugucgacuauguuguaauacacuguggcauuacuggacauauaugccucguaaagcacucaaugcgggcuuauaaauaucucuaaaugcagccaaaguauuaaucuuagcuuuaacucuugauauuuuucaggauuuguguauaauuuauugaaaguuuaguuaaauauuacauuuuucuucgauuuuguucauuuaggcaaucucacauuauuuauauguaacauguuauugaaucauacccgccauagccaauuauauacccgccauagucuaauuucagagauuauuuuuuuguuuuauugacgugaguacauuuuuuaagaaauucguuucaacauuuuugucucuccucuauucauucauucauuaaagccggauuuacauauacaaguacuuggauccgagauaucaguagaacugcgaauuuuuacauuaaauaccagugauucaaaggaaauauucauuauaucugcaauagcuucauaugcauuauuuauuuuguugauaucacaauauuccuugaguuuacauuccacaaacauucaaaugaucaguaaaaacuaauaaacugucccaucuguucuccagacaaucuuaguuccacuuuuuuuuuuauuuguaaauuacaugugcucuuguauugcuacuugguuuguguucaacuuccauccaugcucguauccaaguaacaagaggcccugagaaggcguuuauacuucugacaugcacucugaguagaguaacucgaauucaaguacuuguaagggcgacuuuagaguuacaaucccuagggauuauaucuaaugccaugguguucaaaauccuaugcuuaggugagguggauuauuccuuugugucauguauaguuuccuguaugucuuugcugcuguugugacucuuuuucauuucuuccuguccuugcaccgaaauuucgagucuuguuuugaauuuaauaguauuuuguauuuugacaacgaaacccgauuugggcuucgaaacguuaauaaauucauuuuuuaguaaaauuguggcuuauuucccauaaaaaauacguaauuguuuugaauuauuugcucaaucuuaucaauuguuacauuuacauauuuuuuauuugggucgauuuucuuuguuuauucacaaaauuguuauaaccugucugagaaaucuuucaaauguuuuuuuacauuuccauuauucaauauuacacaauaucucuauugggaacgugcuuaguuuaucucuauuaggaagacuaguuuuaaaaacuaaaaauguuuucuucgauaguaaaauaaacaaccaguguuuaacaucuuugccacauuuaaagauauuuauaacacuacguugggugcgucaugaggcauauaugucccgaauacccccauuugccacuguguuacgacacaguugacguagccaagcguuaguguaguacgaggugucccguaacacagccaagauguuaacaauaaacauaaacaaaauacaauuuugugcuacuaucuaaauaagaugucaaugaguuauagaacaaagaauauuguaugaaucuauucgaccauaucauaaacugacuuuuucauguaguaaacaacguuucugcaacuccuucuucugugaauucacuuuuuguucuaccuagauuuaaauauuuauauaaauaaguuuccuauuauauacuacaguggaaccucgauaacucggauuaaucgggaccgcggccgauccggguuaucgaaaauccggguuagccggagaauauaguaaaaauuaauaaauaaccuccauuauaauuacaaaaacaugaaacacauaugcacaguacacaucuaaauuacguauaguuguauagaguguagaguuuuguucauuucuugguaaaaaacucagucauacuguagagauguaccgacaccauaauaugguaggucuggauccugcguacaaaaaaaaauugauaaauagcaagccgaaaauuuguuguuagcuugggggugucuagucggacagacauugauauaug

SEQ ID NO: 198 - RNA, 1212 nt, Diabrotica virgifera
caacacugcuaucugggcuguugcacuaaucauauggguugguuguuaguucacuuuuuucuacaguaccaugugucacuagcauaauacucaaaaaauuaauauuuaauuuugugaacuuaguuaacaucauuuugaaaaaugcgguacacuuugaguuacaucggugcuacccuagcguccacuguaacacugauuuuugcccucuacuacugccucacgggaaaaggagagcaaguuaguuuagcaugguuauuguugaaugugucuccccacaugugggcaggucuaggaauuggccuugcuguaucauuaucaguuguaggagcugcugcaggaauucacacuacaggagucaguaucguaggagcugguguuaaagcccccagaaucaaaaccaaaaauuuaauuucuauuauuuucugugaagcuguggcuaucuauggguuaauuauggcuauaguacucuguggaaguuggaagaauuucgauguagaccuauucaaccucaaaacucauaacuuugcucaaaaccauuauggaucacauguuauuuuuggauccgguuuaacuguuggauuuguaaaucuauuauguggauuuuguguuggaguaguugguucuggugcagccauuucugaugcagccaauucaucauuauucgucaaaauuuugauuauugagauuuuuggaagugccauuggucucuucggucugauuguuggaguauacuugacgucaagaggcucuaugguuuaaauguucaguaaaugaacaugaaaaauaaaaugaauaauuuguuaaaagugugugugaugaagauacaacuauuuucacuaguuuacgccccaaaauauuucauugugguuuuugaacaauauuuaaaauccauucuaaauuaaaauucuucaaauaauuguugaaaacagauucaagggucuuuaaucaguguauauaauaguauuaaauaaauauuccacuaauuuuguuaaucauuguuaaacauuguauaguuacaaaagaaauuuuuaauuuagaauaauaugcuuguacagcauguagucccacuaguauuaguauuuaaaaacuguauuaaaaauugucuuuucuaugucuaauaaaauuacagacgauuuuguuaaauuuuaauacagaucuaaaaaaaaaaaaaaaaaaaaaaaaaaaaaaaaaaaaaaaaaaaaaaaaaaaaaa

SEQ ID NO: 199 - RNA, 7646 nt, Diabrotica virgifera
cggauuucggagaguucgauucguugucgagccuucaaaauggcuaccaacgauaguaaagcuccguugaggacaguuaaaagagugcaauuuggaauacuuaguccagaugaaauuagacgaaugucagucacagaagggggcauccgcuucccagaaaccauggaagcaggccgccccaaacuaugcggucuuauggaccccagacaaggugucauagacagaagcucaagaugccagacaugugccggaaauaugacagaauguccuggacauuucggacauaucgagcuggcaaaaccaguuuuccacguaggauucguaacaaaaacaauaaagaucuugagaugcguuugcuucuuuugcaguaaauuauuagucaguccaaauaauccgaaaauuaaagaaguuguaaugaaaucaaagggacagccacguaaaagauuagcuuucguuuaugaucuguguaaagguaaaaauauuugugaagguggagaugaaauggauguggguaaagaaagcgaagaucccaauaaaaaagcaggccauggugguuguggucgauaucaaccaaauaucagacgugccgguuuagauuuaacagcagaauggaaacacgucaaugaagacacacaagaaaagaaaaucgcacuaucugccgaacgugucugggaaauccuaaaacauaucacagaugaagaauguuucauucuugguauggaucccaaauuugcuagaccagauuggaugauaguaacgguacuuccuguuccuccccuagcaguacgaccugcuguaguuaugcacggaucugcaaggaaucaggaugauaucacucacaaauuggccgacauuaucaaggcgaauaacgaauuacagaagaacgagucugcaggugcagccgcucauauaaucacagaaaauauuaagauguugcaauuucacgucgccacuuuaguugacaacgauaugccgggaaugccgagagcaaugcaaaaaucuggaaaaccccuaaaagcuaucaaagcucggcugaaagguaaagaaggaaggauucgagguaaccuuaugggaaagcguguggacuuuucugcacguacugucaucacaccagaucccaauuuacguaucgaccaaguaggagugccuagaaguauugcucaaaacaugacguuuccagaaaucgucacaccuuucaauuuugacaaaauguuggaauugguacagagagguaauucucaguauccaggagcuaaguauaucaucagagacaauggagagaggauugauuuacguuuccacccaaaaccgucagauuuacauuugcagugugguuauaagguagaaagacacaucagagacggcgaucuaguaaucuucaaccgucaaccaacccuccacaagaugaguaugaugggccacagagucaaagucuuacccuggucgacguuccguaugaaucucucgugcaccucucccuacaacgccgauuuugacggcgacgaaaugaaccuccaugugccccaaaguauggaaacucgagcugaagucgaaaaccuccacaucacucccaggcaaaucauuacuccgcaagcuaaccaacccgucauggguauuguacaagauacguugacagcuguuaggaagaugacaaaaagggauguauucaucgagaaggaacaaaugaugaauauauugauguucuugccaauuugggaugguaaaaugccccguccagccauccucaaacccaaaccguuguggacaggaaaacagauauuuucccugaucauuccuggcaauguaaauaugauacguacccauucuacgcauccagacgacgaggacgacggucccuauaaauggauaucgccaggagauacgaaaguuaugguagaacauggagaauuggucauggguauauuguguaagaaaagucuuggaacaucagcagguucccugcugcauauuuguauguuggaauuaggacacgaagugugugguagauuuuaugguaacauucaaacuguaaucaacaacugguuguuguuagaaggucacagcaucgguauuggagacaccauugccgauccucagacuuacacagaaauucagagagccaucaggaaagccaaagaagauguaauagaagucauccagaaagcucacaacauggaacuggaaccgacucccgguaauacguugcgucagacuuucgaaaaucaaguaaacagaauucuaaacgacgcucgugacaaaacuggugguuccgcuaagaaaucuuugacugaauacaauaaccuaaaggcuauggucguaucgggauccaagggauccaacauuaauauuucccagguuauugcuugcgugggucaacagaacguagaagguaaacguauuccauuuggcuucagaaaacgcacguugccgcacuucaucaaggacgauuacgguccugaauccagagguuucguagaaaauucguaucuugccggucucacuccuucggaguucuauuuccacgcuaugggaggucgugaaggucuuaucgauacugcuguaaaaacugccgaaacugguuacauccagcgucgucugaucaaggcuauggagaguguaaugguacacuacgacgguaccguaagaaauucuguaggacaacuuauccaguugagauacggugaggacggacucuguggagagaugguagaguuucaauauuuagcaacggucaaauuaaguaacaaggcguuugagagaaaauucagauuugauccgaguaaugaaagguauuugagaagaguuuucaaugaagaaguuaucaagcaacugauggguucaggggaagucauuuccgaacuugagagagaaugggaacaacuccagaaagacagagaagccuuaagacaaaucuucccuagcggagaauccaaaguaguacuccccuguaauuuacaacguaugaucuggaauguacaaaaaauuuuccacauaaacaaacgagccccgacagaccuguccccguuaagaguuauccaaggcguucgagaauuacucaggaaaugcgucaucguagcuggcgaggaucgucuguccaaacaagccaacgaaaacgcaacguuacucuuccagugucuagucagaucgacccucugcaccaaaugcguuucugaagaauucaggcucagcaccgaagccuucgagugguugauaggagaaaucgagacgagguuccaacaagcccaagccaauccuggagaaauggugggcgcucuggccgcgcagucacugggagaacccgcuacucagaugacacugaacacuuuccauuuugcugguguauccuccaagaacguaacccuggguguaccuagauuaaaggaaauuauuaauauuuccaagaaacccaaggcuccaucucuaaccguguuuuuaacuggugcggcugcuagagaugcggaaaaagcgaagaauguguuaugcagacuugaacacaccacucuucguaaaguaaccgccaacaccgccaucuauuacgauccugacccacaaaauaccgucauuccugaggaucaggaguucguuaacgucuacuaugaaaugcccgauuucgauccuacccguauaucgccgugguugcuucguaucgaacuggacagaaagagaaugacagauaagaaacuaacuauggaacaaauugcugaaaagaucaacgcuggguucggggacgauuugaauuguauuuucaacgacgacaaugcugaaaaguuggugcugcguaucagaaucaugaacagcgacgauggaaaauucggagaaggugcugaugaggacguagacaaaauggaugacgacauguuuuugagaugcaucgaagcgaacaugcugagcgauaugaccuugcaagguauagaagcgauuuccaagguauacaugcacuugccacagacugacucgaaaaaaaggaucgucaucacugaaacaggcgaauuuaaggccaucgcagaauggcuauuggaaacugacgguaccagcaugaugaaaguacugucagaaagagacgucgauccggucaggacguuuucuaacgacauuugugaaauauuuucgguacuugguaucgaggcugugcguaagucuguagagaaggaaaugaacgcuguccuuucguucuacggucuguauguaaacuaucgccaucuugccuugcuuugugacguaaugacagccaaaggucacuuaauggccaucacccgucacgguaucaacagacaagacacuggagcucugaugagguguuccuucgaggaaacuguagauguauugauggacgcugccagucaugcggaggucgacccaaugagaggaguaucugaaaacauuauccucggucaacuaccaagaaugggcacaggcugcuucgaucuuuugcuggacgccgaaaaauguaaaaugggaauugccauaccucaagcgcacagcagcgaucuaauggcuucaggaauguucuuuggauuagccgcuacacccagcaguaugaguccagguggugcuaugaccccauggaaucaagcagcuacaccauacguuggcaguaucuggucuccacagaauuuaaugggcaguggaaugacaccagguggugccgcuuucuccccaucagcugcgucagaugcaucaggaaugucaccagcuuauggcgguuggucaccaacaccacaaucuccugcaaugucgccauauauggcuucuccacauggacaaucgccuuccuacaguccaucaaguccagcguuccaaccuacuucaccauccaugacgccgaccucuccuggauauucucccaguucuccugguuauucaccuaccagucucaauuacaguccaacgagucccaguuauucacccacuucucagaguuacuccccaaccucaccuaguuacucaccgacuucuccaaauuauucaccuacuuccccaagcuacaguccaacauccccuaacuauucaccaacaucucccaacuauucacccacuucaccuaguuauccuucaacuucgccagguuacagccccacuucacgcagcuacucacccacaucuccuaguuacucaggaacuucgcccucuuauucaccaacuucgccaaguuacuccccuacuucuccuaguuauucgccgucgucuccuaauuacucucccacuucuccaaauuacagucccacuucuccuaauuacucaccguccucuccuagguacacgcccgguucuccuaguuuuuccccaaguucgaacaguuacucucccacaucuccucaauauucuccaacaucuccaaguuauucgccuucuucgcccaaauauucaccaacuucccccaauuauucgccaacaucuccaucauuuucuggaggaaguccacaauauucacccacaucaccgaaauacucuccaaccucgcccaauuacacucugucgaguccgcagcacacuccaacagguagcagucgauauucaccgucaacuucgaguuauucuccuaauucgcccaauuauucaccgacgucuccacaauacuccauccacaguacaaaauauuccccugcaaguccuacauucacacccaccaguccuaguuucucucccgcuucacccgcauauucgccucaaccuauguauucaccuucuucuccuaauuauucucccacuagucccagucaagacacugacuaaauauaaucauaagauuguagugguuaguuguauuuuauacauagauuuuaauucagaauuuaauauuauuuuuuacuauuuaccagggacauuuuuaaaguuguaaaaacacuuacauuuguuccaacggauuuuugcacaaacguaacgaaguuaaaucaaaacauuacaacugaaacauacgucgguauguacugucaaugugaucauuaggaaauggcuauuaucccggaggacguauuuuauaaaguuauuuuauugaaguguuugaucuuuuuucacuauugaggagauuuauggacucaacauuaaacagcuugaacaucauaccgacuacuacuaauauaaagauaaauauagaacgguaagaaauagauuaaaaaaaaauacaauaaguuaaacaguaaucuuaaaaauaaacaaauaaguuuccguuccgacagaacuauagccagauucuuguaguauaaugaaaauuuguagguuaaaaauauuacuugucacauuagcuuaaaaauaaauaauuaccggaaguaaucaaauaagagagcaacaguuagucguucuaacaauuagguuugaaaauaaaaauuauaaugaaauauacaaagacuaaaaguuuaaauaauaugaaaaccauuuuuaacccuccguuagucgcuaucggugucacacaccgacgacagaauuauucauucgggauuuacaaaauaacuguuuuuuucuauaccacuuucuguaccucuuccuaucaacuaaccucguguuaguauacauucuuaccuacuuggguacaguugugcgaguuuuauagcuauuuucggugcaagacuccguagcgacuaacggugugguuauuacuuuauuggcguaacuugcaguucagguaaagauuuagcaucuuauuauugcauuuuaucgguaaguuuuggucaaauuuuauuuauagauguccuuugaagccgaacgaaaacguuuguuggaauuaugggaaaugguuccuagcgaauauuuuuauauuacaaauaacaauauguuuauuguuauacuaggguuuuucauuuuauaucugucguuuuucaaugaagcauuuucaaaaauauuuuuuagucauuccuauacacaauauaaaauauuuguaugaauuuuauagcaauaacuuuuuuuuuuaaauugucggucucugacaccguagcgacucuugaugcuccguuuauccuagcgacuaacggggguuaaugugaauuuaucacaagcacaaagcacauagauaaaaccaaaccuauaaguuacuucuaauacaaaauaauuaaaugguuuugaagucauaaucucgccaguuuugugaagaauuauuaaggaccagacaggucuucaagucgugccguuuucugucugcuugucucugcaauaaaucuuaaacgaaaaguccuaaugauuuaaaacuuaauauuauguauauuuguacauaggauauuuacgauccaaggaagaacaaaggaauuuggugucaaugggucaaaguuuaccaaaaaaaguuacugauaauuccgauuagaaaggacguagagccuuuuaccaucaguuagagguuagagguucggcaaaggcuuuuguuacuuuacauaauucugucagguccuuugguacucauuauacaaaagcuuuuaauuuugcaccuuucacuuccuuccacuaauucgguaccacggacuaucgaaggaccaaauauaauuuuuuaacauaccuaaagcccuugccgaaugucuaaccgaagauucaauauuucacaucccaucuggaguucucuguaacuuugagaugauuuuuaaucuauugacgcgaaauucaauaguuuucuuggaccaaaaggacgcuguguaccaaguuucaugacuuuuccugaaagaguu

SEQ ID NO: 200 - RNA, 2600 nt, Diabrotica virgifera
guugacauugacaguggggacaccuauucuggcaauuggaauuaauuugucaauaucauuuuuugucgugaaauuguguguuguaggagaaaguaauauuauuaaaaaaugucgucaaauauucaaaaggcccagcaguugauggcggaugcagaaaagaaaguaacaucucgagguuucuucggaucucuauuugggggaucaagucguauugaagaugcaguggaauguuacacaagagcugcaaaccuuuuuaaaauggccaagagcugggaugcugccgguaaagccuuuugugaggcugcuaauuugcauuccagaacuggugcucgucaugacgcugccacuaauuauauagaugcugcaaauuguuacaaaaaagccgauguauuugaggcuguaaacugcuuuauaaaagcuauagacauuuauaccgaaaugggucgcuuuacaauggcugcaaaacaccaucagacuauugcagaaauguaugagacugaugcuguggacaucgaaagggcuguucaacacuaugaacaggcggcugauuacuucagaggagaagaaagcaaugcuuccgccaauaagugucuucuuaaaguggcucaauaugcagcccaacuugaaaacuaugaaaaagcagugggaauuuaucaagaaguggcuuaugcagcucuggaaagcucucuuuuaaaauacagugcaaaggaauacuuauucagagcugcccuuugucaccuuuguguugauguacucaaugcacaacaugcuauagaaagcuauauuucaagguaucccgcauuucaagauucccgugaauacaaacuuuugaaaacccucauagaaaacaucgaagagcaaaacguagauggauauacagaagccgucaaagauuacgauucaauuucucgucuugaucagugguauacuacaauucuuuuacguauuaagaaacaaguaagcgaaagcccugacuuacguuaagacguauuuagaaauuucuuauuaauauuucuuuaaguguuauuuaagagacccauaguuuauuuauuguuggauuauuauuugaguaugucgguauacaugaagugauuuaaccuucauauagcauuuuuaaauauggaagcgauacauuuaacauucauuuuuuacaaaaaaaauuaguauagcauuuugcaauuuaugguuucuaaggauaaaguuaucaaaugagcaaauucacuuaaguucuaacauguucgauaguauaauaagauuaacgauacuuuuaacgauauaucuguauaaauauucaaugaaaauacguaaguauuaagcaauuaccaacuaauauuauuauuugcucauuuagguucucauuuuucaauuuuugaaauuguuauucuugcuauuagaguaaaaagacuuaacaauaacaguugguacuacaauugcuuacuauauuagcaggcaaaucuugguuaucacaacaccaucuuauaucagacuuuagaaacagcuuguaaaauuauauauaaugagcacuguuauauugauucuuuuaaauauuuucucaucaucuaaaggugcgaacugacuugagcauuuuucuccgaacgaacugaaugagcagagcgugcuguagcaaauugcucuagucucuagucaguacguguucacgagcaaaauucugcugaacugaauuuucggaggagugucgguaaucguugaaagaucaaaagcuaucauuuguucuuuugugcucaauagcuacagacauuuaguguauuugguguguuccgauugauuuauaugagcccuuuucuguaguauguaauccaaucaucauaaucauucaacucggaucuauccacugcuggauguaggucucccucagucuguuccauguauuucuguuuugugcuguuugcauccaauuuuugucgagccguuuuaugucaucagaccaucuuguuggcggaugaccucuacuucgauauucuuguugucaugguauccaguguauuauucgcuguguccauuuguuauucgacauucuagcuauguguccagcccaguuccacuuuagggucauaauuauuucuauagcaucugucacuccuguucuucgucuuauuuccuuguuuguaauucgaucccuacgagaaauguguaauauugagcguuccauggcucuuuggguaacacaaauuuuauuucuuacuuuguugguuauuguuaaaguuucugcaccauauguaaguacuggcaacacacacugaucaaagacuuuccuuuugagacauacauauguguaauccaauacacgggcaauauuaucauaaaauauagaaaaugaugcauggauauuaauagcugaaguaggauguaccuccaaagaagcaaagaaaaagucauauuuauaugaaucuccugucgcuauuaaccacaaaguaacaguuaagcgaucucgaagagauauauauuauaccuuuucgaaauguuuugcaauuguaggaccuacuauaguccgucccaucuugacuuuacaguauggggagcaggggcggugcaguccuuaugagaguuuuuagcgcggugaugccgauuuuaccaaaaac

SEQ ID NO: 201 - RNA, 4648 nt, Diabrotica virgifera
uuggcaauacgucuaauacgucagucaaaugaaucaaguuguugggauugggauuuaacaaauacgucguucuaaauuucuaaaacugcauacaaaaaauccuuaaaaaacuagugaagauuacuaaagugcaauagauacaaaaacaauauuuuuacguauucaccauaauaauauaaacguagaaaaugaaucccgaguaugauuauuuauucaaacuucugcugauuggagauucaggaguaggaaaaucuugucuucuacugagauuugcagaugauaccuacacagaaagcuauauuaguaccauuggcguagauuuuaaaaucaggacaaucgauuuagauggaaagacaauuaaauugcaaauuugggauacagcaggucaggaaagguuuagaacgauuacaucaaguuauuaccgaggagcacaugguauuauuguaguguacgauugcacagaccaagauucauucaauaacguuaaacaguggcucgaagaaaucgaccguuaugcgugugacaauguaaacaaauuacugguagggaauaaaagcgauuugacaacuaagaaaguugucgacuucacuacagccaaggaguaugccgaccaauuggguauaccauuuuuggaaaccucagcuaagaaugcaaccaauguagaacaggccuuuaugacuauggccgcugaaauaaaaaauagaguaggaccuccaucuucugcgguagaccaaggaaauaagguuagguucgaucaaagucgcccagucgaaacaaccaaauccgguugcugcugaaaauauuuaguucucuuugguugaagguggucacucaugucaucucuuuuguacgguagcagcgcuacaguuacucuuauucuaguaauauccgucuguauguauaguuauuuuaaaguuuuugauuugauuuucacuuuagauuuuuuuuucaaaaauauagaaaacaccaauaucucuuagccuaaucuuaacucccuuagauaaauauguaauuaacaaaaauauuuauaaaaccauuuuuacuaaacuauuaguccacaguauaucguuccuuuucaaaaaugauuacaaaagucuuaaaaaugguuuuaauuguuucguuuaaacgagucuaccacuuuaugaaaaauuucagaaagauuauacguuaaaauaaaauaaauuauuuuugucggacaaaaaagaggggcaaacacccgcaagaugucacucuagcgcgguacaguuuuguuaauuuauuaguccacaguauaucguuccuuuucaaaaaugauuacaaaagucuuaaaaaugguuuuaauuguuucguuuaaacgagucuaccacuuuaugaaaaauuucagaaagauuauacguugaaauaaaaugaauuauuuuugucggacaaaaaagaggggcaaacacccgcaagaugucacucuagcgcgguacaguuuuguuaauuacaaaacucauaauuuucaauugaaaguauugauaauuacuaaauauuguggaucucuuguccgacagauuugcagccucguuucuuuagugggacagcggaaauauguuaauuaugguagguuaugguuugcgguuacaaaagucauaauuuuaauuuuuuaacguucaaacuguucaaaaaguaauuguccgacaaaaaaauuguucgacgcaaacuuauaacaaaaacagugacagucuacuuguguuugucgguauaaucgcaaauuaccuaccucuaguauagaugcuuagucuaguaauuagucuguuccgcucugcggaauugaguaaauggaauuaauuaaaaauaaguguaaacuaugaacaauaauauauuuauuugugucgauaaaaagacguguuuugcucgaaaggguguguuguugacgcaccgcaucuaucccgacgcccgcuggggugugacugccgacgggagguuguaaaucgacccgugguuauguaucgacugacgcuguggcugaaaaaaaccacaaacgcuaauauguuuguacuucuaacaucuggccagaaaagcgauaaauuacuacaucuguaguuuuaagaaacgucuucgaaugcauuuccaugcauugacauggaaucugauaagguaauuaauucauggggaauucaguuuguuaaacugaacacaccaaaaauaugguuagggauacauuucguuucguaaaacuucuuagcaucugguuuguauauuaaaugugaauuuauaugaccguuuugagaaaugauauuaaaaaauaaaauuagcaaaaauaguaauuaaagcgugucgccacaagucuauguuuacauucgguuucuauacauuuucugacguucuagacgucacuagacauaaaacuaaacagugcaacaaguaaaggguccagggcuauguuagcucaauuuaucuaguacuguggcgguugcaguaaaaaaaguuuugaaauuaauuugucggacuggcuaucugauacccaauuuuugucggacauugcucuauuuggauaaagaacccuccaaucuaucacuuucguguucgaaauuuuauaaucgcaaucugcauaauuguuaauuauuaacauauuuacguuguucgacuaaagaggggaggcugcgaauuugucugacaagagaucaacaaucuaguaauuaauuauuaauaaacuaaaaguggguaguucccuggguuggcuguauaccauguaguuaaaaaacucuaaacauguaguaaaaccacuucuauauaaaagguauauguacugaaauuuuaaaaaucccgauggauugaauaaacuagguucauucagaacguuuucggaccugucaguccaucaucagugaauucauauauacuugcgcuaacuagucccagaaccaaacaaugguuguacuuauaaaucaaucuacauuauaguguuauguauuuggaaaggcuaagcgccgauguucugauggacugauagguccgaaaacguucugaaugaaccuauuuuauuuaauucauugggauuuuuaaaauuguaguaaauauaccuuuuacacagaagugguuuuauuucauguuuuuuguccgacgaaaauaauuuauuuuauucuaacgcguaauuuuuugauuaaaauuaaaaauuauaguuuugcaaucucaaaacaaccgcgcuagagcuacagguuuaaaaggaagucaucaaaaccauuuuuaagacuuuuguaaucauuuuugaaaaacgacgauauacuguggacuacauauaugagaucauuagaacgagaaagauauacucaucgacuguuaauuuuguauuguuuuuaguauaccuuucucuguuuuauaaauccagaucaugauagauugacguuagaaauuauuguucgcuuuucaaacguguauuagcagaauuuuggcaaauuaaauauaaaaauucgagauccuuuauaaaaagguauauuuaaaauucccuaaguaaggguuauauuaaguaacagaacguuuucggauuaaaaaauccaccaucaguguuacaaaaaaaaaauagcaugccugaggcaccgaaauguuacggguaaaaacccuuuaaauguugacaguuguugucuuauacuaugauguugcuaauauuccuggauauuacccagggcaacacaggacucuucccacgugguugaaauuugagaauuucaaucacgaauuugacacuuccaaaauacguaaaguuuuccacaucuuuaaucugcauguugugaauaguaaauagagugucguuccuugcauugguucucauggacuugauuuuacuaauauuaauuuucaaaucuauuuuauuggcuucaguggaaagugaauuuugaaaaaaaaucugcuaaaaauaguuuaaaaaauauuuuuuuuuaguugugacacuauacagauaaaauaacgaaaaauuuacgaaauaauauuuaucaaaaguggcucuugcaguacagaauauaauaucauuauuauuaucuaguuaauagacucguacuuuuuaacgauguaacuuuaaaaacaauuaacaacguuaaaaaaauacugcaaaaaggaagauaaauaagugauaaaugugucacuuuucuugagcacuacgcagauuguuuggaauaaagcuaucacguauuauacuguguaaacuccacaaacgacacuuuauacacaucuuagaugaacaaaugccuggcacaagucgauaugugacacuguuuauguucggugcaccgucaguuuugguuaaucauuagugucagcuaacgaaaauacuuuaacuauaaacuugacacuuuuugagauaagauuuuguaguuuuauuaaacuguuggugcaauaaaaccguuuuuaaaauuuuuugaguugugacacuauuugagcggaggugcgacauguggaaaauauuuacaauuauuucauauaucacuuguucuauacaauaaaaaaacgucauuucuaacaugauuuuuagugauuucuguugguccaaaacaauuuauuauauuauccauauauacuuguuauuuuuuauguaaugauuuuucuuauucgcuacacauuaacgucauuuuuuguuuauaucaaauaaaaaguaauauuguaaguaauaugugauuguaaaucuuggucaaua

SEQ ID NO: 202 - RNA, 1577 nt, Diabrotica virgifera
agaaaacaagcgguuuauuugccgccauuuugacaagcuuacguaccuacauuuuuaauucguuguuuuauuuuuaguuuucaauaauacucuaaaaaauggcagacgcugaugaucuauuagauuaugaagaugaggaacagacagaacaaaccgcaacugaaacggcaacuacagagguacagaaaaagggugucaagggcacauauguaucaauacacaguucuggguuuagagauuuucuguuaaaaccagcaauucucagagcuauaguggacugcggguucgaacauccuucagaaguucaacaugaauguauuccucaagcugucauuggcauggauauucugugccaagcuaaauccgguaugggaaaaacggcuguuuuuguauuagcuacacuccaaguaauagauccuacagaaaauguuguauauguucucgucaugugccauaccagagaguuagccuuccagauaagcaaagaguacgaacguuucaguaaauauaugcccaauauuaaaguaggggucuucuuugguggcuugccuauccagaaagaugaggaaacguuaaaaaauaauugcccgcauaucguuguggguacuccaggaagaauuuuagcauuggucagaucgaaaaaacuuaaucucaaacaucuaaagcauuuuauuuuggaugaaugugauaaaauguuggaguuauuagacaugagacgugauguucaagaaauauaucguaacacuccccacgaaaaacaagucaugauguucagugccaccuuaaguaaagaaauuagaccaguuugcaagaaauuuaugcaagauguaauucaaaauucuuauaauacacaauuuuguaaugacgcacccacucgcaauguuugaaaaguucuaauucaaaauacgggccaaaguuuuauuaaaucgaaucauuuaaaagaugaggguguuggguuuaaaucauuagcgcauuguagcccaacgaguaggcaggggugggccgggccaagaagaggacacuauacaggauuuucuaaauuuccguaaaggguuccgcgaccacauuuucggccgagcuacaauguagaaaaauuuacacacccuccaaguccaauagaauuaaugggguuaauguuugaaguaccgauggaguucuuuguugauuuauuuagaagaguauuuuuggauaauuguucagagaucauuucaauggugaugcagcgaugaugggaugcgagauaucaacugacuguauguacgauggauucagucaagacuccugagccucguaacugcagacuauuucauuauuuuauuaacuaaaguaauaauugaguuuuuuaucgccucaauuauuauuucaaauaauuuauauaaagauuuagaaaacuaugauuuuguugaugguuauauuuaacugucggugaguauuuucauucacaaaaguguguguguuauugcguugccgcuccugaaauaucuagaggccaccuaucgauggaucaagaguaaacuaccuuuauaguucauaacauuuaaauuagaaugauauaauugcacauuaaaacauaauua

SEQ ID NO: 203 - RNA, 1039 nt, Diabrotica virgifera
gcaucuggucugcccgcauaagcuuguuggaacugccagugaguacacagugauguucacagggaagacagugaacggugagguguggaguaaugagaaucaugaguuucaucaggugcuaaugcaacuuccaacaaaaacagauuuuuuucguaaaaugugaacguguuagucagucaaaauagcaucaaaaugccggucauugaugguuauaaaguacuuuacauuuuauuacacaguuuauauacaauuuuugaaaauauuuggaggacucuuuuauuuauuuaucaaaauuguauaaggguuauaaacccugaaucuacauucgaugaugcugaccaguuaaagaaaagacugucuagacuaacaaaaaagccucaacauuuaacuaucauuauugguguggaagaauauucauugguagauuuggcuaaccucguauauugguguuuaggucuuaauauuccguacguuaguuucuaugauuauaaagguaauuuaaaaaagcaugaagagaaguugcaacaaauuguagaauccagaaaaucagagaauaucaacauaauuuggcacacccaugcagaacaaaggcauaaaaauggauuuuuggguccaaaaauccacguaaaaguguuaacacacgcggacggaaagcaaaguauaguaaauguuacuaaaaaauuagcucuaaauaaagaaaaagacauuaguaaagaaaaaauuagugaauuacuauuaaggcaguaugaauuuccagauccagaaauggcuauuauuuguggaaagaaacugaacauuuauaauuauccuccuuggcaguuaagacucacagaauucuuuaaagucaacaaagucaacaacaucacauucccaguguuuguggaaaaauuggaaaaguacagcaaaugugaacagagggugggaaaauaauuguuuuauaaaaaacuguuuuuguuuugguuaucuuuauuauuaagaaaaaaaaaaaaaaaaaaaaaaaaaaaaaaaaaaaaaaaaaaaaaaaagaacagaggguggg

SEQ ID NO: 204 - RNA, 697 nt, Diabrotica virgifera
cucgaauaaacgccacauccaaacaagaguugacguugacagcgacauuuccuccgccauuuccuuuuacuguguuucuuaggauuuggaagguacaaaaugaccaacucuaaagguuaccgccgaggaaccagggaucuauuugcccgcaaguuuaaaaaacgugguguaauuccacuuuccacauauuugagagucuacaaaguuggagauauuguagauaucaaggguaauggugcaguucaaaaggguaugccccacaaaguguaccaugguaagacaggacguguuuucaauguuacugcacaugcauuagguguaauuguaaacaaaaggguucgaggaagaaucauccccaaaagaaucaaucuccguauugaacauguaaaccacuccaagugucgucaagacuucuugcaaagaguaaaauccaacgaaaagcuacguaaagaagcuaaagaaaagaacauuaaaguagaacuuaggagacaaccugcccaaccuaggccagcacauauuguuagcggaaagguuccagcacaggugcuugcuccuaucccauaugaauucauugcuuagguuuguuuaucuuaaaauaaaauccuuuauauaauaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 205 - RNA, 784 nt, Diabrotica virgifera
cugacuucaauuaaauggccucugucaaugucagaguaaaugucaaaguguaaaaaauaaaaaaaguaacaacacaacagauuuuauuaaguuguagacguguaauuuuuguaaauuuauucuaagccauuauuaauuaaaguaauacuaaaauggaaggaauacuacuggaaccaacauuguauaccauaaaagguauugcuauauuggacuaugaugguaauagagugcuggcuaaauacuacgauaaagauauauuuccuacagcaaaagagcagaaagcuuuugagaaaaauuuguucaauaaaacucauagggcagacgcagaaauuaucauguuggaugguuuaacuuguguguauagaaguaauguagauuuauucuuuuauguuaugggcaguucacaugaaaaugagcuaauuuuaaugaguguuuuaaauugcuuguaugacucaguaagucaaauauugaagaaaaauaugcaaaaacgagcugucuuggaaucacuagauauuguuaugcuggcuauggaugaaauuguugauggaggaauaauuauagauucugauucaaguucaguaguaucuagaauagcauuaaggacugaugauauuccauuaggagaacaaacuguagcucagguauuccaaacggccaaagaacagcugaaauggucauugcugaaauaaagugcguauuuuaaaacaagguaaucgguauuuauuucauguacaauuuaauuauuaaguguaaauaaauuuuuucuguuuaaagaugaaaaaaa

SEQ ID NO: 206 - RNA, 1850 nt, Diabrotica virgifera
guuugacgcauugcagguacaagccuaccaaucacaaacacacauuguacgaguagacguuguaaacaagccgcacccgauguucaaacuugacauaacauacgccacgucaacuagaucgcccaacacuacacaauuucccucacgaaaaaccaccgcauccaacacaguuuaaaauuguacagcgaaaauaccaucuucaauaguuuguaaagcugggaaaacaaauaggaaaauuuaaacguuucguguguuggcguaggaauauuucuaccgguuucugucauugccgcacuauugcgcuuuggugguuucgcagcagcaguuccccccucucuucucuuacgucgauccuaaacaaaacgagaaaagacaguuggcuggugcacuuugacgagaggcguuguuaguuacuucguugugucgacauuuuucuguuuaguguaccgauuaaucuucaaauauuacaauggcugaccaacucaccgaagaacaaauugcugaauucaaagaagcuuucucacuauucgauaaagauggugaugguacaauuacgacuaaagaauuaggaacaguaaugagaucucuaggacaaaauccaacagaggcugaauuacaggauaugaucaaugaaguagaugccgaugguaacggcacgaucgauuucccagaauuuuuaacgaugauggcacguaaaaugaaagauaccgauagugaggaagaaauucgugaagcauuccgaguguucgacaaagacggcaaugguuucaucucagcagcagaauugcgccacgucaugaccaacuugggugaaaaauugacagacgaagaagucgaugaaaugauucgggaggccgauaucgauggugauggucaagucaauuacgaagaguucgucaccaugaugacuucaaagugaggaaaccagaguugcauuuucagcuuuccauuguuucaucccggcucauugucuacauuuuucaacaccucgaacuuuuugcuuuguggccggguccacuaaagugaauaacuuaaccguuauuugauuuacaagacaaauuuaauuaauaaauauauuuauaaauuaauauaauuguguaaauuuuuguaauauauuuguuuuucuuucgcaggagucguuggcgagaucuagucgcuggcgauucuuguacuguaucaaauucugugcuacaugucuaguugaaguuuaaaaagaguuacaaagucacaaaguaugaaaacacguauauauaaugaagauaaaaaaaucguucuauuugauguuaguuuaggggauacagcauuagacacuuucaauuuuuuucauacuuugugauguaaauuacaucugaucugugcacuuacuauauaguauguauccccuuuuuugacauguucauucauuuuuccccuuucuuguacuguagguuuuacauuuuuagucuaggauuugaauuugcuggaaaguaagcacauuuuuauugucauccugugaugcguauggaaauguuaauauuuuuuggcucuuacuguaucacaggaaauggucuuuagaaauugaucgaaauuuuuuacaacaaaaauuuuauucuaauuucuuaagaccacucccuuucuuauuguaagauuucguuuuuauucaagccuguuaauuuuuuuauuuauucuuacuauuuaagauccaguaacgcauccguuacaaugugaaauuauuagauguuuaaauuugaaauaaaugugacuaaaauauuuuccugugacuuacugguacccaauauauuauuacuauaaaguuuuuauguuagaauuuuuuucuuuauuuaaaauaaaauauuugaauuaaaaaaaaa

SEQ ID NO: 207 - RNA, 3245 nt, Diabrotica virgifera
cagcgccauuuuaguauuuagcucugaagauugaguuuuuacuuccgugaaaaauuuuuacgaguuuuuauugucaucguuauaauauuuucgacaacugcaguucuguauauagucgguuugaaaaucuuaaagaaaauaaugaggguaugaauuuaaacgugcuccaauauuagcgcuuagcguguauuugccgguguauuguguucaacgucaccaucaaccaugaugcaagcaaacaaucgagucccaccuauaaaguuggaaaacgauauagaucuuuacgccgaugauaucgaggauuucgcacaagaugacuuugguggugaaaauguugaucuauaugacgauguaauauccgcuccuccuggaaauaaugacaacccaggugauucaaaucaucaugcuccuccuggugcuggugaagaugguggagguaauuuuguugggucaggaggagcacccaauaauauaaauucuucuggaagaagacaucagcuguauguuggaaaucugacuugguggacaacugaucaagauauagaaaaugcagugcaugauauagggguaaccgacuuccaugaaguuaaguuuuuugaacacagagcaaauggucaauccaagggauucugugucauaucuuugggaucugagggaagcaugagacucugccuggaacuccuaucuaaaaaagagaucaauggccaaaauccccuuguuacccuucccacaaaacaagcucuuaguaacuuugaaagucagucuaaaacacgcccuucuccuacuaauaauucuaacucacguccuccccauccuaauaauaauguucauucagguccuaugcagaauuauggagguagaaugccuaugaacccuuccaugcgucccaugcccccagguaugcaaggugcuccaagaaugcaggguccaccuggauuuaauggaccaccaaacaugaaucagcaaccccccagguuccaagguaauccacaauggaauggaccuagaccuaaugguccugggcccaauaugggaaugagacccauggggccaccucauggacaacaagggcccccaagaccaccaaugcagggaccaccgcagcaagguccuccaagaggaaugccgccacaagguccaccgcagaugcguccagaauggaaucgaccaccaaugcaacaaggguacccucaaggcccgccgcauaugcaaggaccuaacauggguccaagagguccaccccaaaugggaccacccggggcgccucaacagcaaggaccagcuccgcacguaaauccagcauucuuucaacaaggaggaggaccaccgcccccaaugcaacacaugccuggaccagggcccgucaugccuccucaaggacccccgcaagguccaccacacggacccguuggaccuccacacggcccaccauuggguccagcgaauguuccgccucauggaccaccucacggauaugguccaccugcagcgaugccacagccgccauacgguggcccaccuccagaccaccgcgcugagauuccucaguuaacagagcaagaguuugaggauauaaugucccggaauagaacaguuuccaguucggcgauugggcgggccguauccgacgccgcagcuggagaauuugcaagcgccauugagacuuugguuacugcuauuucacucaucaaacaauccaaaguggcuaacgacgaucguugcaagauccuuauaaguucgcugcaagauacuuugcguggugucgaagacaaaagcuacagcuccagccgcagagaccggucaagauccagggacagaucacauagaagaacuagaagagaacgauccucgucacgguacagagacagaagcagagagagggagcgugaacgcgauagagaucgugaucgugaacgugacagauauuaugauagauacagcgaaagagaaagagaccgagaucguucaagaagcagagaaagaacagaaagggauagagaacgagauuauagagaccgggaacccgaagagacagauaaagaaaaaucuaaaguauccagagucucaagaucaagaaacaaaucuccggaaccugucgaaccuagcagcgagguaccgaaaucaucccgcuauuaugaggauagguaucgggaacgagagagagaaggucgacgagagagcgaucgcgaaagagaaagagauagaagaggggaagacagccauaggucucgacacuagcauauaguuagcgguucacagaaacaacaacaaacaaguuauaguuggaguucaaacaaagauuaucguguuuaauuuagagauagguuauaauaugugauguuacuuuacauaaauuuaaacagguuccgaauguguaaguuaaaagagcaaaggaaacauucacuaacucauauuuugcuacuuguuucauuuugcaauggaacuucuggcuaucuuaaauguauucauaaaaaggauaaaaaacauuuaccuguuuauuguuguauuaaguaaaucauugacuauaauauugcgauaaauguuagauauuuuacuucaugggguuauacuuuugauguaaaaguuucacauauuuagcuuuaagcaaacgugacuuacuaugaauauaauaaauuauguaagaacaaaauuaaguaaugaauauaccacauaauucuucaacuaaugaguuacuauguacaguauauucuucuuacuauuucuucaguuuucuccuggaguagcaaaacuuguuuuuguaaaaugaauuuauuguuuaggcugugcuauauuaacacugggucgucuauuaaucuguuaaaaaacaguuuuugagagaauucuguucuuuauaaaaaauuacaggagggugaugaaaggcuuuauuguuacgugcauguugguaaaucgggaugcuucuaaaugauaaauaacuuuaggcaacuuguuguuucugugagcuguauuuuuucacauuuuuauguaaucauacauaaguaaaucauguaacguauaguugauaaguacaauauagauggaaaucaaguuuuguuuuuguguguaguauauggauuuaaaacauguaucuuuuaauaagaugauguuucaacauacaaugaauaauuuguguaauaaauugaauacuauucaacaugaauugucaaauucuuacaaaaaaucaugauaauuugaaggcaauugaccaugaacuacucacaaugcaucuaaaugugaguguauccucc

SEQ ID NO: 208 - RNA, 1023 nt, Diabrotica virgifera
gucgucagucgucaacaucaauuucaaguuucaagaaaaagcaaaucacuacgacuugccggauuuuguaguaguguuaauuuuguauuaaaaaaucaaaaugaguucuauuggaacuggguacgauuuaucagcuucccaauucucuccugauggaagaguauuucaaguugaauaugcaaugaaagcaguugaaaauaguggcaccguaauaggccuccgagguacagauggcauuguauuggcugcugaaaagcucauuaugucaaaauugcaugaaccaaguacaaauaaacgaauuuucaacauugauaaacacauaggaauggcauuuucaggcuuaauagcugaugcaaggcaaaucguugagauugcuagaaaagaagcaucaaauuauagacaucaauaugguucaaauauuccucuuaaauaccuaaaugauagaguaagcauguacaugcaugcauacacuuuauacagugcuguuagaccauuugguugcagugucaucuuggccaguuaugaagauagugacccaucuauguaucugauugauccaucuggaguuagcuauggauacuuuggaugugcuacagguaaagcaaaacagucugcaaagacugaaauagaaaaauugaagauggggaaucuaacaugcaaagaacuuguuaaagaagcagccaaaaucauuuauuugguccaugaugagcugaaggauaagaauuuugaacuggaacuuucauggguaugcaaagauacgaaugguuuacauaccaaagugccugaaucaguguuugcugaugcagaaaaagcugccaaacaagcaauggaagcagauucagaaucagauacagaagauauguaauaacuacauuuaguuuuuaauauuucgcugaugguggcuguucuuacaauauuucguguguuauguucauauauuauguaauacugugagaauuuccauuucaaggauagguuuauaacuuuuuuuucuaauaaauacauaacuuuaaaaaaaaaaaa

SEQ ID NO: 209 - RNA, 1439 nt, Diabrotica virgifera
aaagugucaggugcuguuggugucagcugacuuuuuacuuuuuguuccaguccuauauucaacacacuuuggauauuaaauauuuuauucuaaaucucaagaauggcuucaaaagacagauugaugauuuuuccaucuagaggagcccaaaugaugaugaaauccaggcuaaagggagcccaaaagggacauaguuuauuaaagaagaaagcugaugcuuuacaaaugagauuuagaaugauuuugaacaaaauuauugagaccaaaacucucaugggugaaguaaugaaagaagcugccuuuucuuuagcugaagcaaaguuugcaacuggugacuucaaucaaguuguucuucaaaaugucaccaaggcucaaauaaaaauaagaacuaagaaagacaacguugcugguguuacuuuaccaguguuugaaugcuaccaagaugguacagauacauaugaguuggcugguuuggcuaggggaggucaacaauugacaaaacucaagaagaauuaucaaagugcuguuaaacuguugguugaauuagccucuuugcaaacuucuuuuguaacucuugaugauguaaucaaaauaacaaacagaagagucaaugccauugaacauguuaucauuccaagaauagagcguacuuuggcuuacaucauauccgaacuggacgaguuagaaagagaggaguucuauagauuaaagaagauccaggacaaaaagaagaucagcagagcaaaggccgagaaacaaaaacaagcucuucuccaagcugggcuacuuaaagagucccaggcaaacaugcuuuuggaugagggcgaugaagaucuacuuuucuagaacaucaaacagccugaagugugguucuguacauaugaauaaauauauaacgcuaacuuguuuuuagacgguaacuguuuauuuuucgcauuaauuaauacauuuuuaagauauaucuuuauuuuuaacugguuuuuauucuuaugccuugcaauaguaaaagauauacgaacccggauauuuccuauauaaauaacuuccuacuuuuuauuaacuccaguuuuuaggauuuuaauacaauauucaacacaucgugcaauagaauuugaagugaauauuacuugcgaaaauuaaaaagguaccaaauauuuuucuuaauauugaacguauauuccagaauaguauuauaaaaguuuugauugaaauucuuguacgugacacuaugaacuuguagauuuuagagaaagcagcuuuucaauggaaaaugcuuuauugauauggacacuagauaauguaaauacuuguuaauauacuuugaacagauaaaauaguuuauucguuuuaaauucuuuaaaacucuaaauuguuuuucaucaaauguauacauggccuguaaauuguugguuagaaauaaaacucuguucaaaaaaaaa

SEQ ID NO: 210 - RNA, 5226 nt, Diabrotica virgifera
gcgauugcgucugcugaaugcacccauaguauuguggugacugugacaggugacagucggagucgacagugaaguacaaaucaaaaucauucccuauuugucaaacaaaaaugaaauuacguguauuauuauuuacccaaguaacuuuuuaauaauuuaagccgauuauugcacuguuaguacguauguacaccacaaauaaccaauauauucuuuguuucccaugauaacacaguucuaauuucacuguuagaugcauguuacauguuuuuaguggaaucauuacuggauaaaguccucucccauaauuaaauaaauacuuaaaaaauggaggcggcucccaaguuaccgaugcucucguucgaguuaaauacuuguacagagaacguccacuuuggcccccaguuaaaacaguauauugcugcuuuuuauggugaagauccagaauccuacauuacagaaaucagcaaucuugaauccuuaagaucagcugcaguucgaccaucaacggauguaaaugguguacaacuguugaaaaaguauuucugucagcuucguuuucucaaaucuagguuucccauggaagagaaucaagaugcugcaguucuauuuucauggaaaaauaaugaauuagacauaacuucaacauccagugauaucagauaugaauuaaugguaauaauguauaacauuggagccuuacacacuuuucuuggagccaacgacucaagaaacaauccggaugguaugaaaauggcauguacucauuuucaaugugcugcaugggcuuuucaaaacguaaaagaaaaguaccaccaauucauaucaaacaucucauugguagaacugguucauuuuuuucaacaagucuguuuagcucaggcucaggaguguauauuagagaagagcauguuugacaauaggaaaccuaccaucauugcaaaaguugcuauccaagucuacaguuauuacagacagucuuuacgugucuuggaaucaguaaaugaagccuacuuuagggauaaaaccuacaaggaguggaugaaauaucuucaauucaagcugaccuacuacaaaugcaucucguuccuauuccaagggcaacaagcugaggaacaacagaaaaugggagaaaggguugcauucuaucaagcugcaugugaacagcuggacgaggcaaagaaaauugcugcuacauuaaaaaaccaacaccaccagcaagaaauaaaugagggacuagcauucacuacugaugugguugaagguaaaagaaaagcagcuaaaaaugaaaaugaguucaucuaccaugaaucagugccugauaaagaccaauugccagagguuaagggugcuucauuagucaaaggaauaccauucaguauaaaugauauagaaguuucaggaccagauauuuucucccgauuggucccaauggaggcacacgaagcagcuuccuuguacagcgagaagaaagcucagagauuaagacagaucggggaacuuauugaaaauaaagaucaaacauuggcugaauuuaugucgucaaugcagcuagaucuauugaccaagaugcaccaggcuacuggaauaccgcaggaguugauugauagagcagcggcucuaucugcuaaaccuaacgccauucaagaucuuauaagugcuaugggaaagcuaucuaauauauaccaagacguugaagcaaguuugaaugagauugauucuuuauuaaaggccgaagaacaaagugaacaaaaguaccaagaaacgauugguaaaagaccaccgagcauuuuagcuacagauuuaacuagggaagcggcaaaauacagggaggcucauacuaaagcgaacgacucaaaccaaacuuuacacagggcgaugauggcucacguggcuaaucugaaaauacuccaacaaccgcuaaagcagcugcaacaucagcugcccuuugucgaguuuccaaauccaaauaucgacgaaaaaucuuugaaagaucuggaagcgcuaguugcaaaaguagacgaaaugagaacccaaagagccaugcuaugggcucaacuucgagaaucuauucaccaagacgauauuacaaguucccuuguaacgaaacaaccaaaucagucgcuggaacagcuguuccagcaagaacuucaaaagcaucaaaaucugauuucguugauugaacaaaacaccucggcacaagaaaacauuaagagcgccuuagucgauucuuacgcuuacgcuguaaauucaagaaaauacauccaagauauacuccaaaagagaaccacaaccauaacgucacugauagcaucguucgacucuuacgaagacuuauuggcaaaagcuaacaaagggauagaguuuuacucaaaacuugaaacgaacguauccaaguuacugcaaagaauaaggaguaccugcaaaguucaacaagaagagcgagaucagaugaugucgacugcgcaagugccucaaugggagagucauacgucacuugccgcuccuaaacugaaagauuacuuggacuccaggaagaagagugcugcguauucggagccgaguguucaaccacaacagccaacuuuaaguuacucagcugcuauggaucugccuccugguauuaggccgacuccaguuggaucagaaauaacggauguaccgaaaaauauucaaggugaaccacaagguuauauuccauauaauuaccaacaaccuucuguuccugccucacagaauauugaugaagagacuauuaaaaaaaugaacgcauugaugccaggugcuaagacgucagugccuagucaguacggauacagcaacuacauuccaccaacauacccucaaagugcguaccaaccagguaaucagucuuacggaaaagaaacuccagauauuaacucaccguacgacccuaccaaggcguucacggcuacuacuaacgcuuaucguucggugcagagcuccucaacucaaggauacguaccguacgcagaaucuaacguuucgaauguugacagaguuggauauccuagcagguaucaguaccaacaaguaccugagauagcuacuacuccagcugaucccaauauuaaugcguacuacccacauggguacucaccgagccagaauuuaccgaaugcuaauacucaacauauuaccggccaacugaaguaccauucgguggaguacgcuucuucugugccgaacaacaucaauuauaacagcucuaccuacucgucgccgcuuucuaauaugucuaguaccaauuccucaaauccuaguaacuugaauaauucuuacgaguacuacuaugacccgaauaccaguaguggugcaguaccgaaugcuucaaagccucaacagucgagcgccagcucugcaaacccgaguaccgcuaugaacaacuacaauuauuacuacaauacaaguaccagcgguaguguagcagcggauacuucaaaaauacaacaacaacaacaguacccagguacucagaugagucaagcgcaguacuaucccgccaaugccaguuauuacucaaccaguacuuacaauaccaacguccaaggugguaccaaucccucguacgcaacuggacaaacauauaaucaagugacaccagugaccucucaaaauguuucucaaaauuacaacuuuaaccaaguugguucuggagcaggacaccagcaucaguacuacucguccgcuaacgccgcaguaccaucccaacaagcuguaaauaacaguucauuaccaaacuacggauacgaucaguauuacggcaacaacuauaauuccagucaaccgaguaccuacagcgcaaaccaagcaccuccugcagcacaagcugcuccaaguaauauuccugcugccaccaaauccuccucuaauguggaucugcucaguggcuuggacuucagcauaagccaagcuccucuagugccucaacaaaacauuacgauaaaaccccaagaaaaggaaacaaaaccaccggcuguuucuucugaaaccaaaaaccaagauccaacaccaguaaccacgcccaaacaacccacuggaccagaaguaaagcgcuuguacgucaaaauccugccgagcaaacccuuaaacaacgaugaugugaagaaauuguucggccaagagcuggacagguaugagaaguucguggagaccuugacccacaaaacuuugagcgguccgaccacucuggauauuaaauggaaggagauccaagaccagcaggauugcgagccgcagaagaagaucauuuccgucgcuagauguuauccuaugaagaauagguucccggauaucuugccuuacgacuuuuccaggguggaguugugcgauaguaaagaugauuauaucaacgcuucauacauuaaggauaucucgccauaugcuccgucauuuauuguuacacaagugccguugucuucaacuguuggugauauguggacgaugauuagagaacaacaggucgaacugauccucuguuugguaaacgacaaugagaucggugaagauauuuacuggcccaaagaaaaaggcaguagucuuaacauacuuaacauggucauaacguugcaaaacguuauaguuaagucucauuggacugaaagacugauagcgauaaacuuaccugaaaaacgggagucccgugugauaaugcaucuacaauuuacaucguggccuggcagcuuguuuccaacaaauccugaaccguucgucagcuacaccuuggaauccaucaaccuauaccaacaacagaagaccaacacccauccgguggugguccauuguucaucuggcauaggaagaagcggccugcucuguuuacugacagcugcuauguucgaugcugccaacaaugcuaacucgauaccagaucuuacagcuuugaguaucaaguuguccaauugcaggaagaauauucucagagaucgagagcauuugaaguuugguuacgaaaguuuuuuggcguauauuaggcauauaguuugugaagauaaagccagaaagaaacugaacgagauccagcccaagguuaaggaggaaccacuggaaccaccugucauaguuccagaaccaaauauagauccuuuaaguacuuuagacccauuuugggcuaguaaaagauaagcuuuacauaguaaauauuuauacaaugauguauuauuauuuugaauguuaucuacaccuucauuaauauuaaauucccug

SEQ ID NO: 211 - RNA, 633 nt, Diabrotica virgifera
uaccgccugcuacucucuuucuuugaccuccuccuuuucuucuccuuuguccggcuuuaacuugcguuuucccggcuccagucccgagcauaccuucuccggaguuuucgguucuuccgguucuuuccaaaguacuggggacucucuuucuucuuugaauccaaugacaacucuuucuuucggcggcuucuuaauuucuuucuuguucuugcguuucgucggcuuuccuccgcauaguaacuucuuuccacgccauuuggguuugaacagcuacugcguuuacuuccggguaauuuuguucauacacucccuauagugucugcauaacaucuggaucuuuuauucuuuaaacuggagcuuuuucuucaccuuaagucucuaaaagucuagaggcuuaacuugucgguucauuugcuggaaucuccguuuaagcaguuugguuggaacuucuuccauagguuuaugcuuuuguuuaagcgguuugaaguuuucuuccgucgacuuaaauugaaggcauugguugaguuucaacaguucuucuuucuuaaguggaaucuucuucuucuguuucuuuucuuuggucugaccaguuucuucccucugcuuuucuuccauguucuccgacuucguacu

SEQ ID NO: 212 - RNA, 603 nt, Diabrotica virgifera
uacacacuucuucuucaacggcgaaaucagcaucuguuaccuaggccauacacguuucgaccaaaacgaccccuacuacguggagcacgacauaagggaaguuaacaaccugcggguucuguagucccacacuaccauccuuacccuguuuuucuaaggauacauccacuacuucgaguuucauuuucuccauaggaauggaauuuuaugggguagcucgugccuuaucaguguuugacccuacuauaccucuuuuaaaccguaguauguaagauguuacuugagucucaucggggucuucuugugggacaagacaacugucuucgaggagaguugggguuccgguugucccuuuucuacuguguuuauuacaaacuuugaaaguuguggggucgguacauacaacgguagguccgacaugagaggaacauacguagaccagcauguugaccauaacacaaccuaagaccacuaccacauagggugugacaggguuagauacuuccaauacgagaaggaguacguuaggaagcaaaccugaaucgaccaucucugaacugacuaauggaguacuuuuaaaacugacuugcaccgaugagaaag

SEQ ID NO: 213 - RNA, 2742 nt, Diabrotica virgifera
uacggugaagcuaaucuauauuuuucuuucgauugucgagcgagucuggcccauuuuacacaccuagaagugggaugucuuggaaccuacgacacaagagaaaugucgccuuuauauuugcaaaccuuguggcuuuuagucguugaccaauucugaaaacuucauacacuacauggacaagccugucgauucaaaaacggguccuucuugaccuaucagucacccagacuacuauacgucuaagcucaaaaguuaauguuauggaaucuagcccauguaagaaaacuccgaguaagccuaauacacucuacauaacagcaugugggauguguuggaauauauaauuguucaucacuacuauacgaauaguucgaaaccuuaacccuuuuucguacccgaacagucguucaaaagcuuccugugugaguaauauaauacguuuagcgguauuuagguuuucuguuguuguguaaacggucacguagggaucuaucuuguaacuuucauaccguuaacccucgcaggugucgcuuaaagugugaucuuccaguacucuuuccgcaauugacacaccugauaauagugccaccucuauuuggaauaaauuagaguccgcgacuacuaucuaaucauuuuuagacccuaauaguuuuguuuugaacacaaguuugaaaccuuccuguacgaguuuuacauuggcgacguacaaagguaggucuugaaggacaucgagaaugaccuucacuucuaccaugacagucucacaccguacgguuguggguauccaaucuuucaucgaauuuaauaccgaaacuuucucauaccugauaaaagacggauuucccuagguuauugcaccguaacccaauacuacuuccaucguaaaaccaauuucaaccaucucuucuuggucgacaaucauaccuacggucaccuccguuuuaauaaacccggucugugagacuugaaguuguccguuuagaguuccgcaaucgacuuccacgccuuuauucucuaccucuugcggaaggucaaagacauuuucuauacccacgaacgcucuauaugggagucuguuaaguuguguuaggguuaccggcaaaacaacaacagacaccccuaccucuuauguauuagaugugucguuaccgaaauucuuuguuucgcaaaccaucgcguguucuuaaacacacccgaguucuaaggucgcuuauacgguagucucuuaggccuagaugauagucuuaaaaauucuuaaaguuucucuucuucuuaaaauucaggcuaaaaccucgacuuccauauaugccaccuauggaaaacccucaguuuagccaaagaccaaacugaaagauacuaacccuuugagagcuaaaucagucuucuuagcucuauguugguuuucgucaaaugaccagucuaucaccauuuaaucauacaaaccggugucuucuaucgaugaaauaagaaagaauacuaagacuacuucaaguuuuucggucucuauuguuaguccaacgccuacuaccucaucuuagccgaaaguuagaagauccacuuuauuugcuuagucacgcuugaccagagacccauccgcugacaaaauagaugugcuuaagacaauuagcauaguugaugaagcaaccuccacuugaccaauguuaacgaguaaaccuggccggaaacauacagaacccuauacacggauuucugcuaucuaauauggagcaucuauuucucaacgcgcaucauucgaugguuaaugaagaaagacaagaacuuauaguuugacggcaguacucuucucugaaagguugucgucugucucaugaaggcagguaaggauuccucgugucuugcucucaccguguaaagaaucuuuucguuccgaaguuugucguucgaaaccggcauucaugucuaggucucgugucuaagcucgaccgucaucguaaucuccuagaauuauaucgguuuugagaucgaguucuucgcuugucaggcguuuucaccuuaguugaucgucuuaaccgucgacgaugauuauuacauucgcaucgguuccuuacauacguuuuucgcguucuaauaccuccgaacaacgaagaacgaugcucgaggccacuacuuuuaaaucaggcaugagauccucuuugcuguguucgacuuucguuuguauugaaucguaaaaacagugugaacaaucauccacuaaauuuguuuacagaucuguaagaauaauuauggccaucuaacggucuucgacguaaaaagcggucuagaauggaaggacuauucuaaugucuucagcaccuugacaccuucugagucaauagaagucaguuaguuuuucgaccugucucggaacggcuaggauuuuugaugcuuuuagacaagggaccaaauguucuccgccaccaucgagucuuuaaaaaccuugucgucuuauuuccaaaucgcgggcguucucaacgguggugguaaggaggauuagugcuguccuuacaacaucggcuucaaguucguguuagcuuugugcuacaugguaguaaaucaagaagcaaauaaaguagucuuuaucuucguguuuguuccucaagacgauuuggacuucuuagaaguuuguaauaugucgaccugguucuacugcugcuauagcuaaaucuaaaccugccacauuuauagcuacucuuguaacugugcugccuauaguuguagcuacuacuaaacgacucacuaacu

SEQ ID NO: 214 - RNA, 1173 nt, Diabrotica virgifera
cacaauaacuuuccguuuuuaguuuauuacgucuagaaacauuuuugugagugaccauuuugguagugggagcuccaacuugguagucuaugguagcucuuacaguuucgauuuuaaguucuguuucuuccauaagguggucuaguugucucuaauuagaaacgaccuuucgucaaucuucuaccggcaugagagagucugauguuguaagucuuucuuagaugugaugugaaucacgaagcagaaucuccuccauacguguagaaacauuuuugagagugaccauucugguagugggaacuccaacuugguagucuaugguagcucuuacaguuucgauuuuaaguucuguuucuuccauaagguggucuaguugucucuaauuagaaacgaccuuucgucaaccuucuaccggcaugagagagucugauguuguaaguuuuucucagaugggagguaaaccaugaagcagaaucuccuccauacgucuaaaaacaauuuugaaauugaccuuucugguagugggaacuucaucuuggaagacuaugguagcuuuuacaguuucgguuuuaaguucuguuucuuccauaagguggucuaguuguuucuaauuagaaacggccuuucguuaaccuucuaccagcaugugagagucugauguuguaaguuuuccuuagaugggagguaaaccaugaagcagaaucuccuccauacguuuagaaacauuuuugugagugaccauucugguagugggagcuccaacuugguagucuaugguagcucuuacaguuucgauuuuaaguucuguuucuuccauaagguggucuaguugucucuaauuagaagcgaccuuucgucaaccuucuaccggcaugagagagucugauguuauaagucuuucucagaugggagguaaaccaugaagcagaaucuccuccauacguuuagaaacauuuuugagagugaccauucugguagugggagcuccaacuugguagucuaugguagcucuuacaguuucgauuuuaaguucuguuucuuccauaagguggucuaguuguuucuaauuagaaacggccuuucgucaaccuucuaccggcaugagagagucugauguuguaaguuuuucucagaugggaagugaaccaugaagcaaauucuccuccuuuaauu

SEQ ID NO: 215 - RNA, 1131 nt, Diabrotica virgifera
uacacacugcugcuacaucgccgagaacagcagcuguuaccgaggccuuacacguuucggccaaagcggccacuacugcggggagcacgacagaaagguagguagcauccagcagggucugugguuccacaguaccacccauacccaguuuuucugaggaugcauccucugcuucggguuucguuuucuccauaggaguggaauuuuaugggguaacuugugccuuaauagugauugacccugcuauaccuuuucuagaccguaguguggaagauguuacuugaaucucaucgggggcuucuuguaggguaagaaaacugacuucgaggugaauuggguuuucgguugucucuuuucuacugaguuuaguacaaacuuugaaaguuauggggacgguacauacaacgguaaguucgacauaacagagacaugcgaaggccagcauggugaccauaacaugaacuaagaccucuaccacauagggugugucauggguagauacuuccaaugcgagagggugugcgguagaacgcaaaccugaaccggccaucucugaacugacugauggaauacuucuagaauuggcuuucuccaaugagaaagugguggugucgacuuucucuuuaucaagcacuguaguuccuuuuuaacacgauacaucgaaaccugaagcuuguccuuuaccggugucgucggucgagguggaggaaucuuuucucaauacuugaaggacugccaguucaguagugguaaccauuacuuuccaaggcaacgggacuucgagagaagguuggaaggaagaacccauaccuuagaacgccauaggugcuuugacagauguugagguaguacuucacgcuacagcuguaggcauuucugaacaugcgguugugacaggaaagaccuccaugguguuacaugggaccauaacggcuagcauacguuuuccuuuagugacggaaccgagguaguugguaguuuuaguucuaguagcgagggggucuuucuuucaugaggcaaaccuagccaccgagguagaaccggagggagagguggaagguugucuacaccuagagguuuguucuuaugcugcuuaggccgggaccuuaacaaguggcguuuacgaagauu

SEQ ID NO: 216 - RNA, 2640 nt, Diabrotica virgifera
uacccaugaaaauuuucucuaugaguacuacuccugcccccuaguucacgaaaaguuuuagaccucuuuugaugacaaaacguccuucgaucucaaaaauuacuuugaucacauuuagguucuuuuacauguggcuaugauuguuuugacaacaugaauaacuuggucccacuuugaaauucacgguuucuccggugucuacaaaagaaacgguacugguuugacaagguuaguuuucuacauuauaacucuuccuaccaaauaaacccuuaauuucuugagucaagacaacgacuacuacaguaauaacauuguaggucagaauguuuucuauacugaccauuucuucuguacaugucucgucgacgauauucucguaauacgucauaaugacuacgaugauacgaaguucgauaucuugcaauauacuucguucgauaacaucuaucuuugcgucgacagucaagucgucgugauuaaucaaguaauguauacucguuuaaucgaggucuacaucauuuuucuacccauuuacuucgaguccuucgucauuuaucacuauuacguuaccaugucauagugcguaauccagaagauaugguauaauccuucugacuauucgaucgucacuguuuuaacuaaagguucgacuuaaguuacccaaauuucucgggaauacgaaacacauacaacuauucuuagugacguuuugaaaaucuucuucuccugguucucagugaggaguugagggguauauguuauuauaaauguuacccgaauuccuuguuuagacuuuaccaccacauacuucgacguguacgguaccaauuggacuucaagugcucaucauuacacgaucgugggcgauauucacaagauguugauaaaacaccuagaggauuucggugugagucuaaacgacgacaaucuugaaauuuaguucaccgguggugggugggacgcagucacugucgaacauuaaaccuagaucuuuuaaacuaaugacuaggauuauccaguuaacgaugugaccgguaaugaugagaaaacuuuuguccacggcuuagaagacaacugucugauuacuuuguuuagcgaugaaaacauagacuuuagucacuacuuaaauuucaccaacaguaaguccguuaauuccauaaucgaaacuuuaaagguuccuuuguaucgugcgaauacuuaaaggauaggcgguacaauucucuacucccuccaaaucuuauauuucguagguaucgucuaugguaauauugggauuagcuucuauuagggcuucgauuucuuagaccaaaccgcguagaaacgcucaaguaacuucugacacuuguacaaagaaaccgacacucuuagaacguaaacaauccuuuccuuccuggguucugguuuguugguagcucuauguaggcaaaauagauguuagcgcaguauaaccuuacaggaagacauucucgacgacgucagaggcgguaccguguuaagccucggagaacagggcuaaacaaucuuuuauagguuuauaaugaaagcuccacagucuaccuaagucugcuacuucaaucccugucucgauguauaauaucauuauaugaauuguuuuuacuauuuucaaauauguuguuaauguaaaaccuaagaaacguccaaaguuaaggaagugaucuuucuagcgaaucucuuauguaaguuuuagguugacugcuugguaaacuguaauucaggcauggacaucgucgucacgguugucgucuucuugcucuucaauuuuuguuuagacuuccugacgaucagagaguuccaggucaggcuggaggaggccacagaucucuucuuuugaagcggcuuuuugaaucauugcaaggcccauauguugucaauccuggaaacaaguuuugaaggcugcagcaacuugagugacuuagacuuugucucauaaaacaggcgacauaguucgugacaaaguuuguaguguagcaggagguuaagcuaacagacuuauggaacggucuggucgaaaaucuuuugcaaucucaccucuaucugcggccacuuuggaagcuuuaaaaccgucuuuauggaacacuuuucaacgugauauugcuuuggccaugguguauacaucaucaauucaacggacuacuacuagagggguugagacaaccaugcacaccucggcacaacuucaagaaucacuuucuaacacuagguaguugcccuuauggucuaagacuacucccaaugcuacuacuuauaugugaccuucuguagcuuuauuguaauccccugguuuaaguuuuucauucguuucauuuaacccgacgucggacccuucuucgacgucgaugaauacaucuuuuucuccuauguaugaggaacugguaguuaugcgauucaccgcgacaauucuuauaauaagucaagaacccuaaugucggacgccuuuccugacugucucauggccucccauuuagaugcguguguaaugaagaacgaccacauaaguccccuccauaacuguaugaucauucucgcuuugaucgaaacccgcuuacacaaugcuacguugauugucaguccagcggucuaggacugcaacgacucgaauauugaaguugacauccaauu

SEQ ID NO: 217 - RNA, 651 nt, Diabrotica virgifera
uaccgccguuugucuugaccuggacgagucucugguuuaccgcgauggguuccuuucuauacagucaaguuugaccaggaugauccgcuuucacggcagccauucagcucaaaccaugacuccaagcaguuuccugucaaggugcuuaugguccucucaugguauccucgucgaaaggaauguguuugguauacggagcugcuauguugacaauuuaaacuuuaaacccugugucgcccaguucuuuccauggugucaaaucgaggauacaugauauccccgcguguccgucgauaucagcagaugcuguauugguuaguucuguguaagccgucccgcuuuugcacccacuuccuugaaguuuccguccggucaggcugcuagcacuaucgaaaccggccguuguucguucuaaaccgguuguuugcauaccaucuuaugcuucuccgcgucugcauacgacugcuuuugccgaaugaaaaauaccuuugaaggcguuucugccguuacuugcaguugcuauauaaaaaucguuaucgauucuuugacggguucuuacuuguuugguguccaguuccgccgucacggguuccguccgccgaucgccucccgcuaagcccgcgguuccgugggccuuuaacaacguucacu

SEQ ID NO: 218 - RNA, 279 nt, Diabrotica virgifera
uacuucaaaaauucuagcugucacacgauguaacgguagaaccguuaagagaaaugggagacacggcuacuccaacuuccuuccucuuuuuaaaacuaccccgcuuuuucguaaugguccuguauagaagcaccuuuacgacaaggacgcauacacuauuauuaggaacauccuuaaccaguuuaguaggacccucccuauaacaugcaacguaacuccuucuucuaguaacgacguggacauugccguaguauacgucaccgaucuguucuugguauu

SEQ ID NO: 219 - RNA, 474 nt, Diabrotica virgifera
uacgucuagaagcaauuuuggaauugcccauucugguagugagaacuccagcucgggagucuaugauagcuuuuacacuuucgauuuuagguccuauuucuuccuuaagggggucuggucguugcagaguagaagcgaccuuuuguugagcuucuaccagcauggaacagacugauauuauaaguuuuucuuaguugggaagugaaccacaacucuaacucuccuccacgauucuuugcauucuucuucuuaaugagguggggguucuuuuaguucguguucuucuucuuccaauucaaucgacauaacuuuaaaauauuccaacugcuuuuaccauuuuagguggcuaacucugcacuuacggggcgacuuguuacaccucgaccacagaaguaccgucgguaccuucuguccguaaugacaccguucacgccaaugugagaacagaagagguuugguccucuacucuuuauc

SEQ ID NO: 220 - RNA, 747 nt, Diabrotica virgifera
uacuacagguuucgucuguguguccuucuacggaggaagcgguuuaaccuuuuagucuaacgauaguaguuuaugcauuaugagaaaugguugcaaaacguuacccgagagccacgucguuagaagcgagaaacggaaaccgaugcuaagcuccucccguaaguucuuaccgaggucuuuaaccuaagucuuguuaaaauguagccucauauacaugaauaucagcgaagugacuagcaguacuaacacaggaaauauccuacauaaucacgggacguccucucaugguaccgggaaaaucacauguagccguggguucacgagucaaaauauaagccaaauaggccaagccgccaagaagaccuauugucgcggucucuaagggugaagguuggcuccuaggcucucucauacgcugcagaauaguacuuacgaguagugcugguuaggucuguuugugaucgguacuaaguccuuuuacaaccaacgacgccucgacuaccgcguugucugauggagagagaagucgucggggaagguucagucacgucucuguggcaaugaccuuuggguaagaaggugccuacacaucuacuugaguggaccaagaagcuucuuuuuacaccaaccuaucguccaaaucgauaccgcuauacguacuaauugcaggaaucauaacaaaauagaugccaugaguagguccguaacuuuuuucuucuucuucguaggcuaaguauguccucuauc

SEQ ID NO: 221 - RNA, 375 nt, Diabrotica virgifera
uacagaccugcaccguucccuccguuucauuucccuuuucguuucagggcuaguuuagcacgaccaaauguuaaaggacauccagcauaaguagcaaauaacucuuuuccuuuaauacggcuuucucaaccacgaccucgaggacauaugaaccgucgacaauaccuuauaaaucgacgacuucaaaaccuuaaccguccuuuacgucgaucucuauuguuuuucugggcauauuaaggaucuguaaauguuaaccgguauucuuuacugcuccuuaacuuguuuaaugacaguccucaaugguagcggguuccaccucauaacggauuauauguucgucaugacaauggauuuuuuugacuuuucuuucgaauu

SEQ ID NO: 222 - RNA, 864 nt, Diabrotica virgifera
uacuucaaccuaccacaucuagacgggggugguuaaucgaagcuguaacgccuucucguuggcaaugguggaacgguugucugcaagaauacauuaccacuaccuccuagguaucacgcugucaaagagcucgacauaaagcauuauauacuaagucuauuauccgucagggaagaaguccguauagugcuuuuucgguguaaaaguuacuguuaccggaugggcaugccgauaagguuucugucauuuccucauagcaccaacuuaaccauacgguggcuauuaucuuuaaauaaugcucaaguucuaggucugucuucuuuguucaacaauucugucccuguucaacgacaucaaagcaagaacguucuauacggcgugugcuucgugcuauaagugucaaaaugucaucuaaacugucaaaaauguggggucuacaauacaaacugucaccgaccauacaaauuucuuaacuuuucaccgguguuucauggaggaaauucuauaaagaagucuugggaacauuaacauggacgaccuaguccaaaaacguaucguuuacuucuugaaguguauagguuacguugaggccugguucguuuucuacgaaaguucugguggcaauuacaucgaggccguggucggggacacuaauggagaggaccugggucauaugguguugggcgacacggucuacuacgauguuuuguucuuuaccauuuugucuacaggcgucauaggccuuacuuagagcucaccagcgaugucacagagcuucuuuguguuacccugauggucuuucgguauuaccauaagguuuuaaauuugcguguuccacaacaugguguucgucguaaauaauuu

SEQ ID NO: 223 - RNA, 2868 nt, Diabrotica virgifera
uacugacgccaucuuguuggaacaaugugugauuauuugaacgguugucuaagccucgggauguuacuuuacguugauuuuuaccuaaaucuuuucccacuccaauuucauuuuuauucucguaaucuuuuuuauuaaguguacuaagaccguccucuuuccaacggcuuaccuaaagauuacugguaguauucuuugcaaaauggaaauguucuaguaaaccguuuuuuugauaauaacuaaaagacccuuuaucaagguuuuuguuuaggucuuccauuugaugauguucucuacuaaaaccauacacuacggauaucuuuucuagacguuguggguuuacuuaaaaacucuccaagaugugaagcgaagaacacguuugacuuccuuggucuuaacaaccuugguaauuacgggucauaaucucgaacaaaccuaguauccgugucgauacacuccuccuuacgacaugaccguuaaaaaugguaaauguuuuuaaaacuucgggaguaaggucuacgaggacuugacuagagguuaauaaaccuaccacucguucuguacagaacauuuucuuuacgcaaaaauuacgaagaaguacgacugguucuuucccgcaacagcauaaaccguaguacaaaucuaguucauuuaaguaaaccucuauaagauguugaccaguagcaacucaacuauauauuccacacaguaagguuaggacgccuuucuagaucuaaauaaucuacauauauauugaacaacuugaguucgucaggacgacaguccaugcuucgacguccuugaaaucagugggagaggucacggggcugacggcaauuucgacgacgaucgacaauguaacucaauuaauaguuucuuucacuguuguuacauuuugaguagcaaaaccuguccgacuaucgugaauuccucgaaggauuagugcuuucuuaagacguccuaaaucaauaccuguaugacucucaugagagacgaggacugaaucuucaggcguucuucugaaauucagaucgggaacuuaaucagagaagugccuuguaucuucuuuaccauaaucauaauuguuuccuucacucauuuugccaucugucacuuguacuccuauguccuuucauguccguuaacaaucauuccugagauguaagcacaagguaauucaagggucuauagcgugcaucacaauauggucagaacuaacuuaaaaauaggcuauuauuacuugaccgacggugucuacauaacgacaagaauucccuucgguaugucuucaaauuucuuaacguuggcaauuaauaacucuuugaguagcuuugaaaguuucuguaauuuaaccaguuucagguaucucgucguuaaaccuaaaacccucuuaugcgcucaugacgaaggcuauaucuucaauagcaaccucuuuaauugucuaacaacccacuuccuagggagcaacuucgacucgucuucaauuaucguccucuaugccuucucuuacgaggacguggacgacguccgcgguggugaaaucaaugaaggcuaccuuguauacgauggguuagucgaaaguugugacagucgguuuggugauuucuucgugcuggaggagauucuguuauggaguaccuaccacuaaaaaaguagccucggagaaaccguagauguaauugguuugacagaaacgccauacuccuggaguggagaggacgacgaucguuaccuaaguuacgguuuuaauacgaauaauaccgaccuuaagaagugaacccuuuuaguccugaaggguguuuuaguuauugguugcugcuauuucugguguaagacaagacaaaugcucaggauagacuagcaagagguuaguaacaacuuuaaaaguuuuuuaacacggcgagccgugauuuacucuacgaagaucgauuccuuagccaucuucgcuagagcguuuucucguuucuuuuuuuguucgcaugcuaaguuugacugcugcgauauucgaaggacguuaaucucugucuauuuucaccucucgauccucuuuugcauaagcucuacagcgacaguguucgaaaucauccuccagcuugcccuccaccgcuuagucauaauucaagguuauuuaaucuauuuuaguguguugacugaccaaaaaggcuaggucaaauaaggcuucguaugcaagugcacuuagucaugcuauagcacgaacuacagaauuagcauuugguuugauugcuaugaaauguuuugacgugcgaucucgaccgaugaaauccgcuaaacuucaaccaucucuucgguguuggacagcauaaccgcggguuucugaaaacguuguaauuucgauugcacuuucaccggaguugacuuuugccuuaauauaaaccguuguaacacauacuacaguauccucgccccagucuauccuuacaacaucaaaacuuacuauauguguaucuauauuaccugauauaucacggacgaucaacaugucuaucgcucaaauacucuuacacccgccuuaaacuuacccuuuuauuccauuggcaauuguguggggagugccuugaaagucuuauggagcuuguagaugaguuuucguguuuaaacuuuacaaauuguagucuuuuucgagacucgcccgucacaccaaaauaccgucgguuaaauauacguuuuagguaaaaaccucuucugcgaaaccgguugaauucauaucucuuuggaaaauuauuugggcuacgcggucauucgccaguauaaucuuauucccgguuuucagucccguaccggaauucaaauccucuguuucaguuauacugugucuucucguguguuguauuucaucaucgacguauu

SEQ ID NO: 224 - RNA, 549 nt, Diabrotica virgifera
uacccauuacacaaacguuuaaauaaguuuccggagaaaccguuuuuccuuuacuccuauaacuaccauccugagcuacgucgaccauuuugguguuaaaauauauuugaauuuaauccucuuuaacauuguugauaagguuguuaaccuaaauuacaccucugacaucuuauauucuuguaaucaaaaugucauacccuacauccaccaguucuauuuuaauccgguaacaccucugugauaaagguuuuguguguuccggauuaaaagcaucaucugucauugcugucccuugcauagugacuccgauuucuacuuaauuacgcauacaaccggcuucuacuugaaucucuacggcaugaagaguaaaagcgguuguuuguucuaaacggguuacguuacuugcgacgucuuuaguggcuguuugagccagagguaagugaugcguuggcguugaccauguaaguucgauggacacguugaucgccucuaccagagauacuuccagaccugaccaacagguuaguuaauuucuugcgguuagcgauc

SEQ ID NO: 225 - RNA, 4425 nt, Diabrotica virgifera
uacccugccaacgugacaaauaaaacauaaaaggauacaaaauggcaguaguugugcgucuguugcuuauauguaccuaagagccuuuuaaggcaccuauguaaagauaguggaguacccuuuucacguuuuaagcacgugguuuuaguguuuggguuuuaacuuuuaacagucuugauguuugggagccacuuucuucuugucgguccgccuugcaugcauuguugccaauggcgauagcuacugcuaggaucccucccuccuccuuguuaaucaauguuugauuaaguaucucuuccucuuguacaaaauaaacuguaucuguugcaaugaccacaaaacuguugauaggucgguaaacuagcccuacuuggucauuccgucuuccuugaaauacauuggcauguucgaagucuguugccguccggugguaaucgucuacagacauguaaaugucaaugguaauggcuguaauuacuauuacgcggugucgaacuauuugacuuuaugcuacguguucaaagacuucuaaauuuucauccuucacuucacuacucucaaaaacgaaugcuguaacuacuaccccuuuuaaguucuaauagcauauugaaaaguuugcuuuuacgaguuaagugggucauaaaguccuaucuagcucuaugaccgcaacacauaaauuuccuucgaaauugucuguuuuucuuaugaucuaaauugucacgacaaauacaucggcuauuaccgcaauugcuaguucuuccgguuuuucuaaguuggcgauucuauagauauugucaucaucccagacuauuugucggagggucuaaaugaguuuuuuacggacuaccuuagaaccucuaggggcuucuaaaauuucugaaaagauuuguauaacagugucagcuucguugcagguuguaacgccuagguguugaacgaaaacuuaaccacuucccuuucuguauaguuugguuauuucugguuugcaaagaaaaccuccgucuuccuuuauuucgcguguauuucgaucacgcaggugaccuaauacuuugucauugccuuauaugagauugauaagcucauuuuuuguuucuaaauuaccuaagaagguauuuauaugguuaauuucauaaucuacaauuacuacuuuaaggauuaaaggaacuuaaagaauuuccaucacagcaccuuuuacuguucgguccacguguucgcuaaguucauucucguuagcuauuucugccuugacgacgauuguuguaacacucgauacuugagcaacuguuaugucuaaacaaacguuauuuggcuagaugcccucauuaaugcagcucucaccucaaacuagcacuuugacauggagauauagugcauuugcaauuucgaauacuauugagaggcagacgaaacauauugugcuguaacggauuguaacauuaagucuguaagguuuagucauaucuucuaguuuuacuguuguuuggacauaaaugaguagguuaaauagucaagucauuauaaugacucgaacgacuauuuagcucauaacaaccacuucaguuucgaaaucuauuacugugccgaagucaauauucaauaucauaauguuuaccuuuauaacugcuacgcaaauacuaacuuuuaagauggccgucuuauucucaauuaccuuuugaccuaaugcucuuuuagcuuguuauguugaauuggcaagcgcguaaacuaccccguaaacuucuaaaacguuaacaaaauuaaagguaugaauuacuuuuacugcuuggaggucaaaaacugcugauauagucucuuuaaguuuaauuucuccuucuuggauacuauaggccuacgcaacaaucucacugacgaguacuaggucuauaauuucuguccguacgacuaguuguguaucauauacuccagcgcuuucuugucuuucuaaaaaacuggcauagacggcuaccuacgcauguucauuguuuuggagagcuggcucuaggcggaaagccaucggguugugcuguucagaaguagauacgagcacuauuacuaccuccguguuuaaguaacaaccggugacgucuuuaacuuuaauaaaauuaucuauauuugcuauuacgagggaaaaauuuacaaugucuuuaacaaauaauacuuuugguccuagguccaaaauauccauuggauucacggcuacuaaugcuaccaggacuauuaccuggaggcaaacgaaaagcuaauagucugugacgaagucuaucauaaucuagcuuuaaaagguaauagccuuuggucgaaaagcgaaaucuuuacaaacuaucucuucucguuuuuauaauacuguaacgguaacuguaaugucuaucaccucauggaggugauuguccuugaucauaagaaucucaauauuagccucuacauuuacuauuaggucgaugucugccuuugucgugcuagaaacacauauucaugcaguuacccggucuuuuaaaguaccuuuagccugcacauauacaaugucuggaucugcuaacccuaaauuuacuguuucagaaacaaguucuucuauugaaacuacuuaaacacaauuuggucguauuguugccauacuaagacuacuuugguuguugccgacucccuugaauacuccaaguaaugucccagugacuuuggguacuuggguguuaugugcuuguaugucaauuacguuaucagugcuaaugucaauuucaugaaggucuccuucgccaacauuuuaguccuaguuaagcuaacucuccuuguugauuccuucuuaaguaucuuuuaaguaacuuaccuuucucguuuucucuguauaaugugguucuugagagguuuuauaauuuauguagaaaucgcuuacaacuacauaaaugacaaaauuuaagugggguggucuuaucaagcaaacaccuacaagcuaaaagacgaguaccuagagguauaauacgaggucucuuugagcuuuuguuucaaugucuaguaguuuaccucgaacuuguuuuuaaucuacaccuuaagauguacuaguugcauuugcucacggaauugcuuuguugcacaccucgacuuuugaguacaugcuuguuuaauuuguauugugcucuuggucgacaucacaaaugauugucuuguaggaaacagccacauuuacguaaauaacuaggacacacacggcgaaaugguucucuacaauaccuuacaaaguugccuccgcaggaauagcuuuugugucgcacauuaacaggacguccuaaacuuccugguguaacacuuuaggaucgauauccuaaauguccuugaccaacccgauacauagguaggaaacugcgauguuuguccugacucuaauaugacguauaaaauaguguuugacuauuaccaaacuauaaaauguuaccuggaaauuuauauucuguuugaagaaacagauuucuaauauauaguaaucuugaauuucugccuauagguaaugaaguuuaaacguggccgaguucgugaguucuuuaaauagacuuucucgcguaaguguuuaacucgcuaccuagcaauguguuuuauuuuuauccuagaccuaaacugcuauauagggaccuucaucugcugacaccuuguugcacaaguuaaaccugauuauuugauguauuuccacaauaggcucguuuaccgggggaaguugacccuccauacuuuuugucuaaguggcuaguucuuaaguuugcuuaaacccugguaaacgguggcugacggugggcaaagagaccaacauaaucuuuaaacugcauauuacuuaaaaugauguuggagccacguggaagacuacguaagguucgcauagggcugacauugauacgucacuacguucgacacugaaagccauagcugagguuaaagaaccaacgauaagaccaaacacaucguuaaaacuauuaagaagaagaccgucaucaacauguaucugcauuugugcuguugaaauugcuuuuucuuuagcuacuaugagcgcuuuuguaauaguugaugcuucuacuuccaccgccgcuuacacugugguugaugcuggacagacaaaagguagucuuguuguaacaccugcuuuuugguaacuacucucuguuggggcuacauggacgucuauauucaccgaaaaaucuauuguucuuucuguugacacuauuucuggggcuauuaaacggaauacugcugcaagcgguaauacggaugcucccucugccuuuaucguggccuaggaauagaagagagcgaaguacaugccugcuuccucuaaauuucaaguugaugaauaguucaaaaccugggucuaagucuuucaaucggcuguacauaccucuucuagguucgcuacuucugagugugcuaccuuugcuucuuaggaccacgauc

SEQ ID NO: 226 - RNA, 291 nt, Diabrotica virgifera
uacggaaagacaccaggguuuaacagggagacgccggacuaauagucacguaccccauaguaggucaacccaaaguacccacauaagauaauguaaccccgacaccgaaaucgucuucuauaaggucuccaacucaaauucccgcuaaaucuguuuaaaauaucgcugcaguugugcccaaagugugucuuacgaauguugacgaccuaacgacgagaggauauggacuauuguaaucgucauagucgagugguuaagacccgguuguugucuaguaguaacuugcagauu

SEQ ID NO: 227 - RNA, 540 nt, Diabrotica virgifera
uacccagaaugguauagucgucacaaauuauccaacaaaucauuuuucggauacucuuaaaauuaccauccuaaucuacggcguccauuuugguguuagaauauguuuaacuucgaaccacuuuagcauugauguuaugguugguagccgaaguuacaucuuuggcaacucauguucuuauauagaaagugccauacccuacauccaccggucugcucuuagucuuuugagaccucugugauaaagcgguugugacuaccugaguaaaaacaccaacuaagguugcuggcucuggcauagcgccuucggcuucuucuuaacguguuauacaauccucuccugcuaaauucucugacguaaaacaauuauaagcgguuguuuguucuaaauggcuugagcuacaggugacgacuuaacuggcuauucgaauucaacgugugaaacuucuuauccuccaccauguauguucgguguacacgaugaguucccuuaccaaacaugcuuccugaucuaaccaacagcuuacuuaaccgguucacu

SEQ ID NO: 228 - RNA, 618 nt, Diabrotica virgifera
uacgccaugugaaacucaauguagccacgaugggaucgcaggugacauugugacuaaaaacgggagaugaugacggagugcccuuuuccucucguucaaucaaaucguaccaauaacaacuuacacagagggguguacacccguccagauccuuaaccggaacgacauaguaauagucaacauccucgacgacguccuuaagugugauguccucagucauagcauccucgaccacaauuucgggggucuuaguuuugguuuuuaaauuaaagauaauaaaagacacuucgacaccgauagauacccaauuaauaccgauaucaugagacaccuucaaccuucuuaaagcuacaucuggauaaguuggaguuuugaguauugaaacgaguuuugguaauaccuaguguacaauaaaaaccuaggccaaauugacaaccuaaacauuuagauaauacaccuaaaacacaaccucaucaaccaagaccacgucgguaaagacuacgucgguuaaguaguaauaagcaguuuuaaaacuaauaacucuaaaaaccuucacgguaaccagagaagccagacuaacaaccucauaugaacugcaguucuccgagauaccaaauu

SEQ ID NO: 229 - RNA, 5694 nt, Diabrotica virgifera
uaccgaugguugcuaucauuucgaggcaacuccugucaauuuucucacguuaaaccuuaugaaucaggucuacuuuaaucugcuuacagucagugucuucccccguaggcgaagggucuuugguaccuucguccggcgggguuugauacgccagaauaccuggggucuguuccacaguaucugucuucgaguucuacggucuguacacggccuuuauacugucuuacaggaccuguaaagccuguauagcucgaccguuuuggucaaaaggugcauccuaagcauuguuuuuguuauuucuagaacucuacgcaaacgaagaaaacgucauuuaauaaucagucagguuuauuaggcuuuuaauuucuucaacauuacuuuaguuucccugucggugcauuuucuaaucgaaagcaaauacuagacacauuuccauuuuuauaaacacuuccaccucuacuuuaccuacacccauuucuuucgcuucuaggguuauuuuuucguccgguaccaccaacaccagcuauaguugguuuauagucugcacggccaaaucuaaauugucgucuuaccuuugugcaguuacuucuguguguucuuuucuuuuagcgugauagacggcuugcacagacccuuuaggauuuuguauagugucuacuucuuacaaaguaagaaccauaccuaggguuuaaacgaucuggucuaaccuacuaucauugccaugaaggacaaggaggggaucgucaugcuggacgacaucaauacgugccuagacguuccuuaguccuacuauagugaguguuuaaccggcuguaauaguuccgcuuauugcuuaaugucuucuugcucagacguccacgucggcgaguauauuagugucuuuuauaauucuacaacguuaaagugcagcggugaaaucaacuguugcuauacggcccuuacggcucucguuacguuuuuagaccuuuuggggauuuucgauaguuucgagccgacuuuccauuucuuccuuccuaagcuccauuggaauacccuuucgcacaccugaaaagacgugcaugacaguaguguggucuaggguuaaaugcauagcugguucauccucacggaucuucauaacgaguuuuguacugcaaaggucuuuagcaguguggaaaguuaaaacuguuuuacaaccuuaaccaugucucuccauuaagagucauagguccucgauucauauaguagucucuguuaccucucuccuaacuaaaugcaaagguggguuuuggcagucuaaauguaaacgucacaccaauauuccaucuuucuguguagucucugccgcuagaucauuagaaguuggcaguugguugggagguguucuacucauacuacccggugucucaguuucagaaugggaccagcugcaaggcauacuuagagagcacguggagagggauguugcggcuaaaacugccgcugcuuuacuuggagguacacgggguuucauaccuuugagcucgacuucagcuuuuggagguguagugaggguccguuuaguaaugaggcguucgauugguugggcaguacccauaacauguucuaugcaacugucgacaauccuucuacuguuuuucccuacauaaguagcucuuccuuguuuacuacuuauauaacuacaagaacgguuaaacccuaccauuuuacggggcaggucgguaggaguuuggguuuggcaacaccuguccuuuugucuauaaaagggacuaguaaggaccguuacauuuauacuaugcauggguaagaugcguaggucugcugcuccugcugccagggauauuuaccuauagcgguccucuaugcuuucaauaccaucuuguaccucuuaaccaguacccauauaacacauucuuuucagaaccuuguagucguccaagggacgacguauaaacauacaaccuuaauccugugcuucacacaccaucuaaaauaccauuguaaguuugacauuaguuguugaccaacaacaaucuuccagugucguagccauaaccucugugguaacggcuaggagucugaaugugucuuuaagucucucgguaguccuuucgguuucuucuacauuaucuucaguaggucuuucgaguguuguaccuugaccuuggcugagggccauuaugcaacgcagucugaaagcuuuuaguucauuugucuuaagauuugcugcgagcacuguuuugaccaccaaggcgauucuuuagaaacugacuuauguuauuggauuuccgauaccagcauagcccuagguucccuagguuguaauuauaaaggguccaauaacgaacgcacccaguugucuugcaucuuccauuugcauaagguaaaccgaagucuuuugcgugcaacggcgugaaguaguuccugcuaaugccaggacuuaggucuccaaagcaucuuuuaagcauagaacggccagagugaggaagccucaagauaaaggugcgauacccuccagcacuuccagaauagcuaugacgacauuuuugacggcuuugaccaauguaggucgcagcagacuaguuccgauaccucucacauuaccaugugaugcugccauggcauucuuuaagacauccuguugaauaggucaacucuaugccacuccugccugagacaccucucuaccaucucaaaguuauaaaucguugccaguuuaauucauuguuccgcaaacucucuuuuaagucuaaacuaggcucauuacuuuccauaaacucuucucaaaaguuacuucuucaauaguucguugacuacccaaguccccuucaguaaaggcuugaacucucucuuacccuuguugaggucuuucugucucuucggaauucuguuuagaagggaucgccucuuagguuucaucaugaggggacauuaaauguugcauacuagaccuuacauguuuuuuaaaagguguauuuguuugcucggggcugucuggacaggggcaauucucaauagguuccgcaagcucuuaaugaguccuuuacgcaguagcaucgaccgcuccuagcagacagguuuguucgguugcuuuugcguugcaaugagaaggucacagaucagucuagcugggagacgugguuuacgcaaagacuucuuaaguccgagucguggcuucggaagcucaccaacuauccucuuuagcucugcuccaagguuguucggguucgguuaggaccucuuuaccacccgcgagaccggcgcgucagugacccucuugggcgaugagucuacugugacuugugaaagguaaaacgaccacauaggagguucuugcauugggacccacauggaucuaauuuccuuuaauaauuauaaagguucuuuggguuccgagguagagauuggcacaaaaauugaccacgccgacgaucucuacgccuuuuucgcuucuuacacaauacgucugaacuuguguggugagaagcauuucauuggcgguuguggcgguagauaaugcuaggacuggguguuuuauggcaguaaggacuccuaguccucaagcaauugcagaugauacuuuacgggcuaaagcuaggaugggcauauagcggcaccaacgaagcauagcuugaccugucuuucucuuacugucuauucuuugauugauaccuuguuuaacgacuuuucuaguugcgacccaagccccugcuaaacuuaacauaaaaguugcugcuguuacgacuuuucaaccacgacgcauagucuuaguacuugucgcugcuaccuuuuaagccucuuccacgacuacuccugcaucuguuuuaccuacugcuguacaaaaacucuacguagcuucgcuuguacgacucgcuauacuggaacguuccauaucuucgcuaaagguuccauauguacgugaacggugucugacugagcuuuuuuuccuagcaguagugacuuuguccgcuuaaauuccgguagcgucuuaccgauaaccuuugacugccauggucguacuacuuucaugacagucuuucucugcagcuaggccaguccugcaaaagauugcuguaaacacuuuauaaaagccaugaaccauagcuccgacacgcauucagacaucucuuccuuuacuugcgacaggaaagcaagaugccagacauacauuugauagcgguagaacggaacgaaacacugcauuacugucgguuuccagugaauuaccgguagugggcagugccauaguugucuguucugugaccucgagacuacuccacaaggaagcuccuuugacaucuacauaacuaccugcgacggucaguacgccuccagcuggguuacucuccucauagacuuuuguaauaggagccaguugaugguucuuacccguguccgacgaagcuagaaaacgaccugcggcuuuuuacauuuuacccuuaacgguauggaguucgcgugucgucgcuagauuaccgaaguccuuacaagaaaccuaaucggcgaugugggucgucauacucagguccaccacgauacugggguaccuuaguucgucgaugugguaugcaaccgucauagaccagaggugucuuaaauuacccgucaccuuacugugguccaccacggcgaaagagggguagucgacgcagucuacguaguccuuacaguggucgaauaccgccaaccagugguugugguguuagaggacguuacagcgguauauaccgaagagguguaccuguuagcggaaggaugucagguaguucaggucgcaagguuggaugaagugguagguacugcggcuggagaggaccuauaagagggucaagaggaccaauaaguggauggucagaguuaaugucagguugcucagggucaauaagugggugaagagucucaaugagggguuggaguggaucaaugaguggcugaagagguuuaauaaguggaugaagggguucgaugucagguuguaggggauugauaagugguuguagaggguugauaagugggugaaguggaucaauaggaaguugaagcgguccaaugucggggugaagugcgucgaugaguggguguagaggaucaaugaguccuugaagcgggagaauaagugguugaagcgguucaaugaggggaugaagaggaucaauaagcggcagcagaggauuaaugagagggugaagagguuuaaugucagggugaagaggauuaaugaguggcaggagaggauccaugugcgggccaagaggaucaaaaagggguucaagcuugucaaugagaggguguagaggaguuauaagagguuguagagguucaauaagcggaagaagcggguuuauaagugguugaaggggguuaauaagcgguuguagagguaguaaaagaccuccuucagguguuauaaguggguguaguggcuuuaugagagguuggagcggguuaaugugagacagcucaggcgucgugugagguuguccaucgucagcuauaaguggcaguugaagcucaauaagaggauuaagcggguuaauaaguggcugcagagguguuaugagguaggugucauguuuuauaaggggacguucaggauguaaguguggguggucaggaucaaagagagggcgaagugggcguauaagcggaguuggauacauaaguggaagaagaggauuaauaagagggugaucagggucaguucugugacugauu

SEQ ID NO: 230 - RNA, 876 nt, Diabrotica virgifera
uacagcaguuuauaaguuuuccgggucgucaacuaccgccuacgucuuuucuuucauuguagagcuccaaagaagccuagagauaaacccccuaguucagcauaacuucuacgucaccuuacaauguguucucgacguuuggaaaaauuuuaccgguucucgacccuacgacggccauuucggaaaacacuccgacgauuaaacguaaggucuugaccacgagcaguacugcgacggugauuaauauaucuacgacguuuaacaauguuuuuucggcuacauaaacuccgacauuugacgaaauauuuucgauaucuguaaauauggcuuuacccagcgaaauguuaccgacguuuugugguagucugauaacgucuuuacauacucugacuacgacaccuguagcuuucccgacaaguugugauacuuguccgccgacuaaugaagucuccucuucuuucguuacgaaggcgguuauucacagaagaauuucaccgaguuauacgucggguugaacuuuugauacuuuuucgucacccuuaaauaguucuucaccgaauacgucgagaccuuucgagagaaaauuuuaugucacguuuccuuaugaauaagucucgacgggaaacaguggaaacacaacuacaugaguuacguguuguacgauaucuuucgauauaaaguuccauagggcguaaaguucuaagggcacuuauguuugaaaacuuuugggaguaucuuuuguagcuucucguuuugcaucuaccuauaugucuucggcaguuucuaaugcuaaguuaaagagcagaacuagucaccauaugauguuaagaaaaugcauaauucuuuguucauucgcuuucgggacugaaugcaauu

SEQ ID NO: 231 - RNA, 609 nt, Diabrotica virgifera
uacuuagggcucauacuaauaaauaaguuugaagacgacuaaccucuaaguccucauccuuuuagaacagaagaugacucuaaacgucuacuauggaugugucuuucgauauaaucaugguaaccgcaucuaaaauuuuaguccuguuagcuaaaucuaccuuucuguuaauuuaacguuuaaacccuaugucguccaguccuuuccaaaucuugcuaauguaguucaauaauggcuccucguguaccauaauaacaucacaugcuaacgugucugguucuaaguaaguuauugcaauuugucaccgagcuucuuuagcuggcaauacgcacacuguuacauuuguuuaaugaccaucccuuauuuucgcuaaacuguugauucuuucaacagcugaagugaugucgguuccucauacggcugguuaacccauaugguaaaaaccuuuggagucgauucuuacguugguuacaucuuguccggaaauacugauaccggcgacuuuauuuuuuaucucauccuggagguagaagacgccaucugguuccuuuauuccaauccaagcuaguuucagcgggucagcuuuguugguuuaggccaacgacgacu

SEQ ID NO: 232 - RNA, 789 nt, Diabrotica virgifera
uaccgucugcgacuacuagauaaucuaauacuucuacuccuugucugucuuguuuggcguugacuuugccguugaugucuccaugucuuuuucccacaguucccguguauacauaguuaugugucaagacccaaaucucuaaaagacaauuuuggucguuaagagucucgauaucaccugacgcccaagcuuguaggaagucuucaaguuguacuuacauaaggaguucgacaguaaccguaccuauaagacacgguucgauuuaggccauacccuuuuugccgacaaaaacauaaucgaugugagguucauuaucuaggaugucuuuuacaacauauacaagagcaguacacgguauggucucucaaucggaaggucuauucguuucucaugcuugcaaagucauuuauauacggguuauaauuucauccccagaagaaaccaccgaacggauaggucuuucuacuccuuugcaauuuuuuauuaacgggcguauagcaacacccaugagguccuucuuaaaaucguaaccagucuagcuuuuuugaauuagaguuuguagauuucguaaaauaaaaccuacuuacacuauuuuacaaccucaauaaucuguacucugcacuacaaguucuuuauauagcauugugaggggugcuuuuuguucaguacuacaagucacgguggaauucauuucuuuaaucuggucaaacguucuuuaaauacguucuacauuaaguuuuaagaauauuauguguuaaaacauuacugcgugggugagcguuacaaacu

SEQ ID NO: 233 - RNA, 738 nt, Diabrotica virgifera
uacggccaguaacuaccaauauuucaugaaauguaaaauaaugugucaaauauauguuaaaaacuuuuauaaaccuccugagaaaauaaauaaauaguuuuaacauauucccaauauuugggacuuagauguaagcuacuacgacuggucaauuucuuuucugacagaucugauuguuuuuucggaguuguaaauugauaguaauaaccacaccuucuuauaaguaaccaucuaaaccgauuggagcauauaaccacaaauccagaauuauaaggcaugcaaucaaagauacuaauauuuccauuaaauuuuuucguacuucucuucaacguuguuuaacaucuuaggucuuuuagucucuuauaguuguauuaaaccguguggguacgucuuguuuccguauuuuuaccuaaaaacccagguuuuuaggugcauuuucacaauugugugcgccugccuuucguuucauaucauuuacaaugauuuuuuaaucgagauuuauuucuuuuucuguaaucauuucuuuuuuaaucacuuaaugauaauuccgucauacuuaaaggucuaggucuuuaccgauaauaaacaccuuucuuugacuuguaaauauuaauaggaggaaccgucaauucugagugucuuaagaaauuucaguuguuucaguuguuguaguguaagggucacaaacaccuuuuuaaccuuuucaugucguuuacacuugucucccacccuuuuauu

SEQ ID NO: 234 - RNA, 480 nt, Diabrotica virgifera
uacugguugagauuuccaauggcggcuccuuggucccuagauaaacgggcguucaaauuuuuugcaccacauuaaggugaaagguguauaaacucucagauguuucaaccucuauaacaucuauaguucccauuaccacgucaaguuuucccauacgggguguuucacaugguaccauucuguccugcacaaaaguuacaaugacguguacguaauccacauuaacauuuguuuucccaagcuccuucuuaguagggguuuucuuaguuagaggcauaacuuguacauuuggugagguucacagcaguucugaagaacguuucucauuuuagguugcuuuucgaugcauuucuucgauuucuuuucuuguaauuucaucuugaauccucuguuggacggguuggauccggucguguauaacaaucgccuuuccaaggucguguccacgaacgaggauaggguauacuuaaguaacgaauc

SEQ ID NO: 235 - RNA, 537 nt, Diabrotica virgifera
uaccuuccuuaugaugaccuugguuguaacauaugguauuuuccauaacgauauaaccugauacuaccauuaucucacgaccgauuuaugaugcuauuucuauauaaaggaugucguuuucucgucuuucgaaaacucuuuuuaaacaaguuauuuugaguaucccgucugcgucuuuaauaguacaaccuaccaaauugaacacacauaucuucauuacaucuaaauaagaaaauacaauacccgucaaguguacuuuuacucgauuaaaauuacucacaaaauuuaacgaacauacugagucauucaguuuauaacuucuuuuuauacguuuuugcucgacagaaccuuagugaucuauaacaauacgaccgauaccuacuuuaacaacuaccuccuuauuaauaucuaagacuaaguucaagucaucauagaucuuaucguaauuccugacuacuauaagguaauccucuuguuugacaucgaguccauaagguuugccgguuucuugucgacuuuaccaguaacgacuuuauu

SEQ ID NO: 236 - RNA, 450 nt, Diabrotica virgifera
uaccgacugguugaguggcuucuuguuuaacgacuuaaguuucuucgaaagagugauaagcuauuucuaccacuaccauguuaaugcugauuucuuaauccuugucauuacucuagagauccuguuuuagguugucuccgacuuaauguccuauacuaguuacuucaucuacggcuaccauugccgugcuagcuaaagggucuuaaaaauugcuacuaccgugcauuuuacuuucuauggcuaucacuccuucuuuaagcacuucguaaggcucacaagcuguuucugccguuaccaaaguagagucgucgucuuaacgcggugcaguacugguugaacccacuuuuuaacugucugcuucuucagcuacuuuacuaagcccuccggcuauagcuaccacuaccaguucaguuaaugcuucucaagcagugguacuacugaaguuucacu

SEQ ID NO: 237 - RNA, 2013 nt, Diabrotica virgifera
uacuacguucguuuguuagcucaggguggauauuucaaccuuuugcuauaucuagaaaugcggcuacuauagcuccuaaagcguguucuacugaaaccaccacuuuuacaacuagauauacugcuacauuauaggcgaggaggaccuuuauuacuguuggguccacuaaguuuaguaguacgaggaggaccacgaccacuucuaccaccuccauuaaaacaacccaguccuccucguggguuauuauauuuaagaagaccuucuucuguagucgacauacaaccuuuagacugaaccaccuguugacuaguucuauaucuuuuacgucacguacuauauccccauuggcugaagguacuucaauucaaaaaacuugugucucguuuaccaguuagguucccuaagacacaguauagaaacccuagacucccuucguacucugagacggaccuugaggauagauuuuuucucuaguuaccgguuuuaggggaacaaugggaaggguguuuuguucgagaaucauugaaacuuucagucagauuuugugcgggaagaggaugauuauuaagauugagugcaggagggguaggauuauuauuacaaguaaguccaggauacgucuuaauaccuccaucuuacggauacuugggaagguacgcaggguacggggguccauacguuccacgagguucuuacgucccagguggaccuaaauuaccuggugguuuguacuuagucguugggggguccaagguuccauuagguguuaccuuaccuggaucuggauuaccaggacccggguuauacccuuacucuggguaccccgguggaguaccuguuguucccggggguucuggugguuacgucccugguggcgucguuccaggagguucuccuuacggcgguguuccagguggcgucuacgcaggucuuaccuuagcuggugguuacguuguucccaugggaguuccgggcggcguauacguuccuggauuguacccagguucuccaggugggguuuacccuggugggccccgcggaguugucguuccuggucgaggcgugcauuuaggucguaagaaaguuguuccuccuccugguggcggggguuacguuguguacggaccuggucccgggcaguacggaggaguuccugggggcguuccagguggugugccugggcaaccuggaggugugccgggugguaacccaggucgcuuacaaggcggaguaccugguggagugccuauaccagguggacgucgcuacggugucggcgguaugccaccggguggaggucugguggcgcgacucuaaggagucaauugucucguucucaaacuccuauauuacagggccuuaucuugucaaaggucaagccgcuaacccgcccggcauaggcugcggcgucgaccucuuaaacguucgcgguaacucugaaaccaaugacgauaaagugaguaguuuguuagguuucaccgauugcugcuagcaacguucuaggaauauucaagcgacguucuaugaaacgcaccacagcuucuguuuucgaugucgaggucggcgucucuggccaguucuaggucccugucuaguguaucuucuugaucuucucuugcuaggagcagugccaugucucugucuucgucucucucccucgcacuugcgcuaucucuagcacuagcacuugcacugucuauaauacuaucuaugucgcuuucucuuucucuggcucuagcaaguucuucgucucuuucuugucuuucccuaucucuugcucuaauaucucuggcccuugggcuucucugucuauuucuuuuuagauuucauaggucucagaguucuaguucuuuguuuagaggccuuggacagcuuggaucgucgcuccauggcuuuaguagggcgauaauacuccuauccauagcccuugcucucucucuuccagcugcucucucgcuagcgcuuucucuuucucuaucuucuccccuucugucgguauccagagcugugauc

SEQ ID NO: 238 - RNA, 765 nt, Diabrotica virgifera
uacucaagauaaccuugacccaugcuaaauagucgaaggguuaagagaggacuaccuucucauaaaguucaacuuauacguuacuuucgucaacuuuuaucaccguggcauuauccggaggcuccaugucuaccguaacauaaccgacgacuuuucgaguaauacaguuuuaacguacuugguucauguuuauuugcuuaaaaguuguaacuauuuguguauccuuaccguaaaaguccgaauuaucgacuacguuccguuuagcaacucuaacgaucuuuucuucguaguuuaauaucuguaguuauaccaaguuuauaaggagaauuuauggauuuacuaucucauucguacauguacguacguaugugaaauaugucacgacaaucugguaaaccaacgucacaguagaaccggucaauacuucuaucacuggguagauacauagacuaacuagguagaccucaaucgauaccuaugaaaccuacacgauguccauuucguuuugucagacguuucugacuuuaucuuuuuaacuucuaccccuuagauuguacguuucuugaacaauuucuucgucgguuuuaguaaauaaaccagguacuacucgacuuccuauucuuaaaacuugaccuugaaaguacccauacguuucuaugcuuaccaaauguaugguuucacggacuuagucacaaacgacuacgucuuuuucgacgguuuguucguuaccuucgucuaagucuuagucuaugucuucuauacauu

SEQ ID NO: 239 - RNA, 744 nt, Diabrotica virgifera
uaccgaaguuuucugucuaacuacuaaaaagguagaucuccucggguuuacuacuacuuuagguccgauuucccucggguuuucccuguaucaaauaauuucuucuuucgacuacgaaauguuuacucuaaaucuuacuaaaacuuguuuuaauaacucugguuuugagaguacccacuucauuacuuucuucgacggaaaagaaaucgacuucguuucaaacguugaccacugaaguuaguucaacaagaaguuuuacagugguuccgaguuuauuuuuauucuugauucuuucuguugcaacgaccacaaugaaauggucacaaacuuacgaugguucuaccaugucuauguauacucaaccgaccaaaccgauccccuccaguuguuaacuguuuugaguucuucuuaauaguuucacgacaauuugacaaccaacuuaaucggagaaacguuugaagaaaacauugagaacuacuacauuaguuuuauuguuugucuucucaguuacgguaacuuguacaauaguaagguucuuaucucgcaugaaaccgaauguaguauaggcuugaccugcucaaucuuucucuccucaagauaucuaauuucuucuagguccuguuuuucuucuagucgucucguuuccggcucuuuguuuuuguucgagaagagguucgacccgaugaauuucucaggguccguuuguacgaaaaccuacucccgcuacuucuagaugaaaagauc

SEQ ID NO: 240 - RNA, 4818 nt, Diabrotica virgifera
uaccuccgccgaggguucaauggcuacgagagcaagcucaauuuaugaacaugucucuugcaggugaaaccgggggucaauuuugucauauaacgacgaaaaauaccacuucuaggucuuaggauguaaugucuuuagucguuagaacuuaggaauucuagucgacgucaagcugguaguugccuacauuuaccacauguugacaacuuuuucauaaagacagucgaagcaaaagaguuuagauccaaaggguaccuucucuuaguucuacgacgucaagauaaaaguaccuuuuuauuacuuaaucuguauugaaguuguaggucacuauagucuauacuuaauuaccauuauuacauauuguaaccucggaaugugugaaaagaaccucgguugcugaguucuuuguuaggccuaccauacuuuuaccguacaugaguaaaaguuacacgacguacccgaaaaguuuugcauuuucuuuucauggugguuaaguauaguuuguagaguaaccaucuugaccaaguaaaaaaaguuguucagacaaaucgaguccgaguccucacauauaaucucuucucguacaaacuguuauccuuuggaugguaguaacguuuucaacgauagguucagaugucaauaaugucugucagaaaugcacagaaccuuagucauuuacuucggaugaaaucccuauuuuggauguuccucaccuacuuuauagaaguuaaguucgacuggaugauguuuacguagagcaaggauaagguucccguuguucgacuccuuguugucuuuuacccucuuucccaacguaagauaguucgacguacacuugucgaccugcuccguuucuuuuaacgacgauguaauuuuuugguugugguggucguucuuuauuuacucccugaucguaagugaugacuacaccaacuuccauuuucuuuucgucgauuuuuacuuuuacucaaguagaugguacuuagucacggacuauuucugguuaacggucuccaauucccacgaaguaaucaguuuccuuaugguaagucauauuuacuauaucuucaaaguccuggucuauaaaagagggcuaaccaggguuaccuccgugugcuucgucgaaggaacaugucgcucuucuuucgagucucuaauucugucuagccccuugaauaacuuuuauuucuaguuuguaaccgacuuaaauacagcaguuacgucgaucuagauaacugguucuacgugguccgaugaccuuauggcguccucaacuaacuaucucgucgccgagauagacgauuuggauugcgguaaguucuagaauauucacgauacccuuucgauagauuauauaugguucugcaacuucguucaaacuuacucuaacuaagaaauaauuuccggcuucuuguuucacuuguuuucaugguucuuugcuaaccauuuucugguggcucguaaaaucgaugucuaaauugaucccuucgccguuuuaugucccuccgaguaugauuucgcuugcugaguuugguuugaaaugugucccgcuacuaccgagugcaccgauuagacuuuuaugagguuguuggcgauuucgucgacguuguagucgacgggaaacagcucaaagguuuagguuuauagcugcuuuuuagaaacuuucuagaccuucgcgaucaacguuuucaucugcuuuacucuuggguuucucgguacgauacccgaguugaagcucuuagauaagugguucugcuauaauguucaagggaacauugcuuuguugguuuagucagcgaccuugucgacaaggucguucuugaaguuuucguaguuuuagacuaaagcaacuaacuuguuuuguggagccguguucuuuuguaauucucgcggaaucagcuaagaaugcgaaugcgacauuuaaguucuuuuauguagguucuauaugagguuuucucuugguguugguauugcagugacuaucguagcaagcugagaaugcuucugaauaaccguuuucgauuguuucccuaucucaaaaugaguuuugaacuuugcuugcauagguucaaugacguuucuuauuccucauggacguuucaaguuguucuucucgcucuagucuacuacagcugacgcguucacggaguuacccucucaguaugcagugaacggcgaggauuugacuuucuaaugaaccugagguccuucuucucacgacgcauaagccucggcucacaaguugguguugucgguugaaauucaaugagucgacgauaccuagacggaggaccauaauccggcugaggucaaccuagucuuuauugccuacauggcuuuuuauaaguuccacuugguguuccaauauaagguauauuaaugguuguuggaagacaaggacggagugucuuauaacuacuucucugauaauuuuuuuacuugcguaacuacgguccacgauucugcagucacggaucagucaugccuaugucguugauguaaggugguuguaugggaguuucacgcaugguugguccauuagucagaaugccuuuucuuugaggucuauaauugaguggcaugcugggaugguuccgcaagugccgaugaugauugcgaauagcaagccacgucucgaggaguugaguuccuaugcauggcaugcgucuuagauugcaaagcuuacaacugucucaaccuauaggaucguccauagucaugguuguucauggacucuaucgaugaugaggucgacuaggguuauaauuacgcaugauggguguacccaugaguggcucggucuuaaauggcuuacgauuaugaguuguauaauggccgguugacuucaugguaagccaccucaugcgaagaagacacggcuuguuguaguuaauauugucgagauggaugagcagcggcgaaagauuauacagaucaugguuaaggaguuuaggaucauugaacuuauuaagaaugcucaugaugauacugggcuuauggucaucaccacgucauggcuuacgaaguuucggaguugucagcucgcggucgagacguuugggcucauggcgauacuuguugauguuaauaaugauguuauguucauggucgccaucacaucgucgccuaugaaguuuuuauguuguuguuguugucauggguccaugagucuacucaguucgcgucaugauagggcgguuacggucaauaaugaguuggucaugaauguuaugguugcagguuccaccaugguuagggagcaugcguugaccuguuuguauauuaguucacuguggucacuggagaguuuuacaaagaguuuuaauguugaaauugguucaaccaagaccucguccuguggucguagucaugaugagcaggcgauugcggcgucaugguaggguuguucgacauuuauugucaaguaaugguuugaugccuaugcuagucauaaugccguuguugauauuaaggucaguuggcucauggaugucgcguuugguucguggaggacgucguguucgacgagguucauuauaaggacgacggugguuuaggaggagauuacaccuagacgagucaccgaaccugaagucguauucgguucgaggagaucacggaguuguuuuguaaugcuauuuugggguucuuuuccuuuguuuugguggccgacaaagaagacuuugguuuuugguucuagguuguggucauuggugcggguuuguugggugaccuggucuucauuucgcgaacaugcaguuuuaggacggcucguuugggaauuuguugcuacuacacuucuuuaacaagccgguucucgaccuguccauacucuucaagcaccucuggaacuggguguuuugaaacucgccaggcuggugagaccuauaauuuaccuuccucuagguucuggucguccuaacgcucggcgucuucuucuaguaaaggcagcgaucuacaauaggauacuucuuauccaagggccuauagaacggaaugcugaaaaggucccaccucaacacgcuaucauuucuacuaauauaguugcgaaguauguaauuccuauagagcgguauacgaggcaguaaauaacaauguguucacggcaacagaaguugacaaccacuauacaccugcuacuaaucucuuguuguccagcuugacuaggagacaaaccauuugcuguuacucuagccacuucuauaaaugaccggguuucuuuuuccgucaucagaauuguaugaauuguaccaguauugcaacguuuugcaauaucaauucagaguaaccugacuuucugacuaucgcuauuugaauggacuuuuugcccucagggcacacuauuacguagauguuaaauguagcaccggaccgucgaacaaagguuguuuaggacuuggcaagcagucgauguggaaccuuagguaguuggauaugguuguugucuucugguuguggguaggccaccaccagguaacaaguagaccguauccuucuucgccggacgagacaaaugacugucgacgauacaagcuacgacgguuguuacgauugagcuauggucuagaaugucgaaacucauaguucaacagguuaacguccuucuuauaagagucucuagcucucguaaacuucaaaccaaugcuuucaaaaaaccgcauauaauccguauaucaaacacuucuauuucggucuuucuuugacuugcucuaggucggguuccaauuccuccuuggugaccuugguggacaguaucaaggucuugguuuauaucuaggaaauucaugaaaucuggguaaaacccgaucauuuucuauu

SEQ ID NO: 241 - protein, 166 aa, Diabrotica virgifera
MTPERKKKLRLLLRKKAAEELKKEQERKAAERRRIIEERCGKPKLVDDANEGPLKQVCEGYHRRIVDLENKKFDLEKEVEFRDFQISELNSQVNDLRGKFVKPTLKKVSKYENKFAKLQKKAAEFNFRNQLKVVKKKEFTLEEEDKEKKPDWSKKGDEKKVQEAEA

SEQ ID NO: 242 - protein, 201 aa, Diabrotica virgifera
MCEEEVAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVLDSGDGVSHTVPIYEGYALPHAILRLDLAGRDLTDYLMKILTERGYSF

SEQ ID NO: 243 - protein, 913 aa, Diabrotica virgifera
MPLRLDIKRKLTARSDRVKCVDLHPTEPWMLCSLYSGNINVWNTENQQLVKTFEVCDVPVRTAKFLPRKNWIVSGSDDMQIRVFNYNTLDRVHSFEAHSDYVRCIVVHPTQPYILTSSDDMLIKLWNWEKAWACQQVFEGHTHYIMQIAINPKDNNTFASASLDRTLKVWQLGASTANFTLEGHEKGVNCVDYYHGGDKPYLISGADDRLVKIWDYQNKTCVQTLEGHAQNVTAACFHPELPVALTGSEDGTVRVWHANTHRLESSLNYGFERVWTIFCLKGSNNVALGYDEGSILVKVGREEPAVSMDASGGKIIWARHSELQQANLKALAEGAEIRDGERLPVSVKDMGACEIYPQTIQHNPNGRFVVVCGDGEYIIYTAMALRNKAFGSAQEFVWAQDSSEYAIRESGSTIRIFKNFKEKKNFKSDFGAEGIYGGYLLGVKSVSGLTFYDWETLDLVRRIEIQPKAVYWSDSGKLVCLATEDSYFILSYDSDEVQKARDNNQVADDGVESAFNLLGEINESVRTGLWVGDCFIYTNSVNRINYFVGGELVTIAHLDRPLYVLGYVPKDDRLYLVDKELRVVSYQLLLSVLEYQTAVMRRDFPTADRVLPSIPKEHRTRVAHFLEKQGFKQQALAVSTDPEHRFELAVALEDLNIAKTLAQEANSPQKWNQLAELAAATNNVSVAKECMQKAQDYGGLLLLATSSGDENLVRTLGETTQAESKHNLAFLSHLLVGDLNKCLDILINTGRLPEAAFFARSYLPDKITEVVELWKTQLSSVNQKAGQSLADPKNYENLFPGLQEAVVAQKFLEQQNKGLAPARVATTIPPNHDRNVVAEVQAQSKHDVPSFSSSFISSEIEAQTRSSAKPEESSNIIQLDQDDDDIDLDLDGVNIDENIDTTDINIDDDLLSD

SEQ ID NO: 244 - protein, 381 aa, Diabrotica virgifera
MQIFVKTLTGKTITLEVEPSDTIENVKAKIQDKEGIPPDQQRLIFAGKQLEDGRTLSDYNIQKESTLHLVLRLRGGMHIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGN

SEQ ID NO: 245 - protein, 376 aa, Diabrotica virgifera
MCDDDVAALVVDNGSGMCKAGFAGDDAPRAVFPSIVGRPRHQGVMVGMGQKDSYVGDEAQSKRGILTLKYPIEHGIITNWDDMEKIWHHTFYNELRVAPEEHPILLTEAPLNPKANREKMTQIMFETFNTPAMYVAIQAVLSLYASGRTTGIVLDSGDGVSHTVPIYEGYALPHAILRLDLAGRDLTDYLMKILTERGYSFTTTAEREIVRDIKEKLCYVALDFEQEMATAASSTSLEKSYELPDGQVITIGNERFRCPEALFQPSFLGMESCGIHETVYNSIMKCDVDIRKDLYANTVLSGGTTMYPGIADRMQKEITALAPSTIKIKIIAPPERKYSVWIGGSILASLSTFQQMWISKQEYDESGPGIVHRKCF

SEQ ID NO: 246 - protein, 879 aa, Diabrotica virgifera
MGTFKRDTHDEDGGSSAFQNLEKTTVLQEARVFNETSVNPRKCTPILTKLLYLLNQGETLSAKEATDVFFAMTKLFQSKDVILRRMVYLGIKELSSVADDVIIVTSSLTKDMTGKEDMYRAAAIRALCSITDATMLQAIERYMKQAIVDRNAAVSSAALISSLHMSKLAPDVVKRWVNEAQEAVNSDNAMVQYHALGLLYHIRKTDKLAVTKLISKLNSMGLKSPYALCMLIRITAKLLEEEDQESLLNSPYTIIFTMGLRNKSEMVVYEAAHAMVNLKFTSSNVLAPAISVLQLFCGSPKATLRFAAVRTLNQVATTHPASVTACNLDLENLITDPNRSIATLAITTLLKTGAESSVDRLMKQIATFVSEISDEFKVVVIQAIKVLALKFPRKHSTLMNFLSAMLRDEGGLEYKASIADTIITLIEDNPEAKESGLAHLCEFIEDCEHVSLAVRILHLLGKEGPKTKQPSRYIRFIYNRVILECPSVRAAAVSAMAQFGASCPDLLENIQILLSRCQMDSDDEVRDRATYYSNILNKNDKSLYNNYILDSLQVSIPSLERSLREYIQNPTDEPFDIKSVPVAAVPTAEEREVKNKSEGLLVSQGPVRPPPVSREENFAEKLSNVPGIQQLGPLFKTSDVVELTESETEYFVRCIKHCFKHHIVLQFDCLNTLPDQLLENVRVEIDAGETFEILAEIPCEKLHYNETGTTYVVVKLPDDDLPNSVGTCGAVLKFLVKDCDPSTGIPDSDEGYDDEYTLEDIEITLGDQIQKVSKVNWAAAWEEAAATYVEKEDTYSLTINTLSGAVKNIIQFLGLQPAERTDRVPEGKSTHTLLLAGVFRGGIDILVRAKLALGECVTMQLTVRSPDPDVAELITSTVG

SEQ ID NO: 247 - protein, 216 aa, Diabrotica virgifera
MAANRTGPAQRPNGATQGKICQFKLVLLGESAVGKSSLVLRFVKGQFHEYQESTIGAAFLTQTICLDDTTVKFEIWDTAGQERYHSLAPMYYRGAQAAIVVYDITNQDTFGRAKTWVKELQRQASPTIVIALAGNKQDLANKRMVEYEEAQTYADENGLLFMETSAKTAMNVNDIFLAIAKKLPKNEQTTGQGGSAQGRRLAEGDSGAKAPGNCCK

SEQ ID NO: 248 - protein, 92 aa, Diabrotica virgifera
MKFLRSTVCYIAILAILFTLCADEVEGRRKILMGRKSITRTYLRGNAVPAYVIIILVGIGQIILGGILYVALRKKIIAAPVTASYAVARQEP

SEQ ID NO: 249 - protein, 157 aa, Diabrotica virgifera
MQIFVKTLTGKTITLEVEPSDTIENVKAKIQDKEGIPPDQQRLIFAGKQLEDGRTLSDYNIQKESTLHLVLRLRGGAKKRKKKNYSTPKKIKHKKKKVKLAVLKFYKVDENGKIHRLRRECPAEQCGAGVFMAAMEDRHYCGKCGYTLVFSKPGDEK

SEQ ID NO: 250 - protein, 248 aa, Diabrotica virgifera
MMSKADTQEDASFAKLENQIAIIKYVILFTNVLQWALGAAIFALCLWLRFEEGIQEWLQKLDSEQFYIGVYVLIVASLIVMIVSFIGCISALQESTMALLVYIGTQVLSFIFGLSGSAVLLDNSARDSHFQPRIRESMRRLIMNAHHDQSRQTLAMIQENVGCCGADGATDYLSLQQPLPSQCRDTVTGNPFFHGCVDELTWFFEEKCGWIAGLAMAICMINVLSIVLSTVLIQALKKEEEASDSYRR

SEQ ID NO: 251 - protein, 124 aa, Diabrotica virgifera
MSGRGKGGKVKGKAKSRSNRAGLQFPVGRIHRLLRKGNYAERVGAGAPVYLAAVMEYLAAEVLELAGNAARDNKKTRIIPRHLQLAIRNDEELNKLLSGVTIAQGGVLPNIQAVLLPKKTEKKA

SEQ ID NO: 252 - protein, 288 aa, Diabrotica virgifera
MKLDGVDLPPPISFDIAEEQPLPPCQQTFLCNGDGGSIVRQFLELYFVIYDSDNRQSLLQAYHEKATFSMTMAYPYGYSKDSKGVSWLNWYATDNRNLLRVQDPDRRNKLLRQGQVAVVSFLQDMPHTKHDIHSFTVDLTVFTPQMLCLTVAGMFKELKSGHKVPPLRYFFRTLVIVPAGSGFCIANEELHISNATPDQAKDAFKTTVNVAPAPAPVITSPGPSIPQPAVPDDATKQEMVKQMSAVSGMNLEWSLQCLEETQWDYQKAIMVFQNLNAQGVVPQAAFIK

SEQ ID NO: 253 - protein, 955 aa, Diabrotica virgifera
MTAVEQPCYTLINLPTDSEPYNEMQLKMDLEKGEVKVKIRALEKIIHMILAGERLPNGFLMTIIRNVLPLQDHLAKKLLLIFWEIVPKTNPEGKLLQEMILVCDAYRKDLQHPNEFLRGSTLRFLCKLKEPELLEPLMPSIRACLDHRHSYVRRNAVLAIFTIYKNFEALIPDAPELISNYLDGEQDMSCKRNAFLMLLHADQERALSYLASCLDQVNSFGDILQLVIVELIYKVCHSNPAERSRFIRCIYNLLNSSSPAVRYEAAGTLVTLSSAPTAVKAAASCYIELIIKESDNNVKLIVLDRLIALKELPNHERILQDLVMDILRVLSAPDLEVRKKTLSLALELVSSRNIEEMVLVLTKEVSKTVDSEHEDTGKYRQLLVRTLHSCSIKFPDIARSVIPVLIEFLSDNNELAATDVLLFLREAIQKFKELQPLIIEKLIETFKDIKLVKVHRAAIWILGEYASTASDIEVIVGEINRLLGEGSLVEAEQKLIAGDTEENAPAPAAGATTLVTSDGTYATQSAFNTVSQTTKEARPPLRQYLMDGDFFIGASLASTLTKLSLRYEDLTSPAASNGFNAKIMLIMAGILHLGKSGLPTKSITNDDKDHILFCLRVLSDRSPIIVEIFKKLCRSALNEMLLAKESVEAISQKSKEKNKRTIQTDDAISFLQLETDKSGELGENVFEMSLSQALVGGRTGGGESVLSSNKLDKITQLTGFSDPVYSEAYVHVNQYDIVLDVLIVNQTNDTLQNCTLELATLGDLKLVEKPQPVVLAPKDFCNIKANVKVASTENGIIFGNIVYDVIGAGSDRNVVVLNDIHIDIMDYIVPASCTDSEFMRMWAEFEWENKVTVNTPLTELSEYLEHLLKSTNLKCLTSEKALSGQCGFMAANLYAKSIFGEDALANLSIEKPFNKPDAPVSGHIRIRAKSQGMALSLGDKVNMTQKSTQHKVVAA

SEQ ID NO: 254 - protein, 447 aa, Diabrotica virgifera
MVYPNSPFKTTFNNKLITNLFLDDSVTKTKTSVSNEELLKIQQWLCSAPKITSIRVNTIKTNTAKVLKVLKTYFAENCNDTPNVYIHPSFTNVIIIDSLNCPAGFKKFDKEIIVDTDCAAAILRGAHIFAPGVLGMVSGCQINENVSIYADVAKKCKKGLQKIYEDDFKIFIGNGIVKMQRHQLFATDNIAPSGIAVEVTETISGCVPISESLLPVGDILLQNIPSIVCVHNLNPKPGDVVLDMCASPGNKTTHIAELMQNKGILIAIDKTPKKVAQLQKRCEDFGAKVYSFQADSTAIISDTLSSKNVIDGPPFAPQSFDKILLDAPCSVLGKRPQLANRSSENEIKSFVPLQRKLFENAAKLLKPGGIMVYSTCTITLSENEGIVAWALKSFNFLELVQPDLTLGEPGWLGTSLSDEARSFVQRFGPNSEVDSVGFFFAVFKKKT

SEQ ID NO: 255 - protein, 1474 aa, Diabrotica virgifera
MGRLHCLFCIFLCFTVINTQTTNIHGFSENSVDTFLSPHGKSAKFVHQNHKPKIENCQNYKPSVKEEQPGGTYVTTVTAIDDDPREGGGTISYKLIHREGEHVLFDIDNVTGVLTTIQPFDRDEPVRQKELYVTVQASDNGRPPLADVCTFTVTITDINDNAPQLDKLKYDAQVSEDLKVGSEVMRVFAYDIDDGENSRLSYNFSNENAQFTQYFRIDRDTGVVYLKEALTDKKNTRFNSAVYVADNGVNDQEGQKDSTAKISITVVGSDKQPPRFTQKMPDGILEIPEDFKDFSKHIVTVEATSNIADPQLAFELVKGKTYQTNKDQTFLLEAEGNKAHIKLVRPLDYETVTEYTLTIRVKNKDLMDSSINIPIKVLDVNDEIPNFLEFLKGSVVENDKPGAQAIQVRAIDKDGTAANNIVSYELVDNTDLFAINRSTGVITSRVEFDRETVPLYHVNVKAYDNSPSALYNTTLPNIVIQTFQISIEDQNDNKPVFTHPIYQFSNITELADKSSIVGEVKALDNDTASVISYSITNGNIDDAFMIENSTGRIRVNGKLDYEKIEQYNLTVRAFDGAFEDFAIVLISILNENDEPPVFDDYIREIQIKEEEPMISGCVVRVTAHDPDIKDRHADQHIVYEVAKEQKDFLTVSADGCVQVTKPLDRDPPFGSPTRQVFIYARDNDGGTNSLLATAEIEIILIDINDNAPFLNVTEIVYYENQDPGFIGNLSADDYDGPDNGPPFAFRLSDTASDSIRSKFSIIGNQLFALEMFDREEQKYYDIAIDITDSGVPPLTGTSILRVIIGDVNDNPATDGNSTIFVYKYVNGPENFMEIGRVYVTDLDDWDLNDKVFVQEDNFDEFVLNQHNNGMILMKPTTAEGTYEVHYRVTETHEPTIHEHTVNAIVTITVKVLPEEAVVKSGSIRLRGTTKEEFIENSLNGKSKRDILHQELSKILNTSLANVDVFTVLNSPHQNSSFVDVRFSAHGSPYYAPEKLENKVTDHQMELEQKLDVEFYMINVNECLNETTCGAENSCTNKLNITREPAVVFTNRTSFVGVNAFIDPVCAALPRDVMECFNGGVLIENTACNCPAGFEGPHCEILAIGFTGTGWAMYPSFDATNRTEIILHILSQTDNGLIFYNGPLNIRQTSLSKDYISLELKDGYPLLQICTGSSTQEIYLKERIHKLSDGSLHKIKIGSGFDDISLEVDDCGTTCSIWTNKLHKGVIRANGPLQLGGMKNRFTDQEFKRIWDHLPPTATRFSGCIRNLTYNEFYYNLGAPSDAFQAYPDCNYAVMQAVTFGIDSNFLVAILVCVAILIILLLAVVVHRRKHDNFNEKEIDDTRENIINYEDEGGGECDTNYDLSVFHQNNIVDEKPLMRDNPDVPADISGFLDNKKDNCDKDPDNLPYDDVRHYAYEGDGNSTGSLSSLASCTDEGDLKFNYLSSFGPRFRKLADMYGEDPSDEDSHDGNEESWC

SEQ ID NO: 256 - protein, 96 aa, Diabrotica virgifera
MPFCGPKLSLCGLIISAWGIIQLGFMGVFYYIGAVALAEDIPEVEFKGDLDKFYSDVNTGFTQNAYNCWIAALLYLITLAVSAHQFWANNRSSLNV

SEQ ID NO: 257 - protein, 179 aa, Diabrotica virgifera
MGLTISAVFNRLFSKKPMRILMVGLDAAGKTTILYKLKLGEIVTTIPTIGFNVETVEYKNISFTVWDVGGQTRIRKLWRHYFANTDGLIFVVDSNDRDRIAEAEEELHNMLGEDDLRDCILLIFANKQDLPNSMSTAELTDKLKLHTLKNRRWYIQATCATQGNGLYEGLDWLSNELAK

SEQ ID NO: 258 - protein, 205 aa, Diabrotica virgifera
MRYTLSYIGATLASTVTLIFALYYCLTGKGEQVSLAWLLLNVSPHMWAGLGIGLAVSLSVVGAAAGIHTTGVSIVGAGVKAPRIKTKNLISIIFCEAVAIYGLIMAIVLCGSWKNFDVDLFNLKTHNFAQNHYGSHVIFGSGLTVGFVNLLCGFCVGVVGSGAAISDAANSSLFVKILIIEIFGSAIGLFGLIVGVYLTSRGSMV

SEQ ID NO: 259 - protein, 1897 aa, Diabrotica virgifera
MATNDSKAPLRTVKRVQFGILSPDEIRRMSVTEGGIRFPETMEAGRPKLCGLMDPRQGVIDRSSRCQTCAGNMTECPGHFGHIELAKPVFHVGFVTKTIKILRCVCFFCSKLLVSPNNPKIKEVVMKSKGQPRKRLAFVYDLCKGKNICEGGDEMDVGKESEDPNKKAGHGGCGRYQPNIRRAGLDLTAEWKHVNEDTQEKKIALSAERVWEILKHITDEECFILGMDPKFARPDWMIVTVLPVPPLAVRPAVVMHGSARNQDDITHKLADIIKANNELQKNESAGAAAHIITENIKMLQFHVATLVDNDMPGMPRAMQKSGKPLKAIKARLKGKEGRIRGNLMGKRVDFSARTVITPDPNLRIDQVGVPRSIAQNMTFPEIVTPFNFDKMLELVQRGNSQYPGAKYIIRDNGERIDLRFHPKPSDLHLQCGYKVERHIRDGDLVIFNRQPTLHKMSMMGHRVKVLPWSTFRMNLSCTSPYNADFDGDEMNLHVPQSMETRAEVENLHITPRQIITPQANQPVMGIVQDTLTAVRKMTKRDVFIEKEQMMNILMFLPIWDGKMPRPAILKPKPLWTGKQIFSLIIPGNVNMIRTHSTHPDDEDDGPYKWISPGDTKVMVEHGELVMGILCKKSLGTSAGSLLHICMLELGHEVCGRFYGNIQTVINNWLLLEGHSIGIGDTIADPQTYTEIQRAIRKAKEDVIEVIQKAHNMELEPTPGNTLRQTFENQVNRILNDARDKTGGSAKKSLTEYNNLKAMVVSGSKGSNINISQVIACVGQQNVEGKRIPFGFRKRTLPHFIKDDYGPESRGFVENSYLAGLTPSEFYFHAMGGREGLIDTAVKTAETGYIQRRLIKAMESVMVHYDGTVRNSVGQLIQLRYGEDGLCGEMVEFQYLATVKLSNKAFERKFRFDPSNERYLRRVFNEEVIKQLMGSGEVISELEREWEQLQKDREALRQIFPSGESKVVLPCNLQRMIWNVQKIFHINKRAPTDLSPLRVIQGVRELLRKCVIVAGEDRLSKQANENATLLFQCLVRSTLCTKCVSEEFRLSTEAFEWLIGEIETRFQQAQANPGEMVGALAAQSLGEPATQMTLNTFHFAGVSSKNVTLGVPRLKEIINISKKPKAPSLTVFLTGAAARDAEKAKNVLCRLEHTTLRKVTANTAIYYDPDPQNTVIPEDQEFVNVYYEMPDFDPTRISPWLLRIELDRKRMTDKKLTMEQIAEKINAGFGDDLNCIFNDDNAEKLVLRIRIMNSDDGKFGEGADEDVDKMDDDMFLRCIEANMLSDMTLQGIEAISKVYMHLPQTDSKKRIVITETGEFKAIAEWLLETDGTSMMKVLSERDVDPVRTFSNDICEIFSVLGIEAVRKSVEKEMNAVLSFYGLYVNYRHLALLCDVMTAKGHLMAITRHGINRQDTGALMRCSFEETVDVLMDAASHAEVDPMRGVSENIILGQLPRMGTGCFDLLLDAEKCKMGIAIPQAHSSDLMASGMFFGLAATPSSMSPGGAMTPWNQAATPYVGSIWSPQNLMGSGMTPGGAAFSPSAASDASGMSPAYGGWSPTPQSPAMSPYMASPHGQSPSYSPSSPAFQPTSPSMTPTSPGYSPSSPGYSPTSLNYSPTSPSYSPTSQSYSPTSPSYSPTSPNYSPTSPSYSPTSPNYSPTSPNYSPTSPSYPSTSPGYSPTSRSYSPTSPSYSGTSPSYSPTSPSYSPTSPSYSPSSPNYSPTSPNYSPTSPNYSPSSPRYTPGSPSFSPSSNSYSPTSPQYSPTSPSYSPSSPKYSPTSPNYSPTSPSFSGGSPQYSPTSPKYSPTSPNYTLSSPQHTPTGSSRYSPSTSSYSPNSPNYSPTSPQYSIHSTKYSPASPTFTPTSPSFSPASPAYSPQPMYSPSSPNYSPTSPSQDTD

SEQ ID NO: 260 - protein, 291 aa, Diabrotica virgifera
MSSNIQKAQQLMADAEKKVTSRGFFGSLFGGSSRIEDAVECYTRAANLFKMAKSWDAAGKAFCEAANLHSRTGARHDAATNYIDAANCYKKADVFEAVNCFIKAIDIYTEMGRFTMAAKHHQTIAEMYETDAVDIERAVQHYEQAADYFRGEESNASANKCLLKVAQYAAQLENYEKAVGIYQEVAYAALESSLLKYSAKEYLFRAALCHLCVDVLNAQHAIESYISRYPAFQDSREYKLLKTLIENIEEQNVDGYTEAVKDYDSISRLDQWYTTILLRIKKQVSESPDLR

SEQ ID NO: 261 - protein, 202 aa, Diabrotica virgifera
MNPEYDYLFKLLLIGDSGVGKSCLLLRFADDTYTESYISTIGVDFKIRTIDLDGKTIKLQIWDTAGQERFRTITSSYYRGAHGIIVVYDCTDQDSFNNVKQWLEEIDRYACDNVNKLLVGNKSDLTTKKVVDFTTAKEYADQLGIPFLETSAKNATNVEQAFMTMAAEIKNRVGPPSSAVDQGNKVRFDQSRPVETTKSGCC

SEQ ID NO: 262 - protein, 262 aa, Diabrotica virgifera
MADADDLLDYEDEEQTEQTATETATTEVQKKGVKGTYVSIHSSGFRDFLLKPAILRAIVDCGFEHPSEVQHECIPQAVIGMDILCQAKSGMGKTAVFVLATLQVIDPTENVVYVLVMCHTRELAFQISKEYERFSKYMPNIKVGVFFGGLPIQKDEETLKNNCPHIVVGTPGRILALVRSKKLNLKHLKHFILDECDKMLELLDMRRDVQEIYRNTPHEKQVMMFSATLSKEIRPVCKKFMQDVIQNSYNTQFCNDAPTRNV

SEQ ID NO: 263 - protein, 245 aa, Diabrotica virgifera
MPVIDGYKVLYILLHSLYTIFENIWRTLLFIYQNCIRVINPESTFDDADQLKKRLSRLTKKPQHLTIIIGVEEYSLVDLANLVYWCLGLNIPYVSFYDYKGNLKKHEEKLQQIVESRKSENINIIWHTHAEQRHKNGFLGPKIHVKVLTHADGKQSIVNVTKKLALNKEKDISKEKISELLLRQYEFPDPEMAIICGKKLNIYNYPPWQLRLTEFFKVNKVNNITFPVFVEKLEKYSKCEQRVGK

SEQ ID NO: 264 - protein, 159 aa, Diabrotica virgifera
MTNSKGYRRGTRDLFARKFKKRGVIPLSTYLRVYKVGDIVDIKGNGAVQKGMPHKVYHGKTGRVFNVTAHALGVIVNKRVRGRIIPKRINLRIEHVNHSKCRQDFLQRVKSNEKLRKEAKEKNIKVELRRQPAQPRPAHIVSGKVPAQVLAPIPYEFIA

SEQ ID NO: 265 - protein, 178 aa, Diabrotica virgifera
MEGILLEPTLYTIKGIAILDYDGNRVLAKYYDKDIFPTAKEQKAFEKNLFNKTHRADAEIIMLDGLTCVYRSNVDLFFYVMGSSHENELILMSVLNCLYDSVSQILKKNMQKRAVLESLDIVMLAMDEIVDGGIIIDSDSSSVVSRIALRTDDIPLGEQTVAQVFQTAKEQLKWSLLK

SEQ ID NO: 266 - protein, 149 aa, Diabrotica virgifera
MADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGFISAAELRHVMTNLGEKLTDEEVDEMIREADIDGDGQVNYEEFVTMMTSK

SEQ ID NO: 267 - protein, 670 aa, Diabrotica virgifera
MMQANNRVPPIKLENDIDLYADDIEDFAQDDFGGENVDLYDDVISAPPGNNDNPGDSNHHAPPGAGEDGGGNFVGSGGAPNNINSSGRRHQLYVGNLTWWTTDQDIENAVHDIGVTDFHEVKFFEHRANGQSKGFCVISLGSEGSMRLCLELLSKKEINGQNPLVTLPTKQALSNFESQSKTRPSPTNNSNSRPPHPNNNVHSGPMQNYGGRMPMNPSMRPMPPGMQGAPRMQGPPGFNGPPNMNQQPPRFQGNPQWNGPRPNGPGPNMGMRPMGPPHGQQGPPRPPMQGPPQQGPPRGMPPQGPPQMRPEWNRPPMQQGYPQGPPHMQGPNMGPRGPPQMGPPGAPQQQGPAPHVNPAFFQQGGGPPPPMQHMPGPGPVMPPQGPPQGPPHGPVGPPHGPPLGPANVPPHGPPHGYGPPAAMPQPPYGGPPPDHRAEIPQLTEQEFEDIMSRNRTVSSSAIGRAVSDAAAGEFASAIETLVTAISLIKQSKVANDDRCKILISSLQDTLRGVEDKSYSSSRRDRSRSRDRSHRRTRRERSSSRYRDRSRERERERDRDRDRERDRYYDRYSERERDRDRSRSRERTERDRERDYRDREPEETDKEKSKVSRVSRSRNKSPEPVEPSSEVPKSSRYYEDRYREREREGRRESDRERERDRRGEDSHRSRH

SEQ ID NO: 268 - protein, 254 aa, Diabrotica virgifera
MSSIGTGYDLSASQFSPDGRVFQVEYAMKAVENSGTVIGLRGTDGIVLAAEKLIMSKLHEPSTNKRIFNIDKHIGMAFSGLIADARQIVEIARKEASNYRHQYGSNIPLKYLNDRVSMYMHAYTLYSAVRPFGCSVILASYEDSDPSMYLIDPSGVSYGYFGCATGKAKQSAKTEIEKLKMGNLTCKELVKEAAKIIYLVHDELKDKNFELELSWVCKDTNGLHTKVPESVFADAEKAAKQAMEADSESDTEDM

SEQ ID NO: 269 - protein, 246 aa, Diabrotica virgifera
MASKDRLMIFPSRGAQMMMKSRLKGAQKGHSLLKKKADALQMRFRMILNKIIETKTLMGEVMKEAAFSLAEAKFATGDFNQVVLQNVTKAQIKIRTKKDNVAGVTLPVFECYQDGTDTYELAGLARGGQQLTKLKKNYQSAVKLLVELASLQTSFVTLDDVIKITNRRVNAIEHVIIPRIERTLAYIISELDELEREEFYRLKKIQDKKKISRAKAEKQKQALLQAGLLKESQANMLLDEGDEDLL

SEQ ID NO: 270 - protein, 1605 aa, Diabrotica virgifera
MEAAPKLPMLSFELNTCTENVHFGPQLKQYIAAFYGEDPESYITEISNLESLRSAAVRPSTDVNGVQLLKKYFCQLRFLKSRFPMEENQDAAVLFSWKNNELDITSTSSDIRYELMVIMYNIGALHTFLGANDSRNNPDGMKMACTHFQCAAWAFQNVKEKYHQFISNISLVELVHFFQQVCLAQAQECILEKSMFDNRKPTIIAKVAIQVYSYYRQSLRVLESVNEAYFRDKTYKEWMKYLQFKLTYYKCISFLFQGQQAEEQQKMGERVAFYQAACEQLDEAKKIAATLKNQHHQQEINEGLAFTTDVVEGKRKAAKNENEFIYHESVPDKDQLPEVKGASLVKGIPFSINDIEVSGPDIFSRLVPMEAHEAASLYSEKKAQRLRQIGELIENKDQTLAEFMSSMQLDLLTKMHQATGIPQELIDRAAALSAKPNAIQDLISAMGKLSNIYQDVEASLNEIDSLLKAEEQSEQKYQETIGKRPPSILATDLTREAAKYREAHTKANDSNQTLHRAMMAHVANLKILQQPLKQLQHQLPFVEFPNPNIDEKSLKDLEALVAKVDEMRTQRAMLWAQLRESIHQDDITSSLVTKQPNQSLEQLFQQELQKHQNLISLIEQNTSAQENIKSALVDSYAYAVNSRKYIQDILQKRTTTITSLIASFDSYEDLLAKANKGIEFYSKLETNVSKLLQRIRSTCKVQQEERDQMMSTAQVPQWESHTSLAAPKLKDYLDSRKKSAAYSEPSVQPQQPTLSYSAAMDLPPGIRPTPVGSEITDVPKNIQGEPQGYIPYNYQQPSVPASQNIDEETIKKMNALMPGAKTSVPSQYGYSNYIPPTYPQSAYQPGNQSYGKETPDINSPYDPTKAFTATTNAYRSVQSSSTQGYVPYAESNVSNVDRVGYPSRYQYQQVPEIATTPADPNINAYYPHGYSPSQNLPNANTQHITGQLKYHSVEYASSVPNNINYNSSTYSSPLSNMSSTNSSNPSNLNNSYEYYYDPNTSSGAVPNASKPQQSSASSANPSTAMNNYNYYYNTSTSGSVAADTSKIQQQQQYPGTQMSQAQYYPANASYYSTSTYNTNVQGGTNPSYATGQTYNQVTPVTSQNVSQNYNFNQVGSGAGHQHQYYSSANAAVPSQQAVNNSSLPNYGYDQYYGNNYNSSQPSTYSANQAPPAAQAAPSNIPAATKSSSNVDLLSGLDFSISQAPLVPQQNITIKPQEKETKPPAVSSETKNQDPTPVTTPKQPTGPEVKRLYVKILPSKPLNNDDVKKLFGQELDRYEKFVETLTHKTLSGPTTLDIKWKEIQDQQDCEPQKKIISVARCYPMKNRFPDILPYDFSRVELCDSKDDYINASYIKDISPYAPSFIVTQVPLSSTVGDMWTMIREQQVELILCLVNDNEIGEDIYWPKEKGSSLNILNMVITLQNVIVKSHWTERLIAINLPEKRESRVIMHLQFTSWPGSLFPTNPEPFVSYTLESINLYQQQKTNTHPVVVHCSSGIGRSGLLCLLTAAMFDAANNANSIPDLTALSIKLSNCRKNILRDREHLKFGYESFLAYIRHIVCEDKARKKLNEIQPKVKEEPLEPPVIVPEPNIDPLSTLDPFWASKR

SEQ ID NO: 271 - DNA, 1707 bp, Diabrotica barberi
atgggtacttttaaaagagatactcatgatgaggacgggggatcaagtgcttttcaaaatttggagaaaactactgttttgcaggaagctagagtttttaatgaaactagtgtaaatccaagaaaatgtacaccgatactaacaaaactgttgtacttattgaaccagggtgaaactttgagtgccaaagaggccacagatgttttcttcgccatgaccaaactattccaatcaaaagatgtaatattaagaaggatggtttatttgggaattaaagaactcagttctgttgctgatgatgttattatcgtaacatccagtcttacaaaagatatgactggtaaagaagacatgtacagagcagctgctataagagcattatgcagtattactgatgctactatgcttcaagctatagagcgatatatgaagcaagctattgtagatagaaacgcagctgtcagttcagcagcactaattagttcattacatatgagcaaattagctccagatgtagtaaagagatgggtaaatgaagctcaggaagcagtaaatagtgataatgcaatggtacagtatcacgcattaggtcttctataccatattaggaagactgataagctagcagtgacaaaattgatttccaagctgaattcaatgggtttaaagagcccatatgctttatgtatgttgataagaatcactgcaaaacttttagaagaagaggaccaagagtctctcctcaactccccatatacaataatatttacaatgggcttaaggaacaaatctgaaatggtggtgtatgaagctgcacatgccatggttaacctgaagttcacgagtagtaatgtgctagcacccgctataagtgttctacaactattttgtggatctcctaaagccacactcagatttgctgctgttagaactttaaatcaagtagccaccacccaccctgcgtcagtgacagcttgtaatttggatctagaaaatttgattactgatcctaatagatcaattgctacactggccattactactcttttgaaaacaggtgccgaatcttctgttgacagactaatgaaacaaatcgctacttttgtatctgaaatcagtgatgaatttaaagtggttgtcattcaggcaattaaggtattagctttgaaatttccaaggaaacatagcacgcttatgaatttcctatccgccatgttaagagatgagggaggtttagaatataaagcgtccatagcagataccattataactctaatcgaagataatcctgaagctaaagaatctggtttggcgcatctttgcgagttcattgaagactgtgaacatgtttctttggctgtgagaatcttgcatttgttaggaaaggaaggacccaagaccaaacaaccatcgagatacatccgttttatctacaatcgcgtcatattggaatgtccttctgtaagagctgctgcagtctccgccatggcacaatttggagcttcttgtcccgatttgttagaaaatatccaaatattactttcgaggtgtcagatggattcagacgatgaagttagggacagagctacatattatagtaatatacttaacaaaaatgataaaagtttatacaacaattacattttggattctttgcaggtttcaattccttcactagaaagatcgcttagagaatacattcaaaat

SEQ ID NO: 272 - DNA, 867 bp, Diabrotica barberi
atgaagttggatggtgtagatctacccccaccaattagcttcgacattgcggaagaacaaccgttaccaccttaccagcagacgttcttatgtaatggtgatggaggatccatagtgcgacagtttctcgaacagtatttcgtaatatacgattcagataataggcagtcccttcttcaggcatatcacgaaaaagccgcattttcaatgacaatggcttacccgtatggctattccaaagacagtaaaggagtatcgtggttgaattggtatgccaccgataatagaaatttattacgagttcaagatccagacagaagaaacaagttgttaagacagggacaagttgctgtagtttctttcttgcaagatatgccgcacacgaagcacgatattcacagttttacagtagatttgacagtttttacaccccagatgttatgtttgacagtggctggtatgtttaaagaattgaaaagtggccacaaagtacctcctttaagatatttcttcagaacccttgtaattgtacctgctggatcaggtttttgcatagcaaatgaagaacttcacatatccaatgcaactccagaccaagcaaaagatgctttcaagaccaccgttaatgtagctccggcaccagctcctgtggttacctctcctggacccagcataccacaaccagctgtgccagatgatgctacaaaacaagaaatggtaaaacagatgtcagcagtatccggaatgaatctcgagtggtcgctacagtgtctcgaagaaacacaatgggactaccagaaagccataatggtattccaaaatttaaacgcacaaggtgttgtaccacaagcagcatttattaaatga

SEQ ID NO: 273 - DNA, 609 bp, Diabrotica barberi
atgaatcccgagtatgattatttattcaaacttctgctgattggagattcaggagtaggaaaatcttgtcttctactgagatttgcagatgatacctacacagaaagctatattagtaccattggcgtagattttaaaatcaggacaatcgatttagatggaaagacaattaaattgcaaatttgggatacagcaggtcaggaaaggtttagaacgattacatcaagttattaccgaggagcacatggtattattgtagtgtacgattgcacagaccaagattcattcaataacgttaaacaatggctcgaagaaatcgaccgttatgcgtgtgacaatgtaaacaaattactggtagggaataaaagcgatttgacaactaagaaagttgtcgacttcactacagccaaggagtatgccgaccaattgggaataccatttttggaaacctcagctaagaatgcaaccaatgtagaacaggcctttatgactatggccgctgaaataaaaaatagagtaggacctccatcttctgcagtagaccaaggaaataaggttaggttcgatcaaagtcgcccagtcgaaacaaccaaatctggttgctgctga

SEQ ID NO: 274 - RNA, 1707 nt, Diabrotica barberi
auggguacuuuuaaaagagauacucaugaugaggacgggggaucaagugcuuuucaaaauuuggagaaaacuacuguuuugcaggaagcuagaguuuuuaaugaaacuaguguaaauccaagaaaauguacaccgauacuaacaaaacuguuguacuuauugaaccagggugaaacuuugagugccaaagaggccacagauguuuucuucgccaugaccaaacuauuccaaucaaaagauguaauauuaagaaggaugguuuauuugggaauuaaagaacucaguucuguugcugaugauguuauuaucguaacauccagucuuacaaaagauaugacugguaaagaagacauguacagagcagcugcuauaagagcauuaugcaguauuacugaugcuacuaugcuucaagcuauagagcgauauaugaagcaagcuauuguagauagaaacgcagcugucaguucagcagcacuaauuaguucauuacauaugagcaaauuagcuccagauguaguaaagagauggguaaaugaagcucaggaagcaguaaauagugauaaugcaaugguacaguaucacgcauuaggucuucuauaccauauuaggaagacugauaagcuagcagugacaaaauugauuuccaagcugaauucaauggguuuaaagagcccauaugcuuuauguauguugauaagaaucacugcaaaacuuuuagaagaagaggaccaagagucucuccucaacuccccauauacaauaauauuuacaaugggcuuaaggaacaaaucugaaauggugguguaugaagcugcacaugccaugguuaaccugaaguucacgaguaguaaugugcuagcacccgcuauaaguguucuacaacuauuuuguggaucuccuaaagccacacucagauuugcugcuguuagaacuuuaaaucaaguagccaccacccacccugcgucagugacagcuuguaauuuggaucuagaaaauuugauuacugauccuaauagaucaauugcuacacuggccauuacuacucuuuugaaaacaggugccgaaucuucuguugacagacuaaugaaacaaaucgcuacuuuuguaucugaaaucagugaugaauuuaaagugguugucauucaggcaauuaagguauuagcuuugaaauuuccaaggaaacauagcacgcuuaugaauuuccuauccgccauguuaagagaugagggagguuuagaauauaaagcguccauagcagauaccauuauaacucuaaucgaagauaauccugaagcuaaagaaucugguuuggcgcaucuuugcgaguucauugaagacugugaacauguuucuuuggcugugagaaucuugcauuuguuaggaaaggaaggacccaagaccaaacaaccaucgagauacauccguuuuaucuacaaucgcgucauauuggaauguccuucuguaagagcugcugcagucuccgccauggcacaauuuggagcuucuugucccgauuuguuagaaaauauccaaauauuacuuucgaggugucagauggauucagacgaugaaguuagggacagagcuacauauuauaguaauauacuuaacaaaaaugauaaaaguuuauacaacaauuacauuuuggauucuuugcagguuucaauuccuucacuagaaagaucgcuuagagaauacauucaaaau

SEQ ID NO: 275 - RNA, 867 nt, Diabrotica barberi
augaaguuggaugguguagaucuacccccaccaauuagcuucgacauugcggaagaacaaccguuaccaccuuaccagcagacguucuuauguaauggugauggaggauccauagugcgacaguuucucgaacaguauuucguaauauacgauucagauaauaggcagucccuucuucaggcauaucacgaaaaagccgcauuuucaaugacaauggcuuacccguauggcuauuccaaagacaguaaaggaguaucgugguugaauugguaugccaccgauaauagaaauuuauuacgaguucaagauccagacagaagaaacaaguuguuaagacagggacaaguugcuguaguuucuuucuugcaagauaugccgcacacgaagcacgauauucacaguuuuacaguagauuugacaguuuuuacaccccagauguuauguuugacaguggcugguauguuuaaagaauugaaaaguggccacaaaguaccuccuuuaagauauuucuucagaacccuuguaauuguaccugcuggaucagguuuuugcauagcaaaugaagaacuucacauauccaaugcaacuccagaccaagcaaaagaugcuuucaagaccaccguuaauguagcuccggcaccagcuccugugguuaccucuccuggacccagcauaccacaaccagcugugccagaugaugcuacaaaacaagaaaugguaaaacagaugucagcaguauccggaaugaaucucgaguggucgcuacagugucucgaagaaacacaaugggacuaccagaaagccauaaugguauuccaaaauuuaaacgcacaagguguuguaccacaagcagcauuuauuaaauga

SEQ ID NO: 276 - RNA, 609 nt, Diabrotica barberi
augaaucccgaguaugauuauuuauucaaacuucugcugauuggagauucaggaguaggaaaaucuugucuucuacugagauuugcagaugauaccuacacagaaagcuauauuaguaccauuggcguagauuuuaaaaucaggacaaucgauuuagauggaaagacaauuaaauugcaaauuugggauacagcaggucaggaaagguuuagaacgauuacaucaaguuauuaccgaggagcacaugguauuauuguaguguacgauugcacagaccaagauucauucaauaacguuaaacaauggcucgaagaaaucgaccguuaugcgugugacaauguaaacaaauuacugguagggaauaaaagcgauuugacaacuaagaaaguugucgacuucacuacagccaaggaguaugccgaccaauugggaauaccauuuuuggaaaccucagcuaagaaugcaaccaauguagaacaggccuuuaugacuauggccgcugaaauaaaaaauagaguaggaccuccaucuucugcaguagaccaaggaaauaagguuagguucgaucaaagucgcccagucgaaacaaccaaaucugguugcugcuga

SEQ ID NO: 277 - DNA, 2637 bp, Diabrotica undecimpunctata
atgggtacttttaaaagggatactcatgatgatgatgggggatcaagtgcttttcaaaacctggaaaaaaccactgttttgcaagaagctagagtttttaatgaaactagtgtaaatccaagaaaatgtacaccaatactaacaaaactgttgtatttattgaaccagggtgaaactttgagtgccaaagaggccacggatgttttcttcgctatgaccaaactgttccaatcaaaagatgtaatattaagaaggatggtttatttgggaattaaagaactcagttctgttgctgatgatgtcattattgtaacatccagtcttacaaaagatatgactggtaaagaagacatgtacagagcagcagctattagagcattgtgcagtattactgatgctactatgcttcaagctatagaacgttatatgaagcaagctattgtagatagaaacccagctgtcagttcagcagcactaattagttcattacatatgagcaaattggctccagatgtagtaaaaagatgggtaaatgaagctcaagaagcagtaaatagtgataatgcaatggtacagtatcacgcattgggtcttctataccatattaggaagactgataagctagcagtgacaaaattgatttccaaattgaatcaaatgggtttaaagagcccttacgctttatgtatgttgataagaatcactgcaaaacttttagaagaagaggaccaagagtctctcctcaactcaccatatacaatgatatttacaatgggcttgagaaacaaatctgaaatggtagtgtatgaagctgcacatgccatggttaacctgaagctcacaagtagtacggtgctagcacctgctataagtgttctgcaactattttgtggatctcctaaagctacactcagatttgctgctgttagaactttaaatcaagtagccaccacccaccctgcctcagtgacagcttgtaatttggatctagaaaacttgattactgattctaatagatcaattgctacactggccattactactcttttgaaaacaggtgctgaatcttctgttgacagactaatgaaacaaatcgctacttttgtatctgaaatcagtgatgagtttaaagtggttgtgattcaggcaattaaggtattagctttgaaatttccaaggaaacatggcacacttatgaatttcctatccgccatgttaagagatgagggaggcttagaatataaagcatccatagcagataccattataaccctaattgaagataaccccgaagctaaagaatctggattggcgcatctttgcgagttcattgaagattgtgaacatgtttctttggctgtgagaatcttacatttgttaggaaaagaaggtcccaagaccaaacaaccatcacgatatatccgttttatttacaatcgcgtcatattggaatgtccctctgtaagagctgctgcagtctctgccatggcacaattcggagcttcttgtccagatttgttagaaaatatccaaatattactttcgaggtgtcagatggattcagacgatgaagttagagaccgagctacatattatagtaatatacttaacaaaaatgataaaagtttatacaacaattacattttggattctttgcaggtatcaatcccttcactagaaagatcgcttagagaatacattcagaatccaactgacgaaccatttgacattaagtccgtacctatagcagcagtgccaacagcagaagaacgcgaagttaaaaacaaatctgaaggactactagtctctcaaggtccagtccgacctcctccggtgtctagagaagaaaacttcgcagaaaaacttagtaacgttccgggtatacaacagttaggtcctttgttcaaaacttcggacgtcgtggaactcactgaatctgaaacagagtattttgtccgctgtatcaagcactgtttcaaacatcacatcgtccttcaattcgattgtctcaataccttgccagaccagcttttagaaaacgttagagtggaaatagacgccggtgaaaccttcgaaattttggcagaaataccttgtgaaaagttgcattataacgaaactggtaccacatatgtagtagttaagttgcccgatgatgatcttccaaactctgttggtacttgtggagccgtgctgaagttcttagtgaaagattgtgatccatcaactggaataccagattctgacgagggttacgatgatgaatatacgctggaagacatcgaaataactttaggggaccaaatccaaaaagtaagcaaagtaaattgggctgcggcttgggaagaagctgcagctacttatgtagaaaaagaagatacatactcattgaccatcaatacgctaagtggcgctgttaagaatattattcagttcttgggattacaacctgcggaaaggactgacagagtaccggagggtaaatctacgcacaccttacttctagctggtgtattcaggggaggtattgacatactagtaagagcgaaactggctttgggcgaatgtgttacgatgcaactaactgtcaggtcgccagatccagacgttgctgagcttataacttcaactgtaggt

SEQ ID NO: 278 - DNA, 867 bp, Diabrotica undecimpunctata
atgaagttggatggtatagatctacccccaccaattagcttcgacattgccgaagaacaaccgttaccgccttaccaacagacgttcttatgtaatggtgatggaggatccatagtgcgtcagtttctcgagcagtatttcttaatatatgattcagataataggcagtcccttcttcaggcatatcacgaaaaagcaacattttcaatgacaatggcttacccctacggctattccaaagacagtaaaggagtaccttggttgaattggtatgccaccgatagtaggaatttattacgagttcaagatcccgacagaagaaacaagttgttaaaacagggacaagttgctgttgtttcgttcttgcaagatatgccgcaaaccaaacacgacattcacagttttacagtagatttgacagtttttacaccccagatgttttgtttgacagtagctggtatgtttaaagaattgaaaagtggacataaagtaccccctttaagatatttcttcagaacccttgtaattgtgcctgctggatcaggtttttgcatagcaaatgaagaacttcacatatccaatgctactccagaccaaacaaaagatgctttcaagacaacagttaatgtagctccggtagcagcccctgtggttacctcccctggacccagtattccacagatagctgtgccagatgatgctacgaaacaagaaatggtaaaacagatgtcagcagtatccggaatgaatctcgagtggtcgctacagtgtctagaagaaacacaatgggactaccaaaaagccataatggtattccaaaatttaaacgcacaaggtgttgtaccacaagcagcatttattaaatga

SEQ ID NO: 279 - DNA, 609 bp, Diabrotica undecimpunctata
atgaatcccgagtatgactatttattcaaacttctgctgattggagattcaggagtaggaaaatcttgccttctactgagatttgcagatgacacctatacagaaagctatattagtaccattggagtagactttaaaatcaggacaatcgatttagatggaaagacaattaaattgcaaatttgggatacagcaggtcaggagagatttagaacgattacttcaagttattacaggggggcacatggtattattgtagtgtacgattgcacagaccaagattcattcaataacgttaaacaatggcttgaagaaatcgaccgttacgcatgtgacaatgtaaacaaattactggtaggaaataaaagcgatttgacaactaagaaagttgttgacttcactacagccaaggaatatgccgaccaattgggaattccatttttggaaacctcagctaagaacgcaaccaatgtagaacaggcctttatgactatggctgctgaaattaaaaatagagtaggacctccatcttcggcagtagaccaaggaaataaggttaggttcgatcaaagtcgcccagtcgaaacaaccaaatctggttgctgctga

SEQ ID NO: 280 - RNA, 2637 nt, Diabrotica undecimpunctata
auggguacuuuuaaaagggauacucaugaugaugaugggggaucaagugcuuuucaaaaccuggaaaaaaccacuguuuugcaagaagcuagaguuuuuaaugaaacuaguguaaauccaagaaaauguacaccaauacuaacaaaacuguuguauuuauugaaccagggugaaacuuugagugccaaagaggccacggauguuuucuucgcuaugaccaaacuguuccaaucaaaagauguaauauuaagaaggaugguuuauuugggaauuaaagaacucaguucuguugcugaugaugucauuauuguaacauccagucuuacaaaagauaugacugguaaagaagacauguacagagcagcagcuauuagagcauugugcaguauuacugaugcuacuaugcuucaagcuauagaacguuauaugaagcaagcuauuguagauagaaacccagcugucaguucagcagcacuaauuaguucauuacauaugagcaaauuggcuccagauguaguaaaaagauggguaaaugaagcucaagaagcaguaaauagugauaaugcaaugguacaguaucacgcauugggucuucuauaccauauuaggaagacugauaagcuagcagugacaaaauugauuuccaaauugaaucaaauggguuuaaagagcccuuacgcuuuauguauguugauaagaaucacugcaaaacuuuuagaagaagaggaccaagagucucuccucaacucaccauauacaaugauauuuacaaugggcuugagaaacaaaucugaaaugguaguguaugaagcugcacaugccaugguuaaccugaagcucacaaguaguacggugcuagcaccugcuauaaguguucugcaacuauuuuguggaucuccuaaagcuacacucagauuugcugcuguuagaacuuuaaaucaaguagccaccacccacccugccucagugacagcuuguaauuuggaucuagaaaacuugauuacugauucuaauagaucaauugcuacacuggccauuacuacucuuuugaaaacaggugcugaaucuucuguugacagacuaaugaaacaaaucgcuacuuuuguaucugaaaucagugaugaguuuaaagugguugugauucaggcaauuaagguauuagcuuugaaauuuccaaggaaacauggcacacuuaugaauuuccuauccgccauguuaagagaugagggaggcuuagaauauaaagcauccauagcagauaccauuauaacccuaauugaagauaaccccgaagcuaaagaaucuggauuggcgcaucuuugcgaguucauugaagauugugaacauguuucuuuggcugugagaaucuuacauuuguuaggaaaagaaggucccaagaccaaacaaccaucacgauauauccguuuuauuuacaaucgcgucauauuggaaugucccucuguaagagcugcugcagucucugccauggcacaauucggagcuucuuguccagauuuguuagaaaauauccaaauauuacuuucgaggugucagauggauucagacgaugaaguuagagaccgagcuacauauuauaguaauauacuuaacaaaaaugauaaaaguuuauacaacaauuacauuuuggauucuuugcagguaucaaucccuucacuagaaagaucgcuuagagaauacauucagaauccaacugacgaaccauuugacauuaaguccguaccuauagcagcagugccaacagcagaagaacgcgaaguuaaaaacaaaucugaaggacuacuagucucucaagguccaguccgaccuccuccggugucuagagaagaaaacuucgcagaaaaacuuaguaacguuccggguauacaacaguuagguccuuuguucaaaacuucggacgucguggaacucacugaaucugaaacagaguauuuuguccgcuguaucaagcacuguuucaaacaucacaucguccuucaauucgauugucucaauaccuugccagaccagcuuuuagaaaacguuagaguggaaauagacgccggugaaaccuucgaaauuuuggcagaaauaccuugugaaaaguugcauuauaacgaaacugguaccacauauguaguaguuaaguugcccgaugaugaucuuccaaacucuguugguacuuguggagccgugcugaaguucuuagugaaagauugugauccaucaacuggaauaccagauucugacgaggguuacgaugaugaauauacgcuggaagacaucgaaauaacuuuaggggaccaaauccaaaaaguaagcaaaguaaauugggcugcggcuugggaagaagcugcagcuacuuauguagaaaaagaagauacauacucauugaccaucaauacgcuaaguggcgcuguuaagaauauuauucaguucuugggauuacaaccugcggaaaggacugacagaguaccggaggguaaaucuacgcacaccuuacuucuagcugguguauucaggggagguauugacauacuaguaagagcgaaacuggcuuugggcgaauguguuacgaugcaacuaacugucaggucgccagauccagacguugcugagcuuauaacuucaacuguaggu

SEQ ID NO: 281 - RNA, 867 nt, Diabrotica undecimpunctata
augaaguuggaugguauagaucuacccccaccaauuagcuucgacauugccgaagaacaaccguuaccgccuuaccaacagacguucuuauguaauggugauggaggauccauagugcgucaguuucucgagcaguauuucuuaauauaugauucagauaauaggcagucccuucuucaggcauaucacgaaaaagcaacauuuucaaugacaauggcuuaccccuacggcuauuccaaagacaguaaaggaguaccuugguugaauugguaugccaccgauaguaggaauuuauuacgaguucaagaucccgacagaagaaacaaguuguuaaaacagggacaaguugcuguuguuucguucuugcaagauaugccgcaaaccaaacacgacauucacaguuuuacaguagauuugacaguuuuuacaccccagauguuuuguuugacaguagcugguauguuuaaagaauugaaaaguggacauaaaguacccccuuuaagauauuucuucagaacccuuguaauugugccugcuggaucagguuuuugcauagcaaaugaagaacuucacauauccaaugcuacuccagaccaaacaaaagaugcuuucaagacaacaguuaauguagcuccgguagcagccccugugguuaccuccccuggacccaguauuccacagauagcugugccagaugaugcuacgaaacaagaaaugguaaaacagaugucagcaguauccggaaugaaucucgaguggucgcuacagugucuagaagaaacacaaugggacuaccaaaaagccauaaugguauuccaaaauuuaaacgcacaagguguuguaccacaagcagcauuuauuaaauga

SEQ ID NO: 282 - RNA, 609 nt, Diabrotica undecimpunctata
augaaucccgaguaugacuauuuauucaaacuucugcugauuggagauucaggaguaggaaaaucuugccuucuacugagauuugcagaugacaccuauacagaaagcuauauuaguaccauuggaguagacuuuaaaaucaggacaaucgauuuagauggaaagacaauuaaauugcaaauuugggauacagcaggucaggagagauuuagaacgauuacuucaaguuauuacaggggggcacaugguauuauuguaguguacgauugcacagaccaagauucauucaauaacguuaaacaauggcuugaagaaaucgaccguuacgcaugugacaauguaaacaaauuacugguaggaaauaaaagcgauuugacaacuaagaaaguuguugacuucacuacagccaaggaauaugccgaccaauugggaauuccauuuuuggaaaccucagcuaagaacgcaaccaauguagaacaggccuuuaugacuauggcugcugaaauuaaaaauagaguaggaccuccaucuucggcaguagaccaaggaaauaagguuagguucgaucaaagucgcccagucgaaacaaccaaaucugguugcugcuga

SEQ ID NO: 283 - DNA, 592 bp, Diabrotica virgifera; identical to SEQ ID NO: 6

SEQ ID NO: 284 - DNA, 669 bp, Diabrotica virgifera
agagatactcatgatgaggacgggggatcaagtgcttttcaaaatctggagaaaactactgttttgcaggaagctagagtttttaatgaaactagtgtaaatccaagaaaatgtacaccgatactaacaaaactgttgtacttattgaaccagggtgaaactttaagtgccaaagaggccacagatgttttctttgccatgaccaaactgttccaatcaaaagatgtaatattgagaaggatggtttatttgggaattaaagaactcagttctgttgctgatgatgtcattattgtaacatccagtcttacaaaagatatgactggtaaagaagacatgtacagagcagctgctataagagcattatgcagtattactgatgctactatgcttcaagctatagaacgttatatgaagcaagctattgtagatagaaacgcagctgtcagttcagcagcactaattagttcattacatatgagcaaattagctccagatgtagtaaaaagatgggtaaatgaagctcaggaagcagtaaatagtgataatgcaatggtacagtatcacgcattaggtcttctataccatattaggaagactgataagctagcagtgacaaaattgatttccaagctgaattcaatgggtttaaagagcccttatgctttg

SEQ ID NO: 285 - DNA, 794 bp, Diabrotica virgifera
attaggaagactgataagctagcagtgacaaaattgatttccaagctgaattcaatgggtttaaagagcccttatgctttgtgtatgttgataagaatcactgcaaaacttttagaagaagaggaccaagagtcactcctcaactccccatatacaataatatttacaatgggcttaaggaacaaatctgaaatggtggtgtatgaagctgcacatgccatggttaacctgaagttcacgagtagtaatgtgctagcacccgctataagtgttctacaactattttgtggatctcctaaagccacactcagatttgctgctgttagaactttaaatcaagtggccaccacccaccctgcgtcagtgacagcttgtaatttggatctagaaaatttgattactgatcctaataggtcaattgctacactggccattactactcttttgaaaacaggtgccgaatcttctgttgacagactaatgaaacaaatcgctacttttgtatctgaaatcagtgatgaatttaaagtggttgtcattcaggcaattaaggtattagctttgaaatttccaaggaaacatagcacgcttatgaatttcctatccgccatgttaagagatgagggaggtttagaatataaagcatccatagcagataccattataaccctaatcgaagataatcccgaagctaaagaatctggtttggcgcatctttgcgagttcattgaagactgtgaacatgtttctttggctgtgagaatcttgcatttgttaggaaaggaaggacccaa

SEQ ID NO: 286 - DNA, 649 bp, Diabrotica virgifera
ctttggctgtgagaatcttgcatttgttaggaaaggaaggacccaagaccaaacaaccatcgagatacatccgttttatctacaatcgcgtcatattggaatgtccttctgtaagagctgctgcagtctccgccatggcacaattcggagcctcttgtcccgatttgttagaaaatatccaaatattactttcgaggtgtcagatggattcagacgatgaagttagggacagagctacatattatagtaatatacttaacaaaaatgataaaagtttatacaacaattacattttggattctttgcaggtttcaattccttcactagaaagatcgcttagagaatacattcaaaatccaactgacgaaccatttgacattaagtccgtacctgtagcagcagtgccaacagcagaagaacgagaagttaaaaacaaatctgaaggactgctagtctctcaaggtccagtccgacctcctccggtgtctagagaagaaaacttcgccgaaaaacttagtaacgttccgggtatacaacagttaggacctttgttcaaaacttccgacgtcgttgaactcactgaatctgaaacagagtattttgtccgctgtatcaagcactgtttcaaacatcacatcgtcctccaatt

SEQ ID NO: 287 - DNA, 721 bp, Diabrotica virgifera
cgtcgttgaactcactgaatctgaaacagagtattttgtccgctgtatcaagcactgtttcaaacatcacatcgtcctccaattcgattgtctgaataccttgccagaccagcttttagaaaacgttagagtggagatagacgccggtgaaaccttcgaaattttggcagaaataccttgtgaaaagttgcactataacgaaaccggtaccacatatgtagtagttaagttgcctgatgatgatctccccaactctgttggtacgtgtggagccgtgttgaagttcttagtgaaagattgtgatccatcaacgggaataccagattctgatgagggttacgatgatgaatatacactggaagacatcgaaataacattaggggaccaaattcaaaaagtaagcaaagtaaattgggctgcagcctgggaagaagctgcagctacttatgtagaaaaagaggatacatactccttgaccatcaatacgctaagtggcgctgttaagaatattattcagttcttgggattacagcctgcggaaaggactgacagagtaccggagggtaaatctacgcacacattacttcttgctggtgtattcaggggaggtattgacatactagtaagagcgaaactagctttgggcgaatgtgttacgatgcaactaacagtcaggtcgccagatcctgacgttgctgagcttataacttcaactgtaggt

SEQ ID NO: 288 - DNA, 558 bp, Diabrotica virgifera; identical to SEQ ID NO: 12

SEQ ID NO: 289 - DNA, 864 bp, Diabrotica virgifera; identical to SEQ ID NO: 102

SEQ ID NO: 290 - DNA, 197 bp, Diabrotica virgifera
aagttggatggtgtagatctgcccccaccaattagcttcgacattgcggaagagcaaccgttaccaccttgccaacagacgttcttatgtaatggtgatggaggatccatagtgcgacagtttctcgagctgtatttcgtaatatatgattcagataataggcagtcccttcttcaggcatatcacgaaaaagccac

SEQ ID NO: 291 - DNA, 198 bp, Diabrotica virgifera
ttcagataataggcagtcccttcttcaggcatatcacgaaaaagccacattttcaatgacaatggcctacccgtacggctattccaaagacagtaaaggagtatcgtggttgaattggtatgccaccgataatagaaatttattacgagttcaagatccagacagaagaaacaagttgttaagacagggacaagttgc

SEQ ID NO: 292 - DNA, 200 bp, Diabrotica virgifera
gttcaagatccagacagaagaaacaagttgttaagacagggacaagttgctgtagtttcgttcttgcaagatatgccgcacacgaagcacgatattcacagttttacagtagatttgacagtttttacaccccagatgttatgtttgacagtggctggtatgtttaaagaattgaaaagtggccacaaagtacctccttt

SEQ ID NO: 293 - DNA, 236 bp, Diabrotica virgifera
cagatgttatgtttgacagtggctggtatgtttaaagaattgaaaagtggccacaaagtacctcctttaagatatttcttcagaacccttgtaattgtacctgctggatcaggtttttgcatagcaaatgaagaacttcacatatccaatgcaactccggaccaagcaaaagatgctttcaagaccaccgttaatgtagctccggcaccagcccctgtgattacctctcctggacc

SEQ ID NO: 294 - DNA, 248 bp, Diabrotica virgifera
ccaccgttaatgtagctccggcaccagcccctgtgattacctctcctggacccagtataccacaacccgctgtgccagatgatgctacaaaacaagaaatggtaaaacagatgtccgcagtatccggaatgaatctcgagtggtcgctacagtgtctcgaagaaacacaatgggactaccagaaagccataatggtattccaaaatttaaacgcacaaggtgttgtaccacaagcagcatttattaaa

SEQ ID NO: 295 - DNA, 564 bp, Diabrotica virgifera; identical to SEQ ID NO: 21

SEQ ID NO: 296 - DNA, 606 bp, Diabrotica virgifera
atgaatcccgagtatgattatttattcaaacttctgctgattggagattcaggagtaggaaaatcttgtcttctactgagatttgcagatgatacctacacagaaagctatattagtaccattggcgtagattttaaaatcaggacaatcgatttagatggaaagacaattaaattgcaaatttgggatacagcaggtcaggaaaggtttagaacgattacatcaagttattaccgaggagcacatggtattattgtagtgtacgattgcacagaccaagattcattcaataacgttaaacagtggctcgaagaaatcgaccgttatgcgtgtgacaatgtaaacaaattactggtagggaataaaagcgatttgacaactaagaaagttgtcgacttcactacagccaaggagtatgccgaccaattgggtataccatttttggaaacctcagctaagaatgcaaccaatgtagaacaggcctttatgactatggccgctgaaataaaaaatagagtaggacctccatcttctgcggtagaccaaggaaataaggttaggttcgatcaaagtcgcccagtcgaaacaaccaaatccggttgctgc

SEQ ID NO: 297 - DNA, 197 bp, Diabrotica virgifera
tgaatcccgagtatgattatttattcaaacttctgctgattggagattcaggagtaggaaaatcttgtcttctactgagatttgcagatgatacctacacagaaagctatattagtaccattggcgtagattttaaaatcaggacaatcgatttagatggaaagacaattaaattgcaaatttgggatacagcaggt

SEQ ID NO: 298 - DNA, 197 bp, Diabrotica virgifera
gatttagatggaaagacaattaaattgcaaatttgggatacagcaggtcaggaaaggtttagaacgattacatcaagttattaccgaggagcacatggtattattgtagtgtacgattgcacagaccaagattcattcaataacgttaaacagtggctcgaagaaatcgaccgttatgcgtgtgacaatgtaaacaa

SEQ ID NO: 299 - DNA, 197 bp, Diabrotica virgifera
cagtggctcgaagaaatcgaccgttatgcgtgtgacaatgtaaacaaattactggtagggaataaaagcgatttgacaactaagaaagttgtcgacttcactacagccaaggagtatgccgaccaattgggtataccatttttggaaacctcagctaagaatgcaaccaatgtagaacaggcctttatgactatggc

SEQ ID NO: 300 - DNA, 155 bp, Diabrotica virgifera
cagctaagaatgcaaccaatgtagaacaggcctttatgactatggccgctgaaataaaaaatagagtaggacctccatcttctgcggtagaccaaggaaataaggttaggttcgatcaaagtcgcccagtcgaaacaaccaaatccggttgctgc

SEQ ID NO: 301 - RNA, 592 nt, Diabrotica virgifera; identical to SEQ ID NO: 126

SEQ ID NO: 302 - RNA, 669 nt, Diabrotica virgifera
agagauacucaugaugaggacgggggaucaagugcuuuucaaaaucuggagaaaacuacuguuuugcaggaagcuagaguuuuuaaugaaacuaguguaaauccaagaaaauguacaccgauacuaacaaaacuguuguacuuauugaaccagggugaaacuuuaagugccaaagaggccacagauguuuucuuugccaugaccaaacuguuccaaucaaaagauguaauauugagaaggaugguuuauuugggaauuaaagaacucaguucuguugcugaugaugucauuauuguaacauccagucuuacaaaagauaugacugguaaagaagacauguacagagcagcugcuauaagagcauuaugcaguauuacugaugcuacuaugcuucaagcuauagaacguuauaugaagcaagcuauuguagauagaaacgcagcugucaguucagcagcacuaauuaguucauuacauaugagcaaauuagcuccagauguaguaaaaagauggguaaaugaagcucaggaagcaguaaauagugauaaugcaaugguacaguaucacgcauuaggucuucuauaccauauuaggaagacugauaagcuagcagugacaaaauugauuuccaagcugaauucaauggguuuaaagagcccuuaugcuuug

SEQ ID NO: 303 - RNA, 794 nt, Diabrotica virgifera
auuaggaagacugauaagcuagcagugacaaaauugauuuccaagcugaauucaauggguuuaaagagcccuuaugcuuuguguauguugauaagaaucacugcaaaacuuuuagaagaagaggaccaagagucacuccucaacuccccauauacaauaauauuuacaaugggcuuaaggaacaaaucugaaauggugguguaugaagcugcacaugccaugguuaaccugaaguucacgaguaguaaugugcuagcacccgcuauaaguguucuacaacuauuuuguggaucuccuaaagccacacucagauuugcugcuguuagaacuuuaaaucaaguggccaccacccacccugcgucagugacagcuuguaauuuggaucuagaaaauuugauuacugauccuaauaggucaauugcuacacuggccauuacuacucuuuugaaaacaggugccgaaucuucuguugacagacuaaugaaacaaaucgcuacuuuuguaucugaaaucagugaugaauuuaaagugguugucauucaggcaauuaagguauuagcuuugaaauuuccaaggaaacauagcacgcuuaugaauuuccuauccgccauguuaagagaugagggagguuuagaauauaaagcauccauagcagauaccauuauaacccuaaucgaagauaaucccgaagcuaaagaaucugguuuggcgcaucuuugcgaguucauugaagacugugaacauguuucuuuggcugugagaaucuugcauuuguuaggaaaggaaggacccaa

SEQ ID NO: 304 - RNA, 649 nt, Diabrotica virgifera
cuuuggcugugagaaucuugcauuuguuaggaaaggaaggacccaagaccaaacaaccaucgagauacauccguuuuaucuacaaucgcgucauauuggaauguccuucuguaagagcugcugcagucuccgccauggcacaauucggagccucuugucccgauuuguuagaaaauauccaaauauuacuuucgaggugucagauggauucagacgaugaaguuagggacagagcuacauauuauaguaauauacuuaacaaaaaugauaaaaguuuauacaacaauuacauuuuggauucuuugcagguuucaauuccuucacuagaaagaucgcuuagagaauacauucaaaauccaacugacgaaccauuugacauuaaguccguaccuguagcagcagugccaacagcagaagaacgagaaguuaaaaacaaaucugaaggacugcuagucucucaagguccaguccgaccuccuccggugucuagagaagaaaacuucgccgaaaaacuuaguaacguuccggguauacaacaguuaggaccuuuguucaaaacuuccgacgucguugaacucacugaaucugaaacagaguauuuuguccgcuguaucaagcacuguuucaaacaucacaucguccuccaauu

SEQ ID NO: 305 - RNA, 721 nt, Diabrotica virgifera
cgucguugaacucacugaaucugaaacagaguauuuuguccgcuguaucaagcacuguuucaaacaucacaucguccuccaauucgauugucugaauaccuugccagaccagcuuuuagaaaacguuagaguggagauagacgccggugaaaccuucgaaauuuuggcagaaauaccuugugaaaaguugcacuauaacgaaaccgguaccacauauguaguaguuaaguugccugaugaugaucuccccaacucuguugguacguguggagccguguugaaguucuuagugaaagauugugauccaucaacgggaauaccagauucugaugaggguuacgaugaugaauauacacuggaagacaucgaaauaacauuaggggaccaaauucaaaaaguaagcaaaguaaauugggcugcagccugggaagaagcugcagcuacuuauguagaaaaagaggauacauacuccuugaccaucaauacgcuaaguggcgcuguuaagaauauuauucaguucuugggauuacagccugcggaaaggacugacagaguaccggaggguaaaucuacgcacacauuacuucuugcugguguauucaggggagguauugacauacuaguaagagcgaaacuagcuuugggcgaauguguuacgaugcaacuaacagucaggucgccagauccugacguugcugagcuuauaacuucaacuguaggu

SEQ ID NO: 306 - RNA, 558 nt, Diabrotica virgifera; identical to SEQ ID NO: 132

SEQ ID NO: 307 - RNA, 864 nt, Diabrotica virgifera; identical to SEQ ID NO: 162

SEQ ID NO: 308 - RNA, 197 nt, Diabrotica virgifera
aaguuggaugguguagaucugcccccaccaauuagcuucgacauugcggaagagcaaccguuaccaccuugccaacagacguucuuauguaauggugauggaggauccauagugcgacaguuucucgagcuguauuucguaauauaugauucagauaauaggcagucccuucuucaggcauaucacgaaaaagccac

SEQ ID NO: 309 - RNA, 198 nt, Diabrotica virgifera
uucagauaauaggcagucccuucuucaggcauaucacgaaaaagccacauuuucaaugacaauggccuacccguacggcuauuccaaagacaguaaaggaguaucgugguugaauugguaugccaccgauaauagaaauuuauuacgaguucaagauccagacagaagaaacaaguuguuaagacagggacaaguugc

SEQ ID NO: 310 - RNA, 200 nt, Diabrotica virgifera
guucaagauccagacagaagaaacaaguuguuaagacagggacaaguugcuguaguuucguucuugcaagauaugccgcacacgaagcacgauauucacaguuuuacaguagauuugacaguuuuuacaccccagauguuauguuugacaguggcugguauguuuaaagaauugaaaaguggccacaaaguaccuccuuu

SEQ ID NO: 311 - RNA, 236 nt, Diabrotica virgifera
cagauguuauguuugacaguggcugguauguuuaaagaauugaaaaguggccacaaaguaccuccuuuaagauauuucuucagaacccuuguaauuguaccugcuggaucagguuuuugcauagcaaaugaagaacuucacauauccaaugcaacuccggaccaagcaaaagaugcuuucaagaccaccguuaauguagcuccggcaccagccccugugauuaccucuccuggacc

SEQ ID NO: 312 - RNA, 248 nt, Diabrotica virgifera
ccaccguuaauguagcuccggcaccagccccugugauuaccucuccuggacccaguauaccacaacccgcugugccagaugaugcuacaaaacaagaaaugguaaaacagauguccgcaguauccggaaugaaucucgaguggucgcuacagugucucgaagaaacacaaugggacuaccagaaagccauaaugguauuccaaaauuuaaacgcacaagguguuguaccacaagcagcauuuauuaaa

SEQ ID NO: 313 - RNA, 564 nt, Diabrotica virgifera; identical to SEQ ID NO: 141

SEQ ID NO: 314 - RNA, 606 nt, Diabrotica virgifera
augaaucccgaguaugauuauuuauucaaacuucugcugauuggagauucaggaguaggaaaaucuugucuucuacugagauuugcagaugauaccuacacagaaagcuauauuaguaccauuggcguagauuuuaaaaucaggacaaucgauuuagauggaaagacaauuaaauugcaaauuugggauacagcaggucaggaaagguuuagaacgauuacaucaaguuauuaccgaggagcacaugguauuauuguaguguacgauugcacagaccaagauucauucaauaacguuaaacaguggcucgaagaaaucgaccguuaugcgugugacaauguaaacaaauuacugguagggaauaaaagcgauuugacaacuaagaaaguugucgacuucacuacagccaaggaguaugccgaccaauuggguauaccauuuuuggaaaccucagcuaagaaugcaaccaauguagaacaggccuuuaugacuauggccgcugaaauaaaaaauagaguaggaccuccaucuucugcgguagaccaaggaaauaagguuagguucgaucaaagucgcccagucgaaacaaccaaauccgguugcugc

SEQ ID NO: 315 - RNA, 197 nt, Diabrotica virgifera
ugaaucccgaguaugauuauuuauucaaacuucugcugauuggagauucaggaguaggaaaaucuugucuucuacugagauuugcagaugauaccuacacagaaagcuauauuaguaccauuggcguagauuuuaaaaucaggacaaucgauuuagauggaaagacaauuaaauugcaaauuugggauacagcaggu

SEQ ID NO: 316 - RNA, 197 nt, Diabrotica virgifera
gauuuagauggaaagacaauuaaauugcaaauuugggauacagcaggucaggaaagguuuagaacgauuacaucaaguuauuaccgaggagcacaugguauuauuguaguguacgauugcacagaccaagauucauucaauaacguuaaacaguggcucgaagaaaucgaccguuaugcgugugacaauguaaacaa

SEQ ID NO: 317 - RNA, 197 nt, Diabrotica virgifera
caguggcucgaagaaaucgaccguuaugcgugugacaauguaaacaaauuacugguagggaauaaaagcgauuugacaacuaagaaaguugucgacuucacuacagccaaggaguaugccgaccaauuggguauaccauuuuuggaaaccucagcuaagaaugcaaccaauguagaacaggccuuuaugacuauggc

SEQ ID NO: 318 - RNA, 155 nt, Diabrotica virgifera
cagcuaagaaugcaaccaauguagaacaggccuuuaugacuauggccgcugaaauaaaaaauagaguaggaccuccaucuucugcgguagaccaaggaaauaagguuagguucgaucaaagucgcccagucgaaacaaccaaauccgguugcugc

SEQ ID NO: 319 - DNA, 1610 bp, Artificial Sequence
  Derived from Diabrotica virgifera and Arabidopsis thaliana
aggaaaggaaggacccaagaccaaacaaccatcgagatacatccgttttatctacaatcgcgtcatattggaatgtccttctgtaagagctgctgcagtctccgccatggcacaattcggagcctcttgtcccgatttgttagaaaatatccaaatattactttcgaggtgtcagatggattcagacgatgaagttagggacagagctacatattatagtaatatacttaacaaaaatgataaaagtttatacaacaattacattttggattctttgcaggtttcaattccttcactagaaagatcgcttagagaatacattcaaaatccaactgacgaaccatttgacattaagtccgtacctgtagcagcagtgccaacagcagaagaacgagaagttaaaaacaaatctgaaggactgctagtctctcaaggtccagtccgacctcctccggtgtctagagaagaaaacttcgccgaaaaacttagtaacgttccgggtatacaacagttaggacctttgttcaaaacttccgacgtcgttgaactcactgaatctgaaacagagtattttgtccgctgtatcaagcacggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcagtcactccatggcctaggtgcttgatacagcggacaaaatactctgtttcagattcagtgagttcaacgacgtcggaagttttgaacaaaggtcctaactgttgtatacccggaacgttactaagtttttcggcgaagttttcttctctagacaccggaggaggtcggactggaccttgagagactagcagtccttcagatttgtttttaacttctcgttcttctgctgttggcactgctgctacaggtacggacttaatgtcaaatggttcgtcagttggattttgaatgtattctctaagcgatctttctagtgaaggaattgaaacctgcaaagaatccaaaatgtaattgttgtataaacttttatcatttttgttaagtatattactataatatgtagctctgtccctaacttcatcgtctgaatccatctgacacctcgaaagtaatatttggatattttctaacaaatcgggacaagaggctccgaattgtgccatggcggagactgcagcagctcttacagaaggacattccaatatgacgcgattgtagataaaacggatgtatctcgatggttgtttggtcttgggtccttcctttcct

SEQ ID NO: 320 - DNA, 820 bp, Artificial Sequence
  Derived from Diabrotica virgifera and Arabidopsis thaliana
aagttggatggtgtagatctgcccccaccaattagcttcgacattgcggaagagcaaccgttaccaccttgccaacagacgttcttatgtaatggtgatggaggatccatagtgcgacagtttctcgagctgtatttcgtaatatatgattcagataataggcagtcccttcttcaggcatatcacgaaaaagccacggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcagtcactccatggcctaggtggctttttcgtgatatgcctgaagaagggactgcctattatctgaatcatatattacgaaatacagctcgagaaactgtcgcactatggatcctccatcaccattacataagaacgtctgttggcaaggtggtaacggttgctcttccgcaatgtcgaagctaattggtgggggcagatctacaccatccaactt

(19) United States
(12) Patent Application Publication
(10) Pub. No.: US 2019/0177736 A1
(43) Pub. Date: Jun. 13, 2019

(54) CONTROL OF COLEOPTERAN PESTS USING RNA MOLECULES

(71) Applicant: Syngenta Pariticpations AG, Basel (CH)

(72) Inventors: Kevin L. DONOHUE, Research Triangle Park, NC (US); Yann NAUDET, Ghent (BE); Lies DEGRAVE, Ghent (BE); Isabelle MAILLET, Ghent (BE); Pascale FELDMANN, Ghent (BE)

(73) Assignee: Syngenta Pariticpations AG, Basel (CH)

(21) Appl. No.: 16/322,151

(22) PCT Filed: Aug. 1, 2017
(86) PCT No.: PCT/US2017/044832

§ 371 Date: Jan. 31, 2019

Related U.S. Application Data
(60) Provisional application No. 62/371,262, filed on Aug. 5, 2016.

(51) Int. Cl. C12N 15/82; A01N 63/02

(52) U.S. Cl. CPC C12N 15/8218; A01N 63/02; C12N 15/8286

(57) ABSTRACT

Disclosed are double stranded RNA molecules that are toxic to coleopteran insects. In particular, interfering RNA molecules capable of interfering with pest target genes and that are toxic to the target pest are provided. Further, methods of making and using the interfering RNA, for example in transgenic plants or as the active ingredient in a composition, to confer protection from insect damage are disclosed.

### SEQUENCE LISTING

A Sequence Listing in ASCII text format, submitted under 37 C.F.R. § 1.821, entitled “81041_ST25.txt”, 477 kilobytes in size, generated on Jun. 22, 2017 and filed via EFS-Web is provided in lieu of a paper copy. This Sequence Listing is hereby incorporated by reference into the specification for its disclosures.

### FIELD OF THE INVENTION

The invention relates generally to the control of pests that cause damage to crop plants by their feeding activities, and more particularly to the control of coleopteran pests by compositions comprising interfering RNA molecules. The invention further relates to the compositions and to methods of using such compositions comprising the interfering RNA molecules.

### BACKGROUND

Insect species in the genus Diabrotica (corn rootworms and cucumber beetles) are considered some of the most important pests to crop plants. For example, species of corn rootworm, including Diabrotica virgifera virgifera, the western corn rootworm (WCR), D. barberi, the northern corn rootworm (NCR), D. undecimpunctata howardi, the southern corn rootworm (SCR), and D. virgifera zeae, the Mexican corn rootworm (MCR), are the most destructive corn pests in North America causing an estimated loss of over $1 billion annually. The western corn rootworm has also invaded Europe and causes an estimated 0.5 billion euros in damage each year. Diabrotica speciosa (common names include, among others, leaf beetle, little Brazilian beetle, cucurbit beetle and chrysanthemum beetle) is an important pest of corn, soybean and peanuts, in South America.

Most of the damage in corn is caused by larval rootworm feeding. Newly hatched rootworm larvae locate corn roots in the soil and initially begin feeding on the fine root hairs and burrow into root tips of the corn plant. As larvae grow larger, they feed on and tunnel into primary roots. When rootworms are abundant, larval feeding and deterioration of injured roots by root rot pathogens can result in roots being pruned to the base of the stalk. Severe root injury interferes with the roots' ability to transport water and nutrients into the plant, reduces plant growth, and results in reduced grain production. Severe root injury also may result in lodging of corn plants, making mechanical harvest more difficult or impossible. Corn rootworm adults feed primarily on corn silk, pollen, and kernels on exposed ear tips. If corn rootworm adults begin emerging before corn reproductive tissues are present, adults may feed on leaf tissue, scraping away the green surface tissue and leaving a window-pane appearance. Silk feeding by adults can result in pruning of silks at the ear tip, commonly called silk clipping. In field corn, beetle populations may reach a level high enough to cause severe silk clipping during pollen shed, which may interfere with pollination and reduce yield. Thus, unlike lepidopteran pests of corn in which only the larval stages cause damage, both the larval and adult stages of corn rootworm are capable of causing economic damage to corn.

Diabrotica insect pests are mainly controlled by intensive applications of chemical pesticides, which may be active against both larval and adult stages, through inhibition of insect growth, prevention of insect feeding or reproduction, or cause death. Good insect control can thus be reached, but these chemicals can sometimes also affect other, beneficial insects. Additional problems occur in areas of high insecticide use where populations of corn rootworm beetles have become resistant to certain insecticides. This has been partially alleviated by various resistance management practices, but there is an increasing need for alternative pest control agents.

Several native Cry proteins from Bacillus thuringiensis, or engineered Cry proteins, have been expressed in transgenic crop plants and exploited commercially to control certain lepidopteran and coleopteran insect pests. For example, starting in 2003, transgenic corn hybrids that control corn rootworm by expressing a Cry3Bb1, Cry34Ab1/Cry35Ab1 or modified Cry3A (mCry3A) or Cry3Ab (eCry3.1Ab) protein have been available commercially in the US.

The seed industry, university researchers and the US Environmental Protection Agency have worked together to develop management plans to help mitigate the onset of insect resistance to transgenic plants expressing insecticidal proteins. They are based primarily on a high dose and refuge strategy. A high dose strategy for corn is to use corn hybrids that express high enough levels of an insecticidal protein such as a Cry protein to kill even partially resistant insects. The underlying hypothesis is that killing partially resistant insects and preventing their mating greatly delays the development of resistance. The success of a high dose strategy depends in part on the specific activity of the insecticidal protein to the particular insect species and how much of that insecticidal protein can be expressed in the transgenic corn plant. The higher the specific activity of an insecticidal protein to a pest, the less amount of the insecticidal protein is required to be expressed in a transgenic plant to achieve a high dose strategy. For example, corn hybrids expressing the lepidopteran-active Cry protein, Cry1Ab, are considered high-dose against the primary target pest European corn borer (Ostrinia nubilalis). Because Cry1Ab is very toxic to European corn borer larvae with an LC50<10 ng/cm² (i.e. high specific activity), levels of expression of Cry1Ab that are achievable in transgenic plants easily places such corn hybrids in a high dose category. However, unlike the lepidopteran-active products, current rootworm products are not considered high-dose. The proteins they express are not active against adults and have limited activity against late instar larvae. Therefore, the current transgenic rootworm products allow some rootworm larvae to survive and emerge as adults.

Thus, economic levels of silk clipping by corn rootworm adults may still occur even in portions of fields planted to a transgenic corn rootworm hybrid. For example, densities of western corn rootworm adults may exceed economic levels in portions of fields planted to transgenic corn rootworm hybrids due to immigration of beetles as well as direct emergence of adults from transgenic root systems. There have been many reports that confirm western corn rootworm adult emergence from certain corn transgenic rootworm hybrids (Crowder et al. (2005) J. Econ. Entomol. 98:534-551). Another publication suggests that western corn rootworm adults will exhibit similar feeding behaviors when encountering some transgenic corn plants or non-transgenic corn plants in the field and that it is unlikely that certain insecticidal proteins in transgenic plants will have significant effects on adults that might impact resistance management.

Therefore, identifying alternative insect control agents with new modes of action would be beneficial. Particularly useful would be new insect control agents that may be toxic to multiple life stages of the target insect pest. Such insect control agents may include those that target genetic elements, such as genes that are essential to the growth and survival of a target insect pest.

RNA interference (RNAi) occurs when an organism recognizes double-stranded RNA (dsRNA) molecules and hydrolyzes them. The resulting hydrolysis products are small RNA fragments of about 19-24 nucleotides in length, called small interfering RNAs (siRNAs). The siRNAs then diffuse or are carried throughout the organism, including across cellular membranes, where they hybridize to mRNAs (or other RNAs) and cause hydrolysis of the RNA. Interfering RNAs are recognized by the RNA interference silencing complex (RISC) into which an effector strand (or “guide strand”) of the RNA is loaded. This guide strand acts as a template for the recognition and destruction of the duplex sequences. This process is repeated each time the siRNA hybridizes to its complementary-RNA target, effectively preventing those mRNAs from being translated, and thus “silencing” the expression of specific genes from which the mRNAs were transcribed. Most plant microRNAs (miRNAs) show extensive base pairing to, and guide cleavage of, their target mRNAs (Jones-Rhoades et al. (2006) Annu. Rev. Plant Biol. 57, 19-53; Llave et al. (2002) Proc. Natl. Acad. Sci. USA 97, 13401-13406). In other instances, interfering RNAs may bind to target RNA molecules having imperfect complementarity, causing translational repression without mRNA degradation. The majority of the animal miRNAs studied so far appear to function in this manner.

RNAi has been found to be useful for insect control of certain insect pests. RNAi strategies typically employ a synthesized, non-naturally occurring “interfering RNA”, or “interfering RNA molecule” which typically comprises at least a RNA fragment against a target gene, a spacer sequence, and a second RNA fragment which is complementary to the first, so that a double-stranded RNA structure can be formed. This non-naturally double-stranded RNA takes advantage of the native RNAi pathways in the insect to trigger down-regulation of target genes that may lead to the cessation of feeding and/or growth and may result in the death of the insect pest.

Although it is known in the literature that RNAi strategies focused on target genes can lead to an insecticidal effect in Diabrotica species, it is also known that not every target sequence is successful, and that an insecticidal effect cannot be predicted. The overwhelming majority of sequences complementary to corn rootworm DNAs are not lethal in species of corn rootworm when used as dsRNA or siRNA. For example, Baum et al. ((2007) Nature Biotechnology 25:1322-1326), describe the effects of inhibiting several WCR gene targets by RNAi. These authors reported that the 8 of 26 target genes they tested were not able to provide experimentally significant coleopteran pest mortality, even at a very high iRNA (e.g., dsRNA) concentration of more than 520 ng/cm². Additionally, target genes against which a dsRNA molecule is known to give a strong RNAi effect in one insect species may not be a good target for different insect species. Whyard et al. ((2009) Insect Biochemistry and Molecular Biology 39: 824-832) report nearly 100-fold differences in efficacy when testing conspecific dsRNA molecules against a V-ATPase gene in four different insect species.

There is an ongoing need for compositions containing insecticidal active ingredients, and for methods of using such compositions, for instance for use in crop protection or insect-mediated disease control. Novel compositions are required to overcome the problem of resistance to existing insecticides and/or to help mitigate the development of resistance to existing transgenic plant approaches. Ideally such compositions have a high toxicity and are effective when ingested orally by the target pest and have applicability for use against both the larval and adult stages of the pest insect. Thus any invention which provided compositions in which any of these properties was enhanced would represent a step forward in the art.

### SUMMARY

The needs outlined above are met by the invention which, in various embodiments, provides new methods of controlling economically important insect pests. The invention in part comprises a method of inhibiting expression of one or more target genes and proteins in coleopteran insect pests. Specifically, the invention comprises methods of modulating expression of one or more target genes in Diabrotica species, such as Diabrotica virgifera virgifera (western corn rootworm), Diabrotica barberi (northern corn rootworm), Diabrotica undecimpunctata howardi (southern corn rootworm), Diabrotica virgifera zeae (Mexican corn rootworm), Diabrotica speciosa (chrysanthemum beetle), and related species, that causes cessation of feeding, growth, development and reproduction, and eventually results in the death of the insect. The method comprises introduction of an interfering RNA molecule comprising a double-stranded RNA (dsRNA) or its modified forms such as small interfering RNA (siRNA) sequences, into cells or into the extracellular environment, such as the midgut, within a pest insect body wherein the dsRNA or siRNA enters the cells and inhibits expression of at least one or more target genes and wherein inhibition of the one or more target genes exerts a deleterious effect upon the pest insect. The interfering RNA molecule is non-naturally occurring. It is specifically contemplated that the methods and compositions of the invention will be useful in limiting or eliminating pest insect infestation in or on any plant by providing one or more compositions comprising interfering RNA molecules comprising dsRNA or siRNA molecules in the diet of the pest. The invention also provides interfering RNA molecules that when delivered to an insect pest inhibits, through a toxic effect, the ability of the insect pest to survive, grow, feed and/or reproduce, or to limit pest related damage or loss to crop plants. Such delivery may be through production of the interfering RNA in a transgenic plant, for example corn, or by topically applying a composition comprising the interfering RNA to a plant or plant seed, such as a corn plant or corn seed. Delivery may further be through contacting the insect with the interfering RNA, such as when the insect feeds on plant material comprising the interfering RNA, either because the plant material is expressing the interfering RNA through a transgenic approach, or because the plant material is coated with a composition comprising the interfering RNA. The interfering RNA may also be provided in an artificial insect diet which the insect then contacts by feeding. The interfering RNA molecule comprises a nucleotide sequence that is complementary to a nucleotide sequence of a mRNA transcribable from a target gene or a portion of a nucleotide sequence of a mRNA transcribable from a target gene of the pest insect and therefore inhibits expression of the target gene, which causes cessation of feeding, growth, development, reproduction and eventually results in death of the pest insect. The invention is further drawn to nucleic acid constructs, nucleic acid molecules and recombinant vectors that comprise or encode at least a fragment of one strand of an interfering RNA molecule of the invention. The invention also provides chimeric nucleic acid molecules comprising an antisense strand of a dsRNA of the interfering RNA operably associated with a plant microRNA precursor molecule. The invention also provides artificial plant microRNA precursors comprising an antisense strand of a dsRNA of an interfering RNA of the invention.

The invention further provides an interfering ribonucleic acid (RNA) molecule wherein the RNA comprises at least one dsRNA wherein the dsRNA is a region of double-stranded RNA comprising annealed complementary strands, one strand of which comprises a sequence of at least 19 contiguous nucleotides which is at least partially complementary to a target nucleotide sequence within a Diabrotica spp target gene, and (i) is at least 85% identical to at least a 19 contiguous nucleotide fragment of SEQ ID NO: 121-210, SEQ ID NO: 274-276, SEQ ID NO: 280-282, SEQ ID NO: 301-318, or the complement thereof; or (ii) comprises at least a 19 contiguous nucleotide fragment of SEQ ID NO: 121-210, SEQ ID NO: 274-276, SEQ ID NO: 280-282, SEQ ID NO: 301-318, or the complement thereof ; or (iii) comprises at least a 19 contiguous nucleotide fragment of a nucleotide sequence encoding an amino acid sequence encoded by SEQ ID NO: 121-210, SEQ ID NO: 274-276, SEQ ID NO: 280-282, SEQ ID NO: 301-318, or the complement thereof, wherein the interfering RNA molecule has insecticidal activity on a coleopteran plant pest. In some embodiments, the interfering molecule may comprise at least two dsRNAs, wherein each dsRNA comprises a sequence of nucleotides which is at least partially complementary to a target nucleotide sequence within the target gene. In further embodiments, each of the dsRNAs may comprise a different sequence of nucleotides which is complementary to a different target nucleotide sequence within the target gene.

The invention further provides compositions comprising one or more interfering RNA molecules comprising two or more of dsRNA molecules, wherein the two or more RNA molecules each comprise a different antisense strand, or comprising two or more nucleic acid constructs or nucleic acid molecules or artificial plant microRNA precursors of the invention.

The invention further provides insecticidal compositions for inhibiting the expression of a Coleopteran insect gene that comprises a dsRNA of the invention and an agriculturally acceptable carrier. In one embodiment, inhibition of the expression of a Diabrotica gene described here leads to cessation of feeding and growth and ultimately results in the death of the Diabrotica insect.

The invention is further drawn to transgenic plants which produce one or more interfering RNA molecules of the invention that are self-protected from insect feeding damage and to methods of using the plants alone or in combination with other insect control strategies to confer maximal insect control capabilities. Plants and/or plant parts producing one or more interfering RNA molecules of the invention or treated with a composition comprising one or more interfering RNA molecules of the invention are highly resistant to insect pest infestation. For example, economically important coleopteran pests can be controlled by a plant that produces an interfering RNA molecule of the invention or by a plant or plant seed that is treated with a composition comprising an interfering RNA molecule of the invention.

The invention also provides a method of controlling a Coleopteran insect plant pest comprising contacting the Coleopteran insect with a nucleic acid molecule that is or is capable of producing an interfering RNA of the invention for inhibiting expression of a gene in the Coleopteran insect thereby controlling the Coleopteran insect.

In other aspects, the invention provides a method of reducing a Diabrotica insect population on a transgenic plant expressing a second insecticidal agent, for example an insecticidal protein, in addition to an interfering RNA of the invention capable of inhibiting expression of an target gene in a Diabrotica insect, thereby reducing the Diabrotica insect population. The second insecticidal agent may be an insecticidal protein derived from Bacillus thuringiensis. A B. thuringiensis insecticidal protein can be any of a number of insecticidal proteins including but not limited to a Cry1 protein, a Cry3 protein, a Cry7 protein, a Cry8 protein, a Cry11 protein, a Cry22 protein, a Cry 23 protein, a Cry 36 protein, a Cry37 protein, a Cry34 protein together with a Cry35 protein, a binary insecticidal protein CryET33 and CryET34, a binary insecticidal protein TIC100 and TIC101, a binary insecticidal protein PS149B1, a VIP, a TIC900 or related protein, a TIC901, TIC1201, TIC407, TIC417,a modified Cry3A protein, or hybrid proteins or chimeras made from any of the preceding insecticidal proteins. In other embodiments, the B. thuringiensis insecticidal protein is selected from the group consisting of Cry3Bb1, Cry34Ab1 together with Cry35Ab1, mCry3A and eCry3.1Ab.

In other embodiments, the second insecticidal agent may be derived from sources other than B. thuringiensis. The second insecticidal agent can be an agent selected from the group comprising a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus laterosporous insecticidal protein, a Lysinibacillus sphearicus insecticidal protein, a Chromobacterium spp. insecticidal protein, a Yersinia entomophaga insecticidal protein, a Paenibacillus popiliae insecticidal protein, a Clostridium spp. (such as C. bifermentans) insecticidal protein, and a lignin. In other embodiments, the second agent may be at least one insecticidal protein derived from an insecticidal toxin complex (Tc) from Photorhabdus, Xenorhabus, Serratia, or Yersinia. In other embodiments. the insecticidal protein may be an ADP-ribosyltransferase derived from an insecticidal bacteria, such as Photorhabdus spp. In other embodiments, the insecticidal protein may be a VIP protein, such as VIP1 or VIP2 from B. cereus. In still other embodiments, the insecticidal protein may be a binary toxin derived from an insecticidal bacteria, such as ISP1A and ISP2A from B. laterosporous or BinA and BinB from L. sphaericus. In still other embodiments, the insecticidal protein may be engineered or may be a hybrid or chimera of any of the preceding insecticidal proteins.

In other aspects, the invention provides a method of reducing resistance development in a Diabrotica insect population to an interfering RNA of the invention, the method comprising expressing in a transgenic plant fed upon by the Diabrotica insect population an interfering RNA of the invention that is capable of inhibiting expression of a target gene in a larval and adult Diabrotica insect, thereby reducing resistance development in the Diabrotica insect population compared to a Diabrotica insect population exposed to an interfering RNA capable of inhibiting expression of a Diabrotica gene described herein in only the larval stage or adult stage of a Diabrotica insect.

In other aspects, the invention provides a method of reducing the level of a target RNA transcribable from a Diabrotica gene described herein in a Diabrotica insect comprising contacting the Diabrotica insect with a composition comprising an interfering RNA molecule of the invention, wherein the interfering RNA molecule reduces the level of the target RNA in a cell of the Diabrotica insect.

In still other aspects, the invention provides a method of conferring Diabrotica insect tolerance or Coleopteran plant pest tolerance to a plant, or part thereof, comprising introducing into the plant, or part thereof, an interfering RNA molecule, a dsRNA molecule, a nucleic acid construct, a chimeric nucleic acid molecule, an artificial plant microRNA precursor molecule and/or a composition of the invention, thereby conferring to the plant or part thereof tolerance to the Diabrotica insect or Coleopteran plant pest.

In further aspects, the invention provides a method of reducing root damage to a plant fed upon by a Diabrotica insect, comprising introducing into cells of the plant an interfering RNA molecule, a dsRNA, a nucleic acid molecule, a nucleic acid construct, a chimeric nucleic acid molecule, an artificial plant microRNA precursor molecule and/or a composition of the invention, thereby reducing root damage to the plant fed upon by a Diabrotica insect.

In other aspects, the invention provides a method of producing a transgenic plant cell having toxicity to a Coleopteran insect, comprising introducing into a plant cell an interfering RNA molecule, a dsRNA, a nucleic acid molecule, a nucleic acid construct, a chimeric nucleic acid molecule, an artificial plant microRNA precursor molecule and/or a composition of the invention, thereby producing the transgenic plant cell having toxicity to the Coleopteran insect compared to a control plant cell.

In further aspects, the invention provides a method of producing a transgenic plant having enhanced tolerance to Coleopteran insect feeding damage, comprising introducing into a plant an interfering RNA molecule, a dsRNA, a nucleic acid molecule, a nucleic acid construct, a chimeric nucleic acid molecule, an artificial plant microRNA precursor molecule and/or a composition of the invention, thereby producing a transgenic plant having enhanced tolerance to Coleopteran insect feeding damage compared to a control plant.

In other aspects, the invention provides a method of enhancing control of a Coleopteran insect population comprising providing a transgenic plant or transgenic seed of the invention and applying to the transgenic plant or the transgenic seed a chemical pesticide that is insecticidal to a Coleopteran insect, thereby enhancing control of the Coleopteran insect population.

In other aspects, the invention provides a method of providing a corn grower with a means of controlling a Coleopteran insect pest population below an economic threshold in a corn crop comprising (a) selling or providing to the grower transgenic corn seed comprising a dsRNA, a nucleic acid molecule, a nucleic acid construct, a chimeric nucleic acid molecule, an artificial plant microRNA precursor molecule and/or a composition of the invention; and (b) advertising to the grower that the transgenic corn seed produces transgenic corn plants capable of controlling a Coleopteran insect pest population.

In another aspect, the invention provides a method of identifying an orthologous target gene for using as a RNAi strategy for the control of a plant pest, said method comprising the steps of: a) producing a primer pair that will amplify a target selected from the group comprising or consisting of SEQ ID NO: 31-90, or a complement thereof; b) amplifying an orthologous target gene from a nucleic acid sample of the plant pest; c) identifying a sequence of an orthologous target gene; d) producing an interfering RNA molecule, wherein the RNA comprises at least one dsRNA, wherein the dsRNA is a region of double-stranded RNA comprising annealed complementary strands, one strand of which comprises a sequence of at least 19 contiguous nucleotides which is at least partially complementary to the orthologous target nucleotide sequence within the target gene; and e) determining if the interfering RNA molecule of step (d) has insecticidal activity on the plant pest. If the interfering RNA has insecticidal activity on the plant pest target gene, an orthologous target gene for using in the control of a plant pest has been identified.

These and other aspects of the invention are set forth in more detail in the description of the invention below.

### BRIEF DESCRIPTION OF THE SEQUENCES IN THE SEQUENCE LISTING

SEQ ID NOs: 1-30 are fragments of DNA coding sequences used to synthesize interfering RNA molecules to test for insecticidal activity.
SEQ ID NOs: 31-90 are nucleic acid sequences of primers used to identify target genes from Diabrotica spp. for testing for insecticidal activity using a RNAi strategy.
SEQ ID NOs: 91-120 are complete DNA coding sequences of the 30 target genes identified in the RNAi-based screen for insecticidal activity.
SEQ ID NOs: 121-150 are RNA sequences of the fragments of the DNA coding sequences used to synthesize interfering RNA molecules to test for insecticidal activity.
SEQ ID NOs: 151-180 are RNA sequences of the complete DNA coding sequences of the 30 target genes identified in the RNAi-based screen for insecticidal activity.
SEQ ID NOs: 181-210 are complete mRNA sequences, including 5′ and 3′ UTRs, for the 30 target genes identified in the RNAi-based screen for insecticidal activity.
SEQ ID NOs: 211-240 are antisense RNA sequences of the complete DNA coding sequences of the 30 target genes identified in the RNAi-based screen for insecticidal activity.
SEQ ID NOs: 241-270 are amino acid sequences of the proteins encoded by the 30 target genes identified in the RNAi-based screen for insecticidal activity.
SEQ ID NOs: 271-273 are DNA coding sequences of NCR orthologs of three selected WCR target genes identified in the RNAi-based screen for insecticidal activity (BPA_41555, BPA_12879, and BPA_71489).
SEQ ID NOs: 274-276 are RNA sequences of the DNA coding sequences of the NCR orthologs of three selected WCR target genes identified in the RNAi-based screen for insecticidal activity (BPA_41555, BPA_12879, and BPA_71489).

SEQ ID NOs: 277-279 are DNA coding sequences of SCR orthologs of three selected WCR target genes identified in the RNAi-based screen for insecticidal activity (BPA_41555, BPA_12879, and BPA_71489).
SEQ ID NOs: 280-282 are RNA sequences of the DNA coding sequences of the SCR orthologs of three selected WCR target genes identified in the RNAi-based screen for insecticidal activity (BPA_41555, BPA_12879, and BPA_71489).
SEQ ID NOs: 283-287 are DNA sequences of fragments of the BPA_41555 target gene.
SEQ ID NOs: 288-294 are DNA sequences of fragments of the BPA_12879 target gene.
SEQ ID NOs: 295-300 are DNA sequences of fragments of the BPA_71489 target gene.
SEQ ID NOs: 301-305 are RNA sequences of fragments of the BPA_41555 target gene mRNA.
SEQ ID NOs: 306-312 are RNA sequences of fragments of the BPA_12879 target gene mRNA.
SEQ ID NOs: 313-318 are RNA sequences of fragments of the BPA_71489 target gene mRNA
SEQ ID NOs: 319-320 are DNA sequences which encode for a hairpin RNA structure to a target gene.

### DETAILED DESCRIPTION

The following is a detailed description of the invention provided to aid those skilled in the art in practicing the invention. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments of the invention will be apparent to those skilled in the art in light of the instant disclosure, which do not depart from the invention. Hence, the following descriptions are intended to illustrate some particular embodiments of the invention, and not to exhaustively specify all permutations, combinations and variations thereof. Those of ordinary skill in the art will recognize that modifications and variations in the embodiments described herein may be made without departing from the spirit or scope of the invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

For clarity, certain terms used in the specification are defined and presented as follows:

As used herein, “a,” “an” or “the” can mean one or more than one. For example, “a cell” can mean a single cell or a multiplicity of cells.

As used herein, “and/or” refers to and encompasses any and all possible combinations of one or more of the associated listed items, as well as the lack of combinations when interpreted in the alternative (or).

Further, the term “about,” as used herein when referring to a measurable value such as an amount of a compound or agent, dose, time, temperature, and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.” A “coding sequence” is a nucleic acid sequence that is transcribed into RNA such as mRNA, rRNA, tRNA, snRNA, sense RNA or antisense RNA. Preferably the RNA is then translated in an organism to produce a protein.

The terms “sequence similarity” or “sequence identity” of nucleotide or amino acid sequences mean a degree of identity or similarity of two or more sequences and may be determined conventionally by using known software or computer programs such as the Best-Fit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2:482-489 (1981), to find the best segment of identity or similarity between two sequences. Sequence comparison between two or more polynucleotides or polypeptides is generally performed by comparing portions of the two sequences over a comparison window to identify and compare local regions of sequence similarity. The comparison window is generally from about 20 to 200 contiguous nucleotides. Gap performs global alignments: all of one sequence with all of another similar sequence using the method of Needleman and Wunsch, J. Mol. Biol. 48:443-453 (1970). When using a sequence alignment program such as BestFit to determine the degree of DNA sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

The phrase “substantially identical,” in the context of two nucleic acids or two amino acid sequences, refers to two or more sequences or subsequences that have at least about 50% nucleotide or amino acid residue identity when compared and aligned for maximum correspondence as measured using one of the following sequence comparison algorithms or by visual inspection. In certain embodiments, substantially identical sequences have at least about 60%, or at least about 70%, or at least about 80%, or even at least about 90% or 95% nucleotide or amino acid residue identity. In certain embodiments, substantial identity exists over a region of the sequences that is at least about 50 residues in length, or over a region of at least about 100 residues, or the sequences are substantially identical over at least about 150 residues. In further embodiments, the sequences are substantially identical when they are identical over the entire length of the coding regions.

The term “homology” in the context of the invention refers to the level of similarity between nucleic acid or amino acid sequences in terms of nucleotide or amino acid identity or similarity, respectively, i.e., sequence similarity or identity. Homology, homologue, and homologous also refers to the concept of similar functional properties among different nucleic acids or proteins. Homologues include genes that are orthologous and paralogous. Homologues can be determined by using the coding sequence for a gene, disclosed herein or found in appropriate database (such as that at NCBI or others) in one or more of the following ways. For an amino acid sequence, the sequences should be compared using algorithms (for instance see section on “identity” and “substantial identity”). For nucleotide sequences the sequence of one DNA molecule can be compared to the sequence of a known or putative homologue in much the same way. Homologues are at least 20% identical, or at least 30% identical, or at least 40% identical, or at least 50% identical, or at least 60% identical, or at least 70% identical, or at least 80% identical, or at least 88% identical, or at least 90% identical, or at least 92% identical, or at least 95% identical, across any substantial region of the molecule (DNA, RNA, or protein molecule).

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another widely used and accepted computer program for performing sequence alignments is CLUSTALW v1.6 (Thompson, et al. Nuc. Acids Res., 22: 4673-4680, 1994). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there were 100 matched amino acids between a 200 and a 400 amino acid proteins, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

Two nucleotide sequences can also be considered to be substantially identical when the two sequences hybridize to each other under stringent conditions. In representative embodiments, two nucleotide sequences considered to be substantially identical hybridize to each other under highly stringent conditions.

The terms “stringent conditions” or “stringent hybridization conditions” include reference to conditions under which a polynucleotide will hybridize to its target sequence to a detectably greater degree than other sequences (e.g., at least 2-fold over background). Stringent conditions are sequence-dependent and will be different in different circumstances. By controlling the stringency of the hybridization and/or washing conditions, target polynucleotides can be identified which are 100% complementary to the probe (homologous probing). Alternatively, stringency conditions can be adjusted to allow some mismatching in sequences so that lower degrees of similarity are detected (heterologous probing). Typically, stringent conditions will be those in which the salt concentration is less than approximately 1.5 M Na ion, typically about 0.01 to 1.0 M Na ion (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions also may be achieved with the addition of destabilizing agents such as formamide. Exemplary low stringency conditions include hybridization with a buffer solution of 30 to 35% formamide, 1 M NaCl, 1% SDS (w/v; sodium dodecyl sulphate) at 37° C., and a wash in 1× to 2×SSC (20×SSC=3.0 M NaCl/0.3 M trisodium citrate) at 50 to 55° C. Moderate stringency conditions detect sequences that share at least 80% sequence identity. Exemplary moderate stringency conditions include hybridization in 40 to 45% formamide, 1 M NaCl, 1% SDS at 37° C., and a wash in 0.5× to 1×SSC at 55 to 60° C. High stringency conditions detect sequences that share at least 90% sequence identity. Exemplary high stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tm can be approximated from the equation of Meinkoth and Wahl (Anal. Biochem., 138:267-284, 1984): Tm=81.5° C.+16.6 (log M)+0.41 (% GC)−0.61 (% form)−500 /L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel, et al., eds., Greene Publishing and Wiley-Interscience, New York (1995). Methods of stringent hybridization are known in the art which conditions can be calculated by means known in the art. This is disclosed in Sambrook et al., Molecular Cloning: A Laboratory Manual, 2nd Ed., Cold Spring Harbor Laboratory Press, 1989, Cold Spring Harbor, N.Y. and Current Protocols in Molecular Biology, Ausebel et al, eds., John Wiley and Sons, Inc., 2000. Methods of determining percent sequence identity are known in the art, an example of which is the GCG computer sequence analysis software (GCG, Inc, Madison Wis.).

Nucleic acids that do not hybridize to each other under stringent conditions are still substantially identical if the proteins that they encode are substantially identical (e.g., due to the degeneracy of the genetic code).

A further indication that two nucleic acids or proteins are substantially identical is that the protein encoded by the first nucleic acid is immunologically cross reactive with the protein encoded by the second nucleic acid. Thus, a protein is typically substantially identical to a second protein, for example, where the two proteins differ only by conservative substitutions.

A nucleic acid sequence is “isocoding with” a reference nucleic acid sequence when the nucleic acid sequence encodes a polypeptide having the same amino acid sequence as the polypeptide encoded by the reference nucleic acid sequence.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

The terms “complementary” or “complementarity,” refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

As used herein, the terms “substantially complementary” or “partially complementary” mean that two nucleic acid sequences are complementary at least about 50%, 60%, 70%, 80% or 90% of their nucleotides. In some embodiments, the two nucleic acid sequences can be complementary at least at 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “dsRNA” or “RNAi” refers to a polyribonucleotide structure formed either by a single self-complementary RNA strand or at least by two complementary RNA strands. The degree of complementary, in other words the % identity, need not necessarily be 100%. Rather, it must be sufficient to allow the formation of a double-stranded structure under the conditions employed. As used herein, the term “fully complementary” means that all the bases of the nucleotide sequence of the dsRNA are complementary to or ‘match’ the bases of the target nucleotide sequence. The term “at least partially complementary” means that there is less than a 100% match between the bases of the dsRNA and the bases of the target nucleotide sequence. The skilled person will understand that the dsRNA need only be at least partially complementary to the target nucleotide sequence in order to mediate down-regulation of expression of the target gene. It is known in the art that RNA sequences with insertions, deletions and mismatches relative to the target sequence can still be effective at RNAi. According to the current invention, it is preferred that the dsRNA and the target nucleotide sequence of the target gene share at least 80% or 85% sequence identity, preferably at least 90% or 95% sequence identity, or more preferably at least 97% or 98% sequence identity and still more preferably at least 99% sequence identity. Alternatively, the dsRNA may comprise 1, 2 or 3 mismatches as compared with the target nucleotide sequence over every length of 24 partially complementary nucleotides. It will be appreciated by the person skilled in the art that the degree of complementarity shared between the dsRNA and the target nucleotide sequence may vary depending on the target gene to be down-regulated or depending on the insect pest species in which gene expression is to be controlled.

It will be appreciated that the dsRNA may comprise or consist of a region of double-stranded RNA comprising annealed complementary strands, one strand of which, the sense strand, comprises a sequence of nucleotides at least partially complementary to a target nucleotide sequence within a target gene.

The target nucleotide sequence may be selected from any suitable region or nucleotide sequence of the target gene or RNA transcript thereof. For example, the target nucleotide sequence may be located within the 5′UTR or 3′UTR of the target gene or RNA transcript or within exonic or intronic regions of the gene. The skilled person will be aware of methods of identifying the most suitable target nucleotide sequences within the context of the full-length target gene. For example, multiple dsRNAs targeting different regions of the target gene can be synthesised and tested. Alternatively, digestion of the RNA transcript with enzymes such as RNAse H can be used to determine sites on the RNA that are in a conformation susceptible to gene silencing. Target sites may also be identified using in silico approaches, for example, the use of computer algorithms designed to predict the efficacy of gene silencing based on targeting different sites within the full-length gene.

Preferably, the % identity of a polyribonucleotide is determined by GAP (Needleman and Wunsch, 1970) analysis (GCG program) using the default settings, wherein the query sequence is at least about 21 to about 23 nucleotides in length, and the GAP analysis aligns the two sequences over a region of at least about 21 nucleotides. In another embodiment, the query sequence is at least 150 nucleotides in length, and the GAP analysis aligns the two sequences over a region of at least 150 nucleotides. In a further embodiment, the query sequence is at least 300 nucleotides in length and the GAP analysis aligns the two sequences over a region of at least 300 nucleotides. In yet another embodiment, the query sequence corresponds to the full length of the target RNA, for example mRNA, and the GAP analysis aligns the two sequences over the full length of the target RNA.

Conveniently, the dsRNA can be produced from a single open reading frame in a recombinant host cell, wherein the sense and anti-sense sequences are flanked by an unrelated sequence which enables the sense and anti-sense sequences to hybridize to form the dsRNA molecule with the unrelated sequence forming a loop structure. Alternatively, the sense strand and antisense strand can be made without an open reading frame to ensure that no protein will be made in the transgenic host cell. The two strands can also be expressed separately as two transcripts, one encoding the sense strand and one encoding the antisense strand.

RNA duplex formation can be initiated either inside or outside the cell. The dsRNA can be partially or fully double-stranded. The RNA can be enzymatically or chemically synthesized, either in vitro or in vivo.

The dsRNA need not be full length relative to either the primary transcription product or fully processed RNA. It is well-known in the art that small dsRNA of about 19-23 bp in length can be used to trigger gene silencing of a target gene. Generally, higher identity can be used to compensate for the use of a shorter sequence. Furthermore, the dsRNA can comprise single stranded regions as well, e.g., the dsRNA can be partially or fully double stranded. The double stranded region of the dsRNA can have a length of at least about 19 to about 23 base pairs, optionally a sequence of about 19 to about 50 base pairs, optionally a sequence of about 50 to about 100 base pairs, optionally a sequence of about 100 to about 200 base pairs, optionally a sequence of about 200 to about 500, and optionally a sequence of about 500 to about 1000 or more base pairs, up to a molecule that is double stranded for its full length, corresponding in size to a full length target RNA molecule. Bolognesi et al (2012, PLOS One, 7(10): e47534, herein incorporated by reference) teach that dsRNAs greater than or equal to about 60 bp are required for biological activity in artificial diet bioassays with Southern Corn Rootworm (SCR; Diabrotica undecimpunctata howardii).

Mao et al (2007, Nature Biotechnology, 35(11): 1307-1313) teach a transgenic plant expressing a dsRNA construct against a target gene (CYP6AE14) of an insect pest (cotton bollworm, Helicoverpa armigera). Insects feeding on the transgenic plant have small RNAs of about 19-23 bp in size of the target gene in their midgut, with a corresponding reduction in CYP6AE14 transcripts and protein. This suggests that the small RNAs were efficacious in reducing expression of the target gene in the insect pest. Therefore, small RNAs of about 19 bp, about 20 bp, about 21 bp, about 22 bp, about 23 bp, about 24 bp, about 25 bp, about 26 bp, about 27 bp, about 28 bp, about 29 bp, or about 30 bp may be efficacious in reducing expression of the target gene in an insect pest.

Alternatively, the dsRNA may comprise a target dsRNA of at least 19 base pairs, and the target dsRNA may be within a dsRNA “carrier” or “filler” sequence. For example, Bolognesi et al (2012) show that a 240 bp dsRNA encompassing a target dsRNA, which comprised a 21 bp contiguous sequence with 100% identity to the target sequence, had biological activity in bioassays with Southern Corn Rootworm. The present application exemplifies a similar approach in bioassays with Western Corn Rootworm. The target dsRNA may have a length of at least 19 to about 25 base pairs, optionally a sequence of about 19 to about 50 base pairs, optionally a sequence of about 50 to about 100 base pairs, optionally a sequence of about 100 to about 200 base pairs, optionally a sequence of about 200 to about 500, and optionally a sequence of about 500 to about 1000 or more base pairs. Combined with the carrier dsRNA sequence, the dsRNA of the target sequence and the carrier dsRNA may have a total length of at least about 50 to about 100 base pairs, optionally a sequence of about 100 to about 200 base pairs, optionally a sequence of about 200 to about 500, and optionally a sequence of about 500 to about 1000 or more base pairs.

The dsRNA can contain known nucleotide analogs or modified backbone residues or linkages, which are synthetic, naturally occurring, and non-naturally occurring. Examples of such analogs include, without limitation, phosphorothioates, phosphoramidates, methyl phosphonates, chiralmethyl phosphonates and 2-O-methyl ribonucleotides.

As used herein, the term “specifically reduce the level of a target RNA and/or the production of a target protein encoded by the RNA”, and variations thereof, refers to the sequence of a portion of one strand of the dsRNA being sufficiently identical to the target RNA such that the presence of the dsRNA in a cell reduces the steady state level and/or the production of said RNA. In many instances, the target RNA will be mRNA, and the presence of the dsRNA in a cell producing the mRNA will result in a reduction in the production of said protein. Preferably, this accumulation or production is reduced at least 10%, more preferably at least 50%, even more preferably at least 75%, yet even more preferably at least 95% and most preferably 100%, when compared to a wild-type cell.

The consequences of inhibition can be confirmed by examination of the outward properties of the cell or organism or by biochemical techniques such as, but not limited to, Northern hybridization, reverse transcription, gene expression monitoring with a microarray, antibody binding, enzyme linked immunosorbent assay (ELISA), Western blotting, radioimmunoassay (RIA), and other immunoassays.

The interfering RNAs of the current invention may comprise one dsRNA or multiple dsRNAs, wherein each dsRNA comprises or consists of a sequence of nucleotides which is at least partially complementary to a target nucleotide sequence within the target gene and that functions upon uptake by an insect pest species to down-regulate expression of said target gene. Concatemeric RNA constructs of this type are described in WO2006/046148 as incorporated herein by reference. In the context of the present invention, the term ‘multiple’ means at least two, at least three, at least four, etc and up to at least 10, 15, 20 or at least 30. In one embodiment, the interfering RNA comprises multiple copies of a single dsRNA i.e. repeats of a dsRNA that binds to a particular target nucleotide sequence within a specific target gene. In another embodiment, the dsRNAs within the interfering RNA comprise or consist of different sequences of nucleotides complementary to different target nucleotide sequences. It should be clear that combinations of multiple copies of the same dsRNA combined with dsRNAs binding to different target nucleotide sequences are within the scope of the current invention.

The dsRNAs may be arranged as one contiguous region of the interfering RNA or may be separated by the presence of linker sequences. The linker sequence may comprise a short random nucleotide sequence that is not complementary to any target nucleotide sequences or target genes. In one embodiment, the linker is a conditionally self-cleaving RNA sequence, preferably a pH-sensitive linker or a hydrophobic-sensitive linker. In one embodiment, the linker comprises a sequence of nucleotides equivalent to an intronic sequence. Linker sequences of the current invention may range in length from about 1 base pair to about 10000 base pairs, provided that the linker does not impair the ability of the interfering RNA to down-regulate the expression of target gene(s).

In addition to the dsRNA(s) and any linker sequences, the interfering RNA of the invention may comprise at least one additional polynucleotide sequence. In different embodiments of the invention, the additional sequence is chosen from (i) a sequence capable of protecting the interfering RNA against RNA processing, (ii) a sequence affecting the stability of the interfering RNA, (iii) a sequence allowing protein binding, for example to facilitate uptake of the interfering RNA by cells of the insect pest species, (iv) a sequence facilitating large-scale production of the interfering RNA, (v) a sequence which is an aptamer that binds to a receptor or to a molecule on the surface of the insect pest cells to facilitate uptake, or (v) a sequence that catalyses processing of the interfering RNA within the insect pest cells and thereby enhances the efficacy of the interfering RNA. Structures for enhancing the stability of RNA molecules are well known in the art and are described further in WO2006/046148 as incorporated herein by reference.

The interfering RNA may contain DNA bases, non-natural bases or non-natural backbone linkages or modifications of the sugar-phosphate backbone, for example to enhance stability during storage or enhance resistance to degradation by nucleases. Furthermore, the interfering RNA may be produced chemically or enzymatically by one skilled in the art through manual or automated reactions. Alternatively, the interfering RNA may be transcribed from a polynucleotide encoding the same. Thus, provided herein is an isolated polynucleotide encoding any of the interfering RNAs of the current invention.

MicroRNAs (miRNAs) are non-protein coding RNAs, generally of between about 18 to about 25 nucleotides in length (commonly about 20-24 nucleotides in length in plants). These miRNAs direct cleavage in trans of target transcripts, negatively regulating the expression of genes involved in various regulation and development pathways (Bartel, Cell, 116:281-297 (2004); Zhang et al. Dev. Biol. 289:3-16 (2006)). As such, miRNAs have been shown to be involved in different aspects of plant growth and development as well as in signal transduction and protein degradation. In addition, small endogenous mRNAs including miRNAs may also be involved in biotic stress responses such as pathogen attack. Since the first miRNAs were discovered in plants (Reinhart et al. Genes Dev. 16:1616-1626 (2002), Park et al. Curr. Biol. 12:1484-1495 (2002)) many hundreds have been identified. Furthermore, many plant miRNAs have been shown to be highly conserved across very divergent taxa. (Floyd et al. Nature 428:485-486 (2004); Zhang et al. Plant J. 46:243-259 (2006)). Many microRNA genes (MIR genes) have been identified and made publicly available in a database (miRBase, available via the world wide web). miRNAs are also described in U.S. Patent Publications 2005/0120415 and 2005/144669A1, the entire contents of which are incorporated by reference herein.

Genes encoding miRNAs yield primary miRNAs (termed a “pri-miRNA”) of 70 to 300 bp in length that can form imperfect stem-loop structures. A single pri-miRNA may contain from one to several miRNA precursors. In animals, pri-miRNAs are processed in the nucleus into shorter hairpin RNAs of about 65 nt (pre-miRNAs) by the RNaselll enzyme Drosha and its cofactor DGCR8/Pasha. The pre-miRNA is then exported to the cytoplasm, where it is further processed by another RNaselll enzyme, Dicer, releasing a miRNA/miRNA* duplex of about 22 nt in size. In contrast to animals, in plants, the processing of pri-miRNAs into mature miRNAs occurs entirely in the nucleus using a single RNaselll enzyme, DCL1 (Dicer-like 1). (Zhu. Proc. Natl. Acad. Sci. 105:9851-9852 (2008)). Many reviews on microRNA biogenesis and function are available, for example, see, Bartel Cell 116:281-297 (2004), Murchison et al. Curr. Opin. Cell Biol. 16:223-229 (2004), Dugas et al. Curr. Opin. Plant Biol. 7:512-520 (2004) and Kim Nature Rev. Mol. Cell Biol. 6:376-385 (2005).

The term “plant microRNA precursor molecule” as used herein describes a small (˜70-300 nt) non-coding RNA sequence that is processed by plant enzymes to yield a ˜19-24 nucleotide product known as a mature microRNA sequence. The mature sequences have regulatory roles through complementarity to messenger RNA (mRNA). The term “artificial plant microRNA precursor molecule” describes the non-coding miRNA precursor sequence prior to processing that is employed as a backbone sequence for the delivery of a siRNA molecule via substitution of the endogenous native miRNA/miRNA* duplex of the miRNA precursor molecule with that of a non-native, heterologous miRNA (amiRNA/amiRNA*; e.g. siRNA/siRNA*) that is then processed into the mature miRNA sequence with the siRNA sequence.

In the context of the invention, the term “toxic” used to describe a dsRNA of the invention means that the dsRNA molecules of the invention and combinations of such dsRNA molecules function as orally active insect control agents that have a negative effect on an insect. When a composition of the invention is delivered to the insect, the result is typically death of the insect, or the insect does not feed upon the source that makes the composition available to the insect. Such a composition may be a transgenic plant expressing the dsRNA of the invention.

To “control” or “controlling” insects means to inhibit, through a toxic effect, the ability of one or more insect pests to survive, grow, feed, and/or reproduce, or to limit insect-related damage or loss in crop plants. To “control” insects may or may not mean killing the insects, although it preferably means killing the insects. A composition that controls a target insect has insecticidal activity against the target insect.

To “deliver” or “delivering” a composition or dsRNA means that the composition or dsRNA comes in contact with an insect, resulting in a toxic effect and control of the insect. The composition or dsRNA can be delivered in many recognized ways, e.g., orally by ingestion by the insect via transgenic plant expression, formulated composition(s), sprayable composition(s), a bait matrix, or any other art-recognized toxicant delivery system.

The term “insect” as used herein includes any organism now known or later identified that is classified in the animal kingdom, phylum Arthropoda, class Insecta, including but not limited to insects in the orders Coleoptera (beetles), Lepidoptera (moths, butterflies), Diptera (flies), Protura, Collembola (springtails), Diplura, Microcoryphia (jumping bristletails), Thysanura (bristletails, silverfish), Ephemeroptera (mayflies), Odonata (dragonflies, damselflies), Orthoptera (grasshoppers, crickets, katydids), Phasmatodea (walkingsticks), Grylloblattodea (rock crawlers), Mantophasmatodea, Dermaptera (earwigs), Plecoptera (stoneflies), Embioptera (web spinners), Zoraptera, lsoptera (termites), Mantodea (mantids), Blattodea (cockroaches), Hemiptera (true bugs, cicadas, leafhoppers, aphids, scales), Thysanoptera (thrips), Psocoptera (book and bark lice), Phthiraptera (lice; including but not limited to suborders Amblycera, lschnocera and Anoplura), Neuroptera (lacewings, owlflies, mantispids, antlions), Hymenoptera (bees, ants, wasps), Trichoptera (caddisflies), Siphonaptera (fleas), Mecoptera (scorpion flies), Strepsiptera (twisted-winged parasites), and any combination thereof.

As used herein, a “coleopteran insect” refers to any member of the Coleoptera order, including coleopteran plant pests. Insects in the order Coleoptera include but are not limited to any coleopteran insect now known or later identified including those in suborders Archostemata, Myxophaga, Adephaga and Polyphaga, and any combination thereof.

“Diabrotica ” is a genus of beetles (from the Coleoptera order) commonly referred to as “corn rootworms” or “cucumber beetles.” Diabrotica insects that are pests of crop plants, include without limitation, Diabrotica barberi (northern corn rootworm; NCR), D. virgifera virgifera (western corn rootworm; WCR), D. undecimpunctata howardii (southern corn rootworm; SCR), D. virgifera zeae (Mexican corn rootworm; MCR) and D. speciosa. In the context of the invention, the term “corn rootworm” or “cucumber beetle” is interchangeable with the term “Diabrotica.”

Other nonlimiting examples of coleopteran insect pests according to the present invention include Leptinotarsa spp. such as L. decemlineata (Colorado potato beetle); Chrysomela spp. such as C. scripta (cottonwood leaf beetle); Hypothenemus spp. such as H. hampei (coffee berry borer); Sitophilus spp. such as S. zeamais (maize weevil); Epitrix spp. such as E. hirtipennis (tobacco flea beetle) and E. cucumeris (potato flea beetle); Phyllotreta spp. such as P. cruciferae (crucifer flea beetle) and P. pusilla (western black flea beetle); Anthonomus spp. such as A. eugenii (pepper weevil); Hemicrepidus spp. such as H. memnonius (wireworms); Melanotus spp. such as M. communis (wireworm); Ceutorhychus spp. such as C. assimilis (cabbage seedpod weevil); Phyllotreta spp. such as P. cruciferae (crucifer flea beetle); Aeolus spp. such as A. mellillus (wireworm); Aeolus spp. such as A. mancus (wheat wireworm); Horistonotus spp. such as H. uhlerii (sand wireworm); Sphenophorus spp. such as S. maidis (maize billbug), S. zeae (timothy billbug), S. parvulus (bluegrass billbug), and S. callosus (southern corn billbug); Phyllophaga spp. (White grubs); Chaetocnema spp. such as C. pulicaria (corn flea beetle); Popillia spp. such as P. japonica (Japanese beetle); Epilachna spp. such as E. varivestis (Mexican bean beetle); Cerotoma spp. such as C. trifurcate (Bean leaf beetle); Epicauta spp. such as E. pestifera and E. lemniscata (Blister beetles); and any combination of the foregoing.

A “Diabrotica life stage” or “corn rootworm life stage” means the egg, larval, pupal or adult developmental form of a Diabrotica species.

“Effective insect-controlling amount” means that concentration of dsRNA that inhibits, through a toxic effect, the ability of insects to survive, grow, feed and/or reproduce, or to limit insect-related damage or loss in crop plants. “Effective insect-controlling amount” may or may not mean a concentration that kills the insects, although it preferably means that it kills the insects.

The term “agrochemically active ingredient” refers to chemicals and/or biological compositions, such as those described herein, which are effective in killing, preventing, or controlling the growth of undesirable pests, such as, plants, insects, mice, microorganism, algae, fungi, bacteria, and the like (such as pesticidally active ingredients). An interfering RNA molecule of the invention is an agrochemically active ingredient.

An “agriculturally acceptable carrier” includes adjuvants, mixers, enhancers, etc. beneficial for application of an active ingredient, such as an interfering RNA molecule of the invention. Suitable carriers should not be phytotoxic to valuable crops, particularly at the concentrations employed in applying the compositions in the presence of crops, and should not react chemically with the compounds of the active ingredient herein, namely an interfering RNA of the invention, or other composition ingredients. Such mixtures can be designed for application directly to crops, or can be concentrates or formulations which are normally diluted with additional carriers and adjuvants before application. They may include inert or active components and can be solids, such as, for example, dusts, granules, water dispersible granules, or wettable powders, or liquids, such as, for example, emulsifiable concentrates, solutions, emulsions or suspensions. Suitable agricultural carriers may include liquid carriers, for example water, toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methanol, ethanol, isopropanol, amyl alcohol, ethylene glycol, propylene glycol, glycerine, and the like. Water is generally the carrier of choice for the dilution of concentrates. Suitable solid carriers may include talc, pyrophyllite clay, silica, attapulgus clay, kieselguhr, chalk, diatomaceous earth, lime, calcium carbonate, bentonire clay, Fuller's earth, cotton seed hulls, wheat flour, soybean flour, pumice, wood flour, walnut shell flour, lignin, and the like.

For the present invention, an agriculturally acceptable carrier may also include non-pathogenic, attenuated strains of microorganisms, which carry the insect control agent, namely an interfering RNA molecule of the invention. In this case, the microorganisms carrying the interfering RNA may also be referred to as insect control agents. The microorganisms may be engineered to express a nucleotide sequence of a target gene to produce interfering RNA molecules comprising RNA sequences homologous or complementary to RNA sequences typically found within the cells of an insect. Exposure of the insects to the microorganisms result in ingestion of the microorganisms and down-regulation of expression of target genes mediated directly or indirectly by the interfering RNA molecules or fragments or derivatives thereof.

In another embodiment, the interfering RNA molecules may be encapsulated in a synthetic matrix such as a polymer and applied to the surface of a host such as a plant. Ingestion of the host cells by an insect permits delivery of the insect control agents to the insect and results in down-regulation of a target gene in the host.

A composition of the invention, for example a composition comprising an interfering RNA molecule of the invention and an agriculturally acceptable carrier, may be used in conventional agricultural methods. For example, the compositions of the invention may be mixed with water and/or fertilizers and may be applied preemergence and/or postemergence to a desired locus by any means, such as airplane spray tanks, irrigation equipment, direct injection spray equipment, knapsack spray tanks, cattle dipping vats, farm equipment used in ground spraying (e.g., boom sprayers, hand sprayers), and the like. The desired locus may be soil, plants, and the like.

A composition of the invention may be applied to a seed or plant propagule in any physiological state, at any time between harvest of the seed and sowing of the seed; during or after sowing; and/or after sprouting. It is preferred that the seed or plant propagule be in a sufficiently durable state that it incurs no or minimal damage, including physical damage or biological damage, during the treatment process. A formulation may be applied to the seeds or plant propagules using conventional coating techniques and machines, such as fluidized bed techniques, the roller mill method, rotostatic seed treaters, and drum coaters.

“Expression cassette” as used herein means a nucleic acid sequence capable of directing expression of a particular nucleic acid sequence in an appropriate host cell, comprising a promoter operably linked to the nucleic acid sequence of interest which is operably linked to termination signal sequences. It also typically comprises sequences required for proper translation of the nucleic acid sequence. The expression cassette comprising the nucleic acid sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event. The expression of the nucleic acid sequence in the expression cassette may be under the control of, for example, a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

A “gene” is a defined region that is located within a genome and that, besides the aforementioned coding sequence, comprises other, primarily regulatory nucleic acid sequences responsible for the control of the expression, that is to say the transcription and translation, of the coding portion. A gene may also comprise other 5′ and 3′ untranslated sequences and termination sequences. Further elements that may be present are, for example, introns.

As used herein, the term “grower” means a person or entity that is engaged in agriculture, raising living organisms, such as crop plants, for example corn, for food, feed or raw materials.

A “heterologous” nucleic acid sequence is a nucleic acid sequence not naturally associated with a host cell into which it is introduced, including non-naturally occurring multiple copies of a naturally occurring nucleic acid sequence.

A “homologous” nucleic acid sequence is a nucleic acid sequence naturally associated with a host cell into which it is introduced.

“Insecticidal” is defined as a toxic biological activity capable of controlling insects, preferably by killing them.

An “isolated” nucleic acid molecule or nucleotide sequence or nucleic acid construct or dsRNA molecule or protein of the invention is generally exists apart from its native environment and is therefore not a product of nature. An isolated nucleic acid molecule or nucleotide sequence or nucleic acid construct or dsRNA molecule or protein may exist in a purified form or may exist in a non-native environment such as, for example, a recombinant host or host cell such as a transgenic plant or transgenic plant cell.

In the context of the invention, a number in front of the suffix “mer” indicates a specified number of subunits. When applied to RNA or DNA, this specifies the number of bases in the molecule. For example, a 19 nucleotide subsequence of an mRNA having the sequence ACUGGUCGCGUUGCAUGCU is a “19-mer.”

A “plant” is any plant at any stage of development, particularly a seed plant.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

A corn rootworm “transcriptome” is a collection of all or nearly all the ribonucleic acid (RNA) transcripts in a corn rootworm cell.

“Transformation” is a process for introducing heterologous nucleic acid into a host cell or organism. In particular, “transformation” means the stable integration of a DNA molecule into the genome of an organism of interest.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

The nomenclature used herein for DNA or RNA bases and amino acids is as set forth in 37 C.F.R. § 1.822.

The invention is based on the unexpected discovery that double stranded RNA (dsRNA) or small interfering RNAs (siRNA) designed to target a mRNA transcribable from the Diabrotica genes described herein are toxic to the Diabrotica insect pest and can be used to control Diabrotica or Coleopteran infestation of a plant and impart to a transgenic plant tolerance to a Diabrotica or Coleopteran infestation. Thus, in one embodiment, the invention provides a double stranded RNA (dsRNA) molecule comprising a sense strand and an antisense strand, wherein a nucleotide sequence of the antisense strand is complementary to a portion of a mRNA polynucleotide transcribable from a Diabrotica insect gene described in the present disclosure, wherein the dsRNA molecule is toxic to a Diabrotica insect or Coleopteran plant pest.

It is known in the art that dsRNA molecules that are not perfectly complementary to a target sequence (for example, having only 95% identity to the target gene) are effective to control coleopteran pests (see, for example, Narva et al., U.S. Pat. No. 9,012,722). The invention provides an interfering RNA molecule comprising at least one dsRNA, where the dsRNA is a region of double-stranded RNA comprising annealed at least partially complementary strands. One strand of the dsRNA comprises a sequence of at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 contiguous nucleotides which is at least partially complementary to a target nucleotide sequence within a Diabrotica spp target gene. The interfering RNA molecule (i) has at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity, to at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO:121-210, SEQ ID NO: 274-276, SEQ ID NO: 280-282, SEQ ID NO: 301-318, or the complement thereof; (ii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO:121-210, SEQ ID NO: 274-276, SEQ ID NO: 280-282, SEQ ID NO: 301-318, or the complement thereof; (iii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of a nucleotide sequence encoding an amino acid sequence encoded by SEQ ID NO:121-210, SEQ ID NO: 274-276, SEQ ID NO: 280-282, SEQ ID NO: 301-318, or the complement thereof, or (iv) can hybridize under stringent conditions to a polynucleotide selected from the group consisting of SEQ ID NO:121-210, SEQ ID NO: 274-276, SEQ ID NO: 280-282, SEQ ID NO: 301-318, and the complements thereof, wherein the interfering RNA molecule has insecticidal activity on a coleopteran plant pest.

In some embodiments, the interfering RNA molecule comprises at least two dsRNAs, wherein each dsRNA comprises a sequence of nucleotides which is at least partially complementary to a target nucleotide sequence within the target gene. In some embodiments, each of the dsRNAs comprise a different sequence of nucleotides which is complementary to a different target nucleotide sequence within the target gene. In other embodiments, each of the dsRNAs comprise a different sequence of nucleotides which is complementary to a target nucleotide sequence within two different target genes.

In some embodiments, the interfering RNA molecule comprises a dsRNA that can comprise, consist essentially of or consist of from at least 18 to about 25 consecutive nucleotides (e.g. 18, 19, 20, 21, 22, 23, 24, 25, 26, 27, 28, or 29) to at least about 300 consecutive nucleotides. Additional nucleotides can be added at the 3′ end, the 5′ end or both the 3′ and 5′ ends to facilitate manipulation of the dsRNA molecule but that do not materially affect the basic characteristics or function of the dsRNA molecule in RNA interference (RNAi).

In some embodiments, the interfering RNA molecule comprises a dsRNA which comprises an antisense strand that is complementary to at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 consecutive nucleotides of SEQ ID NO:121-210, SEQ ID NO: 274-276, SEQ ID NO: 280-282, SEQ ID NO: 301-318, or the complement thereof. In other embodiments, the portion of dsRNA comprises, consists essentially of or consists of at least from 19, 20 or 21 consecutive nucleotides to at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 consecutive nucleotides of SEQ ID NO:121-210, SEQ ID NO: 274-276, SEQ ID NO: 280-282, SEQ ID NO: 301-318, or the complement thereof.

In other embodiments, an interfering RNA molecule of the invention comprises a dsRNA which comprises, consists essentially of or consists of any 21-mer subsequence of SEQ ID NO: 181-210 consisting of N to N+20 nucleotides, or any complement thereof. For example, an interfering RNA molecule of the invention comprises a dsRNA which comprises, consist essentially of or consists of any 21-mer subsequence of SEQ ID NO: 181, wherein N is nucleotide 1 to nucleotide 776 of SEQ ID NO: 181, or any complement thereof. In other words, the portion of the mRNA that is targeted comprises any of the 776 21 consecutive nucleotide subsequences i.e. 21-mers) of SEQ ID NO: 181, or any of their complementing sequences. It will be recognized that these 776 21 consecutive nucleotide subsequences include all possible 21 consecutive nucleotide subsequences from SEQ ID NO: 121 and from SEQ ID NO: 151, and their complements, as SEQ ID NOs 121, 151, and 181 are all to the same target, namely BPA_15366. It will similarly be recognized that all 21-mer subsequences of SEQ ID NO: 181-210, and all complement subsequences thereof, include all possible 21 consecutive nucleotide subsequences of SEQ ID NOs: 121-180, and the complement subsequences thereof.

Similarly, an interfering RNA molecule of the invention comprises a dsRNA which comprises, consist essentially of or consists of any 21-mer subsequence of SEQ ID NO: 182, wherein N is nucleotide 1 to nucleotide 771 of SEQ ID NO: 182, or any complement thereof. Another interfering RNA molecule of the invention comprises a dsRNA which comprises, consist essentially of or consists of any 21-mer subsequence of SEQ ID NO: 183, wherein N is nucleotide 1 to nucleotide 2907 of SEQ ID NO: 183, or any complement thereof. Another interfering RNA molecule of the invention comprises a dsRNA which comprises, consist essentially of or consists of any 21-mer subsequence of SEQ ID NO: 184, wherein N is nucleotide 1 to nucleotide 1600 of SEQ ID NO: 184, or any complement thereof. Another interfering RNA molecule of the invention comprises a dsRNA which comprises, consist essentially of or consists of any 21-mer subsequence of SEQ ID NO: 185, wherein N is nucleotide 1 to nucleotide 2410 of SEQ ID NO: 185, or any complement thereof. Another interfering RNA molecule of the invention comprises a dsRNA which comprises, consist essentially of or consists of any 21-mer subsequence of SEQ ID NO: 186, wherein N is nucleotide 1 to nucleotide 2802 of SEQ ID NO: 186, or any complement thereof. Another interfering RNA molecule of the invention comprises a dsRNA which comprises, consist essentially of or consists of any 21-mer subsequence of SEQ ID NO: 187, wherein N is nucleotide 1 to nucleotide 3681 of SEQ ID NO: 187, or any complement thereof. Another interfering RNA molecule of the invention comprises a dsRNA which comprises, consist essentially of or consists of any 21-mer subsequence of SEQ ID NO: 188, wherein N is nucleotide 1 to nucleotide 651 of SEQ ID NO: 188, or any complement thereof. Another interfering RNA molecule of the invention comprises a dsRNA which comprises, consist essentially of or consists of any 21-mer subsequence of SEQ ID NO: 189, wherein N is nucleotide 1 to nucleotide 673 of SEQ ID NO: 189, or any complement thereof. Another interfering RNA molecule of the invention comprises a dsRNA which comprises, consist essentially of or consists of any 21-mer subsequence of SEQ ID NO: 190, wherein N is nucleotide 1 to nucleotide 2664 of SEQ ID NO: 190, or any complement thereof. Another interfering RNA molecule of the invention comprises a dsRNA which comprises, consist essentially of or consists of any 21-mer subsequence of SEQ ID NO: 191, wherein N is nucleotide 1 to nucleotide 438 of SEQ ID NO: 191, or any complement thereof. Another interfering RNA molecule of the invention comprises a dsRNA which comprises, consist essentially of or consists of any 21-mer subsequence of SEQ ID NO: 192, wherein N is nucleotide 1 to nucleotide 2458 of SEQ ID NO: 192, or any complement thereof. Another interfering RNA molecule of the invention comprises a dsRNA which comprises, consist essentially of or consists of any 21-mer subsequence of SEQ ID NO: 193, wherein N is nucleotide 1 to nucleotide 3254 of SEQ ID NO: 193, or any complement thereof. Another interfering RNA molecule of the invention comprises a dsRNA which comprises, consist essentially of or consists of any 21-mer subsequence of SEQ ID NO: 194, wherein N is nucleotide 1 to nucleotide 3632 of SEQ ID NO: 194, or any complement thereof. Another interfering RNA molecule of the invention comprises a dsRNA which comprises, consist essentially of or consists of any 21-mer subsequence of SEQ ID NO: 195, wherein N is nucleotide 1 to nucleotide 7611 of SEQ ID NO: 195, or any complement thereof. Another interfering RNA molecule of the invention comprises a dsRNA which comprises, consist essentially of or consists of any 21-mer subsequence of SEQ ID NO: 196, wherein N is nucleotide 1 to nucleotide 1008 of SEQ ID NO: 196, or any complement thereof. Another interfering RNA molecule of the invention comprises a dsRNA which comprises, consist essentially of or consists of any 21-mer subsequence of SEQ ID NO: 197, wherein N is nucleotide 1 to nucleotide 2992 of SEQ ID NO: 197, or any complement thereof. Another interfering RNA molecule of the invention comprises a dsRNA which comprises, consist essentially of or consists of any 21-mer subsequence of SEQ ID NO: 198, wherein N is nucleotide 1 to nucleotide 1192 of SEQ ID NO: 198, or any complement thereof. Another interfering RNA molecule of the invention comprises a dsRNA which comprises, consist essentially of or consists of any 21-mer subsequence of SEQ ID NO: 199, wherein N is nucleotide 1 to nucleotide 7626 of SEQ ID NO: 199, or any complement thereof. Another interfering RNA molecule of the invention comprises a dsRNA which comprises, consist essentially of or consists of any 21-mer subsequence of SEQ ID NO: 200, wherein N is nucleotide 1 to nucleotide 2580 of SEQ ID NO: 200, or any complement thereof. Another interfering RNA molecule of the invention comprises a dsRNA which comprises, consist essentially of or consists of any 21-mer subsequence of SEQ ID NO: 201, wherein N is nucleotide 1 to nucleotide 4628 of SEQ ID NO: 201, or any complement thereof. Another interfering RNA molecule of the invention comprises a dsRNA which comprises, consist essentially of or consists of any 21-mer subsequence of SEQ ID NO: 202, wherein N is nucleotide 1 to nucleotide 1557 of SEQ ID NO: 202, or any complement thereof. Another interfering RNA molecule of the invention comprises a dsRNA which comprises, consist essentially of or consists of any 21-mer subsequence of SEQ ID NO: 203, wherein N is nucleotide 1 to nucleotide 1019 of SEQ ID NO: 203, or any complement thereof. Another interfering RNA molecule of the invention comprises a dsRNA which comprises, consist essentially of or consists of any 21-mer subsequence of SEQ ID NO: 204, wherein N is nucleotide 1 to nucleotide 677 of SEQ ID NO: 204, or any complement thereof. Another interfering RNA molecule of the invention comprises a dsRNA which comprises, consist essentially of or consists of any 21-mer subsequence of SEQ ID NO: 205, wherein N is nucleotide 1 to nucleotide 764 of SEQ ID NO: 205, or any complement thereof. Another interfering RNA molecule of the invention comprises a dsRNA which comprises, consist essentially of or consists of any 21-mer subsequence of SEQ ID NO: 206, wherein N is nucleotide 1 to nucleotide 1830 of SEQ ID NO: 206, or any complement thereof. Another interfering RNA molecule of the invention comprises a dsRNA which comprises, consist essentially of or consists of any 21-mer subsequence of SEQ ID NO: 207, wherein N is nucleotide 1 to nucleotide 3225 of SEQ ID NO: 207, or any complement thereof. Another interfering RNA molecule of the invention comprises a dsRNA which comprises, consist essentially of or consists of any 21-mer subsequence of SEQ ID NO: 208, wherein N is nucleotide 1 to nucleotide 1003 of SEQ ID NO: 208, or any complement thereof. Another interfering RNA molecule of the invention comprises a dsRNA which comprises, consist essentially of or consists of any 21-mer subsequence of SEQ ID NO: 209, wherein N is nucleotide 1 to nucleotide 1419 of SEQ ID NO: 209, or any complement thereof. Another interfering RNA molecule of the invention comprises a dsRNA which comprises, consist essentially of or consists of any 21-mer subsequence of SEQ ID NO: 210, wherein N is nucleotide 1 to nucleotide 5206 of SEQ ID NO: 210, or any complement thereof.

In still other embodiments, the interfering RNA molecule of the invention comprises a dsRNA which comprises, consists essentially of or consists of SEQ ID NO:121-210, SEQ ID NO: 274-276, SEQ ID NO: 280-282, SEQ ID NO: 301-318, or the complement thereof.

In other embodiments of the interfering RNA molecule of the invention, the nucleotide sequence of the antisense strand of a dsRNA of the invention comprises, consists essentially of or consists of the nucleotide sequence of SEQ ID NO: 211-240. The nucleotide sequence of the antisense strand of a dsRNA of the invention can have one nucleotide at either the 3′ or 5′ end deleted or can have up to six nucleotides added at the 3′ end, the 5′ end or both, in any combination to achieve an antisense strand consisting essentially of any 19-mer, any 20-mer, or any 21-mer nucleotide sequence of SEQ ID NO: 211-240, as it would be understood that the deletion of the one nucleotide or the addition of up to the six nucleotides do not materially affect the basic characteristics or function of the double stranded RNA molecule of the invention. Such additional nucleotides can be nucleotides that extend the complementarity of the antisense strand along the target sequence and/or such nucleotides can be nucleotides that facilitate manipulation of the RNA molecule or a nucleic acid molecule encoding the RNA molecule, as would be known to one of ordinary skill in the art. For example, a TT overhang at the 3′ end may be present, which is used to stabilize the siRNA duplex and does not affect the specificity of the siRNA.

In some embodiments of this invention, the antisense strand of the double stranded RNA of the interfering RNA molecule can be fully complementary to the target RNA polynucleotide or the antisense strand can be substantially complementary or partially complementary to the target RNA polynucleotide. The dsRNA of the interfering RNA molecule may comprise a dsRNA which is a region of double-stranded RNA comprising substantially complementary annealed strands, or which is a region of double-stranded RNA comprising fully complementary annealed strands. By substantially or partially complementary is meant that the antisense strand and the target RNA polynucleotide can be mismatched at about 1, 2, 3, 4, 5, 6, 7, 8, 9, or 10 nucleotide pairings. Such mismatches can be introduced into the antisense strand sequence, e.g., near the 3′ end, to enhance processing of the double stranded RNA molecule by Dicer, to duplicate a pattern of mismatches in a siRNA molecule inserted into a chimeric nucleic acid molecule or artificial microRNA precursor molecule of this invention, and the like, as would be known to one of skill in the art. Such modification will weaken the base pairing at one end of the duplex and generate strand asymmetry, therefore enhancing the chance of the antisense strand, instead of the sense strand, being processed and silencing the intended gene (Geng and Ding “Double-mismatched siRNAs enhance selective gene silencing of a mutant ALS-causing Allele1” Acta Pharmacol. Sin. 29:211-216 (2008); Schwarz et al. “Asymmetry in the assembly of the RNAi enzyme complex” Cell 115:199-208 (2003)).

In some embodiments of this invention, the interfering RNA comprises a dsRNA which comprises a short hairpin RNA (shRNA) molecule. Expression of shRNA in cells is typically accomplished by delivery of plasmids or recombinant vectors, for example in transgenic plants such as transgenic corn.

The invention encompasses a nucleic acid construct comprising an interfering RNA of the invention. The invention further encompasses a nucleic acid molecule encoding at least one interfering molecule of the invention. The invention further encompasses a nucleic acid construct comprising at least one interfering molecule of the invention or comprising a nucleic acid molecule encoding the at least one interfering molecule of the invention. The invention further encompasses a nucleic acid construct wherein the nucleic acid construct is an expression vector. The invention further encompasses a recombinant vector comprising a regulatory sequence operably linked to a nucleotide sequence that encodes an interfering RNA molecule of the invention. A regulatory sequence may refer to a promoter, enhancer, transcription factor binding site, insulator, silencer, or any other DNA element involved in the expression of a gene.

The invention further encompasses chimeric nucleic acid molecules comprising an interfering RNA molecule with an antisense strand of a dsRNA operably linked with a plant microRNA precursor molecule. In some embodiments, the chimeric nucleic acid molecule comprises an antisense strand having the nucleotide sequence of any of the 21-mer subsequences of SEQ ID NOs: 181-210, or any complement thereof, operably linked with a plant microRNA precursor molecule. In some embodiments, the plant microRNA precursor molecule is a maize microRNA precursor.

In some embodiments, the invention encompasses an artificial plant microRNA precursor molecule comprising an antisense strand of a dsRNA of an interfering RNA molecule of the invention. In other embodiments, the artificial plant microRNA precursor molecule comprises an antisense strand having the nucleotide sequence of any of the 19-mer, 20-mer, or 21-mer subsequences of SEQ ID NOs: 211-240. The use of artificial plant microRNAs to deliver a nucleotide sequence of interest (e.g an artificial miRNA; siRNA/siRNA*) into a plant is known in the art (see, e.g., Schwab et al. 2006. The Plant Cell 18:1121-1133 and Examples section herein). In the invention, the artificial microRNAs are chimeric or hybrid molecules, having a plant microRNA precursor backbone and an insect siRNA sequence inserted therein. As would be understood by one of ordinary skill in the art, it is typically desirable to maintain mismatches that normally occur in the plant microRNA precursor sequence in any nucleotide sequence that is substituted into the plant microRNA precursor backbone. In still other embodiments, the artificial plant microRNA precursor comprises portions of a corn microRNA precursor molecule. Any corn microRNA (miRNA) precursor is suitable for the compositions and methods of the invention. Non-limiting examples include miR156, miR159, miR160, miR162, miR164, miR166, miR167, miR168, miR169, miR171, miR172, miR319, miR390, miR393, miR394, miR395, miR396, miR397, miR398, miR399, miR408, miR482, miR528, miR529, miR827, miR1432, as well as any other plant miRNA precursors now known or later identified.

In some embodiments, the invention encompasses interfering RNA molecules, nucleic acid constructs, nucleic acid molecules or recombinant vectors comprising at least one strand of a dsRNA of an interfering RNA molecule of the invention, or comprising a chimeric nucleic acid molecule of the invention, or comprising an artificial plant microRNA of the invention. In some embodiments the nucleic acid construct comprises a nucleic acid molecule of the invention. In other embodiments, the nucleic acid construct is a recombinant expression vector.

In some embodiments, the interfering RNA molecules of the invention have insecticidal activity on a Diabrotica insect. In some embodiments the Diabrotica insect selected from the group consisting of Diabrotica barberi (northern corn rootworm), D. virgifera virgifera (western corn rootworm), D. undecimpunctata howardi (southern corn rootworm), D. balteata (banded cucumber beetle), D. undecimpunctata undecimpunctata (western spotted cucumber beetle), D. significata (3-spotted leaf beetle), D. speciosa (chrysanthemum beetle), D. virgifera zeae (Mexican corn rootworm), D. beniensis, D. cristata, D. curvipustulata, D. dissimilis, D. elegantula, D. emorsitans, D. graminea, D. hispanolae, D. lemniscata, D. linsleyi, D. milleri, D. nummularis, D. occlusa, D. porracea, D. scutellata, D. tibialis, D. trifasciata and D. viridula. In further embodiments, the Diabrotica insect is D. virgifera virgifera (western corn rootworm), D. undecimpunctata howardi (southern corn rootworm) or D. barberi (northen corn rootworm). In some embodiments, the coding sequence of the target gene comprises a sequence selected from the group comprising SEQ ID NO: 91-120.

In some embodiments, the invention encompasses a composition comprising one or more or two or more of the interfering RNA molecules of the invention. In some embodiments, the interfering RNA molecules are present on the same nucleic acid construct, on different nucleic acid constructs, or any combination thereof. For example, one interfering RNA molecule of the invention may be present on a nucleic acid construct, and a second interfering RNA molecule of the invention may be present on the same nucleic acid construct or on a separate, second nucleic acid construct. The second interfering RNA molecule of the invention may be to the same target gene or to a different target gene.

In some embodiments, the invention encompasses a composition comprising an interfering RNA molecule which comprises at least one dsRNA wherein the dsRNA is a region of double-stranded RNA comprising annealed complementary strands. One strand of the dsRNA comprises a sequence of at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 contiguous nucleotides which is at least partially complementary to a target nucleotide sequence within a Diabrotica spp target gene. The interfering RNA molecule (i) has at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity, to at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO:121-210, SEQ ID NO: 274-276, SEQ ID NO: 280-282, SEQ ID NO: 301-318, or the complement thereof; (ii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO:121-210, SEQ ID NO: 274-276, SEQ ID NO: 280-282, SEQ ID NO: 301-318, or the complement thereof; (iii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of a nucleotide sequence encoding an amino acid sequence encoded by SEQ ID NO:121-210, SEQ ID NO: 274-276, SEQ ID NO: 280-282, SEQ ID NO: 301-318, or the complement thereof, or (iv) can hybridize under stringent conditions to a polynucleotide selected from the group consisting of SEQ ID NO:121-210, SEQ ID NO: 274-276, SEQ ID NO: 280-282, SEQ ID NO: 301-318, and the complements thereof.

In some embodiments, the invention encompasses compositions comprising an interfering RNA molecule comprising two or more dsRNAs, wherein the two or more dsRNAs each comprise a different antisense strand. In some embodiments the invention encompasses compositions comprising at least two more interfering RNA molecules, wherein the two or more interfering RNA molecules each comprise a dsRNA comprising a different antisense strand. The two or more interfering RNAs may be present on the same nucleic acid construct, on different nucleic acid constructs or any combination thereof. In other embodiments, the composition comprises a RNA molecule comprising an antisense strand consisting essentially of a nucleotide sequence comprising at least a 19 contiguous nucleotide fragment of SEQ ID NO: 211-240, and in some embodiments may further comprise an RNA molecule comprising an antisense strand consisting essentially of a second nucleotide sequence comprising at least a 19 contiguous nucleotide fragment of SEQ ID NO: 211-240; and in some embodiments may further comprise an RNA molecule comprising an antisense strand consisting essentially of a third nucleotide sequence comprising at least a 19 contiguous nucleotide fragment of SEQ ID NO: 211-240, and in some embodiments may further comprise an RNA molecule comprising an antisense strand consisting essentially of a fourth nucleotide sequence comprising at least a 19 contiguous nucleotide fragment of SEQ ID NO: 211-240, and in some embodiments may further comprise an RNA molecule comprising an antisense strand consisting essentially of a fifth nucleotide sequence comprising at least a 19 contiguous nucleotide fragment of SEQ ID NO: 211-240, and in some embodiments may further comprise an RNA molecule comprising an antisense strand consisting essentially of a sixth nucleotide sequence comprising at least a 19 contiguous nucleotide fragment of SEQ ID NO: 211-240, and in some embodiments may further comprise an RNA molecule comprising an antisense strand consisting essentially of a seventh nucleotide sequence comprising at least a 19 contiguous nucleotide fragment of SEQ ID NO: 211-240. In other embodiments, the composition may comprise two or more of the nucleic acid molecules, wherein the two or more nucleic acid molecules each encode a different interfering RNA molecule. In other embodiments, the composition may comprise two or more of the nucleic acid constructs, wherein the two or more nucleic acid constructs each comprise a nucleic acid molecule encoding a different interfering RNA.

In other embodiments, the composition comprises two or more nucleic acid constructs, two or more nucleic acid molecules, two or more chimeric nucleic acid molecules, two or more artificial plant microRNA precursors of the invention, wherein the two or more nucleic acid constructs, two or more nucleic acid molecules, two or more chimeric nucleic acid molecules, or two or more artificial plant microRNA precursors, each comprise a different antisense strand.

In some embodiments, the invention encompasses an insecticidal composition for inhibiting the expression of a Diabrotica insect gene described herein, comprising an interfering RNA of the invention and an agriculturally acceptable carrier. In some embodiments, the acceptable agricultural carrier is a transgenic organism expressing an interfering RNA of the invention. In some embodiments the transgenic organism may be a transgenic plant expressing the interfering RNA of the invention that when fed upon by a target Coleopteran plant pest causes the target Coleopteran plant pest to stop feeding, growing or reproducing or causing death of the target Coleopteran plant pest. In other embodiments, the transgenic plant is a transgenic corn plant and the target pest is a Diabrotica insect pest. In still other embodiments, the Diabrotica insect pest is selected from the group consisting of Diabrotica barberi (northern corn rootworm), D. virgifera virgifera (western corn rootworm), D. undecimpunctata howardi (southern corn rootworm), D. balteata (banded cucumber beetle), D. undecimpunctata undecimpunctata (western spotted cucumber beetle), D. significata (3-spotted leaf beetle), D. speciosa (chrysanthemum beetle), D. virgifera zeae (Mexican corn rootworm).

In other embodiments, the transgenic organism is selected from, but not limited to, the group consisting of: yeast, fungi, algae, bacteria, virus or an arthropod expressing the interfering RNA molecule of the invention. In some embodiments, the transgenic organism is a virus, for example an insect baculovirus that expresses an interfering RNA molecule of the invention upon infection of an insect host. Such a baculovirus is likely more virulent against the target insect than the wildtype untransformed baculovirus. In other embodiments the transgenic organism is a transgenic bacterium that is applied to an environment where a target pest occurs or is known to have occurred. In some embodiments, non-pathogenic symbiotic bacteria, which are able to live and replicate within plant tissues, so-called endophytes, or non-pathogenic symbiotic bacteria, which are capable of colonizing the phyllosphere or the rhizosphere, so-called epiphytes, are used. Such bacteria include bacteria of the genera Agrobacterium, Alcaligenes, Azospirillum, Azotobacter, Bacillus, Clavibacter, Enterobacter, Erwinia, Flavobacter, Klebsiella, Pseudomonas, Rhizobium, Serratia, Streptomyces and Xanthomonas. Symbiotic fungi, such as Trichoderma and Gliocladium are also possible hosts for expression of the inventive interfering RNA molecule for the same purpose.

In some embodiments, an acceptable agricultural carrier is a formulation useful for applying the composition comprising the interfering RNA molecule to a plant or seed. In some embodiments, the interfering RNA molecules are stabilized against degradation because of their double stranded nature and the introduction of Dnase/Rnase inhibitors. For example, dsRNA or siRNA can be stabilized by including thymidine or uridine nucleotide 3′ overhangs. The dsRNA or siRNA contained in the compositions of the invention can be chemically synthesized at industrial scale in large amounts. Methods available would be through chemical synthesis or through the use of a biological agent.

In other embodiments the formulation comprises a transfection promoting agent. In other embodiments, the transfection promoting agent is a lipid-containing compound. In further embodiments, the lipid-containing compound is selected from the group consisting of; Lipofectamine, Cellfectin, DMRIE-C, DOTAP and Lipofectin. In another embodiment, the lipid-containing compound is a Tris cationic lipid.

In some embodiments, the formulation further comprises a nucleic acid condensing agent. The nucleic acid condensing agent can be any such compound known in the art. Examples of nucleic acid condensing agents include, but are not limited to, spermidine (N-[3-aminopropyl]-1,4-butanediamine), protamine sulphate, poly-lysine as well as other positively charged peptides. In some embodiments, the nucleic acid condensing agent is spermidine or protamine sulfate.

In still further embodiments, the formulation further comprises buffered sucrose or phosphate buffered saline.

In some embodiments, the invention encompasses transgenic plants, or parts thereof, comprising an interfering RNA molecule, a nucleic acid construct, a chimeric nucleic acid molecule, a artificial plant microRNA precursor molecule and/or a composition of the invention, wherein the transgenic plant has enhanced resistance to a Coleopteran insect or Diabrotica insect as compared to a control plant. In other embodiments, the transgenic plant, or part thereof, is a transgenic corn plant, or part thereof. The invention further encompasses transgenic seed of the transgenic plants of the invention, wherein the transgenic seed comprises an interfering RNA molecule, a nucleic acid construct, a chimeric nucleic acid molecule, an artificial plant microRNA precursor molecule and/or a composition of the invention. In some embodiments the transgenic seed is a transgenic corn seed.

Transgenic plants expressing an interfering RNA of the invention are tolerant or resistant to attack by target insect pests. When the insect starts feeding on such a transgenic plant, it also ingests the expressed dsRNA or siRNA. This may deter the insect from further biting into the plant tissue or may even harm or kill the insect. A nucleic acid sequence encoding a dsRNA or siRNA of the invention is inserted into an expression cassette, which is then preferably stably integrated in the genome of the plant. The nucleic acid sequences of the expression cassette introduced into the genome of the plant are heterologous to the plant and non-naturally occurring. Plants transformed in accordance with the present invention may be monocots or dicots and include, but are not limited to, corn, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis, and woody plants such as coniferous and deciduous trees. In further embodiments, the transgenic plant is a transgenic corn plant.

Expression of the interfering RNA molecule in transgenic plants is driven by regulatory sequences comprising promoters that function in plants. The choice of promoter will vary depending on the temporal and spatial requirements for expression, and also depending on the insect target species. Thus, expression of the interfering RNAs of this invention in leaves, in stalks or stems, in ears, in inflorescences (e.g. spikes, panicles, cobs, etc.), in roots, and/or seedlings is contemplated. In many cases, however, protection against more than one type of insect pest is sought, and thus expression in multiple tissues is desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the dsRNA or siRNA in the desired cell.

Promoters useful with the invention include, but are not limited to, those that drive expression of a nucleotide sequence constitutively, those that drive expression when induced, and those that drive expression in a tissue- or developmentally-specific manner. These various types of promoters are known in the art.

In some embodiments, tissue-specific/tissue-preferred promoters can be used. Tissue-specific or tissue-preferred expression patterns include, but are not limited to, green tissue specific or preferred, root specific or preferred, stem specific or preferred, and flower specific or preferred. In addition, promoters functional in plastids can be used. In some embodiments of the invention, inducible promoters can be used. In further aspects, the nucleotide sequences of the invention can be operably associated with a promoter that is wound inducible or inducible by pest or pathogen infection (e.g., a insect or nematode plant pest)

In some embodiments of the present invention, a “minimal promoter” or “basal promoter” is used. A minimal promoter is capable of recruiting and binding RNA polymerase II complex and its accessory proteins to permit transcriptional initiation and elongation. In some embodiments, a minimal promoter is constructed to comprise only the nucleotides/nucleotide sequences from a selected promoter that are required for binding of the transcription factors and transcription of a nucleotide sequence of interest that is operably associated with the minimal promoter including but not limited to TATA box sequences. In other embodiments, the minimal promoter lacks cis sequences that recruit and bind transcription factors that modulate (e.g., enhance, repress, confer tissue specificity, confer inducibility or repressibility) transcription. A minimal promoter is generally placed upstream (i.e., 5′) of a nucleotide sequence to be expressed. Thus, nucleotides/nucleotide sequences from any promoter useable with the present invention can be selected for use as a minimal promoter.

In some embodiments, a recombinant nucleic acid molecule of the invention can be an “expression cassette.” As used herein, “expression cassette” means a recombinant nucleic acid molecule comprising a nucleotide sequence of interest (e.g., the nucleotide sequences of the invention), wherein the nucleotide sequence is operably associated with at least a control sequence (e.g., a promoter). Thus, some embodiments of the invention provide expression cassettes designed to express nucleotides sequences encoding the dsRNAs or siRNAs of the invention. In this manner, for example, one or more plant promoters operably associated with one or more nucleotide sequences of the invention are provided in expression cassettes for expression in a corn plant, plant part and/or plant cell.

An expression cassette comprising a nucleotide sequence of interest may be chimeric, meaning that at least one of its components is heterologous with respect to at least one of its other components. An expression cassette may also be one that comprises a native promoter driving its native gene, however it has been obtained in a recombinant form useful for heterologous expression. Such usage of an expression cassette makes it so it is not naturally occurring in the cell into which it has been introduced.

An expression cassette also can optionally include a transcriptional and/or translational termination region (i.e., termination region) that is functional in plants. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and/or the pea rbcs E9 terminator. These can be used in both monocotyledons and dicotyledons. In addition, a coding sequence's native transcription terminator can be used.

An expression cassette of the invention also can include a nucleotide sequence for a selectable marker, which can be used to select a transformed plant, plant part and/or plant cell. As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleotide sequence encoding neo or nptll, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin. One of skill in the art is capable of choosing a suitable selectable marker for use in an expression cassette of the invention.

An expression cassette of the invention also can include polynucleotides that encode other desired traits. Such desired traits can be other polynucleotides which confer insect resistance, or which confer nematode resistance, or other agriculturally desirable traits. Such polynucleotides can be stacked with any combination of nucleotide sequences to create plants, plant parts or plant cells having the desired phenotype. Stacked combinations can be created by any method including, but not limited to, cross breeding plants by any conventional methodology, or by genetic transformation. If stacked by genetically transforming the plants, nucleotide sequences encoding additional desired traits can be combined at any time and in any order. For example, a single transgene can comprise multiple expression cassettes, such that multiple expression cassettes are introduced into the genome of a transformed cell at a single genomic location. Alternatively, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or other composition of the invention, provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853.

Thus, an expression cassette can include a coding sequence for one or more polypeptides for agronomic traits that primarily are of benefit to a seed company, grower or grain processor. A polypeptide of interest can be any polypeptide encoded by a polynucleotide sequence of interest. Non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, and/or fungal resistance. See, e.g., U.S. Pat. Nos. 5,569,823; 5,304,730; 5,495,071; 6,329,504; and 6,337,431.

Vectors suitable for plant transformation are described elsewhere in this specification. For Agrobacterium-mediated transformation, binary vectors or vectors carrying at least one T-DNA border sequence are suitable, whereas for direct gene transfer any vector is suitable and linear DNA containing only the construct of interest may be preferred. In the case of direct gene transfer, transformation with a single DNA species or co-transformation can be used (Schocher et al. Biotechnology 4:1093-1096 (1986)). For both direct gene transfer and Agrobacterium-mediated transfer, transformation is usually (but not necessarily) undertaken with a selectable marker that may provide resistance to an antibiotic (kanamycin, hygromycin or methotrexate) or a herbicide (basta). Plant transformation vectors of the invention may also comprise other selectable marker genes, for example, phosphomannose isomerase (pmi), which provides for positive selection of the transgenic plants as disclosed in U.S. Pat. Nos. 5,767,378 and 5,994,629, herein incorporated by reference, or phosphinotricin acetyltransferase (pat), which provides tolerance to the herbicide phosphinotricin (glufosinate). The choice of selectable marker is not, however, critical to the invention.

In other embodiments, a nucleic acid sequence of the invention is directly transformed into the plastid genome. Plastid transformation technology is extensively described in U.S. Pat. Nos. 5,451,513, 5,545,817, and 5,545,818, in PCT application no. WO 95/16783, and in McBride et al. (1994) Proc. Nati. Acad. Sci. USA 91, 7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Nati. Acad. Sci. USA 87, 8526-8530; Staub, J. M., and Maliga, P. (1992) Plant Cell 4, 39-45). This resulted in stable homoplasmic transformants at a frequency of approximately one per 100 bombardments of target leaves. The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-cletoxifying enzyme aminoglycoside-3′-adenyltransferase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19:4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear- expressed genes to permit expression levels that can readily exceed 10% of the total soluble plant protein. In a preferred embodiment, a nucleic acid sequence of the present invention is inserted into a plastid-targeting vector and transformed into the plastid genome of a desired plant host. Plants homoplastic for plastid genomes containing a nucleic acid sequence of the present invention are obtained, and are preferentially capable of high expression of the nucleic acid sequence.

Transgenic plants or seed comprising an interfering RNA of the invention can also be treated with an insecticide or insecticidal seed coating as described in U.S. Pat. Nos. 5,849,320 and 5,876,739, herein incorporated by reference. Where both the insecticide or insecticidal seed coating and the transgenic plant or seed of the invention are active against the same target insect, for example a Coleopteran pest or a Diabrotica target pest, the combination is useful (i) in a method for further enhancing activity of the composition of the invention against the target insect, and (ii) in a method for preventing development of resistance to the composition of the invention by providing yet another mechanism of action against the target insect. Thus, the invention provides a method of enhancing control of a Diabrotica insect population comprising providing a transgenic plant or seed of the invention and applying to the plant or the seed an insecticide or insecticidal seed coating to a transgenic plant or seed of the invention. Examples of such insecticides and/or insecticidal seed coatings include, without limitation, a carbamate, a pyrethroid, an organophosphate, a friprole, a neonicotinoid, an organochloride, a nereistoxin, or a combination thereof. In another embodiment, the insecticide or insecticidal seed coating are selected from the group consisting of carbofuran, carbaryl, methomyl, bifenthrin, tefluthrin, permethrin, cyfluthrin, lambda-cyhalothrin, cypermethrin, deltamethrin, chlorpyrifos, chlorethoxyfos, dimethoate, ethoprophos, malathion, methyl-parathion, phorate, terbufos, tebupirimiphos, fipronil, acetamiprid, imidacloprid, thiacloprid, thiamethoxam, endosulfan, bensultap, and a combination thereof. Commercial products containing such insecticides and insecticidal seed coatings include, without limitation, Furadan® (carbofuran), Lanate® (methomyl, metomil, mesomile), Sevin® (carbaryl), Talstar® (bifenthrin), Force® (tefluthrin), Ammo® (cypermethrin), Cymbush® (cypermethrin), Delta Gold® (deltamethrin), Karate® (lambda-cyhalothrin), Ambush® (permethrin), Pounce® (permethrin), Brigade® (bifenthrin), Capture® (bifenthrin), ProShield® (tefluthrin), Warrior® (lambda-cyhalothrin), Dursban® (chlorphyrifos), Fortress® (chlorethoxyfos), Mocap® (ethoprop), Thimet® (phorate), AAstar® (phorate, flucythinate), Rampart® (phorate), Counter® (terbufos), Cygon® (dimethoate), Dicapthon, Regent® (fipronil), Cruiser® (thiamethoxam), Gaucho® (imidacloprid), Prescribe® (imidacloprid), Poncho® (clothianidin) and Aztec® (cyfluthrin, tebupirimphos).

The compositions of the invention can also be combined with other biological control agents to enhance control of a coleopteran insect or a Diabrotica insect populations. Thus, the invention provides a method of enhancing control of a Coleopteran insect population or a Diabrotica insect population by providing a transgenic plant that produces an interfering RNA of the invention and further comprises a polynucleotide that encodes a second insecticidal agent. The second insecticidal agent may be an insecticidal protein derived from Bacillus thuringiensis. A B. thuringiensis insecticidal protein can be any of a number of insecticidal proteins including but not limited to a Cry1 protein, a Cry3 protein, a Cry7 protein, a Cry8 protein, a Cryl1 protein, a Cry22 protein, a Cry 23 protein, a Cry 36 protein, a Cry37 protein, a Cry34 protein together with a Cry35 protein, a binary insecticidal protein CryET33 and CryET34, a binary insecticidal protein TIC100 and TIC101, a binary insecticidal protein PS149B1, a VIP, a TIC900 or related protein, a TIC901, TIC1201, TIC407, TIC417,a modified Cry3A protein, or hybrid proteins or chimeras made from any of the preceding insecticidal proteins. In other embodiments, the B. thuringiensis insecticidal protein is selected from the group consisting of Cry3Bb1, Cry34Ab1 together with Cry35Ab1, mCry3A and eCry3.1Ab.

In other embodiments, the transgenic plant may produce an interfering RNA of the invention and a second insecticidal agent which is derived from sources other than B. thuringiensis. The second insecticidal agent can be an agent selected from the group comprising a patatin, a protease, a protease inhibitor, a chitinase, a urease, an alpha-amylase inhibitor, a pore-forming protein, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus laterosporous insecticidal protein, a Lysinibacillus sphearicus insecticidal protein, a Chromobacterium spp. insecticidal protein, a Yersinia entomophaga insecticidal protein, a Paenibacillus popiliae insecticidal protein, a Clostridium spp. (such as C. bifermentans) insecticidal protein, and a lignin. In other embodiments, the second agent may be at least one insecticidal protein derived from an insecticidal toxin complex (Tc) from Photorhabdus, Xenorhabus, Serratia, or Yersinia. In other embodiments. the insecticidal protein may be an ADP-ribosyltransferase derived from an insecticidal bacteria, such as Photorhabdus spp. In other embodiments, the insecticidal protein may be a VIP protein, such as VIP1 or VIP2 from B. cereus. In still other embodiments, the insecticidal protein may be a binary toxin derived from an insecticidal bacteria, such as ISP1A and ISP2A from B. laterosporous or BinA and BinB from L. sphaericus. In still other embodiments, the insecticidal protein may be engineered or may be a hybrid or chimera of any of the preceding insecticidal proteins.

In another embodiment, the transgenic plant and transgenic seed is a corn plant or corn seed. In another embodiment, the transgenic corn plant is provided by crossing a first transgenic corn plant comprising a dsRNA of the invention with a transgenic corn plant comprising a transgenic event selected from the group consisting of MIR604, Event 5307, DAS51922-7, MON863 and MON88017.

Even where the insecticide or insecticidal seed coating is active against a different insect, the insecticide or insecticidal seed coating is useful to expand the range of insect control, for example by adding an insecticide or insecticidal seed coating that has activity against lepidopteran insects to the transgenic plant or seed of the invention, which has activity against coleopteran insects, the treated plant or coated transgenic seed controls both lepidopteran and coleopteran insect pests.

In further embodiments, the invention encompasses a biological sample from a transgenic plant, seed, or parts thereof, of the invention, wherein the sample comprises a nucleic acid that is or encodes at least one strand of a dsRNA of the invention. In other embodiments, the invention encompasses a commodity product derived from a transgenic plant, seed, or parts thereof, of the invention. In some embodiments, the commodity product is selected from the group consisting of whole or processed seeds, beans, grains, kernels, hulls, meals, grits, flours, sugars, sugars, starches, protein concentrates, protein isolates, waxes, oils, extracts, juices, concentrates, liquids, syrups, feed, silage, fiber, paper or other food or product produced from plants. In other embodiments, the biological sample or commodity product is toxic to insects. In other embodiments, the transgenic plant is a transgenic corn plant.

The invention further encompasses a method of controlling a coleopteran insect or a Diabrotica insect comprising contacting the insect with a nucleic acid molecule that is or is capable of producing an interfering RNA molecule of the invention for inhibiting expression of a target gene in the insect thereby controlling the coleopteran insect or the Diabrotica insect. In some embodiments, the target gene comprises a coding sequence(i) having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity, to at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 1-30, SEQ ID NO: 91-120, SEQ ID NO: 271-273, SEQ ID NO: 277-279, SEQ ID NO: 283-300, or a complement thereof; (ii) comprising at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 1-30, SEQ ID NO: 91-120, SEQ ID NO: 271-273, SEQ ID NO: 277-279, SEQ ID NO: 283-300, or a complement thereof; (iii) comprising at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of a nucleotide sequence encoding an amino acid sequence encoded by SEQ ID NO: 1-30, SEQ ID NO: 91-120, SEQ ID NO: 271-273, SEQ ID NO: 277-279, SEQ ID NO: 283-300, or a complement thereof. In some embodiments the target gene coding sequence comprises SEQ ID NO: 1-30, SEQ ID NO: 91-120, SEQ ID NO: 271-273, SEQ ID NO: 277-279, SEQ ID NO: 283-300, or a complement thereof, or (iv) can hybridize under stringent conditions to a polynucleotide selected from the group consisting of SEQ ID NO: 1-30, SEQ ID NO: 91-120, SEQ ID NO: 271-273, SEQ ID NO: 277-279, SEQ ID NO: 283-300, and the complements thereof. In other embodiments, the interfering RNA molecule of the invention is complementary to a portion of a mRNA polynucleotide transcribable from the Diabrotica target genes described herein.

In some embodiments of the method of controlling a coleopteran insect pest or a Diabrotica insect pest, the interfering RNA molecule of the invention comprises at least one dsRNA, wherein the dsRNA is a region of double-stranded RNA comprising annealed complementary strands, one strand of which comprises a sequence of at least 19 contiguous nucleotides which (i) has at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity, to at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 121-210, SEQ ID NO: 274-276, SEQ ID NO: 280-282, SEQ ID NO: 301-318, or the complement thereof; or (ii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 121-210, SEQ ID NO: 274-276, SEQ ID NO: 280-282, SEQ ID NO: 301-318, or the complement thereof; (iii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of a nucleotide sequence encoding an amino acid sequence encoded by SEQ ID NO: 121-210, SEQ ID NO: 274-276, SEQ ID NO: 280-282, SEQ ID NO: 301-318, or the complement thereof, or (iv) can hybridize under stringent conditions to a polynucleotide selected from the group consisting of SEQ ID NO: 121-210, SEQ ID NO: 274-276, SEQ ID NO: 280-282, SEQ ID NO: 301-318, and the complements thereof.

In some embodiments of the method of controlling a coleopteran insect pest or a Diabrotica insect pest, the interfering RNA molecule comprises, consists essentially of or consists of from 18, 19, 20 or 21 consecutive nucleotides to at least about 300 consecutive nucleotides of SEQ ID NO: 181-210. In other embodiments, the interfering RNA of the invention comprises, consists essentially of or consists of any 21-mer subsequence of SEQ ID NO: 181-210 consisting of N to N+20 nucleotides, or any complement thereof. For example, an interfering RNA molecule of the invention comprises a dsRNA which comprises, consist essentially of or consists of any 21-mer subsequence of SEQ ID NO: 181, wherein N is nucleotide 1 to nucleotide 776 of SEQ ID NO: 181, or any complement thereof. In other words, the portion of the mRNA that is targeted comprises any of the 776 21 consecutive nucleotide subsequences i.e. 21-mers) of SEQ ID NO: 181, or any of their complementing sequences. It will be recognized that these 776 21 consecutive nucleotide subsequences include all possible 21 consecutive nucleotide subsequences from SEQ ID NO: 121 and from SEQ ID NO: 151, and their complements, as SEQ ID NOs 121, 151, and 181 are all to the same target, namely BPA_{—} 15366. It will similarly be recognized that all 21-mer subsequences of SEQ ID NO: 181-210, and all complement subsequences thereof, include all possible 21 consecutive nucleotide subsequences of SEQ ID NOs: 121-180, and the complement subsequences thereof.

Similarly, an interfering RNA molecule of the invention comprises a dsRNA which comprises, consist essentially of or consists of any 21-mer subsequence of SEQ ID NO: 182, wherein N is nucleotide 1 to nucleotide 771 of SEQ ID NO: 182, or any complement thereof. Another interfering RNA molecule of the invention comprises a dsRNA which comprises, consist essentially of or consists of any 21-mer subsequence of SEQ ID NO: 183, wherein N is nucleotide 1 to nucleotide 2907 of SEQ ID NO: 183, or any complement thereof. Another interfering RNA molecule of the invention comprises a dsRNA which comprises, consist essentially of or consists of any 21-mer subsequence of SEQ ID NO: 184, wherein N is nucleotide 1 to nucleotide 1600 of SEQ ID NO: 184, or any complement thereof. Another interfering RNA molecule of the invention comprises a dsRNA which comprises, consist essentially of or consists of any 21-mer subsequence of SEQ ID NO: 185, wherein N is nucleotide 1 to nucleotide 2410 of SEQ ID NO: 185, or any complement thereof. Another interfering RNA molecule of the invention comprises a dsRNA which comprises, consist essentially of or consists of any 21-mer subsequence of SEQ ID NO: 186, wherein N is nucleotide 1 to nucleotide 2802 of SEQ ID NO: 186, or any complement thereof. Another interfering RNA molecule of the invention comprises a dsRNA which comprises, consist essentially of or consists of any 21-mer subsequence of SEQ ID NO: 187, wherein N is nucleotide 1 to nucleotide 3681 of SEQ ID NO: 187, or any complement thereof. Another interfering RNA molecule of the invention comprises a dsRNA which comprises, consist essentially of or consists of any 21-mer subsequence of SEQ ID NO: 188, wherein N is nucleotide 1 to nucleotide 651 of SEQ ID NO: 188, or any complement thereof. Another interfering RNA molecule of the invention comprises a dsRNA which comprises, consist essentially of or consists of any 21-mer subsequence of SEQ ID NO: 189, wherein N is nucleotide 1 to nucleotide 673 of SEQ ID NO: 189, or any complement thereof. Another interfering RNA molecule of the invention comprises a dsRNA which comprises, consist essentially of or consists of any 21-mer subsequence of SEQ ID NO: 190, wherein N is nucleotide 1 to nucleotide 2664 of SEQ ID NO: 190, or any complement thereof. Another interfering RNA molecule of the invention comprises a dsRNA which comprises, consist essentially of or consists of any 21-mer subsequence of SEQ ID NO: 191, wherein N is nucleotide 1 to nucleotide 438 of SEQ ID NO: 191, or any complement thereof. Another interfering RNA molecule of the invention comprises a dsRNA which comprises, consist essentially of or consists of any 21-mer subsequence of SEQ ID NO: 192, wherein N is nucleotide 1 to nucleotide 2458 of SEQ ID NO: 192, or any complement thereof. Another interfering RNA molecule of the invention comprises a dsRNA which comprises, consist essentially of or consists of any 21-mer subsequence of SEQ ID NO: 193, wherein N is nucleotide 1 to nucleotide 3254 of SEQ ID NO: 193, or any complement thereof. Another interfering RNA molecule of the invention comprises a dsRNA which comprises, consist essentially of or consists of any 21-mer subsequence of SEQ ID NO: 194, wherein N is nucleotide 1 to nucleotide 3632 of SEQ ID NO: 194, or any complement thereof. Another interfering RNA molecule of the invention comprises a dsRNA which comprises, consist essentially of or consists of any 21-mer subsequence of SEQ ID NO: 195, wherein N is nucleotide 1 to nucleotide 7611 of SEQ ID NO: 195, or any complement thereof. Another interfering RNA molecule of the invention comprises a dsRNA which comprises, consist essentially of or consists of any 21-mer subsequence of SEQ ID NO: 196, wherein N is nucleotide 1 to nucleotide 1008 of SEQ ID NO: 196, or any complement thereof. Another interfering RNA molecule of the invention comprises a dsRNA which comprises, consist essentially of or consists of any 21-mer subsequence of SEQ ID NO: 197, wherein N is nucleotide 1 to nucleotide 2992 of SEQ ID NO: 197, or any complement thereof. Another interfering RNA molecule of the invention comprises a dsRNA which comprises, consist essentially of or consists of any 21-mer subsequence of SEQ ID NO: 198, wherein N is nucleotide 1 to nucleotide 1192 of SEQ ID NO: 198, or any complement thereof. Another interfering RNA molecule of the invention comprises a dsRNA which comprises, consist essentially of or consists of any 21-mer subsequence of SEQ ID NO: 199, wherein N is nucleotide 1 to nucleotide 7626 of SEQ ID NO: 199, or any complement thereof. Another interfering RNA molecule of the invention comprises a dsRNA which comprises, consist essentially of or consists of any 21-mer subsequence of SEQ ID NO: 200, wherein N is nucleotide 1 to nucleotide 2580 of SEQ ID NO: 200, or any complement thereof. Another interfering RNA molecule of the invention comprises a dsRNA which comprises, consist essentially of or consists of any 21-mer subsequence of SEQ ID NO: 201, wherein N is nucleotide 1 to nucleotide 4628 of SEQ ID NO: 201, or any complement thereof. Another interfering RNA molecule of the invention comprises a dsRNA which comprises, consist essentially of or consists of any 21-mer subsequence of SEQ ID NO: 202, wherein N is nucleotide 1 to nucleotide 1557 of SEQ ID NO: 202, or any complement thereof. Another interfering RNA molecule of the invention comprises a dsRNA which comprises, consist essentially of or consists of any 21-mer subsequence of SEQ ID NO: 203, wherein N is nucleotide 1 to nucleotide 1019 of SEQ ID NO: 203, or any complement thereof. Another interfering RNA molecule of the invention comprises a dsRNA which comprises, consist essentially of or consists of any 21-mer subsequence of SEQ ID NO: 204, wherein N is nucleotide 1 to nucleotide 677 of SEQ ID NO: 204, or any complement thereof. Another interfering RNA molecule of the invention comprises a dsRNA which comprises, consist essentially of or consists of any 21-mer subsequence of SEQ ID NO: 205, wherein N is nucleotide 1 to nucleotide 764 of SEQ ID NO: 205, or any complement thereof. Another interfering RNA molecule of the invention comprises a dsRNA which comprises, consist essentially of or consists of any 21-mer subsequence of SEQ ID NO: 206, wherein N is nucleotide 1 to nucleotide 1830 of SEQ ID NO: 206, or any complement thereof. Another interfering RNA molecule of the invention comprises a dsRNA which comprises, consist essentially of or consists of any 21-mer subsequence of SEQ ID NO: 207, wherein N is nucleotide 1 to nucleotide 3225 of SEQ ID NO: 207, or any complement thereof. Another interfering RNA molecule of the invention comprises a dsRNA which comprises, consist essentially of or consists of any 21-mer subsequence of SEQ ID NO: 208, wherein N is nucleotide 1 to nucleotide 1003 of SEQ ID NO: 208, or any complement thereof. Another interfering RNA molecule of the invention comprises a dsRNA which comprises, consist essentially of or consists of any 21-mer subsequence of SEQ ID NO: 209, wherein N is nucleotide 1 to nucleotide 1419 of SEQ ID NO: 209, or any complement thereof. Another interfering RNA molecule of the invention comprises a dsRNA which comprises, consist essentially of or consists of any 21-mer subsequence of SEQ ID NO: 210, wherein N is nucleotide 1 to nucleotide 5206 of SEQ ID NO: 210, or any complement thereof.

In some embodiments of the method of controlling a Diabrotica insect pest, the Diabrotica insect is selected from the group consisting of D. barberi (northern corn rootworm), D. virgifera virgifera (western corn rootworm), D. undecimpunctata howardi (southern corn rootworm), D. balteata (banded cucumber beetle), D. undecimpunctata undecimpunctata (western spotted cucumber beetle), D. significata (3-spotted leaf beetle), D. speciosa (chrysanthemum beetle) and D. virgifera zeae (Mexican corn rootworm).

In other embodiments of the method of controlling a coleopteran insect pest or a Diabrotica insect pest, the contacting comprises (a) planting a transgenic seed capable of producing a transgenic plant that expresses the nucleic acid molecule, wherein the insect feeds on the transgenic plant, or part thereof; or (b) applying a composition comprising the nucleic acid molecule to a seed or plant, or part thereof, wherein the insect feeds on the seed, the plant, or a part thereof. In some embodiments, the transgenic seed and the transgenic plant is a corn seed or a corn plant. In other embodiments the seed or plant is a corn seed or a corn plant.

The invention also encompasses a method of controlling a Diabrotica insect comprising contacting the Diabrotica insect with a nucleic acid molecule that is or is capable of producing the interfering RNA molecule of the invention for inhibiting expression of a target gene in the Diabrotica insect, and also contacting the Diabrotica insect with at least a second insecticidal agent for controlling Diabrotica, wherein said second insecticidal agent comprises a B. thuringiensis insecticidal protein, thereby controlling the Diabrotica insect. The invention also encompasses a method for controlling Diabrotica insect pests on a plant, comprising topically applying to said plant a pesticide composition comprising an interfering RNA of the invention and at least a second insecticidal agent for controlling Diabrotica, wherein said second insecticidal agent does not comprise a B. thuringiensis insecticidal protein, and providing said plant in the diet of said Diabrotica insect. The invention also encompasses a method wherein the second insecticidal agent comprises a patatin, a protease, a protease inhibitor, a urease, an alpha-amylase inhibitor, a pore-forming protein, a lectin, an engineered antibody or antibody fragment, or a chitinase. The second insecticidal agent may also be a Bacillus cereus insecticidal protein, a Xenorhabdus spp. insecticidal protein, a Photorhabdus spp. insecticidal protein, a Brevibacillus laterosporous insecticidal protein, a Lysinibacillus sphearicus insecticidal protein, a Chromobacterium ssp. insecticidal protein, a Yersinia entomophaga insecticidal protein, a Paenibacillus popiliae insecticidal protein, or a Clostridium spp. insecticidal protein.

The invention also encompasses a method of reducing an adult coleopteran insect population or an adult Diabrotica insect population on a transgenic plant expressing a Cry protein, a hybrid Cry protein or modified Cry protein comprising expressing in the transgenic plant a nucleic acid molecule that is or is capable of producing an interfering RNA molecule of the invention capable of inhibiting expression of a target gene as described herein in an adult insect, thereby reducing the adult coleopteran insect population or adult Diabrotica insect population.

In some embodiments, the invention encompasses a method of reducing the level of a target mRNA transcribable from a target gene as described herein in a coleopteran insect or a Diabrotica insect comprising contacting the insect with a composition comprising the interfering RNA molecule of the invention, wherein the interfering RNA molecule reduces the level of the target mRNA in a cell of the insect. In some embodiments, the interfering RNA of the method comprises at least one dsRNA, wherein the dsRNA is a region of double-stranded RNA comprising annealed complementary strands, one strand of which comprises a sequence of at least 19 contiguous nucleotides which (i) has at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity, to at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 121-210, SEQ ID NO: 274-276, SEQ ID NO: 280-282, SEQ ID NO: 301-318, or the complement thereof; (ii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 121-210, SEQ ID NO: 274-276, SEQ ID NO: 280-282, SEQ ID NO: 301-318, or the complement thereof; (iii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of a nucleotide sequence encoding an amino acid sequence encoded by SEQ ID NO: 121-210, SEQ ID NO: 274-276, SEQ ID NO: 280-282, SEQ ID NO: 301-318, or the complement thereof, or (iv) can hybridize under stringent conditions to a polynucleotide selected from the group consisting of SEQ ID NO: 121-210, SEQ ID NO: 274-276, SEQ ID NO: 280-282, SEQ ID NO: 301-318, and the complements thereof, wherein the interfering RNA molecule has insecticidal activity against the target coleopteran insect or a Diabrotica insect. In another embodiment, the contacting is achieved by the target insect feeding on the composition. In other embodiments, production of the protein encoded by the target mRNA is reduced. In other embodiments, the target protein comprises an amino acid having at least about 80%, at least about 85%, at least about 90%, at least about 91%, at least about 92%, at least about 93%, at least about 94%, at least about 95%, at least about 96%, at least about 97%, at least about 98% or at least about 99% identity to SEQ ID NO: 241-270. In other embodiments the target protein comprises SEQ ID NO:241-270. In other embodiments, the interfering RNA is contacted with a coleopteran insect or a Diabrotica insect through a transgenic organism expressing the interfering RNA. In other embodiments, the transgenic organism is a transgenic plant, a transgenic microorganism, a transgenic bacterium or a transgenic endophyte. In other embodiments, the interfering RNA is contacted with a coleopteran insect or a Diabrotica insect by topically applying an interfering RNA in an acceptable agricultural carrier to a plant or plant part on which the insect feeds. In some embodiments, the interfering RNA that reduces the level of a target mRNA transcribable from a target gene described herein is lethal to the coleopteran insect or Diabrotica insect. In some embodiments, the Diabrotica insect is selected from the group consisting of D. barberi (northern corn rootworm), D. virgifera virgifera (western corn rootworm), D. undecimpunctata howardi (southern corn rootworm), D. balteata (banded cucumber beetle), D. undecimpunctata undecimpunctata (western spotted cucumber beetle), D. significata (3-spotted leaf beetle), D. speciosa (chrysanthemum beetle) and D. virgifera zeae (Mexican corn rootworm).

In some embodiments, the invention encompasses a method of conferring coleopteran insect tolerance or Diabrotica insect tolerance to a plant, or part thereof, comprising introducing into the plant, or part thereof, an interfering RNA molecule, a dsRNA molecule, a nucleic acid construct, a chimeric nucleic acid molecule, an artificial plant microRNA precursor molecule and/or a composition of the invention, wherein the dsRNA molecule, nucleic acid construct, chimeric nucleic acid molecule, artificial plant microRNA precursor molecule and/or composition of the invention are toxic to the insect, thereby conferring tolerance of the plant or part thereof to the coleopteran insect or Diabrotica insect. In other embodiments, the introducing step is performed by transforming a plant cell and producing the transgenic plant from the transformed plant cell. In still other embodiments, the introducing step is performed by breeding two plants together.

In other embodiments, the invention encompasses a method of reducing root damage to a plant fed upon by a Diabrotica insect, comprising introducing into cells of the plant an interfering RNA molecule, a dsRNA, a nucleic acid molecule, a nucleic acid construct, a chimeric nucleic acid molecule, an artificial plant microRNA precursor molecule and/or a composition of the invention, wherein the dsRNA, nucleic acid molecule, nucleic acid construct, chimeric nucleic acid molecule, artificial plant microRNA precursor molecule and/or composition of the invention are toxic to the Diabrotica insect, thereby reducing root damage to the plant. In other embodiments, the introducing step is performed by transforming a plant cell and producing the transgenic plant from the transformed plant cell. In still other embodiments, the introducing step is performed by breeding two plants together.

In still other embodiments, the invention encompasses a method of producing a transgenic plant cell having toxicity to a coleopteran insect or Diabrotica insect, comprising introducing into a plant cell an interfering RNA molecule, a dsRNA, a nucleic acid molecule, a nucleic acid construct, a chimeric nucleic acid molecule, an artificial plant microRNA precursor molecule and/or a composition of the invention, thereby producing the transgenic plant cell having toxicity to the insect compared to a control plant cell. In some embodiments, the invention encompasses a plurality of transgenic plant cells produced by this method. In other embodiments, the plurality of transgenic plant cells is grown under conditions which include natural sunlight. In other embodiments, the introducing step is performed by transforming a plant cell and producing the transgenic plant from the transformed plant cell. In still other embodiments, the introducing step is performed by breeding two plants together.

In some embodiments, the invention encompasses a method of producing a transgenic plant having enhanced tolerance to coleopteran or Diabrotica insect feeding damage, comprising introducing into a plant an interfering RNA molecule, a dsRNA, a nucleic acid molecule, a nucleic acid construct, a chimeric nucleic acid molecule, an artificial plant microRNA precursor molecule and/or a composition of the invention, thereby producing a transgenic plant having enhanced tolerance to coleopteran or Diabrotica insect feeding damage compared to a control plant. In other embodiments, the introducing step is performed by transforming a plant cell and producing the transgenic plant from the transformed plant cell. In still other embodiments, the introducing step is performed by breeding two plants together.

In some embodiments, the invention encompasses a method of providing a corn grower with a means of controlling a coleopteran insect pest population or a Diabrotica insect pest population in a corn crop comprising (a) selling or providing to the grower transgenic corn seed that comprises an interfering RNA, a nucleic acid molecule, a nucleic acid construct, a chimeric nucleic acid molecule, an artificial plant microRNA precursor molecule and/or a composition of the invention; and (b) advertising to the grower that the transgenic corn seed produce transgenic corn plants that control a coleopteran or Diabrotica pest population.

In some embodiments, the invention encompasses a method of identifying a target gene for using as a RNAi strategy for the control of a plant pest for RNAi in a coleopteran plant pest, said method comprising the steps of a) producing a primer pair with sequences selected from the group comprising or consisting of SEQ ID NO: 31-90, or a complement thereof; b) amplifying an orthologous target from a nucleic acid sample of the plant pest; c) identifying a sequence of an orthologous target gene; d) producing an interfering RNA molecule, wherein the RNA comprises at least one dsRNA, wherein the dsRNA is a region of double-stranded RNA comprising annealed complementary strands, one strand of which comprises a sequence of at least 19 contiguous nucleotides which is at least partially complementary to a target nucleotide sequence within a coleopteran target gene, is obtained; and e) determining if the interfering RNA molecule has insecticidal activity on the plant peast. If the interfering RNA has insecticidal activity on the coleopteran pest, a target gene for using in the control of the plant pest has been identified. In some embodiments, the plant pest is a coleopteran plant pest.

### EXAMPLES

The invention will be further described by reference to the following detailed examples. These examples are provided for the purposes of illustration only, and are not intended to be limiting unless otherwise specified.

### Example 1

### Identification of RNAi Gene Targets in Diabrotica virgifera virgifera

This example describes the cloning and sequencing of RNAi target genes and coding sequences from Diabrotica insects.

Diabrotica virgifera virgifera pyrosequencing library preparation and sequencing

A whole-body neonate Diabrotica virgifera virgifera (Western Corn Rootworm (WCR)) transcriptome was sequenced by pyrosequencing on a 454 platform (454 Life Sciences, Branford, Conn.) essentially according to the manufacturer's instructions. The resulting reads (i.e., short fragments of nucleic acid sequence) were trimmed and assembled into contigs using a MIRA assembler (See, for example, Chevreux et al. 2004. Genome Res. 14:1147-1159, incorporated herein by reference).

Identification of Lead Target Genes from Diabrotica spp.

Assembled contigs were compared via BLAST to known lethal genes and alleles in other organisms, which were identified based on published disclosures including those in the website wormbase (wormbase.org) and Boutros et al (2004, Science 303: 832-835). From this analysis, 4,608 target genes were identified. Each of these target genes is non-redundant and is known to possess an allele(s) which is lethal, or is known to result in lethality when targeted by RNAi, in either C. elegans, Drosophila, or both. Therefore, each of these targets were considered essential. It was expected that a significantly large percentage of these target genes would have an insecticidal effect in WCR. Surprisingly, that was not the case.

dsRNAs of the 4,608 targets were produced on an 384 well automated library synthesis platform. All the dsRNA samples tested were designed automatically using Primer3, a primer design tool, to synthetize a dsRNA fragment of around 500-600 bp based on the coding sequence of each target gene. Smaller fragments were designed if the size of the coding sequence did not allow a 500 bp fragment. These samples were screened in a 24-well WCR assay, at one concentration (100 ng dsRNA/cm², i.e. 190 ng dsRNA/well) with 10 L2 WCR larvae per well. The mortality was scored after 10 days. The cut-off for candidate hits was 69% mortality. Of the 4,608 candidate dsRNA targets, 183 target genes were identified. These results are surprising, as a person skilled in the art would have expected that a greater number of the 4,608 candidate targets would have conferred toxicity in the bioassays.

In a first assay, 7 targets were tested in laboratory bioassays in a 10-fold dilution series starting from 1 μg dsRNA/well. Bioassays were performed using an RNA-treated artificial diet method. Briefly, molten artificial diet, modified from the diet of Marrone et al. 1985 (J. Econ. Entomol. 78:290-293), was poured into each well of 48-well plates and allowed to solidify. dsRNA molecules were diluted to appropriate concentration so that 20 μl of solution was added to the surface of the diet in half of the wells of a 48-well plate, with a final overlay concentration of 1 μg, 0.1 μg, 0.01 μg and 0.001 μg per well. One or two WCR larvae were added to each well to have between 24 and 48 replicate larvae per concentration of dsRNA tested. Each 48-well plate was maintained at approximately 26° C. with a 16 hour:8 hour light:dark photoperiod. Mortality was recorded at 1, 2, 3, 4, 6 and 7 d post-infestation. dsRNA designed to target green fluorescent protein (GFP) was used in all bioassays as a negative control and dsRNA designed to target an ubiquitin gene of WCR was used as a positive control. From this assay, BPA_46378 (alpha-snap) was confirmed positive. Four candidates were not confirmed positive.

The other 176 target genes were tested simultaneously in a confirmation screen. dsRNA of the 176 targets, as well as positive and negative control dsRNAs, were produced on an automated library synthesis platform. BPA_46378 was also tested under this screen. Bioassays were performed using an RNA-treated artificial diet method. Briefly, molten artificial diet, modified from the diet of Marrone et al. 1985 (J. Econ. Entomol. 78:290-293), was poured into each well of 48-well plates and allowed to solidify. dsRNA molecules were diluted to appropriate concentration so that 20 μl of solution was added to the surface of the diet in half of the wells of a 48-well plate, with a final overlay concentration of 0.5 μg dsRNA per well. One or two WCR larvae were added to each well to have between 24 and 48 replicate larvae per dsRNA tested. Each 48-well plate was maintained at approximately 26° C. and 16:8 light:dark photoperiod. Mortality was recorded at 7 days after treatment.

The results, shown in Table 1, found 29 dsRNA molecules of the initial 176 dsRNA molecules identified were confirmed to be highly toxic to Diabrotica virgifera virgifera (western corn rootworm), in addition to the alpha-snap target which was re-confirmed. SEQ ID NOs: 1-30 are nucleotide sequences of the nucleic acid fragments of each toxic target gene identified in the screen. SEQ ID NOs: 31-90, or a complement thereof, are nucleotide sequences of the primer pairs used to synthesize the nucleic acid fragments of each target gene identified in the screen. SEQ ID NOs: 91-120 are nucleotide sequences of the full-length coding sequences of each target gene identified by this screen.

It has previously been suggested that certain genes of Diabrotica spp. may be exploited for RNAi-mediated insect control. See U.S. Patent Publication No. 2007/0124836, which discloses 906 sequences, and U.S. Pat. No. 7,612,194, which discloses 9,112 sequences. However, as demonstrated here, the ability of any given gene target to confer toxicity through an RNAi approach cannot be predicted, and can only be determined empirically. Similar conclusions have been reached by Narva et al. (U.S. Publication No. 2015/0322456). The present invention identifies 30 target genes which each provide surprising and unexpected superior control of Diabrotica.

TABLE 1
Activity of dsRNA against Diabrotica virgifera virgifera
7 d after treatment

| | Putative | | SEQ | % |
|---|---|---|---|---|
| | Dm | putative gene name or | ID | mortality at d7 |
| Target ID | orthologue | function | NO: | (0.5 μg/well) |
| BPA_15366 | CG7178 | troponin | 1 | 96.00 |
| BPA_16909 | CG12051 | actin 42A | 2 | 100.00 |
| BPA_45189 | CG6699 | beta′-coatomer | 3 | 100.00 |
| BPA_71902 | CG32744 | ubiquitin-5E | 4 | 90.91 |
| BPA_16014 | CG18290 | Actin 87E | 5 | 84.00 |
| BPA_41555 | CG1528 | gamma-coatomer | 6 | 97.14 |
| BPA_71568 | CG3664 | Rab5 | 7 | 100.00 |
| BPA_16830 | NA | unknown function | 8 | 92.00 |
| BPA_15330 | CG5271 | RpS27A | 9 | 85.29 |
| BPA_2526 | CG11415 | tetraspanin | 10 | 97.22 |
| BPA_11606 | CG33865 | histone2A | 11 | 93.94 |
| BPA_12879 | CG1664 | small bristles | 12 | 97.30 |
| BPA_2443 | CG6223 | beta-coatomer | 13 | 93.94 |
| BPA_10976 | CG8385 | ARF1 | 14 | 78.95 |
| BPA_875 | CG3722 | DE-cadherin | 15 | 76.47 |
| BPA_2184 | CG40127 | RNase K | 16 | 75.76 |
| BPA_7931 | CG11027 | ARF102F | 17 | 82.35 |
| BPA_17622 | CG7007 | Vacuolar H[+] ATPase | 18 | 70.00 |
| | | PPA1 | | |
| BPA_450 | CG1554 | RNApol II | 19 | 78.05 |
| BPA_46378 | CG6625 | Alpha snap | 20 | 100.00 |
| BPA_71489 | CG3320 | Rab1 | 21 | 85.00 |
| BPA_4800 | CG7269 | helicase | 22 | 69.57 |
| BPA_880 | CG4775 | Tango14 | 23 | 63.64 |
| BPA_15751 | CG12775 | RpL21 | 24 | 65.79 |
| BPA_41770 | CG3948 | zeta-coatomer | 25 | 71.05 |
| BPA_9438 | CG8472 | calmodulin | 26 | 65.63 |
| BPA_16140 | CG7185 | RNA recognition motif | 27 | 54.80 |
| | | domain | | |
| BPA_65371 | CG1519 | proteasome alpha | 28 | 52.50 |
| BPA_12351 | CG8186 | Vacuolar H[+] ATPase | 29 | 42.60 |
| | | Vha36-1 | | |
| BPA_17046 | CG9311 | myopic | 30 | 47.40 |
| | | GFP repl1 | | 21.21 |
| | | GFP repl2 | | 11.76 |
| | | Dv ubiquitin control | | 100.00 |
| | | repl1 | | |
| | | Dv ubiquitin control | | 100.00 |
| | | repl2 | | |

### Example 2

### Activity of dsRNA against Diabrotica virgifera virgifera—DRC 4 Concentrations

This example describes testing dsRNAs of the invention for biological activity against Diabrotica virgifera virgifera (WCR).

The 30 dsRNA molecules described above were tested for toxicity against WCR in laboratory bioassays in a 10-fold dilution series starting from 1 μg dsRNA/well. Bioassays were performed using an RNA-treated artificial diet method. Briefly, molten artificial diet, modified from the diet of Marrone et al. 1985 (J. Econ. Entomol. 78:290-293), was poured into each well of 48-well plates and allowed to solidify. Synthesized dsRNA molecules were diluted to appropriate concentration so that 20 μl of solution was added to the surface of the diet in half of the wells of a 48-well plate, with a final overlay concentration of 1 μg, 0.1 μg, 0.01 μg and 0.001 μg per well. One or two WCR larvae were added to each well to have between 24 and 48 replicate larvae per concentration of dsRNA tested. Each 48-well plate was maintained at approximately 26° C. and 16:8 light:dark photoperiod. Mortality was recorded at 1, 2, 3, 4, 6 and 7 d post-infestation. dsRNA designed to target GFP was used as a negative control and dsRNA designed to target an ubiquitin gene of WCR was used as a positive control.

The results, shown in Table 2, show that the 30 dsRNA molecules designed to target mRNA transcribable from WCR genes are toxic to highly toxic to WCR. After correction for the control mortality on the GFP dsRNA, the estimated LT₅₀ and LC₅₀ were calculated by curve fitting analysis. LT₅₀ stands for the lethal time to obtain 50% of mortality in the test insects. LC₅₀ stands for the concentration of the dsRNA, which causes the death of 50% of the test insects. In Table 2, the % mortality at day 7 is based on 1 μg dsRNA/well. The LT₅₀ is based on using 1 μg dsRNA/day and is measured in days. The LC₅₀ was measured in μg dsRNA/well.

TABLE 2
Activity of dsRNA against Diabrotica virgifera virgifera,
7 d after treatment

| | | % mortality at | | LC₅₀ |
|---|---|---|---|---|
| Target ID | SEQ ID NO: | d7 (1 μg/well) | LT₅₀ (days) | (μg/well) |
| BPA_15366 | 1 | 97.30 | 2.53 | 0.005 |
| BPA_16909 | 2 | 100.00 | 3.52 | 0.009 |
| BPA_45189 | 3 | 100.00 | 5.14 | 0.005 |
| BPA_71902 | 4 | 88.89 | 3.74 | 0.045 |
| BPA_16014 | 5 | 85.71 | 5.21 | 0.077 |
| BPA_41555 | 6 | 100.00 | 5.14 | <0.001 |
| BPA_71568 | 7 | 97.22 | 5.03 | 0.015 |
| BPA_16830 | 8 | 84.21 | 5.73 | 0.008 |
| BPA_15330 | 9 | 58.33 | 6.98 | 0.858 |
| BPA_2526 | 10 | 97.37 | 5.22 | 0.028 |
| BPA_11606 | 11 | 97.22 | 5.03 | 0.061 |
| BPA_12879 | 12 | 93.55 | 4.85 | 0.009 |
| BPA_2443 | 13 | 100.00 | 4.71 | 0.004 |
| BPA_10976 | 14 | 85.00 | 5.49 | 0.008 |
| BPA_875 | 15 | 94.29 | 4.84 | 0.084 |
| BPA_2184 | 16 | 64.44 | 6.71 | 0.763 |
| BPA_7931 | 17 | 90.32 | 5.38 | 0.081 |
| BPA_17622 | 18 | 70.73 | 6.60 | 0.649 |
| BPA_450 | 19 | 65.71 | 6.57 | 0.256 |
| BPA_46378 | 20 | 100.00 | 5.49 | 0.015 |
| BPA_71489 | 21 | 92.68 | 5.29 | 0.009 |
| BPA_4800 | 22 | 51.35 | NA | NA |
| BPA_880 | 23 | 53.13 | NA | NA |
| BPA_15751 | 24 | 85.29 | 5.74 | 0.058 |
| BPA_41770 | 25 | 62.16 | 6.71 | 0.090 |
| BPA_9438 | 26 | 51.35 | NA | NA |
| BPA_16140 | 27 | 76.67 | 6.56 | 0.201 |
| BPA_65371 | 28 | 34.29 | NA | NA |
| BPA_12351 | 29 | 35.29 | NA | NA |
| BPA_17046 | 30 | 51.43 | NA | NA |
| GFP repl1 | | 15.80 | NA | NA |
| GFP repl2 | | 20.00 | NA | NA |
| Dv ubiquitin | | 97.22 | 3.33 | 0.028 |

Based on these results, a sub-set of targets were prioritized for further investigation. The results of these targets are shown below.

### Example 3

### Activity of dsRNA against Diabrotica virgifera virgifera

This example describes testing of a sub-set of the identified target dsRNAs of the invention for biological activity against Diabrotica virgifera virgifera (WCR).

The dsRNA molecules described above were tested for toxicity against WCR in laboratory bioassays in a 3-fold dilution series starting at 0.5 μg dsRNA/well. Bioassays were performed using an RNA-treated artificial diet method. Briefly, molten artificial diet, modified from the diet of Marrone et al. 1985 (J. Econ. Entomol. 78:290-293), was poured into each well of 48-well plates and allowed to solidify. dsRNA molecules were diluted to appropriate concentration so that 20 μl of solution was added to the surface of the diet in half of the wells of a 48-well plate, with a final overlay concentration of 0.5 μg, 0.16 μg, 0.05 μg, 0.02 μg, 0.006 μg, 0.002 μg, 0.0007 μg and 0.0002 μg per well. One or two WCR larvae were added to each well to have between 24 and 48 replicate larvae per concentration of dsRNA tested. Each 48-well plate was maintained at approximately 26° C. and 16:8 light:dark photoperiod. Mortality was recorded at 1, 2, 3, 4, 6 and 7 d post-infestation. dsRNA designed to target GFP was used as a negative control and dsRNA designed to target an ubiquitin gene of WCR was used as a positive control.

The results, shown in Table 3, show that the dsRNA molecules designed to target mRNA transcribable from WCR genes are toxic to highly toxic to WCR. After correction for the control mortality on the GFP dsRNA, the estimated LT₅₀ and LC₅₀ were calculated by curve fitting analysis. LT₅₀ stands for the lethal time to obtain 50% of mortality in the test insects. LC₅₀ stands for the concentration of the dsRNA, which causes the death of 50% of the test insects. In Table 3, the % mortality at day 7 is based on 0.5 μg dsRNA/well. The LT₅₀ is based on using 0.5 μg dsRNA/day and is measured in days. The LC₅₀ was measured in μg dsRNA/well. These results confirm the toxicity of the candidate targets.

TABLE 3
Activity of dsRNA against Diabrotica virgifera virgifera

| | | % mortality at d7 | | LC₅₀ |
|---|---|---|---|---|
| Target ID | SEQ ID NO: | (0.5 μg/well) | LT₅₀ (days) | (μg/well) |
| BPA_41555 | 6 | 100.0 | 4.6 | 0.0005 |
| BPA_12879 | 12 | 91.2 | 5.1 | 0.0054 |
| BPA_71489 | 21 | 84.8 | 5.3 | 0.0013 |
| GFP | | 22.9 | NA | NA |
| Dv ubiquitin | | 100.0 | 3.7 | 0.0065 |

### Example 4

### Activity of dsRNA against Diabrotica undecimpunctata howardi

This example describes testing dsRNAs of the invention for biological activity against Diabrotica undecimpunctata howardi (southern corn rootworm (SCR)).

The dsRNA molecules described above were tested for toxicity against SCR in laboratory bioassays in a 10-fold dilution series starting at 0.5 μg dsRNA/well. Bioassays were performed using an RNA-treated artificial diet method. Briefly, molten artificial diet, modified from the diet of Marrone et al. 1985 (J. Econ. Entomol. 78:290-293), was poured into each well of 48-well plates and allowed to solidify. Synthesized dsRNA molecules were diluted to appropriate concentrations so that 20 μl of solution was added to the surface of the diet in each well, with a final overlay concentration series of 8 concentrations going from 0.5 μg/well down to 0.00022 μg/well in steps of 3× dilution. One or two SCR larvae were added to each well to have between 24 and 48 replicate larvae per concentration of dsRNA tested. Each 48-well plate was maintained at approximately 26° C. and 16:8 light:dark photoperiod. Mortality was recorded at 1, 2, 3, 4, 5, 6, 7, 8, 9, 10, and 14 days post-infestation. dsRNA designed to target s GFP was used as a negative control and dsRNA designed to target the Diabrotica virgifera virgifera (Dv) ubiquitin gene and the Diabrotica undecimpunctata howardi (Du) ubiquitin gene were used as positive controls.

After correction for the control mortality on the GFP dsRNA, the estimated LT₅₀ and LC₅₀ were calculated by curve fitting analysis. LT₅₀ stands for the lethal time to obtain 50% of mortality in the test insects. LC₅₀ stands for the concentration of the dsRNA, which causes the death of 50% of the test insects. In Table 4, the % mortality at day 14 is based on 0.5 μg dsRNA/well. The LT₅₀ is based on using 0.5 μg dsRNA/day and is measured in days. The LC₅₀ was measured in μg dsRNA/well. The results, shown in Table 4, show that the dsRNA molecules designed to target mRNA transcribable from Diabrotica virgifera virgifera (WCR) genes are also toxic to Diabrotica undecimpunctata howardi (SCR). This demonstrates that the targets of the invention are suitable targets for SCR as well, such that dsRNA molecules based on the native SCR mRNAs would be toxic to SCR and other Diabrotica spp. as well.

TABLE 4
Activity of dsRNA against Diabrotica undecimpunctata howardi
14 d after treatment

| | | % mortality | | |
|---|---|---|---|---|
| | | at d14 | LT₅₀ | LC₅₀ |
| Target ID | SEQ ID NO: | (0.5 μg/well) | (days) | (μg/well) |
| BPA_41555 | 6 | 94.12 | 9.68 | 0.0019 |
| BPA_12879 | 12 | 83.78 | 9.91 | 0.0278 |
| BPA_71489 | 21 | 84.62 | 11.25 | 0.0156 |
| GFP | | 8.33 | NA | NA |
| Dv ubiquitin | | 97.14 | 6.75 | 0.0123 |
| Du ubiquitin | | 100.00 | 5.57 | 0.0209 |

### Example 5

### Activity of dsRNA against Diabrotica barberi

This example describes testing dsRNAs of the invention for biological activity against Diabrotica barberi (northern corn rootworm (NCR)).

The dsRNA molecules described above were tested for toxicity against NCR in laboratory bioassays. Bioassays were performed using an RNA-treated artificial diet method. Briefly, molten artificial diet, modified from the diet of Marrone et al. 1985 (J. Econ. Entomol. 78:290-293), was poured into each well of 48-well plates and allowed to solidify. Synthesized dsRNA molecules were diluted to appropriate concentration so that 20 μl of solution was added to the surface of the diet in half of the wells of a 48-well plate, with a final overlay concentration of 0.5 μg dsRNA per well. One or two NCR larvae were added to each well to have between 24 and 48 replicate larvae per dsRNA tested. Each 48-well plate was maintained at approximately 26° C. and 16:8 light:dark photoperiod. Mortality was recorded at 7 d post-infestation. dsRNA designed to target GFP was used in all bioassays as a negative control and dsRNA designed to target the Diabrotica barberi (Dr) ubiquitin gene was used as positive control.

The results, shown in Table 5, show that the dsRNA molecules designed to target mRNA transcribable from Diabrotica virgifera virgifera genes are also toxic to Diabrotica barberi. This demonstrates that the targets of the invention are suitable targets for NCR as well, such that dsRNA molecules based on the native NCR mRNAs would be toxic to NCR and other Diabrotica spp. as well.

TABLE 5
Activity of dsRNA against Diabrotica barberi 7 d after treatment

| | SEQ ID | % mortality |
|---|---|---|
| Target ID | NO: | at day 9 |
| BPA_41555 | 6 | 97.78 |
| BPA_12879 | 12 | 91.00 |
| BPA_71489 | 21 | 95.00 |
| GFP repl1 | | 18.75 |
| GFP repl2 | | 22.00 |
| Dr ubiquitin repl1 | | 85.00 |
| Dr ubiquitin repl2 | | 86.00 |

### Example 6

### Fragment Size Assays in WCR

All dsRNA samples tested in the previous examples were designed automatically using Primer3, a primer design tool, to synthesize a dsRNA fragment of around 500 bp based on the coding sequence of each target gene. Smaller fragments were designed if the size of the coding sequence did not allow a 500 bp fragment.

In the big-to-small experiments, different dsRNA fragments were designed based on the complete coding sequence of each target gene. The complete coding sequence was tested as a whole if available and if not greater than 1000 bp. The coding sequence was also divided into fragments of approximately 200 bp, with some overlap of 25-30 bp between subsequent fragments. For each fragment new primers were designed and dsRNA was synthesized on the automated library synthesis platform. All dsRNA fragments were then tested in a WCR bioassay at two different concentrations (0.1 μg dsRNA and 0.01 μg dsRNA per well) and mortality was scored at day 7.

The dsRNA molecules described above were tested for toxicity against Diabrotica virgifera virgifera in laboratory bioassays. Bioassays were performed using an RNA-treated artificial diet method. Briefly, molten artificial diet, modified from the diet of Marrone et al. 1985 (J. Econ. Entomol. 78:290-293), was poured into each well of 48-well plates and allowed to solidify. Synthesized dsRNA molecules were diluted to appropriate concentration so that 20 μl of solution was added to the surface of the diet in half of the wells of a 48-well plate, with a final overlay concentration of 0.1 μg dsRNA or 0.01 μg dsRNA per well. One or two Diabrotica virgifera virgifera larvae were added to each well to have between 24 and 48 replicate larvae per dsRNA tested. Each 48-well plate was maintained at approximately 26° C. and 16:8 light:dark photoperiod. Mortality was recorded at 7 d post-infestation. dsRNA designed to target GFP was used in all bioassays as a negative control and dsRNA designed to target an ubiquitin gene of Diabrotica virgifera virgifera was used as a positive control.

The results, shown in Table 6, show that the dsRNA fragments designed to target mRNA transcribable from Diabrotica virgifera virgifera genes are toxic to highly toxic to Diabrotica virgifera virgifera.

TABLE 6
Activity of dsRNA sub-fragments against Diabrotica virgifera virgifera
7 d after treatment

| | SEQ ID | Fragment | % mortality at d7 | |
|---|---|---|---|---|
| Target ID | NO: | size (bp) | 0.01 μg | 0.1 μg |
| BPA_41555_screen | 409 | 592 | 79 | 94 |
| BPA_41555_2 | 410 | 669 | 86 | 100 |
| BPA_41555_3 | 411 | 794 | 86 | 88 |
| BPA_41555_4 | 412 | 649 | 86 | 95 |
| BPA_41555_5 | 413 | 721 | 89 | 100 |
| BPA_12879_screen | 422 | 558 | 64 | 84 |
| BPA_12879_1 | 423 | 864 | 8 | 87 |
| BPA_12879_2 | 424 | 197 | 85 | 97 |
| BPA_12879_3 | 425 | 198 | 22 | 74 |
| BPA_12879_4 | 426 | 200 | 82 | 97 |
| BPA_12879_5 | 427 | 236 | 80 | 95 |
| BPA_12879_6 | 428 | 248 | 24 | 89 |
| BPA_71489_screen | 396 | 564 | 86 | 95 |
| BPA_71489_1 | 397 | 606 | 75 | 91 |
| BPA_71489_2 | 398 | 197 | 54 | 94 |
| BPA_71489_3 | 399 | 197 | 53 | 93 |
| BPA_71489_4 | 400 | 197 | 77 | 79 |
| BPA_71489_5 | 401 | 155 | 59 | 89 |
| GFP | | | NA | 3 |
| GFP | | | NA | 24 |
| positive control | | | 79 | 100 |
| positive control | | | 57 | 75 |

### Example 7

### Expression of an Interfering RNA Molecule Comprising Target dsRNA in Corn Plants

This example describes introducing a construct that expresses an interfering RNA molecule into plant cells.

### Vector Construction

Expression vectors designed to produce hairpin RNAs (hpRNA) consisted of a cassette containing a promoter, a sense strand, an intron functioning as a loop sequence, an antisense strand, and terminator. Binary vector 23159 comprises an expression cassette comprising a DNA sequence designed to produce a hpRNA targeting a 592 nucleotide fragment of BPA_41555 (SEQ ID NO: 319). Binary vector 23566 comprises an expression cassette comprising a DNA sequence designed to produce a hpRNA targeting a 197 nucleotide fragment BPA_12879 (SEQ ID NO: 320). Each binary vector also contained a second cassette between the left and right borders, designed to express phosphomannose isomerase (PMI) which provides the ability to metabolize mannose (U.S. Pat. Nos. 5,767,378 and 5,994,629, which are incorporated by reference herein) as a selectable marker during plant transformation. The vectors also contained selectable markers for selection in bacteria.

### Agrobacterium Preparation

Each resulting plasmid containing the hairpin cassette was transformed into Agrobacterium tumefaciens using standard molecular biology techniques known to those skilled in the art. The vectors described above were transformed into maize. Agrobacterium transformation of immature maize embryos was performed essentially as described in Negrotto et al., 2000, Plant Cell Reports 19: 798-803. For this example, all media constituents are essentially as described in Negrotto et al., supra. However, various media constituents known in the art may be substituted. Following transformation, selection, and regeneration, plants were tested for the presence of the pmi gene and the hairpin dsRNA interfering RNA molecule. Positive plants from the PCR assay were transferred to the greenhouse and tested for resistance to at least Western Corn Rootworm.

### Transgenic Maize WCR Insecticidal Assay

Six F1 progeny of transgenic maize plants comprising the transgene of binary vector 23159 or binary vector 23566 were germinated and allowed to grow. A PMI ELISA strip test (Romer Labs SeedChek® PMI (#7000052)) was used to identify plants positive for the transgene and null, non-transgenic segregating sister plants. Each plant was infested with 10 neonate western corn rootworms at its base. Seven days after infestation, the survival and size of the rootworms were evaluated. Additionally, the corn roots from each of the plants were examined for feeding damage. This experiment was repeated at least eight times each for F1 progeny of transgenic maize plants comprising the transgene of binary vector 23160 or binary vector 23564.

Representative results are shown in Tables 7 and 8. Table 7 shows results for transgenic maize transformed with the transgene of binary vector 23159. Table 8 shows results for transgenic maize transformed with the transgene of binary vector 23566. For each transgene, the results from F1 progeny of two different transgenic events are shown. If an F1 progeny failed to germinate, it is noted in the table as “N/A” for all fields. For the BPA_41555 target, which is targeted by the RNAi construct of vector 23159, F1 progeny from transgenic events ID 1575 and 1848 were examined. For the BPA_12879 target, which is targeted by the RNAi construct of vector 23566, F1 progeny from transgenic events ID 4472 and 4543 were examined. The number of Western Corn Rootworms (WCR) recovered seven days after infestation is indicated (#WCR). Recovered rootworm were graded by size (WCR size), as medium (m), medium/big (mb), big (b), or very big (vb). Roots of the corn plants were also analyzed for feeding damage. “Minor” root damage indicates roots appear strong and healthy. “Noticeable” root damage indicates the roots were slightly weaker compared to controls. “Significant” root damage indicates that the smaller roots were damaged or missing. “Severe” root damage indicates only the largest roots remained attached to the plant.

TABLE 7
WCR tolerance in hpRNA BPA_41555 transgenic maize plants

| Plant | | | | Root |
|---|---|---|---|---|
| ID | PMI? | # WCR | WCR size | damage |
| 1575-1 | Yes | 8 | 5vb, 1b, 2m | noticeable |
| 1575-2 | No | 9 | 6vb, 3b | significant |
| 1575-3 | No | 8 | 8vb | significant |
| 1575-4 | Yes | 5 | 4vb, 1b | minor |
| 1575-5 | Yes | 6 | 2vb, 4b | minor |
| 1575-6 | Yes | 5 | 4vb, 1b | minor |
| 1848-1 | No | 8 | 8vb | significant |
| 1848-2 | No | 6 | 1m, 5b | severe |
| 1848-3 | Yes | 5 | 1s, 2m, 2b | minor |
| 1848-4 | No | 10 | 3b, 7vb | noticeable |
| 1848-5 | No | 8 | 6vb, 2b | noticeable |
| 1848-6 | N/A | N/A | N/A | N/A |

TABLE 8
WCR tolerance in hpRNA BPA_12879 transgenic maize plants

| Plant | | | | Root |
|---|---|---|---|---|
| ID | PMI? | # WCR | WCR size | damage |
| 4472-1 | Yes | 1 | 1m | none |
| 4472-2 | Yes | 2 | 2vb | minor |
| 4472-3 | Yes | 2 | 1m, 1b | none |
| 4472-4 | Yes | 2 | 2b | none |
| 4472-5 | No | 2 | 2vb | noticeable |
| 4472-6 | No | 9 | 1b, 8vb | significant |
| 4543-1 | Yes | 8 | 5b, 3vb | noticeable |
| 4543-2 | No | 7 | 7vb | significant |
| 4543-3 | Yes | 8 | 5b, 3vb | noticeable |
| 4543-4 | No | 7 | 1b, 6vb | significant |
| 4543-5 | No | 8 | 8vb | noticeable |
| 4543-6 | Yes | 10 | 5b, 5vb | minor |

The data in Tables 7 and 8 indicate that the transgenic corn plants expressing dsRNAs that target insect genes BPA_41555 or BPA_12879 may suffer less root damage compared to the non-transgenic, negative control sister plants. Tables 7 and 8 show that a transgenic plant comprising an interfering RNA molecule of the invention has enhanced resistance to an insect pest as compared to a non-transgenic control plant.

### Transgenic Maize CRW Root Assay

Transgenic maize expressing the transgene from binary vector 23159 were grown and brace roots or crown roots from the plant were removed. Root pieces were placed on a 2% agar plate and infested with 80 to 100 L1 WCR larvae. Following an incubation in the dark 26° C. for 24 to 48 hours, the L1 larvae were transferred to a 48-well WCR diet plate and incubated in the dark at 26° C. and scored daily for mortality of the WCR larvae, up to 7 days post-infestation. This experiment was performed on three different transgenic maize events, and on a non-transgenic control maize plant. Cumulative results are shown in Table 9. % Mortality indicates the total percent of WCR larvae which died.

TABLE 9
CRW Root Assay for hpRNA BPA_41555 Transgenic Maize

| | % |
|---|---|
| Plant ID | Mortality |
| Non-transgenic | 14 |
| Event 1 | 83 |
| Event 2 | 88 |
| Event 3 | 100 |

The data in Table 9 indicate that the transgenic corn plants expressing dsRNAs that target the insect gene BPA_41555 have an insecticidal effect on insect pests. This further shows that a transgenic plant comprising an interfering RNA molecule of the invention has enhanced resistance to an insect pest as compared to a non-transgenic control plant.

### Example 8

### Interfering RNA Molecules with a Second Insecticidal Agent Bioassays

Double stranded RNA molecules were produced against the BPA_41555 target. Additionally, a second insecticidal agent was prepared. Both the RNA and the second insecticidal agent were tested in combination for toxicity against WCR in laboratory bioassays.

It should be understood that the examples and embodiments described herein are for illustrative purposes only and that various modifications or changes in light thereof of the description will be suggested to persons skilled in the art and are to be included within the spirit and purview of this application and the scope of the appended claims.

All publications and patent applications mentioned in this specification are indicative of the level of skill of those skilled in the art that this invention pertains. All publications and patent applications are herein incorporated by reference to the same extent as if each individual publication or patent application was specifically and individually indicated to be incorporated by reference.

1. A double-stranded RNA (dsRNA) comprising annealed complementary strands, one strand of which comprises a sequence of at least 19 contiguous nucleotides which is at least partially complementary to a target nucleotide sequence within a Diabrotica spp target gene, and (i) is at least 85% identical to at least a 19 contiguous nucleotide fragment of SEQ ID NO: 126, 132, 141, 156, 162, 171, 186, 192, or 201, SEQ ID NO: 274-276, SEQ ID NO: 280-282, SEQ ID NO: 301-318, or the complement thereof; (ii) comprises at least a 19 contiguous nucleotide fragment of SEQ ID NO: 126, 132, 141, 156, 162, 171, 186, 192, or 201, SEQ ID NO: 274-276, SEQ ID NO: 280-282, SEQ ID NO: 301-318, or the complement thereof; (iii) comprises at least a 19 contiguous nucleotide fragment of a nucleotide sequence encoding an amino acid sequence encoded by SEQ ID NO: 126, 132, 141, 156, 162, 171, 186, 192, or 201, SEQ ID NO: 274-276, SEQ ID NO: 280-282, SEQ ID NO: 301-318, or the complement thereof, or (iv) can hybridize under stringent conditions to a polynucleotide selected from the group consisting of SEQ ID NO: 126, 132, 141, 156, 162, 171, 186, 192, or 201, SEQ ID NO: 274-276, SEQ ID NO: 280-282, SEQ ID NO: 301-318, and the complements thereof, wherein the interfering RNA molecule has insecticidal activity on a coleopteran plant pest.

2. The dsRNA molecule of claim 1, wherein said coleopteran plant pest is a Diabrotica insect.

3.-4. (canceled)

5. The dsRNA molecule of claim 1, wherein the interfering RNA molecule comprises SEQ ID NO: 126, 132, 141, 156, 162, 171, 186, 192, or 201, SEQ ID NO: 274-276, SEQ ID NO: 280-282, SEQ ID NO: 301-318, or the complement thereof.

6. The dsRNA molecule of claim 1, wherein the annealed strands are substantially complementary.

7. The dsRNA molecule of claim 1, wherein annealed strands are fully complementary.

8. The dsRNA molecule of claim 2, wherein the Diabrotica insect is selected from the group consisting of D. barberi, D. virgifera, D. undecimpunctata, D. balteata, D. undecimpunctata, D. significata, and D. speciosa.

9. (canceled)

10. A nucleic acid molecule encoding the dsRNA molecule of any of claim 1.

11.-12. (canceled)

13. A recombinant vector comprising a regulatory sequence operably linked to a nucleotide sequence that encodes the dsRNA molecule of claim 1.

14.-24. (canceled)

25. A transgenic plant, or part thereof, comprising the nucleic acid molecule of claim 10, wherein the transgenic plant has enhanced resistance to a Diabrotica insect as compared to a control plant.

26. The transgenic plant, or part thereof, of claim 25, wherein the transgenic plant comprises at least a second insecticidal agent for controlling Diabrotica insects.

27. The transgenic plant, or part thereof, of claim 26, wherein the second insecticidal agent is a Bacillus thuringiensis insecticidal protein, a Bacillus cereus insecticidal protein, a Xenorhabdus spp. insecticidal protein, a Photorhabdus spp. insecticidal protein, a Brevibacillus laterosporous insecticidal protein, a Lysinibacilius sphearicus insecticidal protein, a Chromobacterium spp. insecticidal protein a Yersinia entomophaga insecticidal protein, a Paenibacillus popiliae insecticidal protein, a Clostridium spp. insecticidal protein, a patatin, a protease, a protease inhibitor, a urease, an alpha-amylase inhibitor, a pore-forming protein, a lectin, an engineered antibody or antibody fragment, or a chitinase.

28.-30. (canceled)

31. The transgenic plant, or part thereof, of claim 25, wherein the transgenic plant, or part thereof, is a maize plant or part thereof.

32. Transgenic seed of the transgenic plant of claim 31.

33. (canceled)

34. A commodity product produced from the transgenic plant, or part thereof, of claim 25, wherein the commodity product comprises a detectable amount of said nucleic acid molecule.

35. A The commodity product of claim 34, selected from the group consisting of whole or processed seeds, beans, grains, kernels, hulls, meals, grits, flours, sugars, sugars, starches, protein concentrates, protein isolates, waxes, oils, extracts, juices, concentrates, liquids, syrups, feed, silage, fiber, paper or other food or product produced from plants.

36. A method of controlling a Diabrotica insect comprising contacting the Diabrotica insect with a nucleic acid molecule that is or is capable of producing the dsRNA molecule of claim 1 for inhibiting expression of a target gene in the Diabrotica insect thereby controlling the Diabrotica insect.

37.-38. (canceled)

39. The method of claim 36, wherein the Diabrotica insect is selected from the group consisting of D. barberi, D. virgifera, D. undecimpunctata, D. balteata, D. undecimpunctata, D. significata, and D. speciosa.

40. The method of claim 39, wherein contacting comprises:
a) planting a transgenic seed capable of producing a transgenic plant that expresses the nucleic acid molecule, wherein the Diabrotica insect feeds on the transgenic plant, or part thereof; or
b) applying a composition comprising the nucleic acid molecule to a seed or plant, or part thereof, wherein the Diabrotica insect feeds on the seed, the plant, or a part thereof.

41. The method of claim 40, wherein the transgenic seed and transgenic plant is a corn seed and a corn plant.

42. (canceled)

43. A method of controlling a Diabrotica insect comprising contacting the Diabrotica insect with a nucleic acid molecule that is or is capable of producing the dsRNA molecule of claim 1 for inhibiting expression of a target gene in the Diabrotica insect , and contacting the Diabrotica insect with at least a second insecticidal agent for controlling Diabrotica.

44. The method of claim 43, wherein said second insecticidal agent comprises a B. thuringiensis insecticidal protein, a Bacillus cereus insecticidal protein, a Xenorhabdus spp. insecticidal protein, a Photorhabdus spp. insecticidal protein, a Brevibacillus laterosporous insecticidal protein, a Lysinibacillus sphearicus insecticidal protein, a Chromobacterium spp. insecticidal protein, a Yersinia entomophaga insecticidal protein, a Paenibacillus popiliae insecticidal protein, a Clostridium spp. insecticidal protein, a patatin, a protease, a protease inhibitor, a urease, an alpha-amylase inhibitor, a pore-forming protein, a lectin, an engineered antibody or antibody fragment, or a chitinase.

45.-70. (canceled)
